(19) United States
(12) Patent Application Publication
(10) Pub. No.: US 2019/0210973 A1
(43) Pub. Date: Jul. 11, 2019

(54) COMPOUNDS AND METHODS FOR TREATMENT OF CYSTIC FIBROSIS

(71) Applicants: The Curators of the University of Missouri, Columbia, MO (US); Nanova, Inc., Columbia, MO (US)

(72) Inventors: Lianhai LI, Pierrefonds (CA); Tzyh-Chang HWANG, Columbia, MO (US); Xiaoqin Zou, Columbia, MO (US)

(21) Appl. No.: 16/240,679

(22) Filed: Jan. 4, 2019

Related U.S. Application Data
(60) Provisional application No. 62/613,962, filed on Jan. 5, 2018.

(51) Int. Cl. C07D 231/56; C07C 217/02; C07D 209/30; A61P 11/00; A61K 31/138; A61K 31/416; A61K 31/404; A61K 31/47; A61K 31/443

(52) U.S. Cl. CPC C07D 231/56; C07C 217/02; C07D 209/30; A61P 11/00; A61K 31/443; A61K 31/416; A61K 31/404; A61K 31/47; A61K 31/138

(57) ABSTRACT

Provided are compounds having Formula (1), compositions thereof, and methods of modulating CFTR activity. Also provided are methods of treating a condition associated with decreased CFTR activity comprising administering to a subject an effective amount of a compound of Formula (1), optionally with other therapeutic agent(s).

### CROSS REFERENCE TO RELATED APPLICATION

This application claims priority to U.S. Provisional Application No. 62/613,962 filed on Jan. 5, 2018, which is hereby incorporated by reference in its entirety.

### GOVERNMENT GRANT

The invention was made with U.S. government support under grant no. NIH-R01DK55835 awarded to Dr. Tzyh-Chang Hwang by the National Institutes of Health. The U.S. government has certain rights in the invention.

### FIELD

Described herein relates to a series of compounds identified for the improvement and/or stabilization of ion channel activity of cystic fibrosis transmembrane conductance regulator (CFTR) proteins. Also described herein relates to the treatment of cystic fibrosis (CF) by using these compounds, optionally in combination with other therapeutic agent(s).

### BACKGROUND

Cystic fibrosis (CF), the most common life-shortening hereditary disease in Caucasian populations, is caused by mutations of the cftr gene. The cftr gene encodes cystic fibrosis transmembrane conductance regulator (CFTR), which is a plasma membrane protein that functions as a chloride ion channel (Riordan et al., Science 1989). CFTR, as a channel, plays a major physiological role in promoting salt and water movement across many epithelial cells in human body. Defective function of the CFTR chloride channel results in tissue destruction as well as functional impairment in organs such as pancreas, lung, and gastrointestinal tract. With a median life expectancy of less than forty, most CF patients succumb to respiratory failure at the final stage of the illness (Rowe et al., NEJM, 2005).

Like all other ion channel proteins, the overall function of CFTR in a cell is determined by the following three factors: (1) the number of CFTR proteins in the plasma membrane, N; (2) the activity of individual CFTR proteins, Pₒ (the fraction of time the channel stays open); and (3) the magnitude of chloride currents carried by one single CFTR protein, i. In other words, the functional integrity of CFTR can be mathematically determined by the formula: N×P₀×i.

Depending on the biochemical mechanism of functional perturbations, CF-causing mutations are categorized into the following six different classes (Wang et al., Int. J. Biochem. Cell Biol., 2014):
1) Class I: impaired protein production, e.g., nonsense mutations such as G542X;
2) Class II: defective protein trafficking to the plasma membrane, e.g., the most common pathogenic mutation delF508 (i.e., ΔF508);
3) Class III: defective opening/closing (or gating) of the channel, e.g., G551D;
4) Class IV: Reduced single channel conductance, e.g., R117H;
5) Class V: reduced protein synthesis, e.g., A445E; and
6) Class VI: reduced protein stability, e.g., Q1412X.
   In effect, Classes I, II, V, and VI mutations decrease N; Class III mutations lower the Pₒ; and Class IV mutations reduce i. Of note, many disease-associated mutations actually cause multiple defects. For example, delF508, as the most common disease-associated mutation (present in ˜85% patients with CF worldwide), has been found to display multiple defects. While delF508 is originally categorized into Class II mutation with defective trafficking from the endoplasmic reticulum to the Golgi apparatus (Cheng et al., Cell, 1990), later studies show compelling evidence for gating defects (Class III, Dalemans et al., Nature, 1991; Hwang et al., Am. J. Physiol. 1997; Miki et al., J. Biol. Chem, 2010) and a shorter lifetime in the membrane (Class VI, Lukacs et al., J. Biol. Chem. 1993). Similarly, the R117H mutation (Class IV) also shows reduced Pₒ (Class III). These complexities certainly add extra hurdle for developing therapeutic agents targeting the CFTR protein. Nonetheless, because of the aforementioned mathematic formula of N×Pₒ×i, reagents that can improve any one or ideally a combination of these three factors are expected to achieve therapeutic effects.

Traditional therapies have been aimed at symptomatic treatment. In the past ten years, major breakthroughs in drug development for target therapy of CF have emerged. Although reagents overcoming stop-codon (Class I) mutations are still in the pre-clinical stage, compounds that help protein trafficking (correctors) or increase the activity of CFTR channels (potentiators) have been approved by the FDA. For example, the FDA-approved drugs include corrector VX-809, aka Lumacaftor (3-(6-(1-(2,2-difluorobenzo[d][1,3]dioxol-5-yl) cyclopropanecarboxamido)-3-methylPyridin-2-yl)benzoic acid characterized), from Vertex Pharmaceutical Inc. (see Van Goor et al., PNAS 2011; see, e.g., U.S. Pat. Nos. 7,973,038 and 8,507,534) and potentiator VX-770 (N-[2,4-bis(1,1-dimethylethyl)-5-hydroxyphenyl]-1,4-dihydro-4-oxoquinoline-3-carboxamide), aka Ivacaftor, from Vertex Pharmaceutical Inc. (see Van Goor et al., PNAS 2009; see, e.g., U.S. Pat. Nos. 7,495,103 and 8,754,224). Lumacaftor and Ivacaftor effectively enhance N and Pₒ, respectively. In spite of these breakthroughs, many challenges remain to achieve a functional restoration of CFTR activity to the healthy level, defined as >25% of N×Pₒ×i in normal healthy human beings (Davis, Ped. Rev., 2001). For example, Ivacaftor, which has been shown to improve the activity of the G551D mutant, can only increases the level to ˜10% of normal (Van Goor et al., PNAS 2009; Jih et al., PNAS 2013; Lin et al., Mole. Pharma. 2016). A combination regimen of Ivacaftor plus Lumacaftor, which has been approved by the FDA for treating patients carrying the delF508 mutation, only marginally improves the overall function of delF508 mutants, because Ivacaftor, for unknown reasons, diminishes the effect of Lumacaftor (Veit et al., Sci. Transl. Med. 2015; Cholon et al., Sci. Transl. Med. 2015). Therefore, there is still an urgent need in the field to find more effective compounds targeting CFTR and use such compounds as therapeutic agents, acting alone or in combination with other therapeutic agents (e.g., Ivacaftor and Lumacaftor), to treat CF.

### SUMMARY

A group of compounds were newly identified for their capability of increasing the activities (Pₒ) of G551D and delF508 CFTR through a mechanism distinct from that of Ivacaftor (e.g., Jih et al., PNAS 2013). Exemplary compounds not only work alone, but also synergistically potentiate CFTR functions when applied together with Ivacaftor. A nearly complete functional restoration of G551D channel activity may be accomplished by a combination of Ivacaftor with the exemplary compounds. Furthermore, Class VI defect manifested in delF508 CFTR may be rectified by the exemplary compounds, resulting in an increase of N, in addition to their effects on Pₒ. Unlike Ivacaftor, exemplary compounds exhibit synergistic interactions with Lumacaftor. Thus, the group of compounds described herein may represent a novel category of drugs targeting the CFTR protein with a dual function: CFTR potentiator/stabilizer.

Described herein are compounds of Formula (1), pharmaceutically acceptable salts, pharmaceutically acceptable hydrates, pharmaceutically acceptable solvates, pharmaceutically acceptable clathrates, or pharmaceutically acceptable polymorphs thereof:

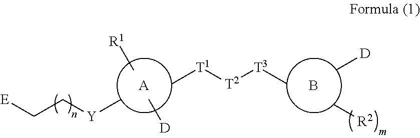

wherein
Ring A may be selected from phenyl, six-membered aromatic ring with 1, 2, or 3 nitrogen ring atoms, or a five-membered ring, aromatic or non-aromatic, with 1, 2, or 3 heteroatoms independently selected from O, S, or N;
Ring B may be a mono or bicyclic ring system, aromatic or non-aromatic, with 5, 6, 7, 8, 9, or 10 ring atoms with 1 to 4 heteroatoms selected from O, S, or N;
each T¹, T², and T³ may be independently absent, or independently selected from C(R^{CT})₂, C(O), S(O)₀₋₂ or NR^{NT}, wherein R^{CT} with R^{CT}, or R^{CT} with R^{NT}, may join together to form a three-, four-, or five-membered aliphatic ring; or R^{CT} and R^{NT} may be each independently selected from H, CH₂OH, C₁₋₄alkyl, C₂₋₆alkenyl, CF₃, or (R^{CT})₂ is ═CHR³, where R³ may be independently selected from H, CH₂OH, C₁₋₄alkyl, or C₂₋₆alkenyl;
Y may be selected from a bond, O, S, NR³, or —C(O)NR³;
D may be each independently selected from F, CF₃, CHₐF₍₃₋ₐ₎, Cl, Br, CN, NO₂, OR⁴, OCF₃, or OC(O)R⁵, where a may be 1 or 2; R⁴ may be H, C₁₋₄alkyl, or R⁵; and R⁵ may be (CH₂)ⱼR⁶ where j may be an integer from 3 to 8, and R⁶ may be H or E, and optionally one or more adjacent CH₂ units may be replaced with O, S, or NR³, and/or one or more hydrogens of the CH₂ units may be substituted with F, Cl, Br, CN, OR⁴, OH, or NHR³;
E may be N(R³)₂;
R¹ and R² may be independently selected from C₁₋₆ alkyl, five- to nine-membered heteroaryl, phenyl, napthyl, —OR⁴, —N(R³)₂, —SR⁴, —SO₂R⁴, —SO₂N(R³)₂, —NR³SO₂R⁴, —NR³C(O)OR⁴, —NR³C(O)R⁴, —C(O)OR⁴, —C(O)N(R³)₂, —OC(═O)R⁴, —C(═O)R⁴, and m may be an integer from 1 to 4; and
n may be an integer from 1-7, and optionally one or more CH₂ units may be replaced with O, S, or NR³, and/or one or more hydrogens of the CH₂ units may be substituted with F, Cl, Br, CN, OR⁴, OH, or NHR³.

In one aspect, each of T¹, T², and T³ may be absent and Ring A may be directly connected to Ring B, and Ring A may be an aromatic six-membered ring structure and Ring B may be a bicyclic ring system having 9 ring atoms with 1 to 2 N ring atoms.

In another aspect, the compound of Formula (1) may be selected from the group consisting of:

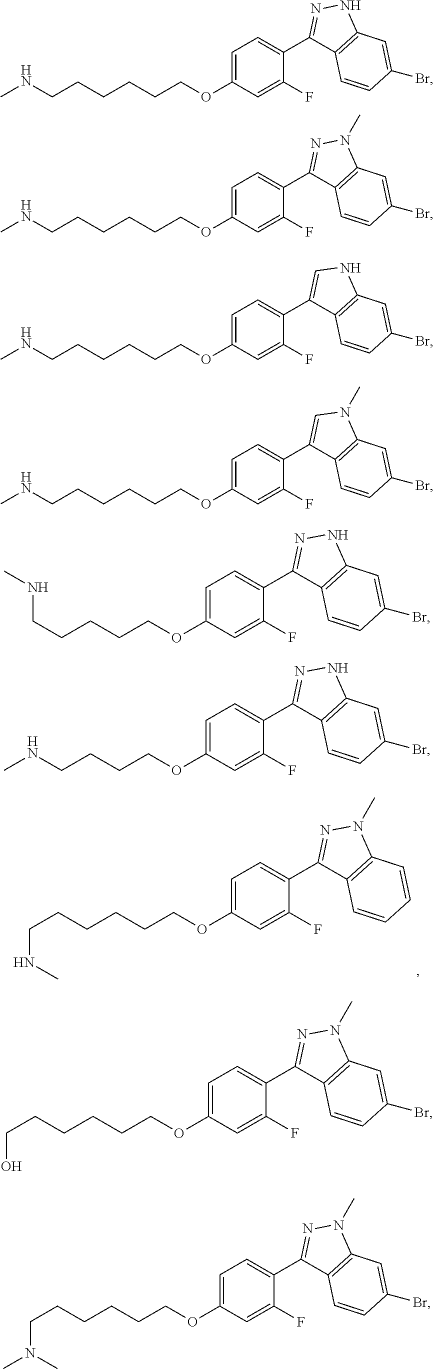

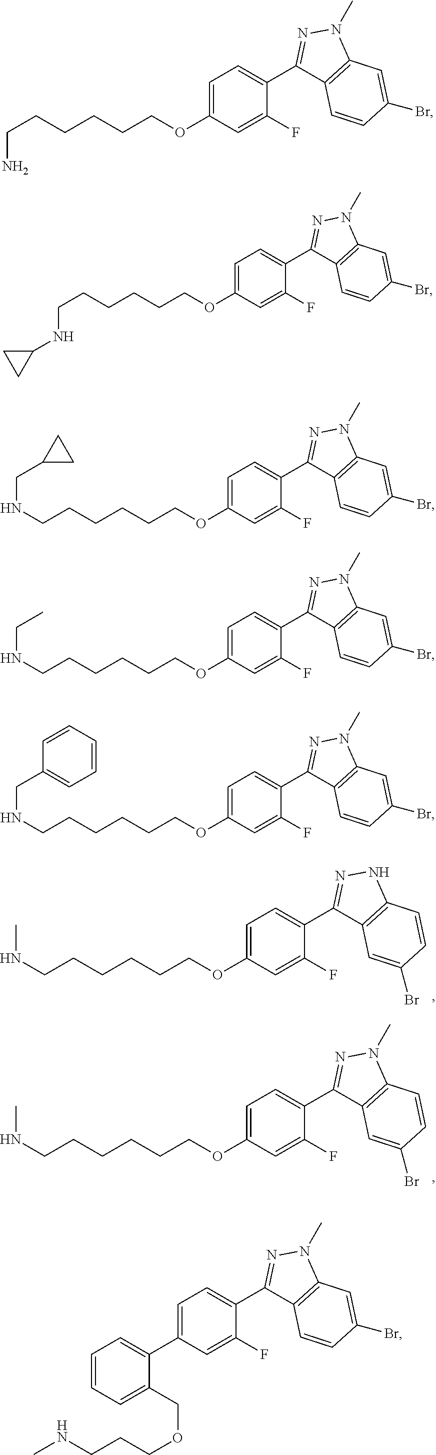

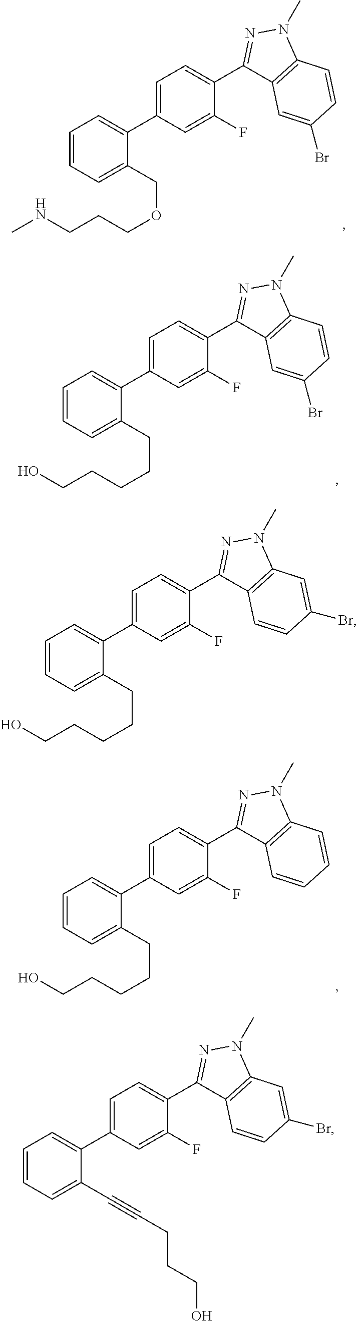 [SMILES: CNCCCOCc1ccccc1-c1ccc(-c2nn(C)c3ccc(Br)cc23)c(F)c1 ; Cn1nc(-c2ccc(-c3ccccc3CCCCCO)cc2F)c2cc(Br)ccc21 ; Cn1nc(-c2ccc(-c3ccccc3CCCCCO)cc2F)c2ccc(Br)cc21 ; Cn1nc(-c2ccc(-c3ccccc3CCCCCO)cc2F)c2ccccc21 ; Cn1nc(-c2ccc(-c3ccccc3C#CCCCO)cc2F)c2ccc(Br)cc21]

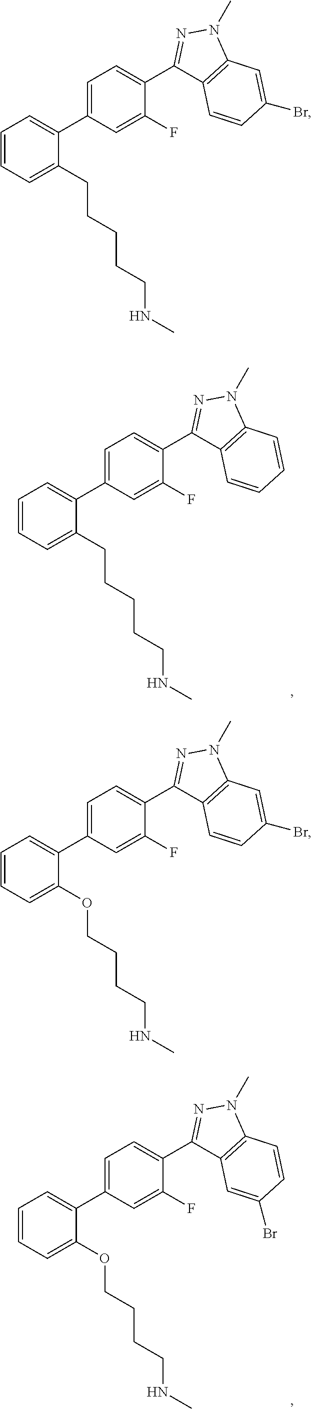

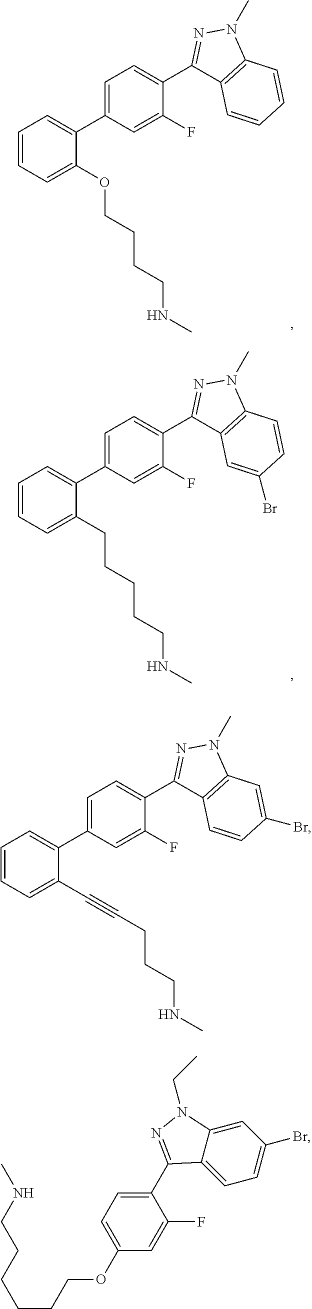

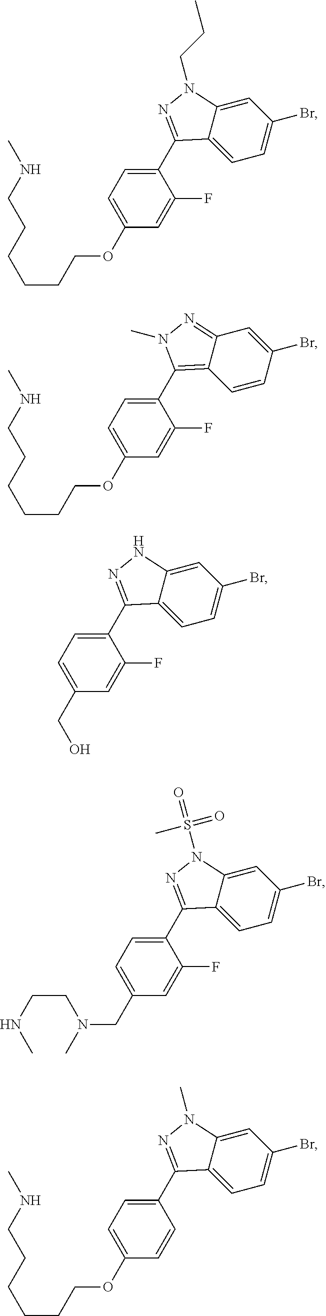 [SMILES: CCCn1nc(-c2ccc(OCCCCCCNC)cc2F)c2ccc(Br)cc21 ; CNCCCCCCOc1ccc(-c2c3ccc(Br)cc3nn2C)c(F)c1 ; OCc1ccc(-c2n[nH]c3cc(Br)ccc23)c(F)c1 ; CNCCN(C)Cc1ccc(-c2nn(S(C)(=O)=O)c3cc(Br)ccc23)c(F)c1 ; CNCCCCCCOc1ccc(-c2nn(C)c3cc(Br)ccc23)cc1]

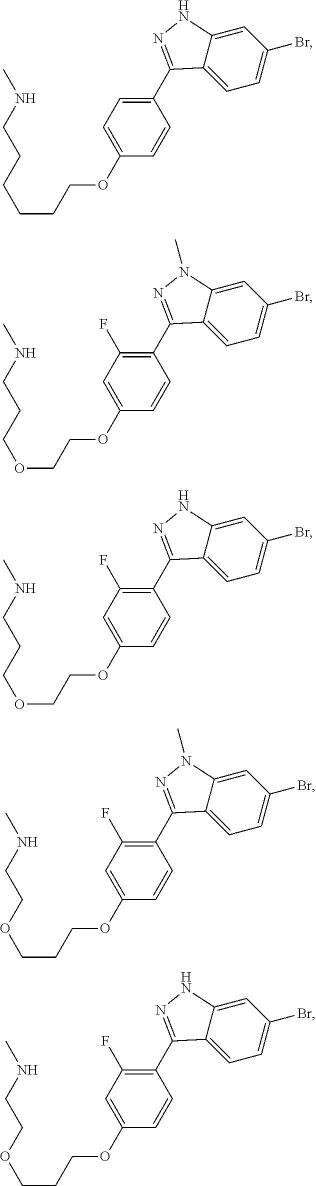 [SMILES: CNCCCCCCOc1ccc(-c2n[nH]c3cc(Br)ccc23)cc1 ; CNCCCOCCOc1ccc(-c2nn(C)c3cc(Br)ccc23)c(F)c1 ; CNCCCOCCOc1ccc(-c2n[nH]c3cc(Br)ccc23)c(F)c1 ; CNCCOCCCOc1ccc(-c2nn(C)c3cc(Br)ccc23)c(F)c1 ; CNCCOCCCOc1ccc(-c2n[nH]c3cc(Br)ccc23)c(F)c1]

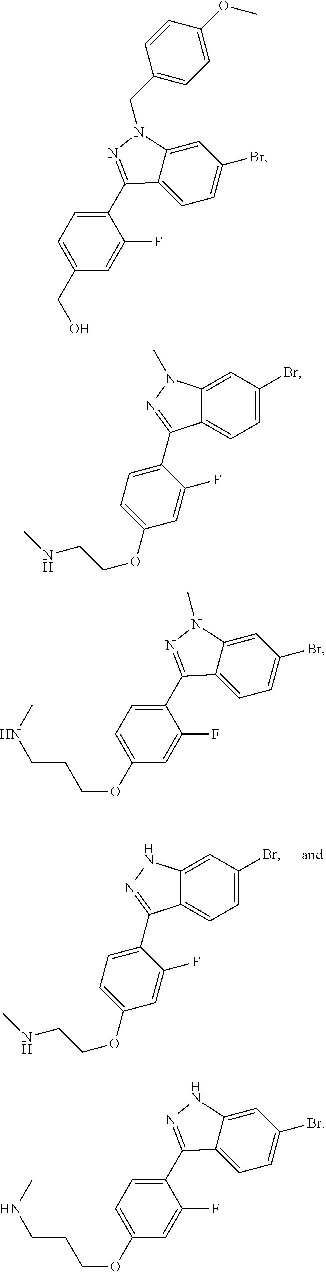

Also described herein are compositions for enhancing cystic fibrosis transmembrane conductance regulator (CFTR) activity. The composition may comprise an effective amount of the compound of Formula (1), the pharmaceutically acceptable salt, the pharmaceutically acceptable hydrate, the pharmaceutically acceptable solvate, the pharmaceutically acceptable clathrate, or the pharmaceutically acceptable polymorph thereof.

Further described herein are methods of treating a CFTR-mediated disease by enhancing cystic fibrosis transmembrane conductance regulator (CFTR) activity or expression in the cells of a subject in need thereof. The method may comprise administering to the subject the composition comprising an effective amount of the compound of Formula (1), the pharmaceutically acceptable salt, the pharmaceutically acceptable hydrate, the pharmaceutically acceptable solvate, the pharmaceutically acceptable clathrate, or the pharmaceutically acceptable polymorph thereof. For the compound of Formula (1), each of T¹, T², and T³ may be absent and Ring A may be directly connected to Ring B, Ring A may be an aromatic six-membered ring structure, and Ring B may be a bicyclic ring system having 9 ring atoms with 1 to 2 N ring atoms.

In one aspect, the subject may comprise a mutant CFTR, and the activity of the mutant CFTR may be enhanced as a result of administering the composition. In another aspect, the mutant CFTR may comprise at least one mutation selected from the group consisting of a Class I mutation, a Class II mutation, a Class III mutation, a Class IV mutation, a Class V mutation, a Class VI mutation, and combinations thereof. In yet another aspect, the mutant CFTR may comprise at least one Class II mutation or one Class III mutation.

In one aspect, the mutant CFTR may be a delF508 CFTR, and the delF508 CFTR activity may be enhanced as a result of administering the composition. In another aspect, the mutant CFTR may be a G551D-CFTR, and the G551D-CFTR activity may be enhanced as a result of administering the composition.

In one aspect, administering the composition may be through a route selected from oral, intravenous, intraperitoneal, intramuscular, transdermal, subcutaneous, topical, sublingual, intravascular, intramammary, rectal means, and combinations thereof.

In one aspect, the compound, the pharmaceutically acceptable salt, the pharmaceutically acceptable hydrate, the pharmaceutically acceptable solvate, the pharmaceutically acceptable clathrate, or the pharmaceutically acceptable polymorph thereof, may be administered as the sole active agent. Optionally, the method may further comprise administering to the subject one or more therapeutic agents. The one or more therapeutic agents may be capable of modulating CFTR activity. For example, the one or more therapeutic agents may be selected from the group consisting of:

a) an effective amount of Ivacaftor;

b) an effective amount of Lumacaftor; and

c) an effective amount of a combination of Ivacaftor and Lumacaftor.

In another aspect, the composition and the one or more therapeutic agents may be administered at substantially the same time.

In one aspect, the subject may be suffering from a disease associated with decreased CFTR activity. In another aspect, the disease may be cystic fibrosis. In yet another aspect, the subject may be a human patient.

### BRIEF DESCRIPTION OF THE DRAWINGS

The accompanying drawings are incorporated into the specification and provide non-limiting illustration of various embodiments.

FIGS. 1A and 1B depict an illustration of Cystic Fibrosis Transmembrane Conductance Regulator (CFTR) and a phosphorylation-activated, ATP-gated chloride channel (adopted from Zhang and Chen, Cell, 2016). FIG. 1A illustrates the topology of CFTR showing two transmembrane domains (TMD1 and TMD2), the regulatory domain (R), and two nucleotide-binding domains (NBD1 and NBD2). FIG. 1B illustrates the Cryo-EM structure of CFTR at 3.7 Å resolution.

FIG. 2 depicts the biochemical/biophysical mechanisms underpinning mutation-induced defects in CFTR (adopted from Wang et al., Int. J. Biochem. Cell Biol., 2014). The cartoon depicts a simple model including synthesis, processing and function of CFTR in epithelial cells. Different pathogenic mutations result in different biochemical/biophysical defects as marked by X. ER represents Endoplasmic reticulum, while N represents nucleus. It is to be noted that the addition of complex sugars to the extracellular domain of CFTR occurs in the Golgi apparatus. It is this mature, high molecular-weight form of CFTR that eventually traffics to the plasma membrane.

FIGS. 3A and 3B depict patch-clamp real-time recording of wild-type (WT) CFTR. FIG. 3A illustrates a simplified description of the patch-clamp technique for recording of CFTR in an inside-out mode. The glass microelectrode is applied gently onto the cell. Once the glass and the cell membrane form a gigaOhm (10⁹ ohm) seal, the patch of membrane with CFTR is excised from the cell to form an inside-out configuration where reagents such as protein kinas A (PKA) and ATP can be applied to directly modulate CFTR from the cytoplasmic side of the membrane. FIG. 3B illustrates a real-time recording of macroscopic WT-CFTR currents showing phosphorylation-dependent action and ATP-dependent gating. When a membrane patch contains numerous WT-CFTR channels, macroscopic currents can be activated gradually by PKA and ATP (i.e., phosphorylation-dependent activation). Once the currents reach a plateau, phosphorylated WT-CFTR channels require the presence of ATP to maintain their activity. It is to be noted that a drop of the currents occurs upon brief removal of ATP, and recovery of the currents occurs upon re-addition of ATP alone without PKA to the superfusion solution. This ATP-induced current with phosphorylated CFTR is defined as ATP-dependent gating. Dashed line: baseline (or zero current line).

FIG. 4 depicts the enhancement of G551D-CFTR currents by LZH-00014. The enhancement was based upon patch-clamp real-time recording as described in Example 48.

FIG. 5 depicts a summary of the fold increases of G551D-CFTR currents by selected compounds. The effect of various compounds was based upon patch-clamp real-time recording as described in Example 48. The magnitude of potentiation for G551D-CFTR was calculated as fold increase (ratio of currents with and without the compound, I_{LZH}/I_{ATP}). The dashed line marks the maximal effect of Ivacaftor (VX-770) under the same experimental conditions. Numerical values in the parentheses indicate the number of independent experiments.

FIGS. 6A and 6B depict the synergistic effects of LZH-00003 and Ivacaftor (VX-770) on G551D-CFTR. The effect of various combinations was based upon patch-clamp real-time recording as described in Example 48. FIG. 6A illustrates a real-time current trace of G551D-CFTR in response to LZH-00003 alone and to LZH-00003 in the presence of Ivacaftor (VX-770). FIG. 6B illustrates a summary of the synergistic effects between a series of the LZH compounds and VX-770. The dashed line marks the maximal effect of Ivacaftor (VX-770) under the same experimental conditions.

FIGS. 7A, 7B, 7C, and 7D depict improved potency of various LZH compounds. The effect of various combinations was based upon patch-clamp real-time recording as described in Example 48. FIG. 7A illustrates a real-time recording of the G551D-CFTR currents in response to LZH-00014 alone and to LZH-00014 in the presence of Ivacaftor (VX-770). FIG. 7B illustrates a summary of the synergistic effects between exemplary LZH compounds and VX-770. The dashed line marks the maximal effect of Ivacaftor (VX-770) alone under the same experimental conditions. FIG. 7C illustrate a real-time recording of the G551D-CFTR currents in response to LZH-00015 alone and to LZH-00015 in the presence of Ivacaftor (VX-770). FIG. 7D illustrates a summary of the synergistic effects between LZH-00015 and VX-770 at sub-micromolar concentrations. The dashed line marks the maximal effect of Ivacaftor (VX-770) under the same experimental conditions.

FIGS. 8A and 8B depict enhancement of delF508-CFTR by LZH-00015. The effect of various compounds was based upon patch-clamp real-time recording as described in Example 48. FIG. 8A illustrates a real-time recording of delF508-CFTR currents in response to LZH-00015 or VX-770. FIG. 8B illustrates a summary of the fold increases of delF508-CFTR by the LZH compounds. The dashed line marks the fold increase by Ivacaftor (VX-770) alone.

FIGS. 9A and 9B depict the synergistic effects of the LZH compounds and Ivacaftor (VX-770) on delF508-CFTR. The effect of various combinations was based upon patch-clamp real-time recording as described in Example 48. FIG. 9A illustrates a real-time recording of the delF508-CFTR currents in response to Ivacaftor alone and to LZH-00015 in the presence of Ivacaftor. FIG. 9B illustrates a summary of the combined effects of Ivacaftor plus the LZH compounds. The dashed line marks the fold increase by Ivacaftor (VX-770) alone.

FIG. 10 depicts LZH-00015's stabilization effect upon delF508-CFTR in the membrane. The effect was based upon patch-clamp real-time recording as described in Example 48.

FIG. 11 depicts a Western blot showing the synergistic effects of the LZH compounds and Lumacaftor (VX-809) on membrane expression of delF508-CFTR.

FIGS. 12A and 12B depicts the enhancement of G551D-CFTR currents by selected LZH compounds as described in Example 49. FIG. 12A illustrates a real-time recording of the G551D-CFTR currents in response to LZH-00014, showing a greater potentiation in contrast to the minimal current activation by PKA and ATP. FIG. 12B illustrates a real-time recording of the G551D-CFTR currents in response to LZH-00015 alone and to LZH-00015 in the presence of Ivacaftor (200 nM VX-770), showing a significant synergism even at 1 μM concentration of LZH-00015.

FIGS. 13A and 13B depict the enhancement of delF508-CFTR by LZH-00015 as described in Example 49. FIG. 13A illustrates a real-time recording of delF508-CFTR currents in response to LZH-00015 or Ivacaftor (VX-770). FIG. 13B illustrates a real-time recording of the delF508-CFTR currents in response to Ivacaftor alone and to LZH-00015 in the presence of Ivacaftor, showing the synergistic effects of LZH-00015 and Ivacaftor (200 nM VX-770) on delF508-CF TR.

FIG. 14 depicts a representative Western blot image showing the effects of selected LZH compounds and Lumacaftor (VX-809) on membrane expression of delF508-CFTR as described in Example 49.

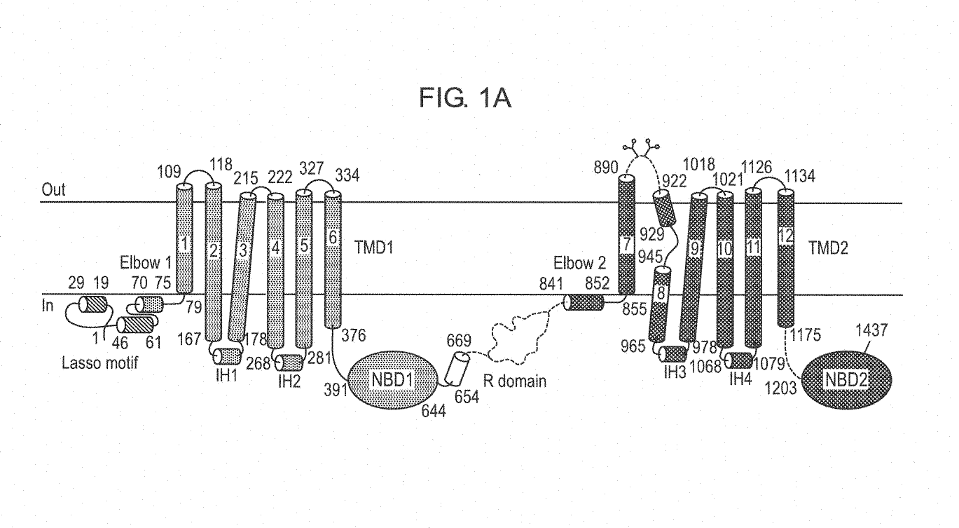

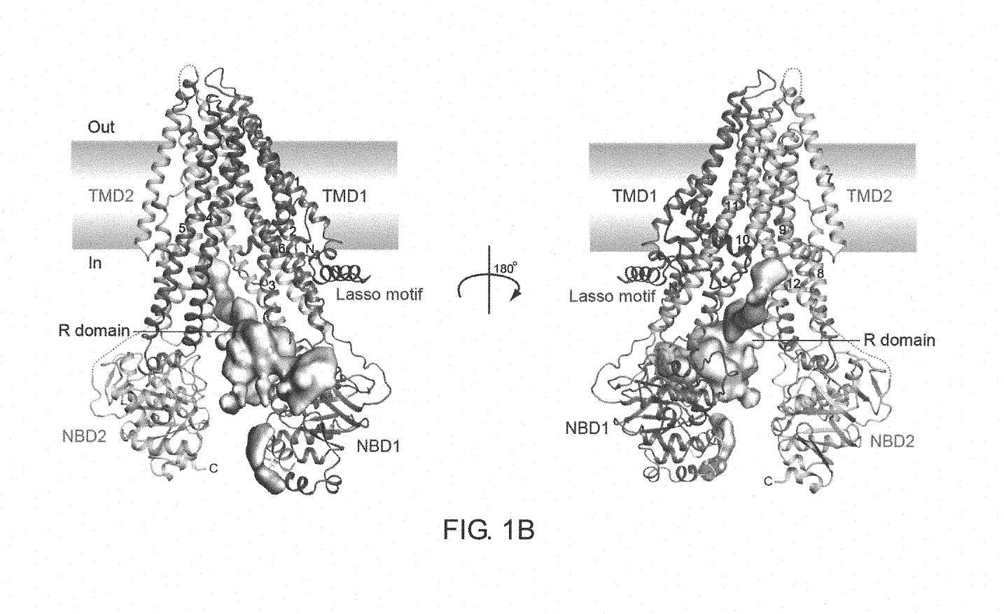

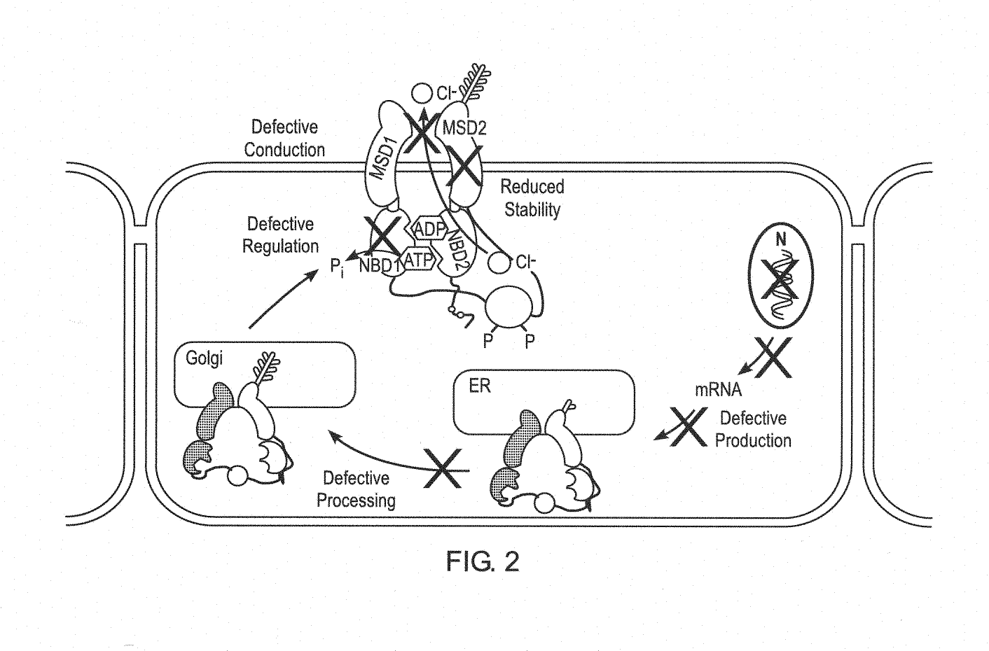

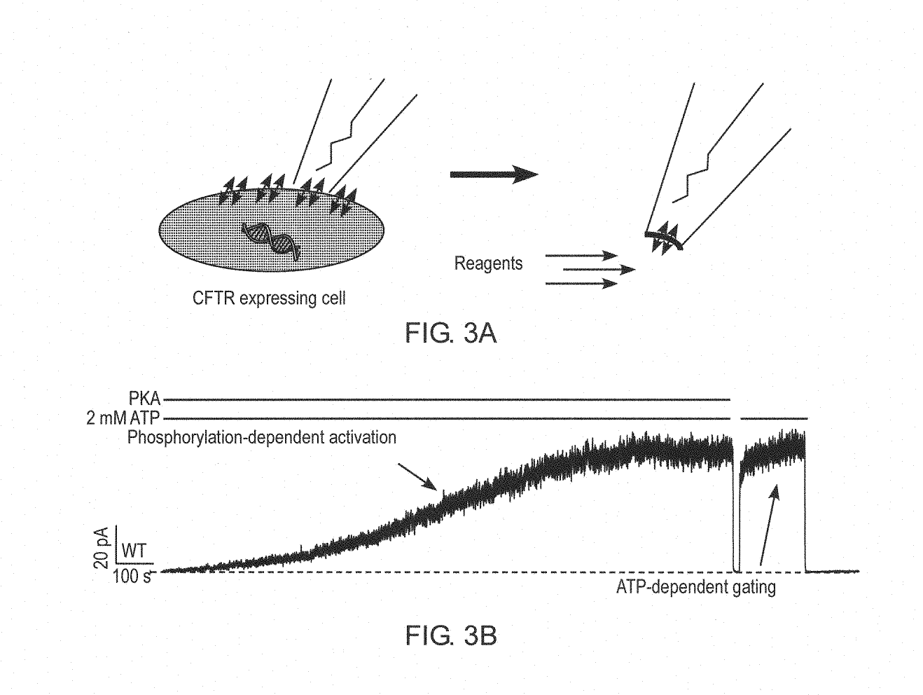

### DETAILED DESCRIPTION

### 1. Abbreviations and Definitions

### 1.1. Abbreviations

CF cystic fibrosis
CFTR cystic fibrosis transmembrane conductance regulator
FDA Food and Drug Administration
CBAVD congenital bilateral absence of vas deferens
COPD chronic obstructive pulmonary disease
EDTA ethylenediaminetetraacetic acid
WT wild-type
PKA protein kinas A
ATP Adenosine triphosphate
TMD transmembrane domains
NBD nucleotide-binding domains
DMF dimethylformamide
DMSO dimethyl sulfoxide
ER endoplasmic reticulum
LCMS liquid chromatographymass spectrometry
NMR nuclear magnetic resonance
SDS sodium dodecyl sulfate
TBME tert-butyl methyl ether
TBST Tris-buffered saline with Tween 20
THF tetrahydrofuran
TFA trifluoroacetic acid

### 1.2. Definitions

Unless defined otherwise, all technical and scientific terms used herein have the same meaning as commonly understood by one of ordinary skill in the art to which the disclosure pertains. The following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

As used herein, the term “hydrate” means a compound provided herein or a salt thereof, further including a stoichiometric or non-stoichiometric amount of water bound by non-covalent intermolecular forces. The hydrates can be crystalline or non-crystalline.

As used herein, the term “solvate” means a solvate formed from the association of one or more solvent molecules to compound provided herein. The term “solvate” includes hydrates (e.g., monohydrate, dihydrate, trihydrate, tetrahydrate, and the like). The solvates can be crystalline or non-crystalline.

As used herein, the term “clathrate” means a compound described herein or a salt thereof in the form of a crystal lattice that contains spaces (e.g., channels) that have a guest molecule (e.g., a solvent or water) trapped within.

As used herein, “polymorph(s)” refer to crystalline form(s) having the same chemical structure/composition but different spatial arrangements of the molecules and/or ions forming the crystals.

The term “alkyl” as used herein refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tertiary butyl, pentyl, isopentyl, hexyl, cyclopropylmethyl, etc.

Alkyl groups can be unsubstituted, or they can be substituted to the extent that such substitution makes sense chemically. Typical substituents include, but are not limited to, halo, ═O, ═N—CN, ═N—OR^{OS}, ═NR^{NS0}, —OR^{OS}, —NR^{NS1}R^{NS2}, —SR^{SS1}, —SO₂R^{SS2}, —SO₂NR^{NS1}R^{NS2}), —NR^{NS1}SO₂R^{SS2}, —NR^{NS1}C(═O)NR^{NS1}R^{NS2}, —NR^{NS1}C(═O)OR^{OS}, —NR^{NS1}C(═O)R^{CS}, —CN, —C(═O)OR^{OS}, —C(═O)NR^{NS1}R^{NS2}, —OC(═O)R^{CS}, —C(═O)R^{CS}, and —NO₂, wherein: each R^{SS1} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; each R^{SS2} is selected from (un)substituted C₁₋₆ alkyl, (un)substituted C₂₋₆ alkenyl, (un)substituted C₂₋₆ alkynyl, (un)substituted C₃₋₄ cycloalkyl (un)substituted 5-10 membered heteroaryl, or (un)substituted 6-10 membered aryl; Each R^{OS} is selected from hydrogen, (un)substituted C₁₋₆ alkyl, (un)substituted C₂₋₆ alkenyl, (un)substituted C₂₋₆alkynyl, (un)substituted C₃₋₈ cycloalkyl, (un)substituted 5-10 membered heteroaryl, or (un)substituted 6-10 membered aryl; each R^{C} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; Each R^{NS1} and R^{NS2} are independently selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl. When chemically making sense, one or two carbon of the (un)substituted C₁₋₆ alkyl, (un)substituted C₂₋₆ alkenyl, (un)substituted C₂₋₆ alkynyl, or (un)substituted C₃₋₈ cycloalkyl may be replaced by —O—, —N(R^{NS0})—, —S(═O)₀₋₂—, —P(R^{PS})(═O)—; each R^{PS} is selected from (un)substituted C₁₋₆ alkyl, (un)substituted C₂₋₆ alkenyl, (un)substituted C₂₋₆ alkynyl, (un)substituted C₃₋₈ cycloalkyl; Each R^{NS0} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, —CN, —OR^{OS}, —O(C(═O)R^{CS}), —C(═O)OR^{OS}), —C(═S)OR^{OS})—O(C(═S)R^{C})—N(R^{NS1})(R^{NS2}), —N(R^{NS1})(S(═O)₁₋₂R^{SS2}), —N(R^{NS1})(S(═O)ₜ₋₂NR^{NS1}R^{NS2}), —N(R^{NS1})(C(═O)R^{CS}), —N(R^{NS1})(C(═O)NR^{NS1}R^{NS2}), —N(R^{NS1})(C(═S)R^{CS}), —N(R^{NS1})(C(═S)NR^{NS1}R^{NS2}), —S(═O)₁₋₂R^{SS2}, —S(═O)₁₋₂NR^{NS1}R^{NS2}, or —C(═O)NR^{NS1}R^{NS2}, (un)substituted 5-10 membered heteroaryl, or (un)substituted 6-10 membered aryl; when exist as a pair, R^{NS1} and R^{NS2}, R^{NS1} and R^{SS2}, or R^{NS1} and R^{CS} may join together to form a 3-8 membered ring system.

The term “alkenyl” as used herein refers to an alkyl as defined above having at least two carbon atoms and at least one carbon-carbon double bond. Thus, particularly contemplated alkenyl groups include straight, branched, or cyclic alkenyl groups having two to ten carbon atoms (e.g., ethenyl, propenyl, butenyl, pentenyl, etc.) or 5-10 atoms for cyclic alkenyl groups. Alkenyl groups are optionally substituted by groups suitable for alkyl groups as set forth herein.

Similarly, the term “alkynyl” as used herein refers to an alkyl or alkenyl as defined above and having at least two (preferably three) carbon atoms and at least one carbon-carbon triple bond. Contemplated alkynyls include straight, branched, or cyclic alkynes having two to ten total carbon atoms (e.g., ethynyl, propynyl, butynyl, cyclopropylethynyl, etc.). Alkynyl groups are optionally substituted by groups suitable for alkyl groups as set forth herein.

The term “cycloalkyl” as used herein refers to a cyclic alkane (i.e., in which a chain of carbon atoms of a hydrocarbon forms a ring), typically including three to eight carbon atoms. Thus, exemplary cycloalkanes include cyclopropyl, cyclobutyl, cyclopentyl, cyclohexyl, cycloheptyl, and cyclooctyl. Cycloalkyls also may include one or two double bonds, which form the “cycloalkenyl” groups. Cycloalkyl groups are optionally substituted by groups suitable for alkyl groups as set forth herein.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group, and are thus termed “fused aryl” or “fused aromatic.”

Aromatic groups containing one or more heteroatoms (typically N, O or S) as ring members can be referred to as heteroaryl or heteroaromatic groups. Typical heteroaromatic groups include monocyclic C₅₋₆ aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C₈₋₁₀ bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle,” “cycloheteroalkyl,” and “heterocyclic moieties” are used interchangeably herein and refer to any compound in which a plurality of atoms form a ring via a plurality of covalent bonds, wherein the ring includes at least one atom other than a carbon atom as a ring member. Contemplated heterocyclic rings may include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically these rings contain 0-1 oxygen or sulfur atoms, at least one and typically 2-3 carbon atoms, and up to four nitrogen atoms as ring members. Also contemplated heterocycles may be fused (i.e., covalently bound with two atoms on the first heterocyclic ring) to one or two carbocyclic rings or heterocycles, and are thus termed “fused heterocycle” or “fused heterocyclic ring” or “fused heterocyclic moieties” as used herein. Where the ring is aromatic, these can be referred to herein as “heteroaryl” or “heteroaromatic” groups.

Heterocyclic groups that are not aromatic can be substituted with groups suitable for alkyl group substituents, as set forth above.

Aryl and heteroaryl groups can be substituted where permitted. Exemplary substituents include, but are not limited to, halo, —OR^{OS}, —NR^{NS1}R^{NS2}, —SR^{SS1}, —SO₂R^{SS2}, —SO₂NR^{NS1}R^{NS}, —NR^{NS1}O₂R^{SS2}, —NR^{NS1}C(═O)NR^{NS1}R^{NS2}, —NR^{NS1}C(═O)OR^{OS}, —NR^{NS1}C(═O)R^{CS}, —CN, —C(═O)OR^{OS}, —C(═O)NR^{NS1}R^{NS2}, —OC(═O)R^{CS}, —C(═O)R^{CS}, and —NO₂, wherein: each R^{SS1} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; each R^{S2} is selected from (un)substituted C₁₋₆ alkyl, (un)substituted C₂₋₆ alkenyl, (un)substituted C₂₋₆ alkynyl, (un)substituted C₃₋₈ cycloalkyl (un)substituted 5-10 membered heteroaryl, or (un)substituted 6-10 membered aryl; each R^{OS} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; each R^{CS} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; each R^{NS1} and R^{NS2} are independently selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, (un)substituted 5-10 membered heteroaryl, or (un)substituted 6-10 membered aryl; when chemically making sense, one or two carbon of the (un)substituted C₁₋₆ alkyl, (un)substituted C₂₄ alkenyl, (un)substituted C₂₋₆ alkynyl, or (un)substituted C₃₋₈ cycloalkyl may be replaced by —O—, —N(R^{NS0})—, —S(═O)₀₋₂—, —P(R^{PS})(═O)—; each R^{PS} is selected from (un)substituted C₁₋₆ alkyl, (un)substituted C₂₋₆ alkenyl, (un)substituted C₂₋₆ alkynyl, (un)substituted C₃₋₈ cycloalkyl; Each R^{NS0} is selected from hydrogen, (un)substituted C₁₋₆ alkyl, (un)substituted C₂₋₆ alkenyl, (un)substituted C₂₋₆ alkynyl, (un)substituted C₃₋₈ cycloalkyl, —CN, —OR^{OS}, —O(C(═O)R^{CS}), —C(═O)OR^{OS}), —C(═S)OR^{OS})—O(C(═S)R^{CS})—N(R^{NS1})(R^{NS2}), —N(R^{NS1})(S(═O)₁₋₂R^{SS2}), —N(R^{NS1})(S(═O)₁₋₂NR^{NS1}R^{NS2}), —N(R^{NS1})(C(═O)R^{CS}), —N(R^{NS1})(C(═O)NR^{NS1}R^{NS2}), —N(R^{NS1})(C(═S)R^{CS}), —N(R^{NS1})C(═S)NR^{NS1}R^{NS2}), —S(═O)₁₋₂R^{SS2}, —S(═O)₁₋₂NR^{NS1}R^{NS2}, or —C(═O)NR^{NS1}R^{NS2}, (un)substituted 5-10 membered heteroaryl, or (un)substituted 6-10 membered aryl; when exist as a pair, R^{NS1} and R^{NS2}, R^{NS1} and R^{SS2}, or R^{NS1} and R^{CS} may join together to form a 3-8 membered ring system.

As also used herein, the terms “imidazopyridine” or “imidazopyrimidine” or “thiazopyridine” or “thiazopyrimidine” herein refer to any compound in which the two designated heterocyclic rings are fused by any two adjacent atoms on the two heterocyclic rings.

The term “alkoxy” as used herein refers to a hydrocarbon group connected through an oxygen atom, e.g., —O—R^{OS}, wherein the hydrocarbon portion R^{O} may have any number of carbon atoms, typically 1-10 carbon atoms, may further include a double or triple bond and may include one or two oxygen, sulfur or nitrogen atoms in the alkyl chains, and can be substituted with aryl, heteroaryl, cycloalkyl, and/or heterocyclyl groups. For example, suitable alkoxy groups include methoxy, ethoxy, propyloxy, isopropoxy, methoxyethoxy, benzyloxy, allyloxy, and the like. Similarly, the term “alkylthio” refers to alkylsulfides of the general formula —S—R^{SS1}, wherein the hydrocarbon portion R^{SS1} is as described for alkoxy groups. For example, contemplated alkylthio groups include methylthio, ethylthio, isopropylthio, methoxyethylthio, benzylthio, allylthio, and the like.

The term “amino” as used herein refers to the group —NH₂. The term “alkylamino” refers to amino groups where one or both hydrogen atoms are replaced by a hydrocarbon group to form N(R^{NS1})(R^{NS2}) as described above, wherein the amino nitrogen “N” can be substituted by one R^{NS} group (referred as R^{NS1}, as described and defined as above) or two R^{NS} groups (referred as R^{NS1} and R^{NS2}, as described and defined as above). Exemplary alkylamino groups include methylamino, dimethylamino, ethylamino, diethylamino, etc. Also, the term “substituted amino” refers to amino groups where one or both hydrogen atoms are replaced by a hydrocarbon group R^{NS} as described above, wherein the amino nitrogen “N” can be substituted by one or two R^{NS} groups as described above.

The term “acyl” as used herein refers to a group of the formula —C(═O)-D, where D represents an alkyl, alkenyl, alkynyl, cycloalkyl, aryl, heteroaryl, or heterocycle as described above. Typical examples are groups wherein D is a C₁₋₁₀ alkyl, C₂₋₁₀ alkenyl or alkynyl, or phenyl, each of which is optionally substituted. In some embodiments, D can be H, Me, Et, isopropyl, propyl, butyl, C₁₋₄ alkyl substituted with —OH, —OMe, or NH₂, phenyl, halophenyl, alkylphenyl, and the like.

The term “aryloxy” as used herein refers to an aryl group connecting to an oxygen atom, wherein the aryl group may be further substituted. For example, suitable aryloxy groups include phenyloxy, etc. Similarly, the term “arylthio” as used herein refers to an aryl group connecting to a sulfur atom, wherein the aryl group may be further substituted. For example, suitable arylthio groups include phenylthio, etc.

The hydrocarbon portion of each alkoxy, alkylthio, alkylamino, and aryloxy, etc. can be substituted as appropriate for the relevant hydrocarbon moiety.

The term “halogen” as used herein refers to fluorine, chlorine, bromine and iodine.

Where present as a substituent group, halogen or halo typically refers to F or Cl or Br, more typically F or Cl.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

The term “haloalkoxy” refers to the group alkyl-O— wherein one or more hydrogen atoms on the alkyl group have been substituted with a halo group and include, by way of examples, groups such as trifluoromethoxy, and the like.

The term “sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein each alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

The term “sulfonylamino” refers to the group —NR^{NS1}SO₂R^{NS2}, wherein R^{NS1} and R^{NS2} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and wherein R^{NS1} and R^{NS2} may optionally join together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminosulfonyl” refers to the group —SO₂NR^{NS1}R^{NS2}, wherein each R^{NS1} and R^{NS2} are independently selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and wherein R^{NS1} and R^{NS2} may optionally join together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic, which are as defined herein.

The term “acylamino” refers to the groups —NR^{NS1}C(═O)alkyl, —NR^{NS1}C(═O)substituted alkyl, —NR^{NS1}C(═O)cycloalkyl, —NR^{NS1}C(═O)substituted cycloalkyl, —NR^{NS1}C(═O)cycloalkenyl, —NR^{NS1}C(═O)substituted cycloalkenyl, —NR^{NS1}C(═O)alkenyl, —NR^{NS1}C(═O)substituted alkenyl, —NR^{NS1}C(═O)alkynyl, —NR^{NS1}C(═O)substituted alkynyl, —NR^{NS1}C(═O)aryl, —NR^{NS1}C(═O)substituted aryl, —NR^{NS1}C(═O)heteroaryl, —NR^{NS1}C(═O)substituted heteroaryl, —NR^{NS1}C(═O)heterocyclic, and —NR^{NS1}C(O)substituted heterocyclic, each R^{NS1} is selected from hydrogen, substituted or unsubstituted C₁₋₆ alkyl, substituted or unsubstituted C₂₋₆ alkenyl, substituted or unsubstituted C₂₋₆ alkynyl, aryl, fused aryl, heteroaryl, fused heterocycle, a C₃₋₈ carbocyclic ring or a C₄₋₈ heterocyclic ring, saturated or unsaturated, wherein suitable, a substituted or unsubstituted alkyl, alkenyl, alkynyl can optionally contain a heteroatom selected from N, O, P, and S in place of a carbon atom.

The term “alkoxycarbonyl amino” refers to the group —NR^{NS1}C(═O)OR^{NS2}, wherein ach R^{NS1} and R^{NS2} are independently selected from hydrogen, substituted or unsubstituted C₁₋₆ alkyl, substituted or unsubstituted C₂₋₆ alkenyl, substituted or unsubstituted C₂₋₈ alkynyl, aryl, fused aryl, heteroaryl, fused heterocycle, a C₃₋₈ carbocyclic ring or a C₄₋₈ heterocyclic ring, saturated or unsaturated, wherein suitable, a substituted or unsubstituted alkyl, alkenyl, alkynyl can optionally contain a heteroatom selected from N, O, P, and S in place of a carbon atom; wherein R^{NS1} and R^{NS2} may join together to form a 4, 5, 6 or 7-membered heterocyclic ring, when suitable, a carbon atom in the ring can be replaced by a heteroatom selected from N, S, O, and P.

The term “aminocarbonylamino” refers to the group —NR^{NS1}C(═O)NR^{NS1}R^{NS2}: each R^{NS1} and R^{NS2} are independently selected from hydrogen, substituted or unsubstituted C₁₋₈ alkyl, substituted or unsubstituted C₂₋₈ alkenyl, substituted or unsubstituted C₂₋₈ alkynyl, aryl, fused aryl, heteroaryl, fused heterocycle, a C₃₋₈ carbocyclic ring or a C₄₋₈ heterocyclic ring, saturated or unsaturated, wherein suitable, a substituted or unsubstituted alkyl, alkenyl, alkynyl can optionally contain a heteroatom selected from N, O, P, and S in place of a carbon atom; wherein R^{NS1} and R^{NS2} may join together to form a 4, 5, 6 or 7-membered heterocyclic ring, when suitable, a carbon atom in the ring can be replaced by a heteroatom selected from N, S, O, and P.

It should further be recognized that all of the above-defined groups may further be substituted with one or more substituents, which may in turn be substituted with hydroxy, amino, cyano, C₁₋₄ alkyl, halo, or C₁₋₄ haloalkyl. For example, a hydrogen atom in an alkyl or aryl can be replaced by an amino, halo or C₁₋₄ haloalkyl or alkyl group.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(═O)OR, C(═X)OH, etc.), polar groups (e.g., —OH), non-polar groups (e.g., heterocycle, aryl, alkyl, alkenyl, alkynyl, etc.), ionic groups (e.g., —NH₃⁺), and halogens (e.g., —F, —Cl, —Br, —I), NHCOR, NHCONH₂, OCH₂COOH, OCH₂CONH₂, OCH₂CONHR, NHCH₂COOH, NHCH₂CONH₂, NHSO₂R, OCH₂-heterocycles, —PO₃H, —SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, pharmaceutically acceptable salts were discussed in detail in Berge S. M. et al., J. Pharmaceutical Sciences, 1977. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₅alkyl)₄ salts. Representative alkali or alkaline earth metal salts include sodium, lithium, potassium, calcium, magnesium, and the like. Further pharmaceutically acceptable salts may include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “salt thereof” as used herein means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

An “effective amount” as used herein, means an amount which provides the indicated therapeutic benefit, i.e., an amount that results in the treatment of cystic fibrosis. It is understood, however, that the full therapeutic effect does not necessarily occur by administration of one dose, and may occur only after administration of a series of doses. Thus, an effective amount may be administered in one or more administrations. In the context of therapeutic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors.

As used herein, “individual” or “patient” or “subject” (as in the subject of the treatment) means both mammals and non-mammals. Mammals include, for example, humans; non-human primates, e.g. apes and monkeys; cattle; horses; sheep; and goats. Non-mammals include, for example, fish and birds. The individual is, in one embodiment, a human being.

To “treat” a disease or a disorder as the term is used herein, means to reduce the frequency or severity of at least one sign or symptom of a disease or disorder experienced by a subject. Treating may include the postponement of further disease progression, or reduction in the severity of symptoms that have or are expected to develop, ameliorating existing symptoms and preventing additional symptoms.

The term “modulating” as used herein encompasses increasing, enhancing, inhibiting, decreasing, suppressing, and the like. As used herein, the terms “increasing” and “enhancing” mean to cause a net gain by either direct or indirect means. As used herein, the terms “inhibiting” and “decreasing” encompass causing a net decrease by either direct or indirect means.

It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 2. Compounds

The present disclosure is directed to compounds of Formula (1), pharmaceutically acceptable salts, pharmaceutically acceptable hydrates, pharmaceutically acceptable solvates, pharmaceutically acceptable clathrates, or pharmaceutically acceptable polymorphs thereof,

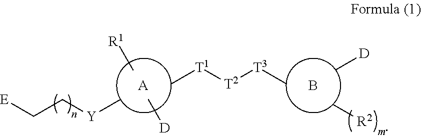

The compounds of Formula (1) can be use for the improvement and/or stabilization of ion channel activity of cystic fibrosis transmembrane conductance regulator (CFTR) proteins. These compounds can be used for the treatment of cystic fibrosis (CF).

In Formula (1), Ring A may be selected from phenyl, six-membered aromatic ring with 1, 2, or 3 nitrogen ring atoms, or a five-membered ring, aromatic or non-aromatic, with 1, 2, or 3 heteroatoms independently selected from O, S, or N;

Ring B may be a mono or bicyclic ring system, aromatic or non-aromatic, with 5, 6, 7, 8, 9, or 10 ring atoms with 1 to 4 heteroatoms selected from O, S, or N;

each T¹, T², and T³ may be independently absent, or independently selected from C(R^{CT})₂, C(O), S(O)₀₋₂ or NR^{NT}, wherein R^{CT} with R^{CT}, or R^{CT} with R^{NT}, may join together to form a three-, four-, or five-membered aliphatic ring; or R^{CT} and R^{NT} may be each independently selected from H, CH₂OH, C₁₋₄alkyl, C₂₋₆alkenyl, CF₃, or (R^{CT})₂ is ═CHR³, where R³ may be independently selected from H, CH₂OH, C₁₋₄alkyl, or C₂₋₆alkenyl;

Y may be selected from a bond, O, S(O)₀₋₂, NR³, or —C(O)NR³;

D may be each independently selected from F, CF₃, CHₐF₍₃₋ₐ₎, C1, Br, CN, NO₂, OR⁴, OCF₃, or OC(O)R⁵, where a may be 1 or 2; R⁴ may be H, C₁₋₄alkyl, or R⁵; and R⁵ may be (CH₂)ⱼR⁶ where j may be an integer from 3 to 8, and R⁶ is H or E, and optionally one or more adjacent CH₂ units may be replaced with O, S, or NR³, and/or one or more hydrogens of the CH₂ units may be substituted with F, Cl, Br, CN, OR⁴, OH, or NHR³;

E may be N(R³)₂;

R¹ and R² may be independently selected from C₁₋₆ alkyl, five- to nine-membered heteroaryl, phenyl, napthyl, —OR⁴, —N(R³)₂, —SR⁴, —SO₂R⁴, —SO₂N(R³)₂, —NR³SO₂R⁴, —NR³C(O)OR⁴, —NR³C(O)R⁴, —C(O)OR⁴, —C(O)N(R³)₂, —OC(═O)R⁴, —C(═O)R⁴, and m may be an integer from 1 to 4; and

n may be an integer from 0-8, and optionally one or more CH₂ units may be replaced with O, S(O)₀₋₂, P(O)R³, or NR³, and/or one or more hydrogens of the CH₂ units may be substituted with R³, F, Cl, Br, CN, OR⁴, OH, ═O, ═NR³, COOR³, CON(R³)₂, S(O)₂N(R³)₂. S(O)₀₋₂R³ or N(R³)₂, and optionally any one pair of existed R³s may join together to form a 3-7 membered ring system of aromatic or non-aromatic nature, and optionally two adjacent CH₂ units may form a double bond or a triple bond.

In Formula (1), any one combination of T¹-T², T²-T³, or T¹-T³ may not be —C(O)—N(R^{NT})—.

In Formula (1), Ring A may be an aromatic six-membered ring structure selected from the group consisting of

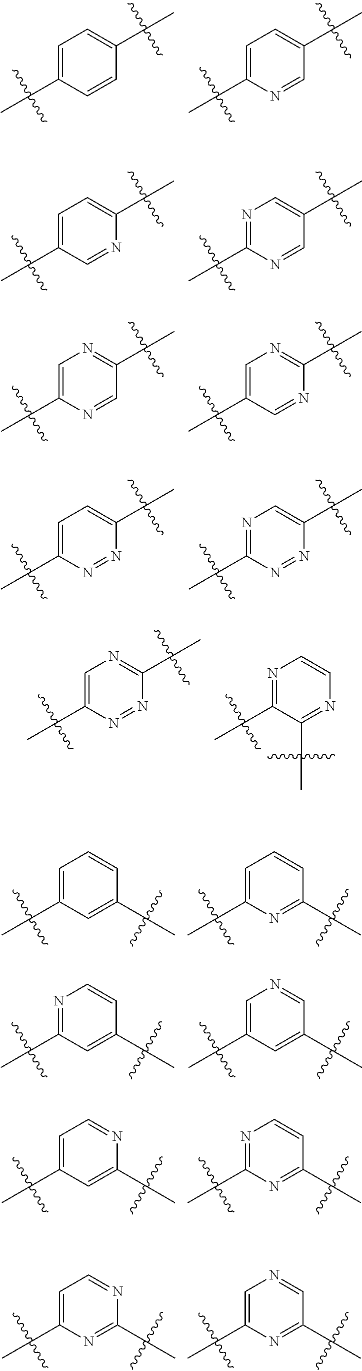

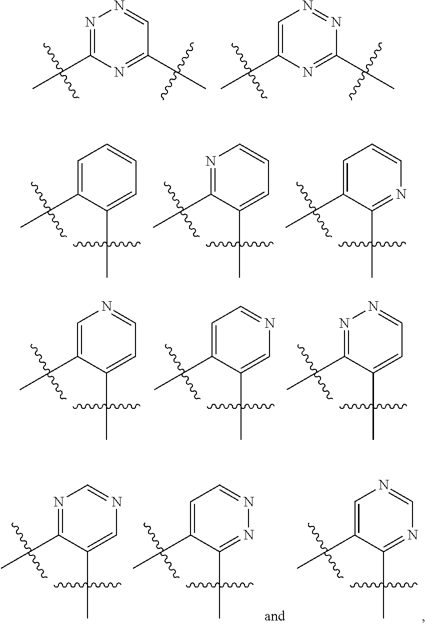

wherein the wavy line to the right indicates the connection of Ring A to T¹, and the wavy line to the left indicates the connection of Ring A to Y.

In Formula (1), Ring A may be a five-membered ring structure selected from the group consisting of

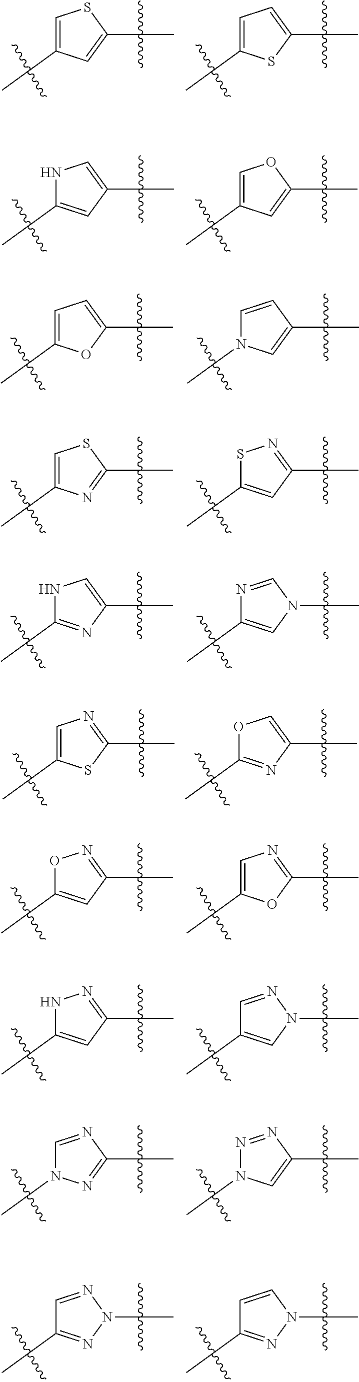

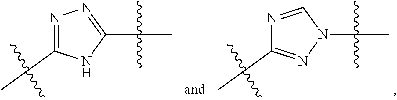 [SMILES: *c1nnc(*)[nH]1 ; *c1ncn(*)n1]

wherein the wavy line to the right indicates the connection of Ring A to T¹, and the wavy line to the left indicates the connection of Ring A to Y.

In Formula (1), Ring B may be a nine-membered ring structure selected from the group consisting of

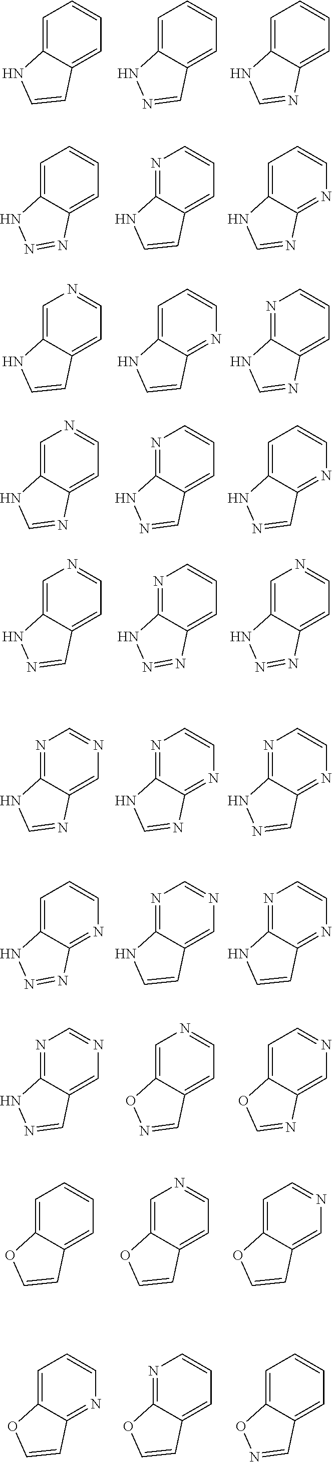

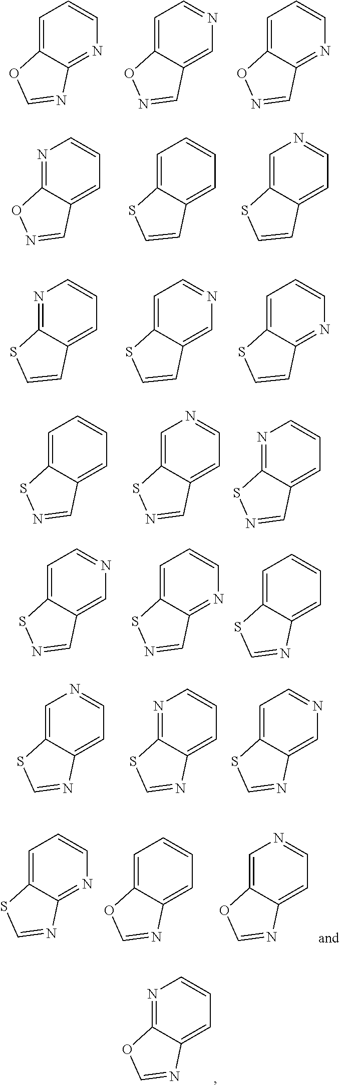

wherein Ring B may be connected to T3 at one of ring carbons of the fused six-membered ring.

Alternatively, in Formula (1), Ring B may be a nine-membered ring structure selected from the group consisting of

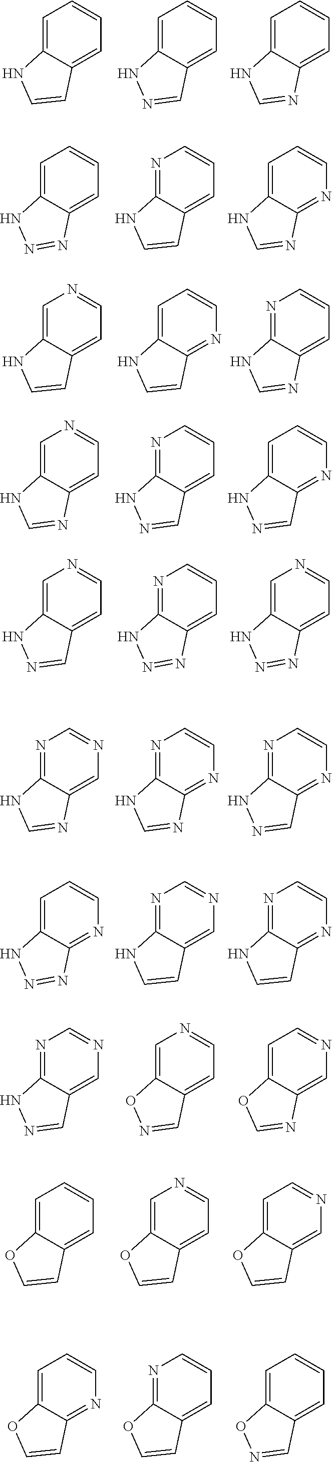

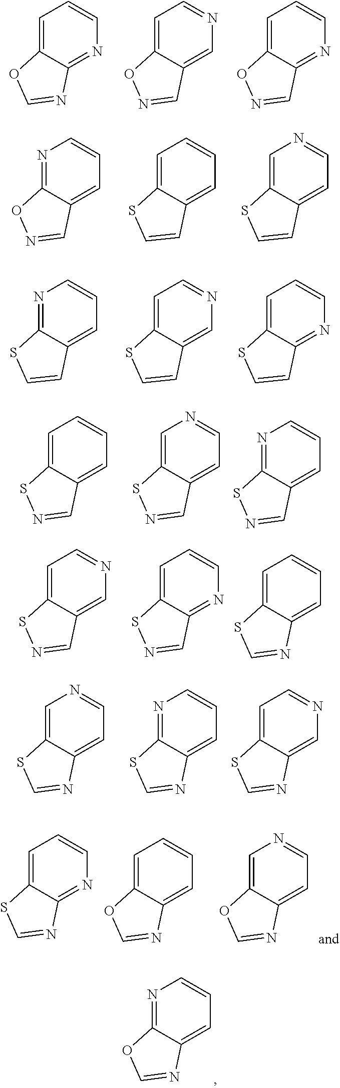

wherein Ring B may be connected to T3 at one of ring carbons of the fused five-membered ring, or the nitrogen of an indicated NH of the fused five-membered ring.

In Formula (1), Ring B also may be a ten-membered ring structure selected from the group consisting of

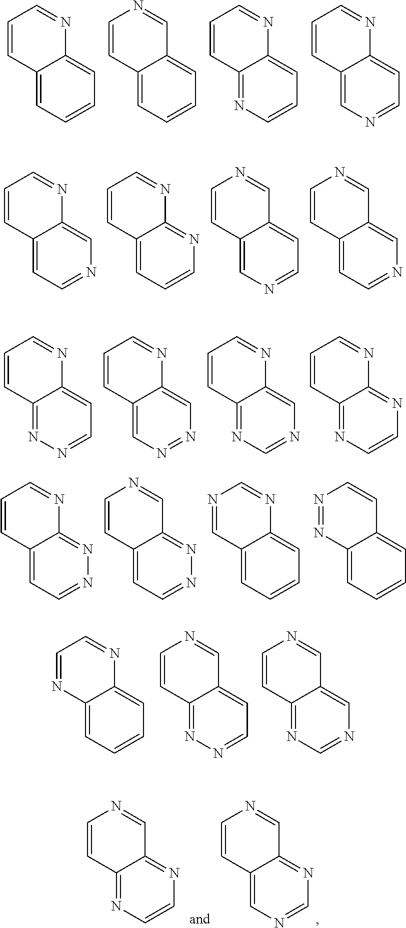

wherein Ring B may be connected to T3 at one of ring carbon of either six-membered ring.

In Formula (1), each of T¹, T², and T³ of may be absent, and Ring A may be directly connected to Ring B.

In Formula (1), Ring A of Formula (1) may be selected from phenyl or pyridyl.

In Formula (1), Ring A may be selected from phenyl or pyridyl, and Ring B may be a bicyclic ring system with 9 ring atoms with 1 to 2 heteroatoms selected from O or N.

The compound of Formula (1) may be selected from the group consisting of Formula (2), Formula (3), and Formula (4)

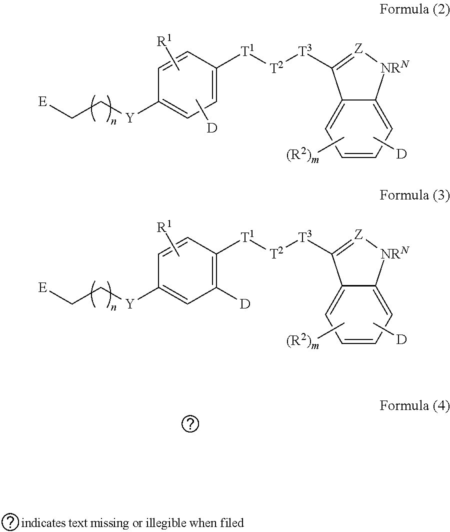

wherein Z is CH or N, and R^{N} is R³.

In Formula (1), T² and T³ may be absent.

In Formula (1), D may be independently selected from the group consisting of F, CF3, CHaF(3-a), Br, and CN.

Representative compounds of Formula (1) may include, but are not limited to the following compounds:

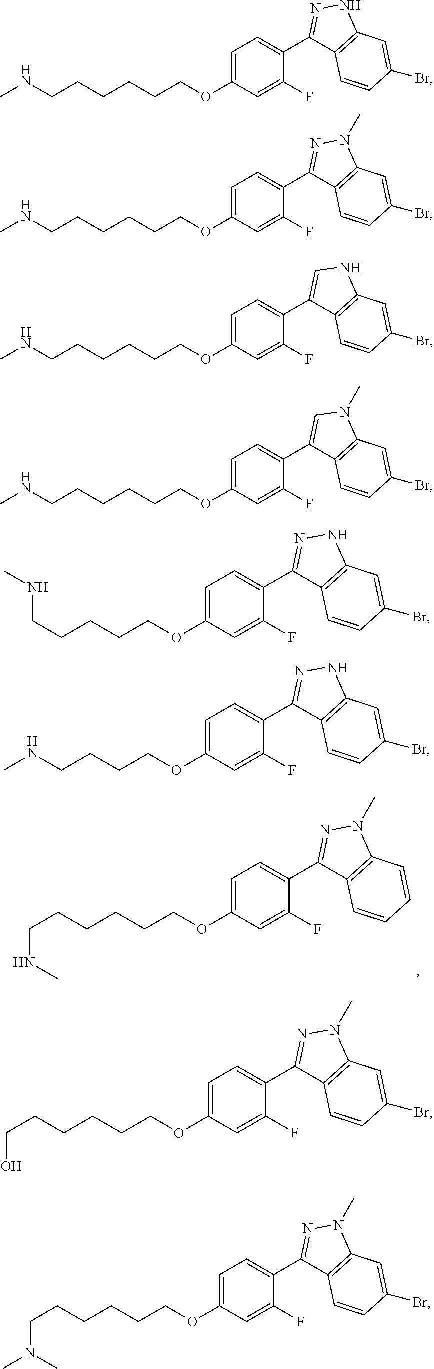

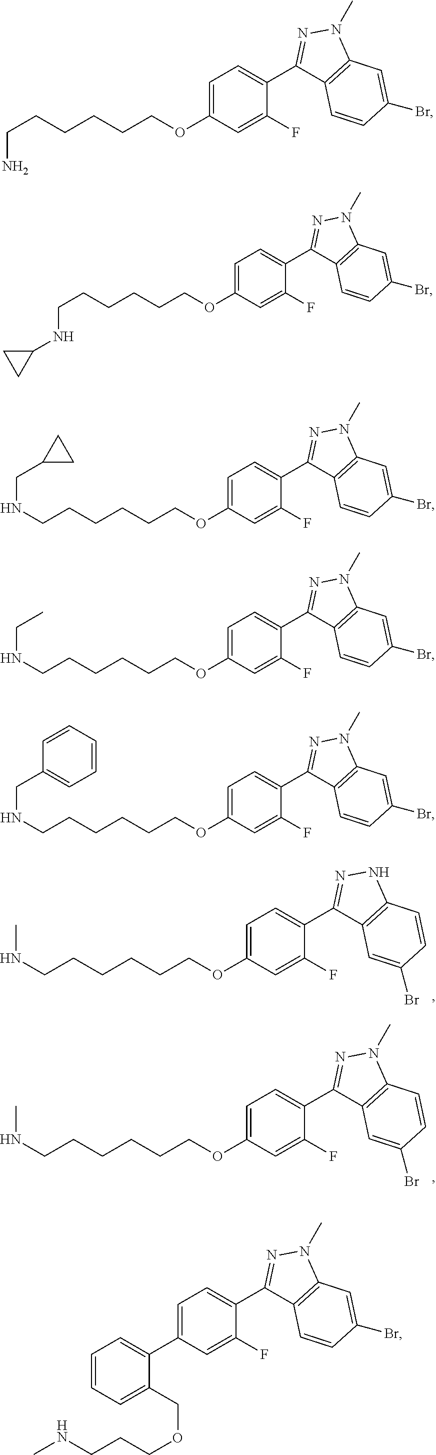

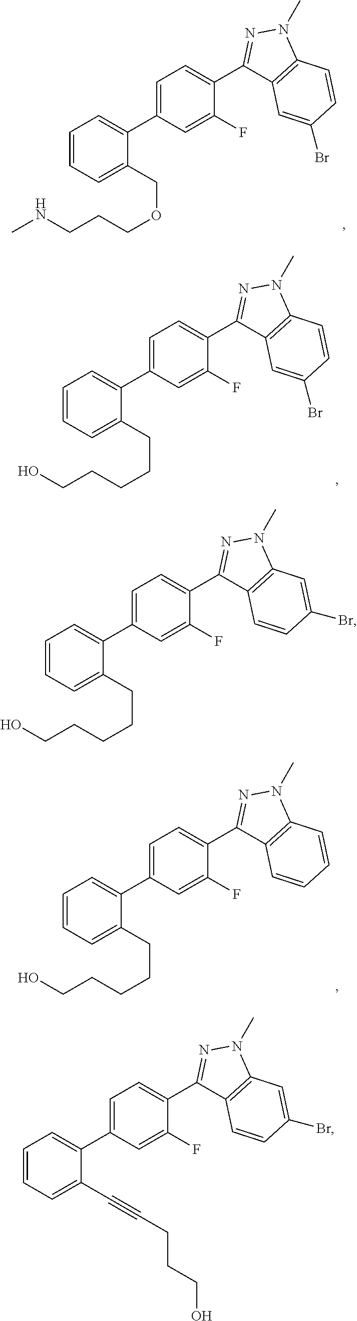 [SMILES: CNCCCOCc1ccccc1-c1ccc(-c2nn(C)c3ccc(Br)cc23)c(F)c1 ; Cn1nc(-c2ccc(-c3ccccc3CCCCCO)cc2F)c2cc(Br)ccc21 ; Cn1nc(-c2ccc(-c3ccccc3CCCCCO)cc2F)c2ccc(Br)cc21 ; Cn1nc(-c2ccc(-c3ccccc3CCCCCO)cc2F)c2ccccc21 ; Cn1nc(-c2ccc(-c3ccccc3C#CCCCO)cc2F)c2ccc(Br)cc21]

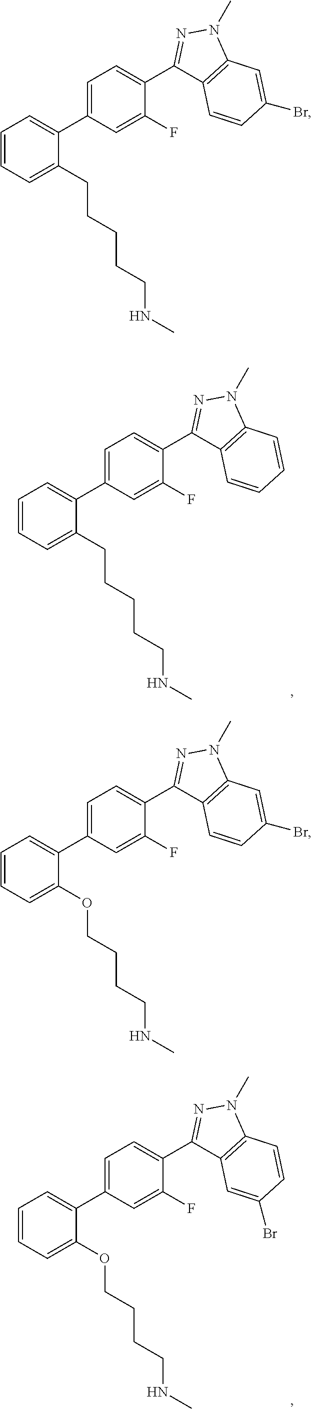

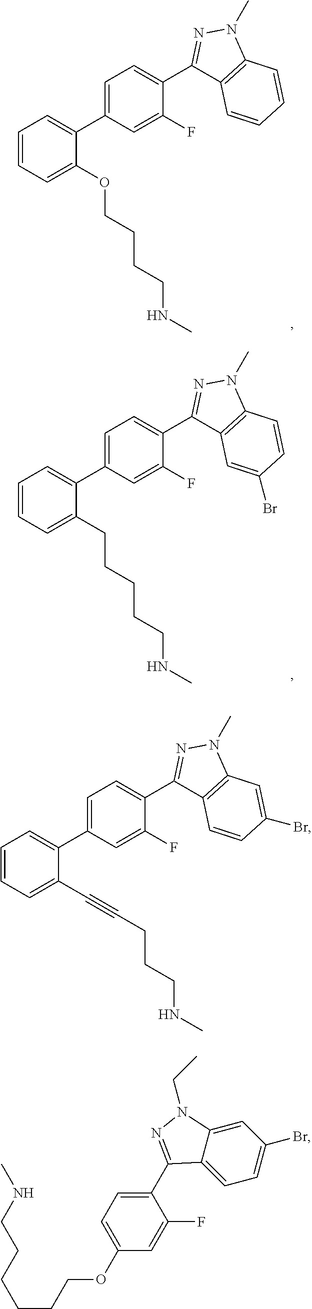

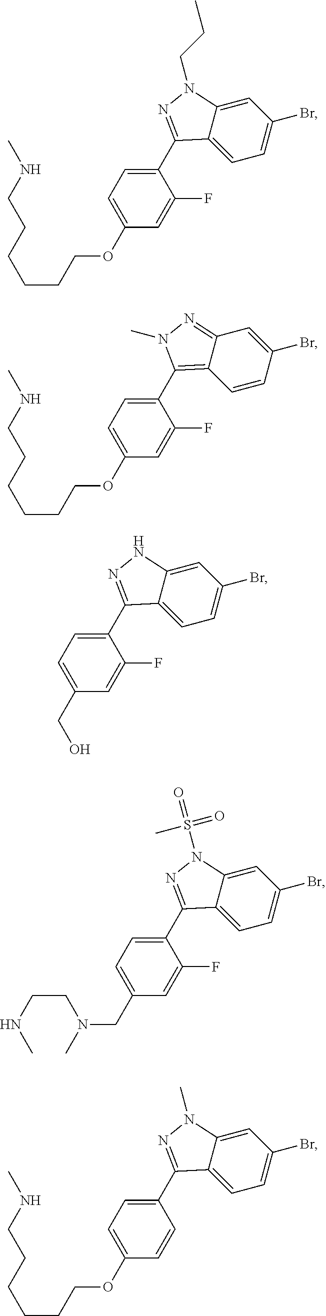 [SMILES: CCCn1nc(-c2ccc(OCCCCCCNC)cc2F)c2ccc(Br)cc21 ; CNCCCCCCOc1ccc(-c2c3ccc(Br)cc3nn2C)c(F)c1 ; OCc1ccc(-c2n[nH]c3cc(Br)ccc23)c(F)c1 ; CNCCN(C)Cc1ccc(-c2nn(S(C)(=O)=O)c3cc(Br)ccc23)c(F)c1 ; CNCCCCCCOc1ccc(-c2nn(C)c3cc(Br)ccc23)cc1]

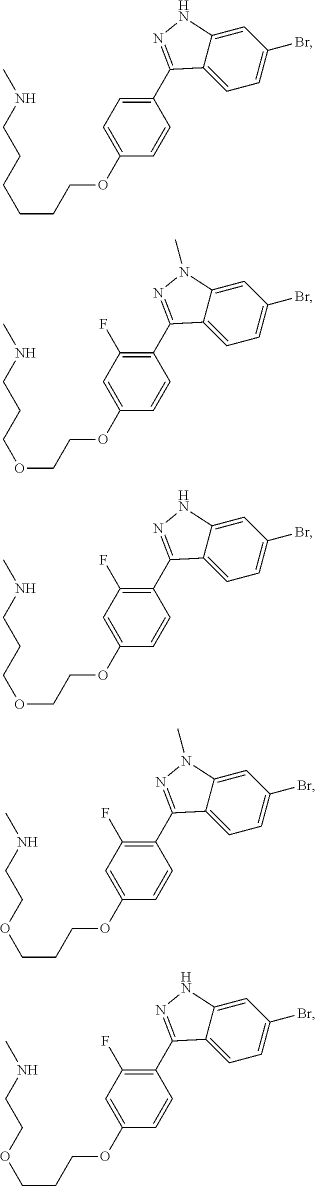 [SMILES: CNCCCCCCOc1ccc(-c2n[nH]c3cc(Br)ccc23)cc1 ; CNCCCOCCOc1ccc(-c2nn(C)c3cc(Br)ccc23)c(F)c1 ; CNCCCOCCOc1ccc(-c2n[nH]c3cc(Br)ccc23)c(F)c1 ; CNCCOCCCOc1ccc(-c2nn(C)c3cc(Br)ccc23)c(F)c1 ; CNCCOCCCOc1ccc(-c2n[nH]c3cc(Br)ccc23)c(F)c1]

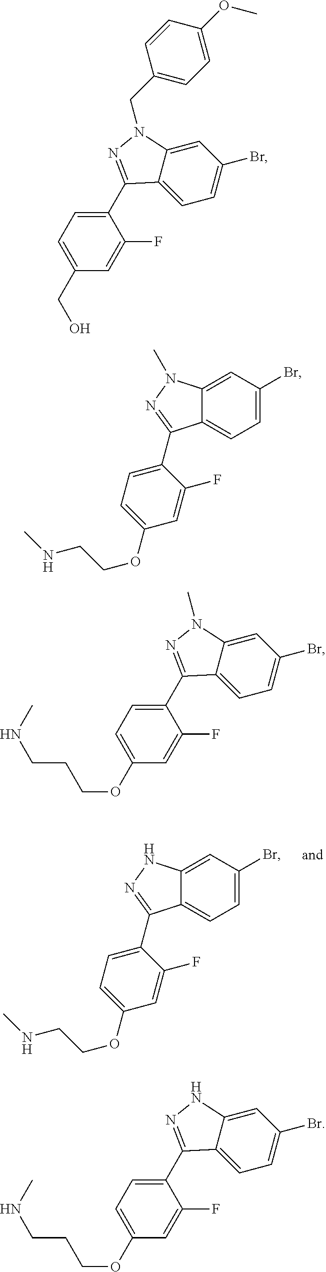

The compound of Formula (I) may be a compound of Formula (5):

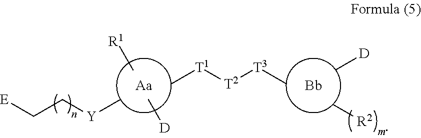

In Formula (5), Ring Aa may be selected from phenyl, six-membered aromatic ring with 1, 2 or 3 nitrogen ring atoms, or a five-membered ring, aromatic or non-aromatic, with 1 to 3 heteroatoms independently selected from O, S, or N;

Ring Bb may be phenyl, or a six-membered aromatic ring with 1 to 2 nitrogen ring atoms;

each T¹, T², and T³ may be independently absent, or independently selected from C(R^{CT})₂, C(O), S(O)₀₋₂ or NR^{NT}, wherein R^{CT} with R^{CT}, or R^{CT} with R^{NT}, may join together to form a three-, four-, or five-membered aliphatic ring; or R^{CT} and R^{NT} may be each independently selected from H, CH₂OH, C₁₋₄alkyl, C₂₋₆alkenyl, CF₃, or (R^{CT})₂ is ═CHR³, where R³ may be independently selected from H, CH₂OH, C₁₋₄alkyl, or C₂₋₆alkenyl;

Y may be selected of from a bond, O, S(O)₀₋₂, NR³, or —C(O)NR³;

D may be each independently selected from F, CF₃, CHₐF₍₃₋ₐ₎, Cl, Br, CN, NO₂, OR⁴, OCF₃, or OC(O)R⁵, where a is 1 or 2; R⁴ is H, C₁₋₄alkyl, or R⁵; and R⁵ is (CH₂)ⱼR⁶ where j is an integer from 3 to 8, and R⁶ is H or E, and optionally one or more adjacent CH₂ units is replaced with O, S, or NR³, and/or one or more hydrogens of the CH₂ units is substituted with F, Cl, Br, CN, OR⁴, OH, or NHR³;

E may be N(R³)₂;

R¹ and R² may be independently selected from C₁₋₆ alkyl, five- to nine-membered heteroaryl, phenyl, napthyl, —OR⁴, —N(R³)₂, —SR⁴, —SO₂R⁴, —SO₂N(R³)₂, —NR³SO₂R⁴, —NR³C(O)OR⁴, —NR³C(O)R⁴, —C(O)OR⁴, —C(O)N(R³)₂, —OC(═O)R⁴, —C(═O)R⁴, and m is an integer from 1 to 4; and

n may be an integer from 0-8, and optionally one or more CH₂ units may be replaced with O, S(O)₀₋₂, P(O)R³, or NR³, and/or one or more hydrogens of the CH₂ units may be substituted with F, Cl, Br, CN, OR⁴, OH, ═O, ═NR³, COOR³, CON(R³)₂, S(O)₂N(R³)₂, S(O)₀₋₂R³, or N(R³)₂, and optionally any one pair of existed R³s may join together to form a 3-7 membered ring system of aromatic or non-aromatic nature, and optionally two adjacent CH₂ units may form a double bond or a triple bond.

In Formula (5), any one combination of T¹-T², T²-T³, or T¹-T³ may not be —C(O)—N(R^{NT})—.

In Formula (5), Ring Aa and Ring Bb may be both phenyl.

In Formula (5), Ring Aa may be phenyl and Ring Bb may be a six-membered aromatic ring with 1 to 2 nitrogen ring atoms, or Ring Aa may be a six-membered aromatic ring with 1 to 2 nitrogen ring atoms and Ring Bb may be phenyl.

In Formula (5), Ring Aa a six-membered aromatic ring with 1, 2 or 3 nitrogen ring atoms, and Ring Bb is a six-membered aromatic ring with 1 or 2 nitrogen ring atoms.

In Formula (5), T² and T³ may be absent.

It will be appreciated by one skilled in the art that the processes described are not the exclusive means by which compounds of Formula (1) may be synthesized and that a repertoire of synthetic organic reactions is available to be potentially employed in synthesizing compounds of the disclosure. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} nd Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

The compounds of Formula (1) and intermediates may be isolated from their reaction mixtures and purified by standard techniques such as filtration, liquid-liquid extraction, solid phase extraction, distillation, recrystallization or chromatography.

The compounds of Formula (1) may take the form of salts when appropriately substituted with groups or atoms capable of forming salts. Such groups and atoms are well known to those of ordinary skill in the art of organic chemistry. The term “salts” embraces addition salts of free acids or free bases which are compounds of the disclosure. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as, for example, utility in process of synthesis, purification or formulation of compounds of the disclosure.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, pivalic, propionic, furoic, mucic, isethionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, camphorosulfonic and galacturonic acid. Examples of pharmaceutically unacceptable acid addition salts include, for example, perchlorates and tetrafluoroborates.

Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, tromethamine, meglumine (N-methylglucamine) and procaine. Examples of pharmaceutically unacceptable base addition salts include lithium salts and cyanate salts.

All of these salts may be prepared by conventional means from the corresponding compound according to Formula (1) by reacting, for example, the appropriate acid or base with the compound according to Formula (1). Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selection, and Use By P. H. Stahl and C. G. Wermuth (Wiley-VCH 2002).

The compounds of Formula (1) can exist in solvated as well as unsolvated forms with pharmaceutically acceptable solvents such as water, ethanol, and the like, and it is intended that the disclosure embraces both solvated and unsolvated forms of disclosed compounds. In one example, a compound of Formula (1) may be amorphous or may be a single polymorph. In another example, a compound of Formula (1) may be a mixture of polymorphs. In another example, a compound of Formula (1) may be in a crystalline form.

### 3. Methods

As discussed above, contemplated herein may also include a method of enhancing CFTR activity in a subject comprising administering a composition comprising an effective amount of a described compound. Also contemplated herein may include a method of treating a patient suffering from a condition associated with CFTR activity comprising administering to the patient a composition comprising an effective amount of a compound described herein.

In some examples, CFTR activity is enhanced after administration of a compound described herein when there is an increase in the CFTR activity as compared to that in the absence of the administration of the compound. CFTR activity encompasses, for example, chloride channel activity of the CFTR, and/or other ion transport activity (for example, HCO₃ transport). In certain of these embodiments, the activity of one or more (e.g., one or two) mutant CFTRs (e.g., delF508, S549N, G542X, G551D, R117H, N1303K, W1282X, R553X, 621+1G>T, 1717-1G>A, 3849+10kbC>T, 2789+5G>A, 3120+1G>A, 1507del, R1162X, 1898+1G>A, 3659delC, G85E, Dl 152H, R560T, R347P, 2184insA, A455E, R334W, Q493X, and 2184delA CFTR) is enhanced (e.g., increased).

Contemplated patients may have a CFTR mutation(s) from one or more classes, such as, without limitation, Class I CFTR mutations, Class II CFTR mutations, Class III CFTR mutations, Class IV CFTR mutations, Class V CFTR mutations, and Class VI mutations. The CFTR genotypes of contemplated subject (e.g., human subject) include, without limitation, homozygote mutations (e.g., delF508/delF508 and R117H/R117H) and compound heterozygote mutations (e.g., delF508/G551D; delF508/A455E; delF508/G542X; delF508/W1204X; R553X/W1316X; W1282X/N1303K, 591Δ18/E831X, delF508/R117H/N1303K/3849+10kbC>T; Δ303K/384; and delF508/G178R).

In some examples, the mutation may be a Class I mutation, e.g., a G542X Class I mutation; or a Class II/I mutation, e.g., a delF508/G542X compound heterozygous mutation. In other examples, the mutation may be a Class III mutation, e.g., a G551D Class III mutation; or a Class II Class III mutation, e.g., a delF508/G551D compound heterozygous mutation. In still other examples, the mutation may be a Class V mutation, e.g., an A455E Class V mutation; or a Class II/Class V mutation, e.g., a delF508/A455E compound heterozygous mutation. Of the more than 1000 known mutations of the CFTR gene, delF508 is the most prevalent mutation of CFTR. The delF508 mutation results in misfolding of the protein and impaired trafficking from the endoplasmic reticulum to the apical membrane. See, e.g., Dormer et al., J. Cell Sci. 2001.

In some examples, delF508 CFTR activity may be enhanced (e.g., increased) after administration of a compound described herein. In certain examples, delF508 CFTR activity and/or G542X CFTR activity and/or G551D CFTR activity and/or A455E CFTR activity may be enhanced (e.g., increased) after administration of a compound described herein. An enhancement of CFTR activity can be measured, using established methods, including for example, Ussing chamber assays, patch clamp assays, and hBE Ieq assay. See, e.g., Devor et al., Am. J. Physiol. Cell Physiol. 2000; Dousmanis et al., J Gen. Physiol. 2002; Bruscia et al., PNAS 2005.

As discussed above, a method of treating cystic fibrosis is contemplated, comprising administering a composition comprising an effective amount of a described compound. Treatment of other conditions associated with CFTR activity, including conditions associated with deficient CFTR activity using disclosed compounds is also contemplated in certain examples.

In some examples, a method of treating a condition associated with deficient or decreased CFTR activity comprising administering a composition comprising an effective amount of a disclosed compound that enhances CFTR activity is provided. Non-limiting examples of conditions associated with deficient CFTR activity are cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, chronic obstructive pulmonary disease (COPD), chronic sinusitis, dry eye disease, protein C deficiency, AP-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

Provided herein are methods of treating a patient having one or more of the following mutations in the CFTR gene: G1244E, G1349D, G178R, G551 S, S1251 N, S1255P, S549N, S549R, G970R, or R117H, and/or e.g., a patient with one or two copies of the delF508 mutation, or one copy of the delF508 mutation and a second mutation that results in a gating effect in the CFTR protein (e.g., a patient that is heterozygous for delF508 and G551 D mutation), a patient with one copy of the delF508 mutation and a second mutation that results in residual CFTR activity, or a patient with one copy of the delF508 mutation and a second mutation that results in residual CFTR activity, comprising administering an effective amount of a described compound.

The described compounds may be administered in the form of a pharmaceutical composition, in combination with a pharmaceutically acceptable carrier. The active ingredient or agent in such formulations (i.e. a compound of Formula (1)) may comprise from 0.1 to 99.99 weight percent of the formulation. “Pharmaceutically acceptable carrier” means any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions. The administration route may be oral, intravenous, intraperitoneal, intramuscular, transdermal, subcutaneous, topical, sublingual, intravascular, intramammary, or rectal means.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet example, the active agent may be combined with carboxymethylcellulose calcium, magnesium stearate, mannitol and starch, and then formed into tablets by conventional tableting methods.

The compounds described herein may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. See, e.g., U.S. Pat. Nos. 5,674,533, 5,059,595, 5,591,767, 5,120,548, 5,073,543, 5,639,476, and 5,733,566. The controlled-release of the active ingredient may be stimulated by various inducers, for example, pH, temperature, enzymes, water, or other physiological conditions or compounds. Various mechanisms of drug release exist. For example, in one example, the controlled-release component may swell and form porous openings large enough to release the active ingredient after administration to a patient. The term “controlled-release component” as described herein is defined herein as a compound or compounds, such as polymers, polymer matrices, gels, permeable membranes, liposomes and/or microspheres, that facilitate the controlled-release of the active ingredient in the pharmaceutical composition. In another example, the controlled-release component is biodegradable, induced by exposure to the aqueous environment, pH, temperature, or enzymes in the body. In another example, sol-gels may be used, wherein the active ingredient is incorporated into a sol-gel matrix that is a solid at room temperature. This matrix is implanted into a patient, for example, a mammal, having a body temperature high enough to induce gel formation of the sol-gel matrix, thereby releasing the active ingredient into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration (FDA). For example, suitable formulations may be sterile and/or substantially isotonic and/or in full compliance with all Good Manufacturing Practice regulations of the U.S. Food and Drug Administration.

The compounds described herein may be administered in a convenient manner. Suitable topical routes include oral, rectal, inhaled (including nasal), topical (including buccal and sublingual), transdermal and vaginal, preferably across the epidermis. The compounds described herein can also be used for parenteral administration (including subcutaneous, intravenous, intramuscular, intradermal, intraarterial, intrathecal and epidural), and the like. It will be appreciated that the preferred route may vary with, for example, the condition of the recipient.

The physician will determine the dosage of the active agent which will be most suitable and it will vary with the form of administration and the particular compound chosen, and furthermore, it will vary depending upon various factors, including, but not limited to, the patient under treatment and the age of the patient, the severity of the condition being treated, the rout of administration, and the like. The physician will generally wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 1000 mg, more typically from about 1 to about 500 mg, more typically, from about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The treatment may be carried out for as long a period as necessary, either in a single, uninterrupted session, or in discrete sessions. The treating physician will know how to increase, decrease, or interrupt treatment based on patient response. The treatment schedule may be repeated as required. For example, a compound of Formula (1) may be administered at least once daily.

In some example, a compound of Formula (1) may be the sole active agent in the described methods to enhance CFTR activity, treat a condition associated with deficient or decreased CFTR activity, or treat cystic fibrosis. In some examples, described methods of enhancing CFTR activity, treating a condition associated with deficient or decreased CFTR activity, or treating cystic fibrosis may further comprise administering one or more additional therapeutic agents. For example, a contemplated method of administering a described compound may include administering at least one additional therapeutic agent, or administering a described compound, and at least two additional therapeutic agents. Additional therapeutic agents may include, for example, mucolytic agents, bronchodilators, antibiotics, anti-infective agents, anti-inflammatory agents, ion channel modulating agents, therapeutic agents used in gene therapy, CFTR correctors, and CFTR potentiators, or other agents that are capable of modulating CFTR activity.

At least one additional therapeutic agent may be selected from the group consisting of a CFTR corrector and a CFTR potentiator. Non-limiting examples of CFTR correctors and potentiators include VX-770 (Ivacaftor), VX-809 (Lumacaftor), VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), VX-983, and Ataluren (PTC 124) (3-[5-(2-fluorophenyl)-1,2,4-oxadiazol-3-yl]benzoic acid), FDL169, GLPG1837/ABBV-974 (for example, a CFTR potentiator), GLPG2222 (for example, a CFTR corrector); and compounds described in, e.g., WO2014/144860 and 2014/176553. Non-limiting examples of modulators include QBW-251, QR-010, NB-124, and compounds described in, e.g., WO2014/045283; WO2014/081821, WO2014/081820, WO2014/152213; WO2014/160440, WO2014/160478, US2014027933; WO2014/0228376, WO2013/038390, WO2011/113894, WO2013/038386; and WO2014/180562. Non-limiting examples of anti-inflammatory agents include N6022 (3-(5-(4-(1H-imidazol-yl) phenyl)-1-(4-carbamoyl-2-methylphenyl)-1H-pyrrol-2-yl) propanoic acid), CTX-4430, N1861, N1785, and N91115.

The methods described herein can further include administering an additional therapeutic agent or administering at least two additional CFTR therapeutic agents. The methods described herein can further include administering an additional CFTR modulator or administering at least two additional CFTR modulators. In some examples, at least one CFTR modulator is a CFTR corrector (e.g., Lumacaftor, VX-661, VX-983 and GLPG2222) or potentiator (e.g., Ivacaftor, genistein and GLPG1837). In some examples, one of the at least two additional therapeutic agents is a CFTR corrector (e.g., Lumacaftor, VX-661 and VX-983) and the other is a CFTR potentiator (e.g., Ivacaftor and genistein). Optionally, one of the at least two additional therapeutic agents is a CFTR corrector (e.g., GLPG2222) and the other is a CFTR potentiator (e.g., GLPG1837). One of the at least two additional therapeutic agents is a CFTR corrector (e.g., Lumacaftor or VX-661) and the other is a CFTR potentiator (e.g., Ivacaftor). In some examples, at least one CFTR modulator is an agent that enhances read-through of stop codons (e.g., NB 124 or ataluren).

The disclosure provides a method of treating a condition associated with deficient or decreased CFTR activity (e.g., cystic fibrosis), which includes administering to a subject in need thereof (e.g., a human patient in need thereof) (1) a compositions comprising an effective amount of a disclosed compound and (2) at least one or two additional CFTR therapeutic agent(s) (e.g., at least one or two additional CFTR therapeutic agents, e.g., in which one of the at least one or two additional therapeutic agents is optionally a CFTR corrector or modulator (e.g., Lumacaftor, VX-661, VX-983, GLPG2222, NB 124, ataluren) and/or the other is a CFTR potentiator (e.g., Ivacaftor, genistein, and GLPG1837); e.g., one of the at least two additional therapeutic agents is GLPG2222, and the other is GLPG1837; or one of the at least two additional therapeutic agents is Lumacaftor or VX-661, and the other is a Ivacaftor).

### EXAMPLES

### Examples 1-48: Synthesis of the Compounds

### LCMS Conditions

Column: Agilent Zorbax Eclipse XDB-C18, 4.6 mm×30 mm, 3.5 μm; temperature: 25° C.; Eluent A: H₂O+0.1% TFA; Eluent B: Acetonitrile+0.1% TFA; Flow Rate: 2.0 mL/min;

Gradient: the gradient start (0 min) with 5% Eluent B and gradually increase to 100% of Eluent B over 2.30 min (2.30 min run), then the gradient was kept at 100% of Eluent B for 1.30 min and the run was ended (total run 4.00 min).

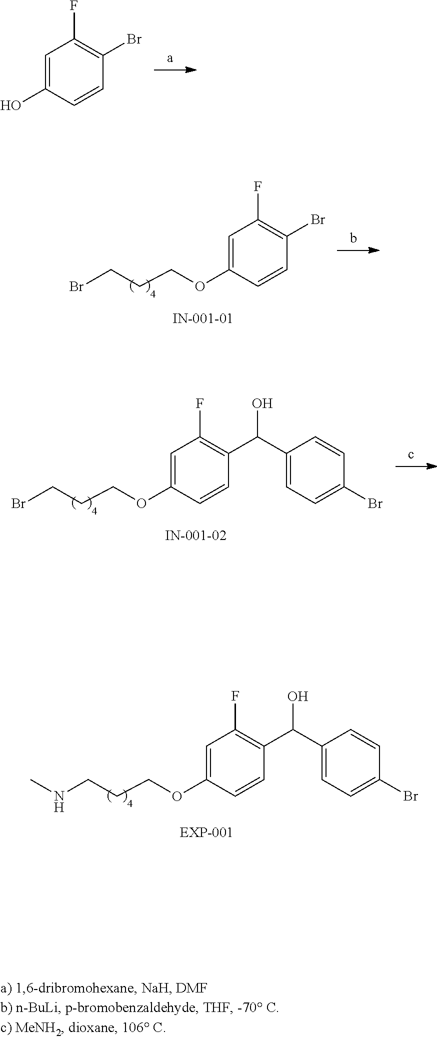

### Step 1

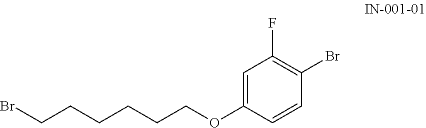 [SMILES: Fc1cc(OCCCCCCBr)ccc1Br]

### IN-001-01: 1-bromo-4-(6-bromohexyloxy)-2-fluorobenzene

To an anhydrous DMF (60 mL) solution 4-bromo-3-fluorophenol (6.66 g, 34.87 mmol) and 1,6-dibromohexane (16.1 mL, 104.71 mmol), NaH (1.39 g NaH at 60%, 0.84 g of NaH, 34.90 mmol) was added portionwise. The reaction mixture was stirred for room temperature for 4 h under N₂ atmosphere. Then, the reaction was quenched adding water (60 mL) and the mixture was extracted with EtOAc (3×60 mL). The organic layers were combined and the combined organic phases were washed with water (3×100 mL). The organic phase was dried over Na₂SO₄ and the solvent was removed in vacuo. The crude was purified by flash chromatography (Hexanes-EtOAc 0-5%) and the product was obtained as a colorless oil (6.75, 59%). The molecular ion was not detected by LCMS. LCMS ret. time: 2.58. Rf: 0.51 (Hexanes/EtOAc 95/5). ¹H NMR (CDCl₃, δ): 1.52 (m, 4H), 1.81 (quint, J=7.35 Hz, 2H), 1.92 (quint, J=7.35 Hz, 2H), 3.44 (t, J=6.15 Hz, 2H), 3.94 (pt, J=6.20 Hz, 2H), 6.62 (ddd, J=9.01 Hz J=3.70 Hz, J=1.00 Hz, 1H), 6.70 (dd, J=10.51 Hz, J=2.95 Hz, 1H), 7.41 (dd, J=8.90 Hz, J=8.00 Hz, 1H).

### Step 2

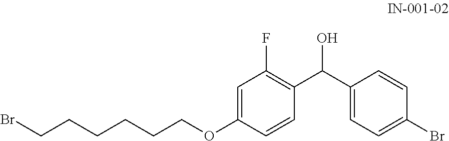 [SMILES: OC(c1ccc(Br)cc1)c1ccc(OCCCCCCBr)cc1F]

### IN-001-02: 4-(6-bromohexyloxy)-2-fluorophenyl)(4-bromophenyl)methanol

A solution of IN-001-01 (1.41 g, 3.49 mmol) in anhydrous THF (15 mL) under N₂ was cooled down in at −70° C., and then, 1.6 M n-BuLi solution in hexanes was added (2.40 mL, 3.84 mmol) dropwise. After stirring for 2 h, the solution at −70° C., the p-bromobenzaldehyde (0.65 g, 3.49 mmol) dissolved in anhydrous THF (15 mL) was added dropwise. The reaction was stirred for 30 min at −70° C. and then for 1 h on an ice-water bath and at room temperature for an overnight. After this time, the reaction became a yellow-brown solution. The reaction was quenched with saturated solution of NH₄Cl (40 mL). The mixture was extracted with EtOAc (3×100 mL) and the organic layers were combined, dried over Na₂SO₄ and the solvent was removed in vacuo. The product was isolated as a white solid (1.26 g, 48%) after purification by flash chromatography (0-40% Hexanes/EtOAc). MS (m/z): 443.0 (M−H₂O+H). LCMS Ret. time: 2.50. Rf: 0.48 (Hexanes/EtOAc 80/20). ¹H NMR (CDCl₃, δ): 1.49 (m, 4H), 1.78 (quint, J=7.65 Hz, 2H), 1.887 (quint, J=7.65 Hz, 2H), 2.28 (br, 1H), 3.41 (t, J=6.55, 2H), 3.92 (t, J=6.55, Hz, 2H), 6.04, (s, 1H), 6.575 (dd, J=12.61 Hz, J=2.55, 1H), 6.663 (dd, J=8.60 Hz, J=2.75 Hz, 1H), 7.245 (m, 1H), 7.269 (d, J=8.60 Hz, 2H), 7.453 (d, J=8.60 Hz, 2H).

### Step 3

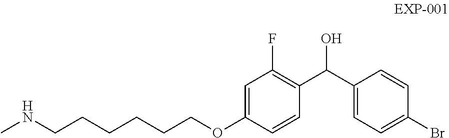 [SMILES: CNCCCCCCOc1ccc(C(O)c2ccc(Br)cc2)c(F)c1]

### EXP-001: 4-bromophenyl)(2-fluoro-4-(6-(methylamino)hexyloxy)phenyl)methanol (LZH-00003)

A solution of IN-001-02 (1.03 g, 2.24 mmol), 40% solution of MeNH₂ (0.6 mL, 6.72 mmol) in dioxane (10 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a light yellow solid (0.38 g, 41%). MS (m/z): 410.2 (M+H), 392.3 (M−H₂O+H), LCMS Ret. time: 1.73. ¹H NMR (CDCl₃, δ): 1.35 (m, 2H), 1.44 (m, 2H), 1.49 (quint, J=7.70 Hz, 2H), 1.753 (quint, J=7.70 Hz, 2H), 2.41 (s, 3H), 2.55 (t, J=7.15 Hz, 2H), 3.903 (t, J=6.70 Hz, 2H), 5.40 (br, 1H), 6.03 (s, 1H), 6.566 (dd, J=12.70 Hz, J=2.75, 1H), 6.655 (dd, J=8.50 Hz, J=2.50 Hz, 1H), 7.26 (m, 3H), 7.442 (d, J=8.50 Hz, 2H).

### Synthesis of Example 2

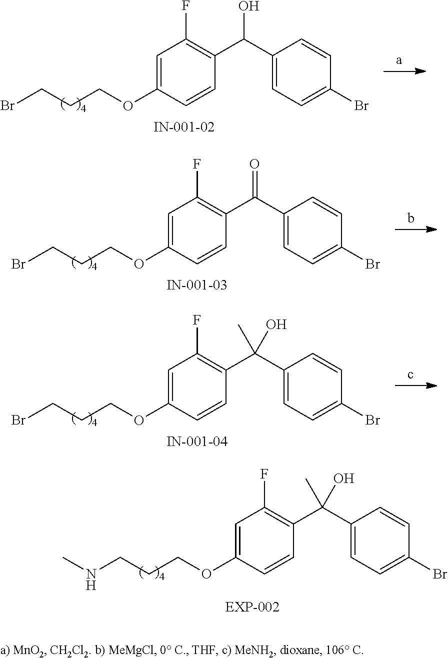

### Step 1

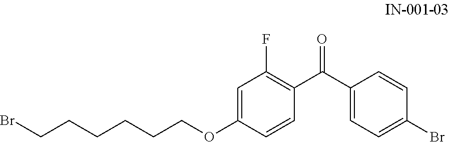 [SMILES: O=C(c1ccc(Br)cc1)c1ccc(OCCCCCCBr)cc1F]

### IN-001-03: 4-(6-bromohexyloxy)-2-fluorophenyl)(4-bromophenyl)methanone

A solution of IN-001-02 (0.88 g, 1.91 mmol) in CH₂Cl₂ (15 mL) containing MnO₂ (3.3 g, 38.2 mmol) was heated at 50° C. for 2 h. Then, the solution was filtered over celite and the solvent was removed in vacuo. The residue was dried in vacuo for 3 h and the product was obtained as a yellow oil (0.70 g, 80%). The product was used without further purification into the next step. MS (m/z): 509.3 (M+H). LCMS Ret. time 3.02. Rf: 0.46 (Hexanes/EtOAc 90/10). ¹H NMR (CDCl₃, δ): 1.48 (m, 4H), 1.79 (quint, J=6.95 Hz, 2H), 1.86 (quint, J=6.95 Hz, 2H), 3.39 (t, J=6.95, 2H), 3.98 (t, J=6.95, Hz, 2H), 6.61 (dd, J=12.05 Hz, J=2.30, 1H), 6.747 (dd, J=8.70 Hz, J=2.45 Hz, 1H), 7.52 (pt, J=8.40 Hz, 1H), 7.56 (d, J=8.40 Hz, 2H), 7.71 (dd, J=8.40 Hz, J=1.55 Hz, 2H).

### Step 2

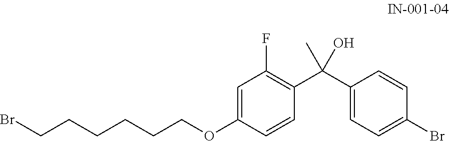 [SMILES: CC(O)(c1ccc(Br)cc1)c1ccc(OCCCCCCBr)cc1F]

### IN-001-04: 1-(4-(6-bromohexyloxy)-2-fluorophenyl)-1-(4-bromophenyl)ethanol

A solution of IN-001-03 (0.40 g, 0.87 mmol) in THF (3 mL) was cooled down using an ice/water bath and then 3M solution of MeMgCl in THF (0.3 mL, 0.87 mmol) was added dropwise. The reaction was stirred for 30 minutes using the ice/water bath and then the reaction was warmed up at room temperature and the mixture was stirred for 2 h. The reaction was quenched with saturated solution of NH₄Cl (15 ml) and diluted with EtOAc (20 mL). The mixture was extracted, the layers were separated and the aqueous phase was extracted with EtOAc (2×25 mL). The combined organic layers were dried over Na₂SO₄ and the solvent was removed in vacuo. The product was obtained as yellow oil (0.37 g, 89%) and it was used into the next step without further purification. MS (m/z): 455.0 (M−H₂O+H). LCMS Ret. time 2.62.

### Step 3

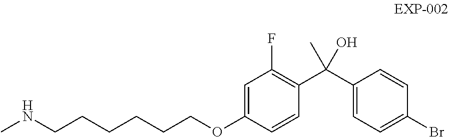 [SMILES: CNCCCCCCOc1ccc(C(C)(O)c2ccc(Br)cc2)c(F)c1]

### EXP-002: 1-(4-bromophenyl)-1-(2-fluoro-4-(6-(methylamino)hexyloxy)phenyl)ethanol (LZH-00004)

A solution of IN-001-04 (0.40 g, 0.84 mmol), 40% solution of MeNH₂ (0.22 mL, 2.53 mmol) in dioxane (7 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a white solid (0.32 g, 89%). MS (m/z): 406.2 (M−H₂O+H). LCMS Ret. time 1.84. ¹H NMR (CDCl₃, δ): 1.39 (m, 2H), 1.47 (m, 2H), 1.57 (quint, J=7.70 Hz, 2H), 1.77 (quint, J=6.75 Hz, 2H), 1.87 (s, 3H), 2.45 (s, 3H), 2.63 (t, J=7.75, Hz, 2H), 3.09 (b, 2H), 3.92 (t, J=16.40 Hz, 2H), 5.51 (b, 1H), 6.53 (dd, J=13.65 Hz, J=2.60, 1H), 6.676 (dd, J=8.90 Hz, J=2.40 Hz, 1H), 7.23 (d, J=8.60 Hz, 2H), 7.40 (d, J=8.60 Hz, 2H), 7.43 (pt, J=9.36 Hz, 1H).

### Synthesis of Example 3

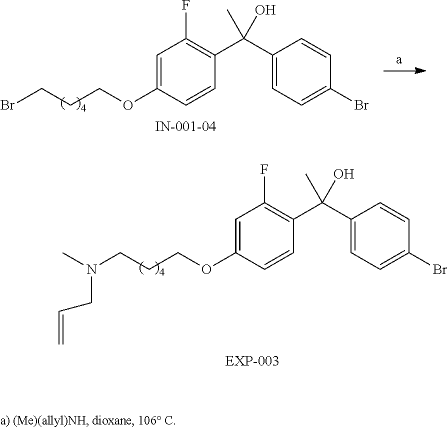

### EXP-003: 1-(4-(6-(allyl(methyl)amino)hexyloxy)-2-fluorophenyl)-1-(4-bromophenyl)ethanol (LZH-00005)

A solution of IN-001-04 (0.40 g, 0.84 mmol), allylmethylamine (243 μL, 2.53 mmol) in dioxane (7 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a white solid (0.36 g, 92%). MS (m/z): 464.2 (M+H), 446.2 (M−H₂O+H). LCMS Ret. time 1.85. ¹H NMR (CDCl₃, δ): 1.36 (quint, J=6.65 Hz, 2H), 1.49 (m, 4H), 1.77 (quint, J=6.65 Hz, 2H), 1.88 (s, 3H), 2.21 (s, 3H), 2.34 (pt, J=7.35, Hz, 2H), 2.74 (br, 1H), 3.00 (d, J=6.65 Hz, 2H), 3.92 (t, J=6.40 Hz, 2H), 5.15 (m, 2H), 5.86 (m, 1H), 6.543 (dd, J=13.80 Hz, J=2.45, 1H), 6.685 (dd, J=8.71 Hz, J=2.40 Hz, 1H), 7.23 (d, J=8.46 Hz, 2H), 7.41 (d, J=8.46 Hz, 2H), 7.425 (pt, J=8.96 Hz, 1H).

### Synthesis of Example 4

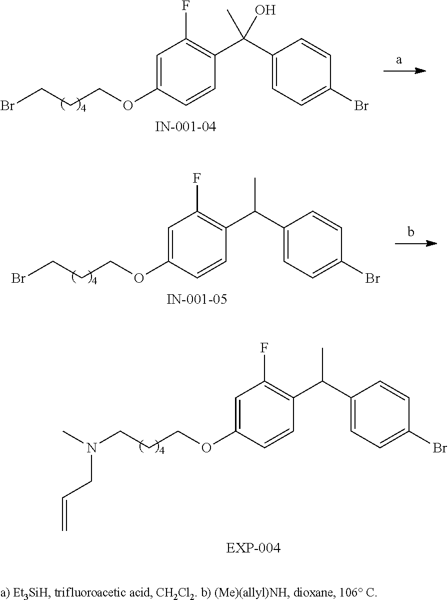

### Step 1

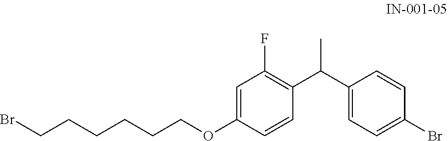 [SMILES: CC(c1ccc(Br)cc1)c1ccc(OCCCCCCBr)cc1F]

### IN-001-05: 4-(6-bromohexyloxy)-1-(1-(4-bromophenyl)ethyl)-2-fluorobenzene

To a solution of IN-001-04 (0.40 g, 0.84 mmol) in CH₂Cl₂ (7 mL), Et₃SiH (1.3 mL, 8.4 mmol) and trifluoroacetic acid (0.8 mL, 8.4 mmol) were added. The reaction was stirred for 2 h at room temperature. Then, the reaction was quenched with saturated solution of NaHCO₃ (aprox. 3 mL), and then the mixture was diluted with CH₂Cl₂ and water (15 mL each). The mixture was extracted, the organic phase separated and the aqueous layer was extracted with CH₂Cl₂ (2×15 mL). The organic layers were combined, dried over Na₂SO₄ and the solvent was removed in vacuo. The residue was purified by flash chromatography (0-45% CH₂Cl₂-Polar solvent, being polar solvent MeOH/NH₄OH 5/1). The product was obtained as a white solid (0.36 g, 92%) MS (m/z): 457.0 (M+H). LCMS Ret. time 2.62.

### Step 2

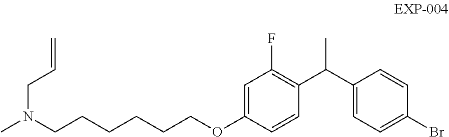 [SMILES: C=CCN(C)CCCCCCOc1ccc(C(C)c2ccc(Br)cc2)c(F)c1]

### EXP-004: N-allyl-6-(4-(1-(4-bromophenyl)ethyl)-3-fluorophenoxy)-N-methylhexan-1-amine (LZH-00006)

A solution of IN-001-05 (0.15 g, 0.33 mmol), allylmethylamine (94.3 μL, 0.98 mmol) in dioxane (5 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a white solid (0.12 g, 82%). MS (m/z): 448.3 (M+H). LCMS Ret. time 2.20. ¹H NMR (CDCl₃, δ): 1.35 (m, 2H), 1.47 (m, 4H), 1.57 (d, J=7.65 Hz, 3H), 1.76 (quint, J=6.30 Hz, 2H), 2.20 (s, 3H), 2.33 (m, 2H), 2.99 (d, J=6.00, Hz, 2H), 3.90 (t, J=6.30 Hz, 2H), 4.32 (q, J=7.65 Hz, 2H), 5.14 (m, 2H), 5.85 (m, 1H), 6.555 (dd, J=12.30 Hz, J=2.45, 1H), 6.625 (dd, J=8.60 Hz, J=2.50 Hz, 1H), 7.05 (pt, J=8.80 Hz, 1H), 7.09 (d, J=8.35 Hz, 2H), 7.385 (d, J=8.35 Hz, 2H).

### Synthesis of Example 5

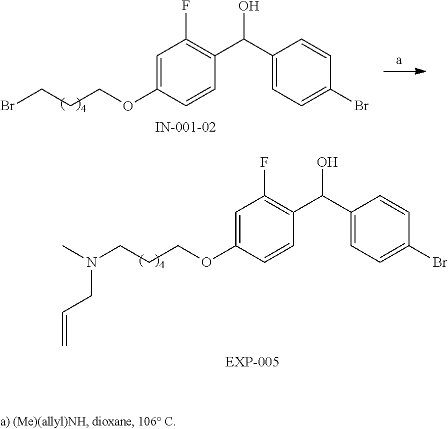

### EXP-005: 4-(6-(allyl(methyl)amino)hexyloxy)-2-fluorophenyl)(4-bromophenyl)methanol (LZH-00007)

A solution of IN-001-02 (0.16 g, 0.35 mmol), allylmethylamine (0.1 mL, 1.05 mmol) in dioxane (5 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a light brown solid (0.13 g, 83%). MS (m/z): 450.3 (M+H), 432.3 (M−H₂O+H), LCMS Ret. time: 1.86. ¹H NMR (CDCl₃, δ): 1.35 (m, 2H), 1.46 (m, 4H), 1.76 (quint, J=6.75 Hz, 2H), 2.19 (s, 3H), 2.32 (m, 2H), 2.55 (br, 1H), 2.98 (d, J=6.30 Hz, 2H), 3.91 (t, J=6.30 Hz, 2H), 5.12 (m, 1H), 5.15 (m, 1H), 5.85 (m, 1H), 6.03 (s, 1H), 6.57 (dd, J=12.20 Hz, J=2.50, 1H), 6.66 (dd, J=8.55 Hz, J=2.45 Hz, 1H), 7.26 (m, 3H), 7.445 (d, J=8.30 Hz, 2H).

### Synthesis of Example 6

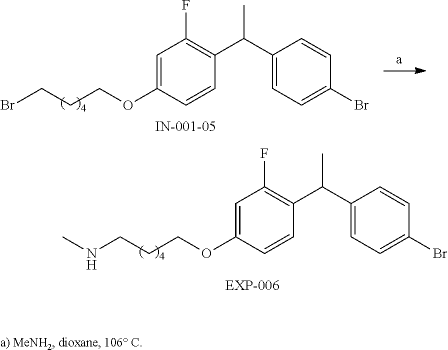

### EXP-006: 6-(4-(1-(4-bromophenyl)ethyl)-3-fluorophenoxy)-N-methylhexan-1-amine (LZH-00008)

A solution of IN-001-05 (0.40 g, 0.84 mmol), 40% solution of MeNH₂ (0.22 mL, 2.53 mmol) in dioxane (7 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a white solid (0.32 g, 89%). MS (m/z): 408.3 (M+H). LCMS Ret. time 1.99. ¹H NMR (DMSO-d₆, δ): 1.35 (m, 4H), 1.44 (quint, J=7.85 Hz, 2H), 1.52 (d, J=7.15 Hz, 3H) 1.675 (q, J=7.17 Hz, 2H), 2.33 (s, 3H), 2.55 (t, J=7.17, Hz, 2H), 3.92 (t, J=6.45 Hz, 2H), 4.28 (q, J=7.20 Hz, 1H), 6.73 (m, 2H), 7.16 (d, J=7.85 Hz, 2H), 7.24 (pt, J=8.66 Hz, 1H), 7.46 (d, J=7.85 Hz, 2H).

### Synthesis of Example 7

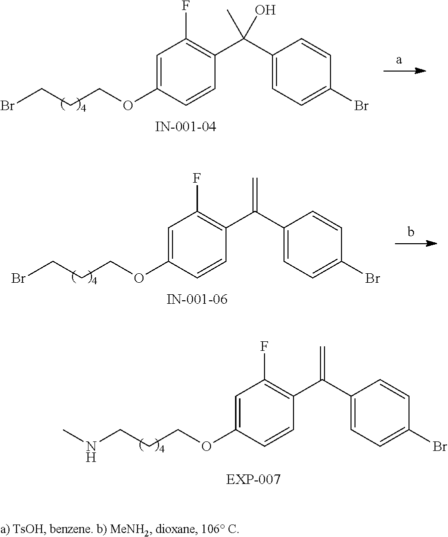

### Step 1

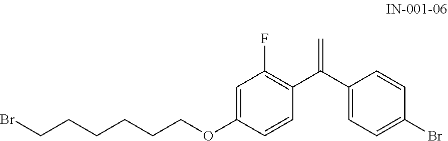 [SMILES: C=C(c1ccc(Br)cc1)c1ccc(OCCCCCCBr)cc1F]

### IN-001-06: 4-(6-bromohexyloxy)-1-(1-(4-bromophenyl)vinyl)-2-fluorobenzene

A solution of IN-001-04 (0.17 g, 0.6 mmol) and p-toluenesulfonic acid (0.018 g, 0.11 mmol) in benzene (9 mL) was stirred at 80° C. f for 1 h and after this time the reaction becomes a brown solution. Then, after cooling down, the reaction was diluted with EtOAc (20 mL) and washed with brine (2×15 mL). The organic layer was dried over Na₂SO₄ and the solvent was removed in vacuo. The product was obtained as a brown oil (0.13 g, 79%).

### Step 2

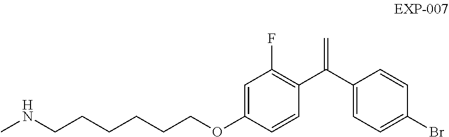 [SMILES: C=C(c1ccc(Br)cc1)c1ccc(OCCCCCCNC)cc1F]

### EXP-007: 6-(4-(1-(4-bromophenyl)vinyl)-3-fluorophenoxy)-N-methylhexan-1-amine (LZH-00009)

A solution of IN-001-06 (0.050 g, 0.11 mmol), 40% solution of MeNH₂ (96 μL, 1.10 mmol) in dioxane (5 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a yellow solid (0.036 g, 81%). MS (m/z): 406.2 (M+H). LCMS Ret. time 2.04. ¹H NMR (CDCl₃, δ): 1.39 (m, 2H), 1.50 (m, 4H), 1.78 (quint, J=6.55 Hz 2H), 2.43 (s, 3H), 2.58 (t, J=6.95 Hz, 2H), 3.94 (t, J=7.00 Hz, 2H), 5.37 (s, 1H), 5.619 (d, J=1.00 Hz, 1H), 6.592 (dd, J=12.06 Hz, J=2.50, 1H), 6.662 (dd, J=8.40 Hz, J=2.30 Hz, 1H), 7.124 (pt, J=8.76 Hz, 1H), 7.17 (d, J=8.51 Hz, 2H), 7.413 (d, J=8.51 Hz, 2H).

### Synthesis of Example 8

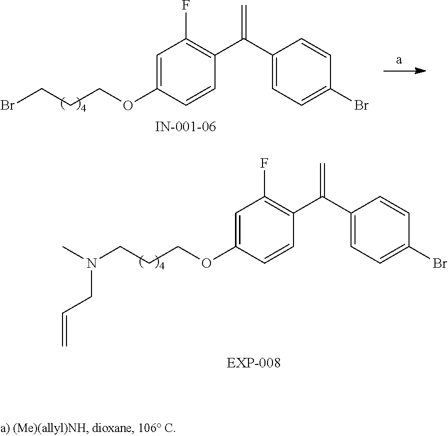

### EXP-008. N-allyl-6-(4-(1-(4-bromophenyl)vinyl)-3-fluorophenoxy)-N-methylhexan-1-amine (LZH-00010)

A solution of IN-001-06 (0.050 g, 0.11 mmol), allylmethylamine (31.5 μL, 033 mmol) in dioxane (5 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a yellow solid (0.035 g, 72%). MS (m/z): 446.3 (M+H). LCMS Ret. time: 2.15. ¹H NMR (CDCl₃, δ): 1.37 (m, 2H), 1.49 (m, 4H), 1.79 (quint, J=6.70 Hz, 2H), 2.215 (s, 3H), 2.345 (m, 2H), 3.00 (d, J=6.35, Hz, 2H), 3.95 (t, J=5.95 Hz, 2H), 5.15 (m, 2H), 5.38 (b, 1H), 5.62 (d, J=0.95 Hz, 1H), 5.86 (m, 1H), 6.607 (dd, J=12.05 Hz, J=2.60, 1H), 6.672 (dd, J=8.50 Hz, J=2.60 Hz, 1H), 7.134 (pt, J=8.60 Hz, 1H), 7.18 (d, J=8.36 Hz, 2H), 7.423 (d, J=8.36 Hz, 2H).

### Synthesis of Example 9

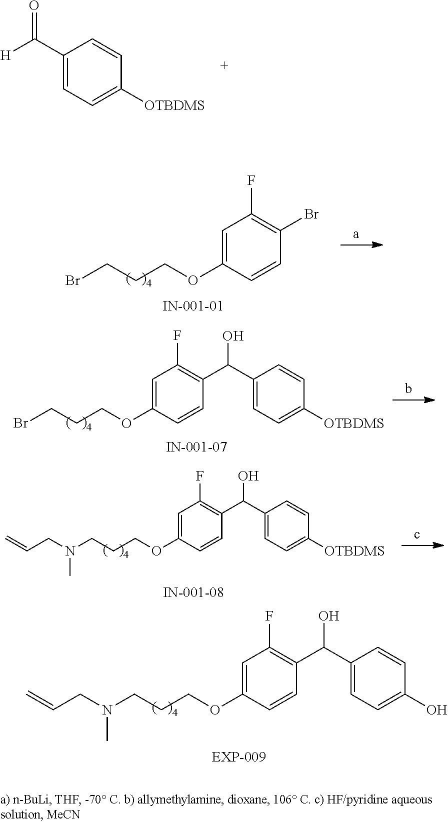

### Step 1

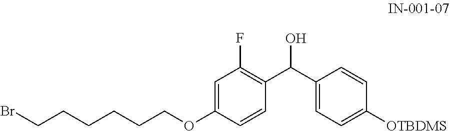 [SMILES: CC(C)(C)[Si](C)(C)Oc1ccc(C(O)c2ccc(OCCCCCCBr)cc2F)cc1]

### IN-001-007: (4-(6-bromohexyloxy)-2-fluorophenyl)(4-(tert-butyldimethylsilyloxy)phenyl)me-thanol

A solution of IN-001-01 (1.5 g, 4.24 mmol) in anhydrous THF (20 mL) under N₂ was cooled down in at −70° C., and then, 1.6 M n-BuLi solution in hexanes was added (2.9 mL, 4.66 mmol) dropwise. After stirring for 2 h, the solution at −70° C., 4-(tert-butyldimethylsilyloxy)benzaldehyde (1 g, 4.24 mmol) dissolved in anhydrous THF (20 mL) was added dropwise. The reaction was stirred for 30 min at −70° C. and then for h on an ice-water bath and at room temperature for an overnight. After this time, the reaction became a yellow-brown solution. The reaction was quenched with saturated solution of NH₄Cl (40 mL). The mixture was extracted with EtOAc (3×60 mL) and the organic layers were combined, dried over Na₂SO₄ and the solvent was removed in vacuo. The product was isolated as a yellow oil (0.62 g, 29%) after purification by flash chromatography (0-25% Hexanes/EtOAc). MS (m/z): 495.1 (M−H₂O+H). LCMS Ret. time: 2.85. Rf: 0.26 (Hexanes/EtOAc 80/20). ¹H NMR (CDCl₃, δ): 0.18 (s, 6H), 0.97 (s, 9H), 1.50 (m, 2H), 1.55 (m, 2H), 1.78 (quint, J=7.05 Hz, 2H), 1.89 (quint, J=6.40 Hz, 2H), 2.14 (br, 1H), 3.42 (t, J=7.05, 2H), 3.92 (t, J=6.40, Hz, 2H), 6.02, (s, 1H), 6.57 (dd, J=12.20 Hz, J=2.35, 1H), 6.666 (dd, J=8.40 Hz, J=2.55 Hz, 1H), 6.79 (d, J=8.65 Hz, 2H), 7.228 (d, J=8.65 Hz, 2H), 7.31 (pt, J=8.65 Hz, 1H).

### Step 2

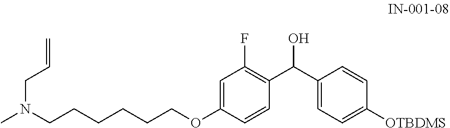 [SMILES: C=CCN(C)CCCCCCOc1ccc(C(O)c2ccc(O[Si](C)(C)C(C)(C)C)cc2)c(F)c1]

### IN-001-008: (4-(6-(allyl(methyl)amino)hexyloxy)-2-fluorophenyl)(4-(tertbutyldimethylsilyloxy)-phenyl)methanol

A solution of IN-001-007 (0.050 g, 0.11 mmol), allylmethylamine (31.5 μL, 0.33 mmol) in dioxane (5 mL) was heated at 106° C. for an overnight. After cooling down the reaction mixture at room temperature, the mixture was diluted with EtOAc and water (25 mL each) and then it was transferred to a separation funnel. The organic phase was separated and the aqueous phase was extracted with EtOAc (2×25 mL). The organic phases were combined, dried over Na₂SO₄ and then the solvent was removed in vacuo. The brown oily residue was dried in vacuo for 2 h, affording the target product as a light yellow solid (0.035 g, 72%), and it was used into the next step without further purification.

### Step 3

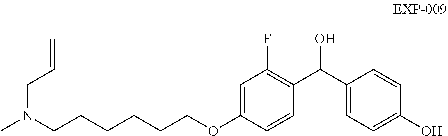 [SMILES: C=CCN(C)CCCCCCOc1ccc(C(O)c2ccc(O)cc2)c(F)c1]

### EXP-009: 4-((4-(6-(allyl(methyl)amino)hexyloxy)-2-fluorophenyl)(hydroxy)methyl)phenol (LZH-00011)

To a solution of IN-001-08 (0.10 g, 0.20 mmol) in MeCN (5 mL), 1.75 M solution of HF/pyridine in MeCN (140 μL, 0.20 mmol) was added. The reaction was stirred at room temperature for 1 h. Then, the reaction was quenched with saturated solution of NaHCO₃ (5 mL). The mixture was then diluted with equal amounts of water and EtOAc (15 mL) and the mixture was extracted. The organic phase was separated and the aqueous phase was extracted with EtOAc (2×15 mL). The combined organic layers were combined, dried over Na₂SO₄ and the solvent was removed in vacuo for 3 h and the product was obtained as a pale yellow solid. MS (m/z): 388.2 (M+H), 370.3 (M−H₂O+H). LCMS Ret. time: 1.53. ¹H NMR (CDCl₃, δ): 1.31 (m, 2H), 1.44 (m, 4H), 1.71 (quint, J=6.50 Hz, 2H), 2.19 (s, 3H), 2.345 (m, 2H), 2.995 (d, J=7.00 Hz, 2H), 3.86 (t, J=5.75 Hz, 2H), 5.13 (m, 2H), 5.86 (m, 1H), 6.49 (m, 1H), 6.63 (m, 1H), 6.72 (m, 2H), 7.14 (m, 2H), 7.35 (m, 1H).

### Synthesis of Example 10

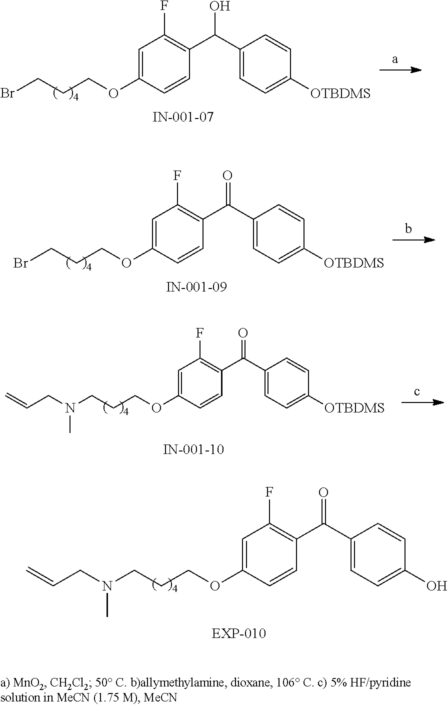

### Step 1

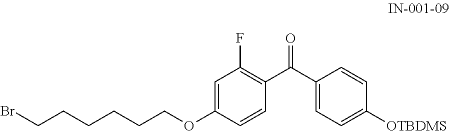 [SMILES: CC(C)(C)[Si](C)(C)Oc1ccc(C(=O)c2ccc(OCCCCCCBr)cc2F)cc1]

### IN-001-09: (4-(6-bromohexyloxy)-2-fluorophenyl)(4-(tert-butyldimethylsilyloxy)phenyl)metha-none

A solution of IN-001-07 (0.88 g, 1.72 mmol) in CH₂Cl₂ (15 mL) containing MnO₂ (2.99 g, 34.4 mmol) was heated at 50° C. for 2 h. Then, the solution was filtered over celite and the solvent was removed in vacuo. The residue was dried in vacuo for 3 h and the product was obtained as a yellow oil (0.65 g, 75%). The product was used without further purification into the next step. MS (m/z): 509.3 (M+H). LCMS Ret. time 3.02. Rf: 0.46 (Hexanes/EtOAc 90/10). ¹H NMR (CDCl₃, δ): 0.21 (s, 6H), 0.96 (s, 9H), 1.50 (m, 4H), 1.807 (quint, J=6.65 Hz, 2H), 1.884 (quint, J=7.15 Hz, 2H), 3.41 (t, J=6.65, 2H), 3.98 (t, J=7.15, Hz, 2H), 6.612 (dd, J=11.56 Hz, J=2.40, 1H), 6.73 (dd, J=7.80 Hz, J=2.20 Hz, 1H), 6.843 (d, J=8.35 Hz, 2H), 7.472 (pt, J=8.35 Hz, 1H), 7.71 (d, J=8.65 Hz, 2H).

### Step 2

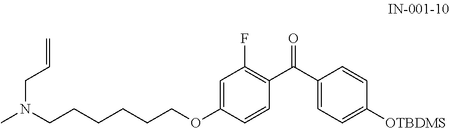 [SMILES: C=CCN(C)CCCCCCOc1ccc(C(=O)c2ccc(O[Si](C)(C)C(C)(C)C)cc2)c(F)c1]

### IN-001-10: (4-(6-(allyl(methyl)amino)hexyloxy)-2-fluorophenyl)(4-(tert-butyldimethylsilyloxy)-phenyl)methanone

A solution of the IN-001-09 (0.1083 g, 0.21 mmol) and allylmethylamine (60.5 μL, 0.63 mmol) in dioxane (5 mL) was heated at 106° C. in a sealed tube for an overnight. After this time, the solvent was removed in vacuo and the residue was purified by flash chromatography (0-100% Hexane-Polar solvent, being polar solvent EtOAc/MeOH 90/10). The target product was obtained as a brown oil (0.094 g, 88%). MS (m/z): 500.3 (M+H). Ret. time LCMS: 2.25. Rf: 0.23 (EtOAc/MeOH 90/10). ¹H NMR (CDCl₃, δ): 0.097 (s, 6H), 0.91 (s, 9H), 1.40 (m, 2H), 1.48 (m, 2H), 1.77 (m, 4H), 2.57 (s, 3H), 2.79 (m, 2H), 3.45 (d, J=7.15 Hz, 2H), 3.95 (t, J=6.20, Hz, 2H), 5.44 (m, 2H), 6.05 (m, 1H), 6.595 (dd, J=11.81 Hz, J=2.45, 1H), 6.715 (dd, J=8.51 Hz, J=2.20 Hz, 1H), 6.90 (d, J=8.80 Hz, 2H), 7.472 (pt, J=8.20 Hz, 1H), 7.73 (d, J=8.80 Hz, 2H).

### Step 3

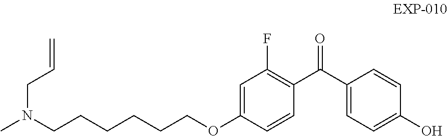 [SMILES: C=CCN(C)CCCCCCOc1ccc(C(=O)c2ccc(O)cc2)c(F)c1]

### EXP-010: (4-(6-(allyl(methyl)amino)hexyloxy)-2-fluorophenyl)(4-hydroxyphenyl)methanone (LZH-00012)

To a solution of IN-001-10 (0.094 g, 0.19 mmol) in MeCN (5 mL), 1.75 M solution of HF/pyridine in MeCN (108.5 μL, 0.19 mmol) was added. The reaction was stirred at room temperature for 1 h. Then, the reaction was quenched with saturated solution of NaHCO₃ (5 mL). The mixture was then diluted with equal amounts of water and EtOAc (15 mL) and the mixture was extracted. The organic phase was separated and the aqueous phase was extracted with EtOAc (2×15 mL). The combined organic layers were combined, dried over Na₂SO₄ and the solvent was removed in vacuo, affording the product as a light brown solid (0.069 g, 95%). MS (m/z): 386.3 (M+H). LCMS Ret. time: 1.58. ¹H NMR (CDCl₃): 1.33 (m, 2H), 1.41 (m, 2H), 2.60 (s, 3H), 2.84 (m, 2H), 3.52 (d, J=7.50 Hz, 2H), 3.88 (t, J=6.40 Hz, 2H), 5.43 (m, 2H), 5.95 (m, 1H), 6.555 (dd, J=11.95 Hz, J=2.40, 1H), 6.672 (dd, J=8.65 Hz, J=2.40 Hz, 1H), 6.89 (d, J=8.80 Hz, 2H), 7.43 (pt, J=8.30 Hz, 1H), 7.71 (d, J=8.80 Hz, 2H).

### Synthesis of Example 11

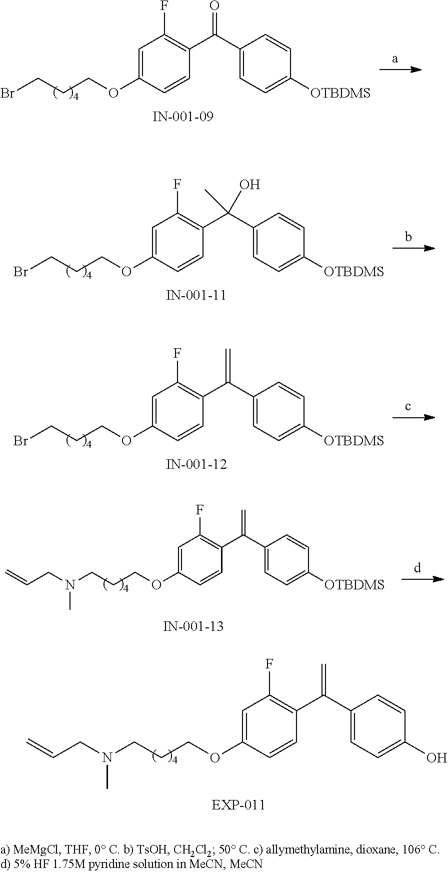

### Step 1

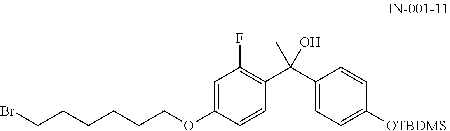 [SMILES: CC(O)(c1ccc(O[Si](C)(C)C(C)(C)C)cc1)c1ccc(OCCCCCCBr)cc1F]

### IN-001-11: 1-(4-(6-bromohexyloxy)-2-fluorophenyl)-1-(4-(tert-butyldimethylsilyloxy)phenyl)-ethanol

A solution of IN-001-09 (0.25 g, 0.49 mmol) in THF (3 mL) was cooled down using an ice/water bath and then 3M solution of MeMgCl in THF (0.2 mL, 0.54 mmol) was added dropwise. The reaction was stirred for 30 minutes using the ice/water bath and then the reaction was warmed up at room temperature and the mixture was stirred for 2 h. The reaction was quenched with saturated solution of NH₄Cl (15 ml) and diluted with EtOAc (20 mL). The mixture was extracted, the layers were separated and the aqueous phase was extracted with EtOAc (2×25 mL). The combined organic layers were dried over Na₂SO₄ and the solvent was removed in vacuo. The product was obtained as yellow oil (0.23 g, 89%) and it was used into the next step without further purification. MS (m/z): 507.6 (M−H₂O+H). LCMS Ret. time 2.74. Rf: 0.3 (Hexanes/EtOAc 90/10). ¹H NMR (CDCl₃): 0.18 (s, 6H), 0.96 (s, 9H), 1.50 (m, 4H), 1.79 (m, 2H), 1.90 (m, 2H), 2.04 (s, 3H), 3.00 (b, 1H), 3.42 (t, J=6.15 Hz, 2H), 3.93 (t, J=6.55 Hz, 2H), 6.54 (dd, J=13.45 Hz, J=2.50, 1H), 6.66 (dd, J=8.60 Hz, J=2.40 Hz, 1H), 6.74 (d, J=8.60 Hz, 2H), 7.20 (d, J=8.60 Hz, 2H), 7.42 (pt, J=9.05 Hz, 1H).

### Step 2

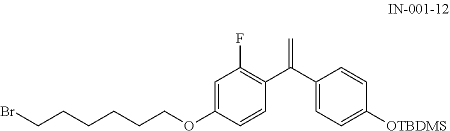 [SMILES: C=C(c1ccc(O[Si](C)(C)C(C)(C)C)cc1)c1ccc(OCCCCCCBr)cc1F]

### IN-001-12: (4-(1-(4-(6-bromohexyloxy)-2-fluorophenyl)vinyl)phenoxy)tert-butyl)dimethylsi-lane

A solution of IN-001-11 (0.23 g, 0.44 mmol) and p-toluenesulfonic acid (0.025 g, 0.14 mmol) in benzene (5 mL) was stirred at 80° C. f for 1 h and after this time the reaction becomes a brown solution. Then, after cooling down, the reaction was diluted with EtOAc (20 mL) and washed with brine (2×15 mL). The organic layer was dried over Na₂SO₄ and the solvent was removed in vacuo. The product was obtained as a brown oil (0.22 g, 100%) and it was used into the next step without further purification. MS (m/z): 507.7 (M−H₂O+H). LCMS Ret. time 2.99. The crude, according to the LCMS also contains the dehydration product with the unprotected phenol moiety. MS (m/z): 393.0 (M−H₂O+H). LCMS Ret. time 2.30.

### Step 3

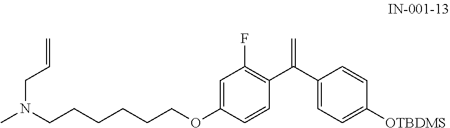 [SMILES: C=CCN(C)CCCCCCOc1ccc(C(=C)c2ccc(O[Si](C)(C)C(C)(C)C)cc2)c(F)c1]

### IN-001-13: N-allyl-6-(4-(1-(4-(tert-butyldimethylsilyloxy)phenyl)vinyl)-3-fluorophenoxy)-N-methylhexan-1-amine

A solution of 22 (0.22 g, 0.44 mmol) and allylmethylamine (126 μL, 13.2 mmol) in dioxane (5 mL) was stirred at 106° C. in a sealed tube for an overnight. After this time, the solvent was removed in vacuo and the residue was purified by flash chromatography (0-100% Hexane-Polar solvent, being polar solvent EtOAc/MeOH 90/10). The target product was obtained as a brown oil (0.217 g, 100%). MS (m/z): 500.3 (M+H). Ret. time LCMS: 2.25. Rf: 0.23 (EtOAc/MeOH 90/10). According to the LCMS, the sample also contains the dehydration product with the unprotected phenol moiety. MS (m/z): 384.2 (M+H). LCMS Ret. time: 1.64.

### Step 4

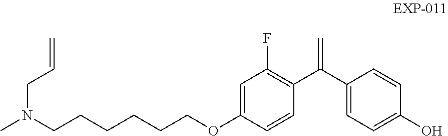 [SMILES: C=CCN(C)CCCCCCOc1ccc(C(=C)c2ccc(O)cc2)c(F)c1]

### EXP-011: 4-(1-(4-(6-(allyl(methyl)amino)hexyloxy)-2-fluorophenyl)vinyl)phenol (LZH-00013)

To a solution of IN-001-13 (0.217 g, 0.144 mmol) in MeCN (5 mL), 1.75 M solution of HF/pyridine in MeCN (228.5 μL, 0.40 mmol) was added. The reaction was stirred at room temperature for 1 h. Then, the reaction was quenched with saturated solution of NaHCO₃ (5 mL). The mixture was then diluted with equal amounts of water and EtOAc (15 mL) and the mixture was extracted. The organic phase was separated and the aqueous phase was extracted with EtOAc (2×15 mL). The combined organic layers were combined, dried over Na₂SO₄ and the solvent was removed in vacuo for 3 h and the product was obtained as a brown oil. MS (m/z): 384.2 (M+H). LCMS Ret. time: 1.64. ¹H NMR (CDCl₃): 1.36 (m, 2H), 1.47 (m, 2H), 1.63 (quint, J=7.05 Hz, 2H), 1.76 (quint, J=7.05 Hz, 2H), 2.41 (s, 3H), 2.61 (m, 2H), 3.27 (d, J=7.50 Hz, 2H), 3.92 (t, J=6.15, Hz, 2H), 5.27 (m, 3H), 5.56 (d, J=1.30, Hz, 1H), 6.58 (dd, J=11.86 Hz, J=2.40, 1H), 6.644 (dd, J=8.40 Hz, J=2.40 Hz, 1H), 6.775 (d, J=8.60 Hz, 2H), 7.14 (pt, J=8.75 Hz, 1H), 7.183 (d, J=8.60 Hz, 2H).

### Synthesis of Example 12

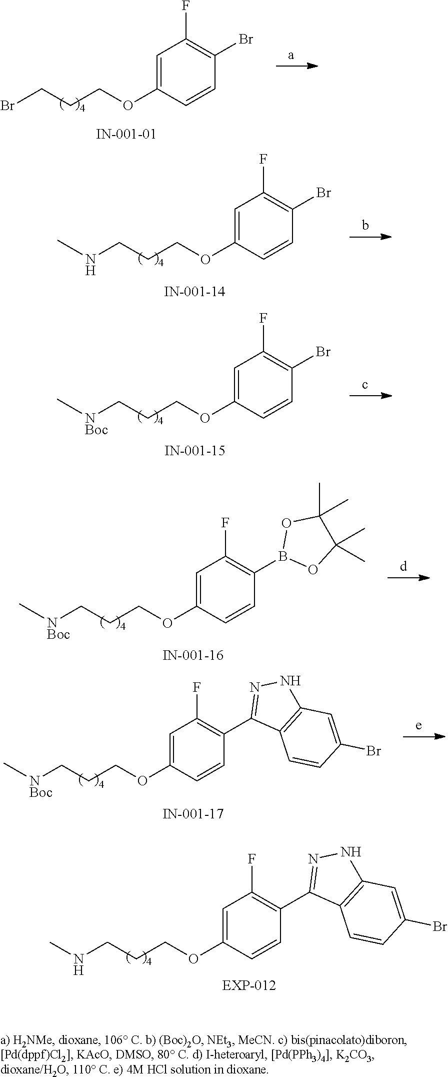

### Step 1

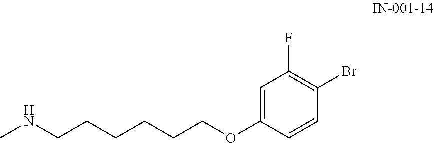 [SMILES: CNCCCCCCOc1ccc(Br)c(F)c1]

### IN-001-14: 6-(4-bromo-3-fluorophenoxy)-N-methylhexan-1-amine

A sealed tube was charged with IN-001-01 (2 g, 5.65 mmol), 40% solution of MeNH₂ (1.5 mL, 16.95 mmol) and dioxane (50 mL). The solution was heated at 106° C. for an overnight. After cooling down the reaction mixture at room temperature, the mixture was diluted with EtOAc and water (50 mL each) and then it was transferred to a separation funnel and the mixture was extracted. The organic phase was separated and the aqueous phase was extracted with EtOAc (2×50 mL). The organic phases were combined, dried over Na₂SO₄ and then the solvent was removed in vacuo. The brown oily residue was dried in vacuo for 2 h, affording the target product as a light brown solid (1.16 g, 93%). MS (m/z): 304.1 (M+H). LCMS Ret. time: 1.67. The product was used in the next step without further purification.

### Step 2

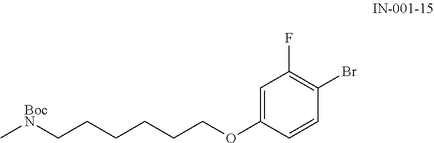 [SMILES: CN(CCCCCCOc1ccc(Br)c(F)c1)C(=O)OC(C)(C)C]

### IN-001-15. Tert-butyl 6-(4-bromo-3-fluorophenoxy)hexyl(methyl)carbamate

To solution of the IN-001-14 (1.67 g, 5.49 mmol) in anhydrous MeCN (35 mL), NEt₃ (1.5 mL, 10.98 mmol) was added. Then, under stirring, a solution of Boc₂O (1.32 g, 6.04 mmol) in MeCN (15 mL) was added. The reaction was stirred at room temperature for 2 h. Then, the reaction was quenched adding water (50 mL) and the mixture was transferred to a separation funnel and the aqueous phase was extracted with EtOAc (3×50 mL) and the combined organic layers were washed with brine (1×70 mL). The organic phase was dried over Na₂SO₄ and the solvent was removed in vacuo. The crude was purified by flash chromatography (0-35% Hexanes-EtOAc), affording the target product as a light yellow oil (1.51 g, 68%). MS (m/z): 404.3 (M+H), 348.1 (M-tBu+H), 304.3 (M+Boc+H). LCMS Ret. time: 2.69. Rf: 0.54 (Hexanes/EtOAc 80/20). ¹H NMR (CDCl₃, δ): 1.23 (m, 2H), 1.35 (s, 9H), 1.41 (m, 4H), 1.67 (quint, J=7.05 Hz, 2H), 2.76 (s, 3H), 3.14 (m, 2H), 3.84 (t, J=6.85 Hz, 2H), 6.52 (ddd, J=8.90 Hz, J=3.10 Hz, J=0.5 Hz, 1H), 6.00 (dd, J=10.60 Hz, J=2.90 Hz, 1H), 7.31 (pt, J=8.40 Hz, 1H).

### Step 3

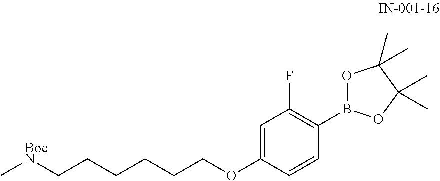

### IN-001-16: Tert-butyl-6-(3-fluoro-4-(4,4,5,5-tetramethyl-1,3,2-dioxaborolan-2-yl)phenoxy)-hexyl(methyl)carbamate

A round-bottom flask was charged with IN-001-15 (1.43 g, 3.54 mmol), KOAc (1.042 g, 10.62 mmol), bis(pinacolato)diboron (0.90 g, 3.54 mmol) and [Pd(dppf)Cl₂].DCM (0.29 g, 0.35 mmol). Then, the system was put under vacuum and under vacuum anhydrous DMSO (30 mL) was added via syringe. Then, the system was put under N₂ atmosphere and the reaction was heated at 80° C. for an overnight. After cooling down, the mixture was diluted with EtOAc (80 mL) and the solution was extracted with water (4×60 mL). The organic phase was dried over Na₂SO₄ and the solvent was removed in vacuo and the residue was then purified by flash chromatography (0-40% Hexanes-EtOAc) and after the evaporation of the solvent the product was obtained as a yellow oil (1.0 g, 64%). MS(m/z): 452.4 (M+H), 396.3 (M-tBu+H), 296.2 (M-tBu-B(OR)₂+H). LCMS Ret. time: 2.77. Rf: 0.5 (Hexanes/EtOAc 80/20). ¹H NMR (CDCl₃, δ): 1.33 (m, 2H), 1.34 (s, 12H) 1.45 (s, 9H), 1.51 (m, 4H), 1.77 (quint, J=6.30 Hz, 2H), 2.83 (s, 3H), 3.20 (b, 2H), 3.95 (t, J=6.35 Hz, 2H), 6.54 (dd, J=11.36 Hz, J=2.20 Hz, 1H), 6.66 (dd, J=8.51 Hz, J=2.40 Hz, 1H), 7.63 (dd, J=7.85 Hz, J=7.40 Hz, 1H).

### Step 4

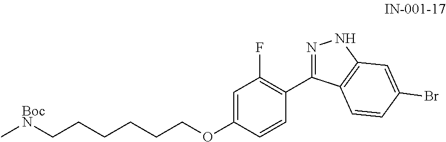 [SMILES: CN(CCCCCCOc1ccc(-c2n[nH]c3cc(Br)ccc23)c(F)c1)C(=O)OC(C)(C)C]

### IN-001-17: Tert-butyl 6-(4-(6-bromo-1H-indazol-3-yl)-3-fluorophenoxy)hexyl(methyl)carba-mate

A scintillation vial was charged with the 6-bromo-3iodo-1H-indazole (0.578 g, 1.79 mmol), K₂CO₃ (0.742 g, 5.37 mmol), and [Pd(PPh₃)₄] (0.103 g, 0.09 mmol). Then, the system was put under vacuum and under vacuum 25 mL of a dioxane solution of the boronic ester IN-001-16 (0.890 g, 1.97 mmol) and water (2.5 mL) were added via syringe. Then, the system was put under N₂ atmosphere and the reaction was heated at 80° C. for an overnight. After this time, the solvent was removed in vacuo and the residue was then purified by flash chromatography (0-30% Hexanes-EtOAc) and after the evaporation of the solvent, the product was obtained as a yellow oil (0.236 g, 25%). MS (m/z): 463.2 (M-tBu+H), 419.3 (M-Boc+H). LCMS Ret. time: 2.77. Rf: 0.48 (Hexanes/EtOAc 80/20). ¹H NMR (CDCl₃, δ): 1.36 (m, 2H), 1.47 (s, 9H), 1.53 (m, 4H), 1.82 (br, 2H), 2.85 (s, 3H), 3.22 (br, 2H), 4.02 (br, 2H), 6.83 (m, 2H), 7.26 (dd, J=8.65 Hz, J=1.55 Hz, 1H), 7.69 (m, 2H).

### Step 5

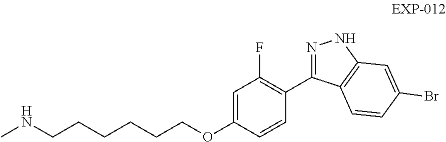 [SMILES: CNCCCCCCOc1ccc(-c2n[nH]c3cc(Br)ccc23)c(F)c1]

### EXP-012: 6-(4-(6-bromo-1H-indazol-3-yl)-3-fluorophenoxy)-N-methylhexan-1-amine (LZH-00014)

The N-Boc protected amine IN-001-17 (0.236 g, 0.45 mmol) was dissolved in 4M HCl solution in dioxane (20 mL), and the mixture was stirred for 1 h at room temperature. The reaction was quenched by addition of saturated solution of NaHCO₃ until no more gas was produced and the pH of the solution was about 8 (aprox. 80 mL). Then, the mixture was diluted with EtOAc (80 mL) and the mixture was extracted. The organic phase was separated and the aqueous phase was extracted with EtOAc (2×80 mL). The organic layers were combined and washed with brine (50 mL), dried over Na₂SO₄ and then the solvent was evaporated in vacuo. The solid yellow residue obtained after the evaporation was purified by flash chromatography (0-40% CH₂Cl₂/Polar solvent, being polar solvent MeOH/NH₄OH 5/1) and the target product was isolated as a white solid (0.188 g, 99%). MS (m/z): 420.2 (M+H). LCMS Ret. time 1.73. Rf: 0.23 (80/20 CH₂Cl₂/Polar solvent, being polar solvent MeOH/NH₄OH 5/1). ¹H NMR (DMSO-d₆, δ): 1.43 (m, 4H), 1.63 (s, 2H), 1.63 (quint, J=7.45 Hz, 2H), 1.76 (quint, J=7.10 Hz, 2H), 2.52 (s, 3H), 2.86 (pt, 2H), 4.07 (t, J=6.50 Hz, 2H), 6.94 (dd, J=8.65 Hz, J=2.50, 1H), 7.01 (dd, J=12.96 Hz, J=2.75 Hz, 1H), 7.30 (dd, J=8.95 Hz, J=1.55 Hz, 1H), 7.67 (m, 2H), 7.82 (d, J=1.65 Hz, 1H), 8.72 (br, 1H).

### Synthesis of Example 13

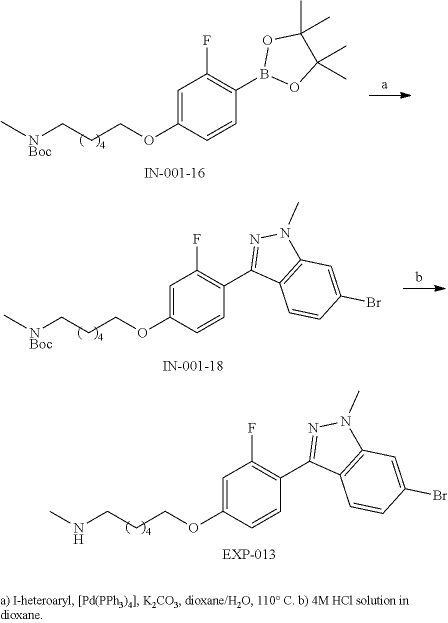

### Step 1

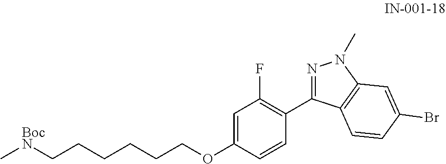 [SMILES: CN(CCCCCCOc1ccc(-c2nn(C)c3cc(Br)ccc23)c(F)c1)C(=O)OC(C)(C)C]

### IN-001-18: Tert-butyl-6-(4-(6-bromo-1-methyl-H-indazol-3-yl)-3-fluorophenoxy)hexyl(methyl)carbamate

Compound IN-001-18 was prepared as it was described for IN-001-017 on using 6-bromo-3-iodo-1-methyl-indazole (0.258 g, 0.77 mmol), boronic ester IN-001-16 (0.265 g, 0.59 mmol), K₂CO₃ (0.245 g, 1.77 mmol), and [Pd(PPh₃)₄] 1 (0.034 g, 0.03 mmol), and water (1 mL) and dioxane (10 mL) as solvents. The product was obtained as a pale yellow oil (0.174 g, 55%) after purification by flash chromatography (0-30% Hexanes-EtOAc). MS (m/z): 534.3 (M+H), 478.2 (M-tBu+H), 434.3 (M-Boc+H). LCMS Ret. time: 2.84. Rf: 0.48 (Hexanes/EtOAc 80/20). ¹H NMR (CDCl₃, δ): 1.36 (m, 2H), 1.46 (s, 9H), 1.53 (m, 4H), 1.82 (m, 2H), 2.84 (s, 3H), 3.22 (br, 2H), 4.00 (t, J=6.10 Hz, 2H), 4.08 (s, 3H), 6.76 (dd, J=12.31 Hz, J=2.40 Hz, 1H), 6.81 (dd, J=8.60 Hz, J=2.40 Hz, 1H), 7.27 (dd, J=8.60 Hz, J=1.55 Hz, 1H), 7.59 (dd, J=1.75 Hz, J=0.45 Hz, 1H), 7.64 (m, 1H), 7.67 (m, 1H).

### Step 2

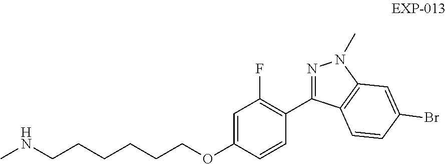 [SMILES: CNCCCCCCOc1ccc(-c2nn(C)c3cc(Br)ccc23)c(F)c1]

### EXP-013: 6-(4-(6-bromo-1-methyl-1H-indazol-3-yl)-3-fluorophenoxy)-N-methylhexan-1-amine (LZH-00015)

Compound EXP-013 was prepared as it was described for EXP-012 on using IN-001-18 (0.386 g, 0.72 mmol) and 4M HCl solution in dioxane (5 mL). The product was obtained as a white solid (0.188 g, 99%) after purification by flash chromatography (0-40% CH₂Cl₂-Polar solvent, being polar solvent MeOH/NH₄OH 5/1). MS (m/z): 434.2 (M+H). LCMS Ret. time 1.85. Rf: 0.28 (80/20 CH₂Cl₂/Polar solvent, being polar solvent MeOH/NH₄OH 5/1). ¹H NMR (CDCl₃, δ): 1.52 (m, 2H), 1.82 (quint, J=6.75 Hz, 2H), 1.926 (quint, J=7.85 Hz, 2H), 2.686 (s, 3H), 2.96 (m, 2H), 3.985 (t, J=6.35 Hz, 2H), 4.07 (s, 3H), 6.74 (dd, J=11.6 Hz, J=2.60, 1H), 6.788 (dd, J=8.20 Hz, J=2.50 Hz, 1H), 7.245 (dd, J=10.1 Hz, J=1.75 Hz, 1H), 7.572 (m, 2H), 7.65 (m, 2H), 9.53 (br, 1H).

### Synthesis of Example 14

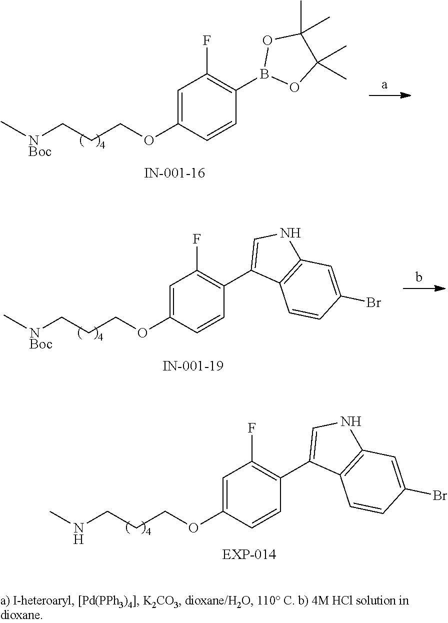

### Step 1

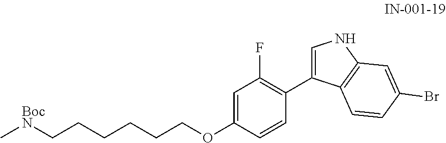 [SMILES: CN(CCCCCCOc1ccc(-c2c[nH]c3cc(Br)ccc23)c(F)c1)C(=O)OC(C)(C)C]

### IN-001-19: Tert-buty-6-(4-(6-bromo-1H-indol-3-yl)-3-fluorophenoxy)hexyl(methyl)carbamate

Compound IN-001-19 was prepared as it was described for IN-001-17 on using 6-bromo-3-iodo-1-tosyl-indole (0.89 g, 1.97 mmol) previously synthesized from 6-bromo-indole (vide infra), boronic ester IN-01-16 (0.72 g, 1.51 mmol), K₂CO₃ (0.63 g, 4.53 mmol), and [Pd(PPh₃)₄] 1 (0.087 g, 0.08 mmol), and water (1.5 mL) and dioxane (15 mL) as solvents. The N-tosyl product was obtained as a pale yellow oil (0.77 g, 76%) after purification by flash chromatography (0-30% Hexanes-EtOAc). MS (for the N-tosyl derivative) (m/z): 617.3 (M-tBu+H), 573.3 (M-Boc+H). LCMS Ret. time: 3.11. Rf: 0.46 (Hexanes/EtOAc 80/20). Then, the N-tosyl indole product (0.110 g, 0.16 mmol) was dissolved in a mixture THF (2 mL) and MeOH (1 mL) and 50% NaOH solution (1 mL) was added and the mixture was stirred at 60° C. for 2 h. Then, the mixture was diluted with water (25 mL) and EtOAc (25 mL) and the mixture was extracted. The organic phase was evaporated and the aqueous phase was extracted with EtOAc (1×25 mL). The organic layers were combined and dried over Na₂SO₄, the solvent was removed in vacuo and the brown residue was purified by flash chromatography (0-60% Hexanes-EtOAc). After the evaporation of the solvent, the residue was dried in vacuo for 2 h, affording the target product as a red brown oil (0.084 g, 99%). MS: 463.2 m/z (M-tBu+H), 419.3 (M-Boc+H). LCMS Ret. time: 2.77. Rf: 0.28. (Hexanes/EtOAc 80/20). ¹H NMR (CDCl₃, δ): 1.36 (m, 2H), 1.46 (s, 9H), 1.53 (m, 4H), 1.80 (m, 2H), 2.84 (s, 3H), 3.22 (pt, J=6.55 Hz 2H), 3.98 (t, J=6.55 Hz, 2H), 6.74 (dd, J=12.05 Hz, J=2.70 Hz, 1H), 6.775 (dd, J=8.30 Hz, J=2.70 Hz, 1H), 7.265 (dd, J=8.45 Hz, J=1.70 Hz, 1H), 7.367 (dd, J=2.45 Hz, J=1.65 Hz, 1H), 7.508 (pt, J=8.55 Hz 1H), 7.58 (d, J=1.80 Hz, 1H), 7.622 (d, J=8.55 Hz, 1H), 8.33 (br, 1H).

### Step 2

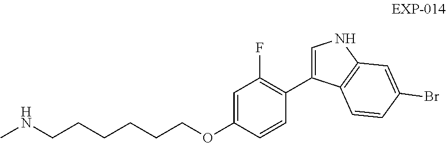 [SMILES: CNCCCCCCOc1ccc(-c2c[nH]c3cc(Br)ccc23)c(F)c1]

### EXP-014: 6-(4-(6-bromo-1H-indol-3-yl)-3-fluorophenoxy)-N-methylhexan-1-amine (LZH-00016)

Compound EXP-014 was prepared as it was described for EXP-012 on using IN-001-19 (0.084 g, 0.16 mmol) and 4M HCl solution in dioxane (5 mL). The product was obtained as a light brown solid (0.054 g, 80%) after the evaporation of the solvent from the work up, and the addition of TBME to the residue. MS (m/z): 420.3 (M+H). LCMS Ret. time: 1.75. ¹H NMR (DMSO-d₆, δ): 1.389 (quint, J=7.55 Hz, 2H), 1.457 (quint, J=7.15 Hz, 2H), 1.624 (quint, J=7.95 Hz, 2H), 1.75 (quint, J=7.15 Hz, 2H), 2.536 (s, 3H), 2.87 (m, 2H), 4.03 (t, J=6.40 Hz, 2H), 6.87 (dd, J=7.15 Hz, J=2.40, 1H), 6.932 (dd, J=12.36 Hz, J=2.40 Hz, 1H), 7.19 (dd, J=8.56 Hz, J=1.80 Hz, 1H), 7.53 (m, 1H), 7.554 (m, 1H), 7.64 (d, J=1.80 Hz, 1H) 8.60 (br, 1H), 11.53 (br, 1H).

### Synthesis of Example 15

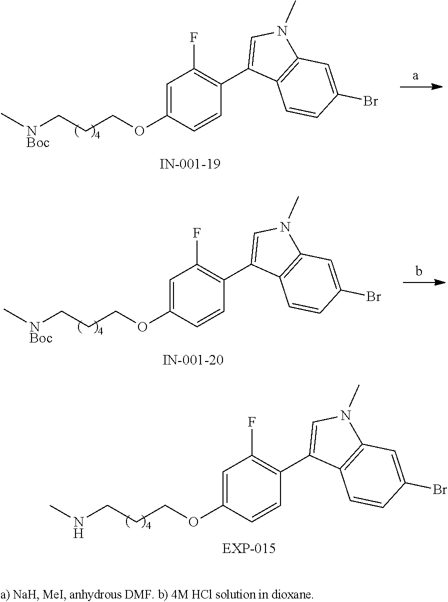

### Step 1

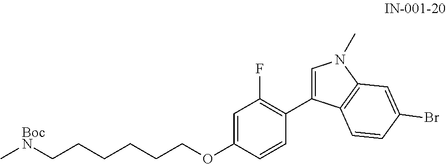 [SMILES: CN(CCCCCCOc1ccc(-c2cn(C)c3cc(Br)ccc23)c(F)c1)C(=O)OC(C)(C)C]

### IN-001-20: Tert-butyl-6-(4-(6-bromo-1-methyl-1H-indol-3-yl)-3-fluorophenoxy)hexyl(methyl)-carbamate

To a stirred solution of IN-001-19 (0.26 g, 0.50 mmol) in anhydrous DMF (10 mL), NaH (0.030 g, containing 0.018 g NaH, 0.75 mmol) was added portionwise. Then, MeI was added (0.034 mL, 0.55 mmol) was added. The reaction was stirred at room temperature for 1 h. The reaction was quenched with water (25 mL) and the mixture was extracted with EtOAc (3×25 mL) and the combined organic phases, after partial evaporation of the solvent to approximately half of the volume, were washed with water (3×25 mL). The organic layer was dried over Na₂SO₄ and the solvent was removed in vacuo. The product was obtained as a yellow oil (0.218 g, 82%) after purification by flash chromatography (0-40% Hexanes-EtOAc). MS: 477.1 m/z (M-tBu+H), 433.1 m/z (M-Boc+H). LCMS Ret. time: 2.95. Rf: 0.47 (Hexanes/EtOAc 80/20). ¹H NMR (CDCl₃, δ): 1.36 (m, 2H), 1.46 (s, 9H), 1.53 (m, 4H), 1.8l(quint, J=7.25 Hz 2H), 2.842 (s, 3H), 3.22 (br, 2H), 3.805 (s, 3H), 3.982 (t, J=6.60 Hz, 2H), 6.75 (dd, J=12.05 Hz, J=2.20 Hz, 1H), 6.77 (dd, J=8.25 Hz, J=2.55 Hz, 1H), 7.235 (dd, J=8.25 Hz, J=1.60 Hz, 1H), 7.9 (m, 2H), 7.615 (d, J=8.25 Hz, 1H).

### Step 2

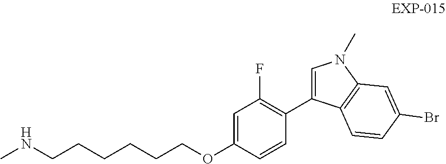 [SMILES: CNCCCCCCOc1ccc(-c2cn(C)c3cc(Br)ccc23)c(F)c1]

### EXP-015. 6-(4-(6-bromo-1-methyl-1H-indol-3-yl)-3-fluorophenoxy)-N-methylhexan-1-amine (LZH-00017)

Compound EXP-015 was prepared as it was described for EXP-012 on using IN-001-20 (0.218 g, 0.16 mmol) and 4M HCl solution in dioxane (5 mL). The product was obtained as a light brown solid (0.135 g, 76%) after the evaporation of the solvent from the work up, and the addition of TBME to the residue. MS (m/z): 433.2 (M+H). LCMS Ret. time: 1.97. ¹H NMR (CDCl₃, δ): 1.39 (m, 2H), 1.452 (m, 1H), 1.73 (quint, J=7.80 Hz, 2H), 1.77 (quint, J=6.75 Hz, 2H), 2.55 (s, 3H), 2.82 (m, 2H), 3.723 (s, 3H), 3.89 (t, J=6.35 Hz, 2H), 6.642 (dd, J=12.50 Hz, J=2.30, 1H), 6.673 (dd, J=8.50 Hz, J=2.55 Hz, 1H), 7.145 (m, 2H), 7.41 (m, 2H), 7.51 (m, 1H), 9.45 (br, 1H).

### Synthesis of Example 16

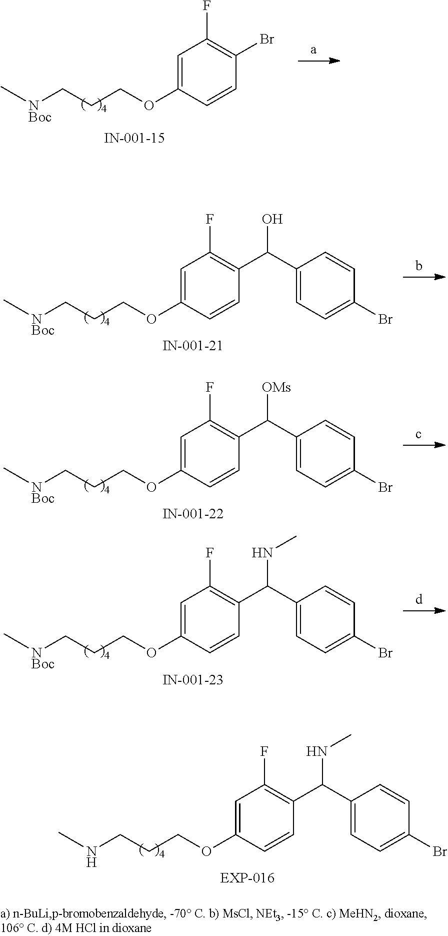

### Step 1

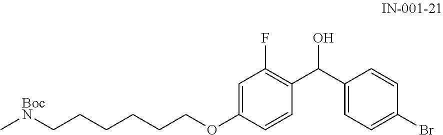 [SMILES: CN(CCCCCCOc1ccc(C(O)c2ccc(Br)cc2)c(F)c1)C(=O)OC(C)(C)C]

### IN-001-21. Tert-butyl-6-(4-((4-bromophenyl)(hydroxy)methyl)-3-fluorophenoxy)hexyl(methyl)-carbamate

To a solution of aryl bromide IN-001-15 (1.41 g, 3.49 mmol) in anhydrous THF (15 mL) at −70° C. and under N₂ atmosphere, 1.6 M solution of n-BuLi in hexanes (2.4 mL, 3.84 mmol) was added dropwise. The mixture reaction was stirred at −70° C. for 2 h and then, a solution of p-bromobenzaldehyde (0.65 g, 3.49 mmol) in THF (15 mL) was added. The reaction was stirred for 30 min at −70° C. and then the reaction was warmed up at room temperature and stirred for 2 h. The reaction was quenched with saturated solution of NH₄Cl (30 mL). The mixture was extracted with EtOAc (3×50 mL) and the organic layers were combined, dried over Na₂SO₄ and the solvent was removed in vacuo. The product was obtained as a light yellow oil (1.56 g, 88%) after purification by flash chromatography (0-45% Hexanes/EtOAc). MS (m/z): 492.2 (M−H₂O+H), 436.1 (M−H₂O-tBu+H). LCMS Ret. time: 2.59. Rf: 0.29 (Hexanes/EtOAc 80/20). ¹H NMR (CDCl₃, δ): 1.32 (m, 2H), 1.447 (s, 9H), 1.51 (m, 2H), 1.763 (quint, J=8.66 Hz), 2.236 (d, J=3.15 Hz, 1H), 2.823 (s, 3H), 3.20 (br, 2H), 3.91 (t, J=6.30 Hz, 2H), 6.042 (d, J=3.15 Hz, 1H), 7.573 (dd, J=12.25 Hz, J=2.45 Hz, 1H), 6.66 (dd, J=8.55 Hz, J=2.65 Hz, 1H), 7.243 (d, J=8.36, 1H), 7.273 (d, J=8.35, 1H), 7.455 (d, J=8.35, 1H).

### Step 2

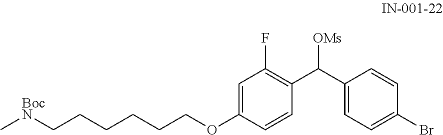 [SMILES: CN(CCCCCCOc1ccc(C(OS(C)(=O)=O)c2ccc(Br)cc2)c(F)c1)C(=O)OC(C)(C)C]

### IN-001-22. (4-bromophenyl)(4-(6-(tert-butoxycarbonyl(methyl)amino)hexyloxy)-2-fluorophenyl) methylmethanesulfonate

To a solution of IN-001-21 (0.136 g, 0.27 mmol) and NEt₃ (0.136 g, 0.27 mmol) in CH₂Cl₂ (5 mL), cooled down at −15° C., a 0.1 M solution of MsCl (4.05 mL, 0.41 mmol) was added dropwise. The reaction was stirred at −15° C. for 30 min and then the reaction was warmed up using an ice/water bath, stirring the reaction of other 30 min. The solvent was removed in vacuo, and the residue was used without further purification into the next step, assuming 100% yield (0.157 g). MS (m/z): 492.3 (M-OMs+H), 436.2, (M-tBu-OMs+H). LCMS Ret. time: 2.94. Rf: 0.29 (Hexanes/EtOAc 80/20).

### Step 3

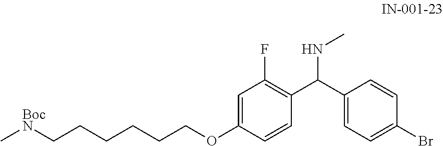 [SMILES: CNC(c1ccc(Br)cc1)c1ccc(OCCCCCCN(C)C(=O)OC(C)(C)C)cc1F]

### IN-001-23. Tert-butyl-6-(4-((4-bromophenyl)(methylamino)methyl)-3-fluorophenoxy)hexyl-(methyl)carbamate

A solution of IN-001-22 (0.157 g, 0.27 mmol) and 40% aqueous solution of methylamine (2 mL) in dioxane (10 mL) was heated for an overnight. After cooling down the reaction mixture at room temperature, the mixture was diluted with EtOAc and water (20 mL each) and then it was transferred to a separation funnel and the mixture was extracted. The organic phase was separated and the aqueous phase was extracted with EtOAc (2×20 mL). The organic phases were combined, dried over Na₂SO₄ and then the solvent was removed in vacuo. The product was obtained as a pale yellow oil (0.064 g, 46%) after purification by flash chromatography (Hexanes-EtOAc 0-60%). MS (m/z): 523.2 (M+H), (M-NHMe+H), 436.1, (M-tBu-NHMe+H). LCMS Ret. time: 2.09. Rf: 0.2 (Hexanes/EtOAc 80/20). ¹H NMR (CDCl₃, δ): 1.32 (quint, J=7.00, 2H), 1.445 (s, 9H), 1.52 (quint, J=7.45, 2H), 1.75 (quint, J=7.00 Hz, 2H), 2.391 (s, 3H), 2.82 (s, 3H), 3.193 (br, 2H), 3.89 (t, J=6.10 Hz, 2H), 4.92, (s, 1H), 6.538 (dd, J=12.01 Hz, J=2.45 Hz, 1H), 6.643 (dd, J=8.86 Hz, J=2.25 Hz, 1H), 7.27 (m, 3H), 7.413 (d, J=8.50, 2H).

### Step 4

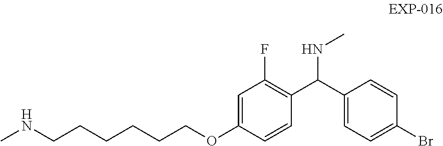 [SMILES: CNCCCCCCOc1ccc(C(NC)c2ccc(Br)cc2)c(F)c1]

### EXP-016. 6-(4-((4-bromophenyl)(methylamino)methyl)-3-fluorophenoxy)-N-methylhexan-1-amine (LZH-00018)

The N-Boc protected amine IN-001-23 (0.064 g, 0.12 mmol) was dissolved in 4M HCl solution in dioxane (5 mL), and the mixture was stirred for 1 h at room temperature. The reaction was quenched by addition of saturated solution of NaHCO₃ until no more gas was produced and the pH of the solution was about 8 (aprox. 25 mL). Then, the mixture was diluted with EtOAc (30 mL) and the mixture was extracted. The organic phase was separated and the aqueous phase was extracted with EtOAc (2×30 mL). The organic layers were combined and washed with brine (50 mL), dried over Na₂SO₄ and then the solvent was evaporated in vacuo. The product was obtained as a white solid (0.0308 g, 60%) after two purifications by flash chromatography (0-100% CH₂Cl₂-Polar solvent, being polar solvent MeOH/NH₄OH 5/1). MS: 423.1 m/z (M+H), 392.2 m/z (M-NHMe+H). Ret. time LCMS: 1.46. ¹H NMR (CDCl₃, δ): 1.40 (m, 4H), 1.72 (m, 4H), 2.373 (s, 3H), 2.62 (s, 3H), 2.865 (m, 2H), 3.85 (t, J=6.20 Hz, 2H), 4.05 (br, 1H), 4.92 (s, 1H), 6.51 (dd, J=12.05 Hz, J=2.85, 1H), 6.617 (dd, J=8.30 Hz, J=2.35 Hz, 1H), 7.26 (d, J=8.30 Hz 2H), 7.295 (pt, J=8.55 Hz, 1H), 7.39 (d, J=8.30 Hz 2H).

### Synthesis of Example 17

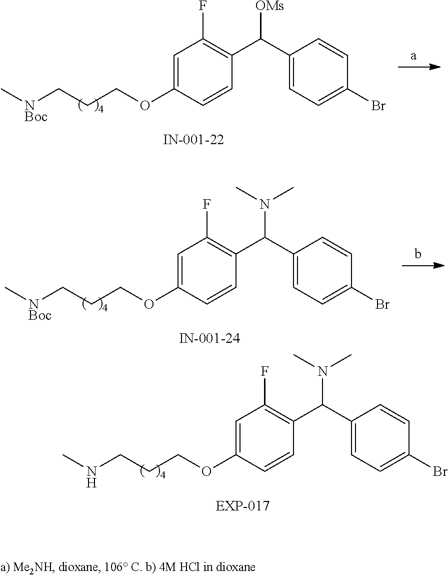

### Step 1

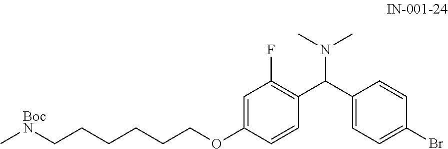 [SMILES: CN(CCCCCCOc1ccc(C(c2ccc(Br)cc2)N(C)C)c(F)c1)C(=O)OC(C)(C)C]

### IN-001-24: Tert-butyl-6-(4-((4-bromophenyl)(dimethylamino)methyl)-3-fluorophenoxy)hexyl-(methyl)carbamate

Compound IN-001-24 was prepared as it was described for IN-001-23 on using IN-001-22 (0.157 g, 0.27 mmol) and 40% aqueous solution of dimethylamine (2 mL) in dioxane (10 mL). The product was obtained as a pale yellow solid (0.072 g, 50%) after purification by flash chromatography (0-40% Hexanes-EtOAc). MS (m/z): 537.3 (M+H), 492.2 (M-NMe₂+H), 436.2 (M-tBu-NMe₂+H). LCMS Ret. time: 2.12. Rf: 0.48 (Hexanes/EtOAc 80/20). ¹H NMR (CDCl₃, δ): 1.32 (m, 2H), 1.44 (s, 9H), 1.515 (m, 2H), 1.743 (m, 2H), 2.19 (s, 6H), 2.82 (s, 3H), 3.19 (br, 2H), 3.88 (t, J=6.65 Hz, 2H), 4.412, (s, 1H), 6.505 (dd, J=10.65 Hz, J=2.35 Hz, 1H), 6.643 (dd, J=9.01 Hz, J=2.70 Hz, 1H), 7.35 (d, J=8.36, 2H), 7.38 (m, 3H).

### Step 2

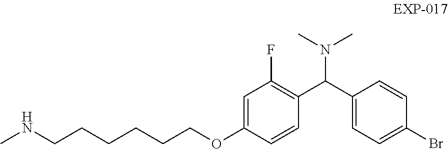 [SMILES: CNCCCCCCOc1ccc(C(c2ccc(Br)cc2)N(C)C)c(F)c1]

### EXP-017: 6-(4-((4-bromophenyl)(dimethylamino)methyl)-3-fluorophenoxy)-N-methylhexan-1-amine (LZH-00019)

Compound EXP-017 was prepared as it was described for EXP-016 on using IN-001-24 (0.072 g, 0.13 mmol) and 4M HCl solution in dioxane (5 mL). The product was obtained as a yellow solid (0.0306 g, 52%) after two purifications by flash chromatography (0-100% CH₂C₂-Polar solvent, being polar solvent MeOH/NH₄OH 5/1). MS: 437.1 m/z (M+H), 392.2 m/z (M-NMe₂+H). Ret. time LCMS: 1.46. ¹H NMR (CDCl₃, δ): 1.44 (m, 4H), 1.72 (quint, J=7.25 Hz, 2H), 1.82 (quint, J=7.25 Hz, 2H), 2.22 (s, 6H), 2.64 (s, 3H), 2.90 (m, 2H), 3.85 (t, J=6.30 Hz, 2H), 4.46, (s, 1H), 6.49 (dd, J=12.10 Hz, J=2.45, 1H), 6.635 (dd, J=8.55 Hz, J=2.45 Hz, 1H), 7.324 (d, J=8.75 Hz, 2H), 7.395 (d, J=8.575 Hz, 2H), 7.2 (m, 1H), 9.04 (br, 1H).

### Synthesis of Example 18

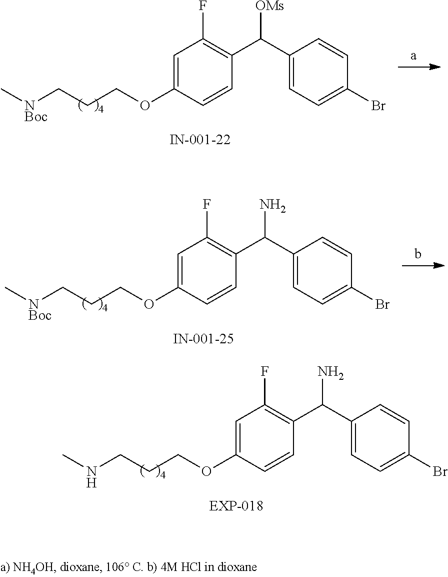

### Step 1

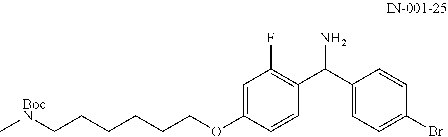 [SMILES: CN(CCCCCCOc1ccc(C(N)c2ccc(Br)cc2)c(F)c1)C(=O)OC(C)(C)C]

### IN-001-25: Tert-butyl-6-(4-(amino(4-bromophenyl)methyl)-3-fluorophenoxy)hexyl(methyl)-carbamate

Compound IN-001-25 was prepared as it was described for IN-001-23 on using IN-001-22 (0.236 g, 0.40 mmol) and 28% aqueous solution of NH₄OH (8 mL) in dioxane (10 mL), heating the mixture reaction for 48 h. The product was obtained as a pale yellow solid (0.050 g, 24%) after purification by flash chromatography (0-65% Hexanes-EtOAc). MS (m/z): 509.3 (M+H), 492.2 (M-NH₂+H), 436.2 (M-tBu-NH₂+H). LCMS Ret. time: 1.99. Rf: 0.34 (Hexanes/EtOAc 80/20). ¹H NMR (CDCl₃, δ): 1.32 (quint, J=6.45 Hz, 2H), 1.44 (s, 9H), 1.52 (t, J=7.10 Hz, 2H), 1.754 (t, J=7.80 Hz, 2H), 2.825 (s, 3H), 3.19 (br, 2H), 3.895 (t, J=5.80 Hz, 2H), 5.384, (s, 1H), 6.552 (dd, J=12.45 Hz, J=2.40 Hz, 1H), 6.639 (dd, J=8.55 Hz, J=2.55 Hz, 1H), 7.242 (d, J=8.85 Hz, 1H), 7.272 (d, J=7.60 Hz, 2H), 7.42 (d, J=7.60 Hz, 2H).

### Step 2

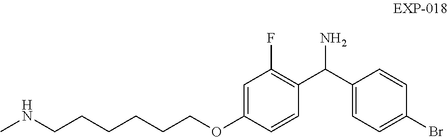 [SMILES: CNCCCCCCOc1ccc(C(N)c2ccc(Br)cc2)c(F)c1]

### EXP-018. 6-(4-(amino(4-bromophenyl)methyl)-3-fluorophenoxy)-N-methylhexan-1-amine (LZH-00020)

Compound EXP-018 was prepared as it was described for EXP-016 on using IN-001-25 (0.050 g, 0.10 mmol) and 4M HCl solution in dioxane (5 mL). The product was obtained as a yellow solid (0.0306 g, 52%) after two purifications by flash chromatography (0-100% CH₂Cl₂-Polar solvent, being polar solvent MeOH/NH₄OH 5/1). MS: 437.1 m/z (M+H), 392.2 m/z (M-NMe₂+H). Ret. time LCMS: 1.46. ¹H NMR (CDCl₃, δ): 1.44 (m, 4H), 1.603 (quint, J=6.75 Hz, 2H), 1.82 (quint, J=6.75 Hz, 2H), 2.49 (s, 3H), 2.68 (pt, 2H), 3.88 (t, J=5.70 Hz, 2H), 5.363, (s, 1H), 6.54 (dd, J=12.35 Hz, J=2.45, 1H), 6.625 (dd, J=8.70 Hz, J=2.40 Hz, 1H), 7.233 (m, 3H), 7.407 (d, J=8.30 Hz, 2H).

### Synthesis of Example 19

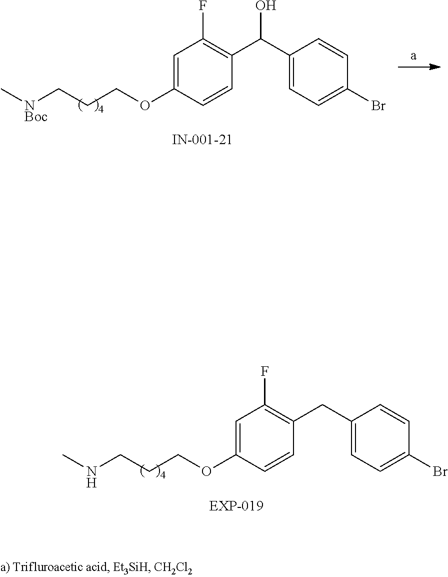

### EXP-019. 6-(4-(4-bromobenzyl)-3-fluorophenoxy)-N-methylhexan-1-amine (LZH-00021)

To a solution of IN-001-21 (0.107 g, 0.21 mmol) in CH₂Cl₂ (5 mL), Et₃SiH (330 μL, 2.10 mmol) and trifluoroacetic acid (195 μL, 2.10 mmol) were added. The reaction was stirred for 2 h at room temperature. The, the reaction was quenched with saturated solution of NaHCO₃ (aprox. 3 mL), and then the mixture was diluted with CH₂Cl₂ and water (15 mL each). The mixture was extracted, the organic phase separated and the aqueous layer was extracted with CH₂Cl₂ (2×15 mL). The organic layers were combined, dried over Na₂SO₄ and the solvent was removed in vacuo. The residue was purified by flash chromatography (0-45% CH₂Cl₂-Polar solvent, being polar solvent MeOH/NH₄OH 5/1). The product was obtained as a white solid (0.075 g, 91%). MS (m/z): 394.3 (M−H₂O+H). LCMS Ret. time: 1.85. Rf: 0.48 (80/20 CH₂Cl₂-Polar solvent, being polar solvent MeOH/NH-4OH 5/1). ¹H NMR (CDCl₃, δ): 1.38 (m, 4H), 1.67 (m, 4H), 2.58 (s, 3H), 2.85 (m, 2H), 3.78 (s, 2H), 3.81 (t, J=6.70 Hz, 2H), 6.51 (m, 2H), 6.92 (pt, J=8.96 Hz, 1H), 6.98 (d, J=8.35 Hz, 2H), 7.31 (d, J=8.35 Hz, 2H).

### Synthesis of Example 20

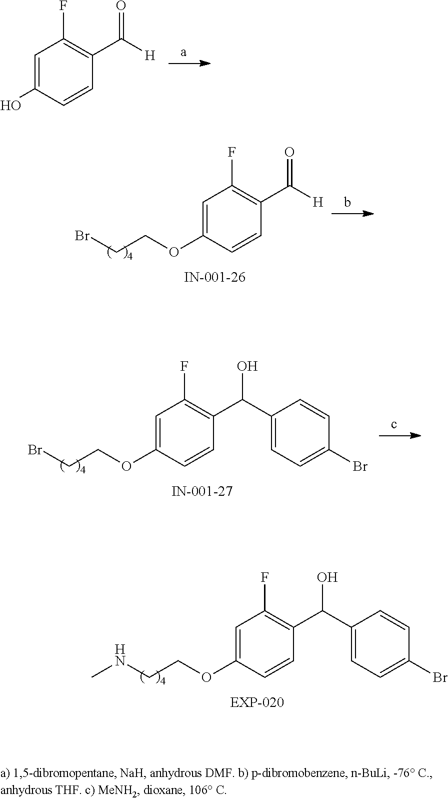

### Step 1

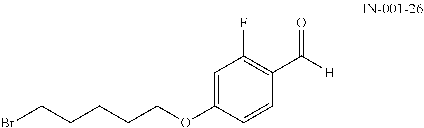 [SMILES: O=Cc1ccc(OCCCCCBr)cc1F]

### IN-001-26. 4-(5-bromopentyloxy)-2-fluorobenzaldehyde

To an anhydrous DMF (50 mL) solution of 2-fluoro-4-hydroxybenzaldehyde (1.98 g, 14.13 mmol) and 1,5-dibromopentane (5.8 mL, 44.39 mmol), NaH (0.57 g NaH at 60%, 0.34 g of NaH, 14.13 mmol) was added portionwise. The reaction mixture was stirred for room temperature for 4 h under N₂ atmosphere. Then, the reaction was quenched adding water (60 mL) and the mixture was extracted with EtOAc (3×60 mL). The organic layers were combined and the combined organic phases were washed with water (3×100 mL). The organic phase was dried over Na₂SO₄ and the solvent was removed in vacuo. The crude was purified by flash chromatography (Hexanes-EtOAc 0-15%) and the product was obtained as a colorless oil (2.39 g, 58%). MS (m/z): 289.1 (M+H). Ret. time LCMS: 2.19. Rf: 0.46 (Hexanes/EtAcO 90/10). ¹H NMR (CDCl₃, δ): 1.640 (m, 2H), 1.848 (m, 2H), 1.942 (m, 2H), 3.441 (t, J=6.55 Hz, 2H), 4.035 (t, J=6.30 Hz, 2H), 6.618 (dd, J=12.31 Hz J=2.45 Hz, 1H), 6.761 (dd, J=9.05 Hz, J=2.45 Hz, 1H), 7.806 (pt, J=9.05 Hz, 1H), 10.198 (s).

### Step 2

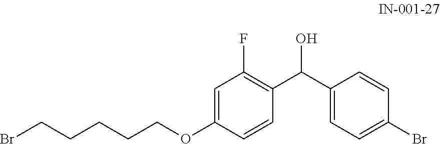 [SMILES: OC(c1ccc(Br)cc1)c1ccc(OCCCCCBr)cc1F]

### IN-001-27. (4-(5-bromopentyloxy)-2-fluorophenyl)(4-bromophenyl)methanol

To a solution of p-dibromobenzene (0.82 g, 3.46 mmol) in anhydrous THF (15 mL) at −70° C. and under N₂ atmosphere, 1.6 M solution of n-BuLi in hexanes (2.4 mL, 3.81 mmol) was added dropwise. The mixture reaction was stirred at −70° C. for 2 h and then, a solution of IN-001-26 (1.00 g, 3.46 mmol) in THF (15 mL) was added. The reaction was stirred for 30 min at −70° C. and then the reaction was warmed up at room temperature and stirred for an overnight. The reaction was quenched with saturated solution of NH₄Cl (50 mL). The mixture was extracted with EtOAc (3×50 mL) and the organic layers were combined, dried over Na₂SO₄ and the solvent was removed in vacuo. The product was obtained as a pale yellow oil (1.02 g, 66%) after a double purification by flash chromatography (0-40% Hexanes/EtOAc). MS (m/Z): 427.1 (M−H₂O+H). LCMS Ret. time: 2.41. Rf: 0.30 (Hexanex/EtOAc 80/20). ¹H NMR (CDCl₃, δ): 1.608 (m, 2H), 1.797 (quint, J=7.60 Hz, 2H), 1.925 (quint, J=7.55 Hz), 2.255 (br, 1H), 3.430 (t, J=7.55 Hz, 2H), 3.932 (t, J=6.75 Hz, 2H), 6.039 (s, 1H), 6.575 (dd, J=12.21 Hz, J=2.50 Hz, 1H), 6.664 (dd, J=8.30 Hz, J=2.50 Hz, 1H), 7.269 (d, J=8.46, 2H), 7.454 (d, J=8.46, 2H).

### Step 3

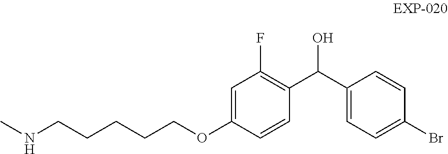 [SMILES: CNCCCCCOc1ccc(C(O)c2ccc(Br)cc2)c(F)c1]

### EXP-020. (4-bromophenyl)(2-fluoro-4-(5-(methylamino)pentyloxy)phenyl)methanol (LZH 00022)

A solution of IN-001-27 (0.110 g, 0.25 mmol) and 40% aqueous solution of methylamine (0.07 mL) in dioxane (5 mL) were heated for an overnight. After cooling down the reaction mixture at room temperature, the mixture was diluted with EtOAc and water (20 mL each) and then it was transferred to a separation funnel and the mixture was extracted. The organic phase was separated and the aqueous phase was extracted with EtOAc (2×20 mL). The organic phases were combined, dried over Na₂SO₄ and then the solvent was removed in vacuo. The product was obtained as a yellow solid (0.068, 70%) after a double purification by flash chromatography (0-20% CH₂Cl₂-Polar solvent, being polar solvent: MeOH/NH₄OH 5/1). MS (m/z): 396.2 (M+H). LCMS Ret. time: 1.69. Rf: 0.34 (CH2Cl2/Polar solvent 80/20, being polar solvent: MeOH/NH₄OH 5/1). ¹H NMR (CDCl₃, δ): 1.406 (m, 2H), 1.478 (m, 2H), 2.354 (s, 3H), 2.511 (pt, J=7.15 Hz, 2H), 3.877 (pt, J=6.45 Hz, 2H), 5.645 (br, 2H), 5.986, (s, 1H), 6.537 (dd, J=12.25 Hz, J=12.45 Hz, 1H), 6.637 (dd, J=8.56 Hz, J=2.45 Hz, 1H), 7.253 (m, 3H), 7.413 (d, J=8.56, 2H).

### Synthesis of Example 21

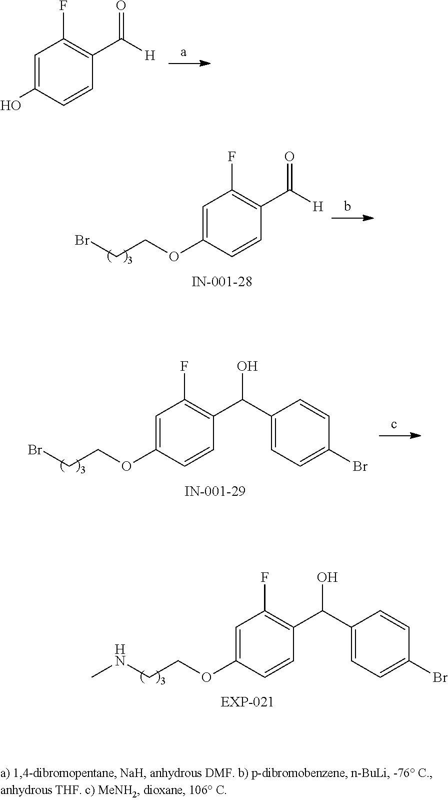

### Step 1

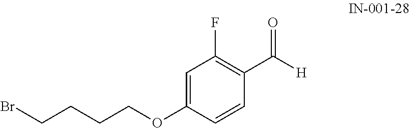 [SMILES: O=Cc1ccc(OCCCCBr)cc1F]

### IN-001-28. 4-(4-bromobutoxy)-2-fluorobenzaldehyde

Compound IN-001-28 was prepared as it was described for IN-001-26 on using an anhydrous DMF (50 mL) solution of 2-fluoro-4-hydroxybenzaldehyde (1.98 g, 14.13 mmol) and 1,4-dibromobutane (5.81 mL, 44.39 mmol), NaH (0.57 g NaH at 60%, 0.34 g of NaH, 14.13 mmol). The crude was purified by flash chromatography (Hexanes-EtOAc 0-15%) and the product was obtained as a colorless oil (2.45 g, 63%). MS (m/z): 275.1 (M+H). Ret. time LCMS: 2.10. Rf: 0.43 (hexanes/EtOAc 90/10). ¹H NMR (CDCl₃, δ): 1.99 (m, 2H), 2.07 (m, 2H), 3.484 (t, J=6.40 Hz, 2H), 4.065 (t, J=5.85 Hz, 2H), 6.623 (dd, J=12.31 Hz, J=2.20 Hz, 1H), 6.764 (dd, J=8.70 Hz, J=2.20 Hz, 1H), 7.810 (pt, J=8.70 Hz, 1H), 10.20 (s).

### Step 2

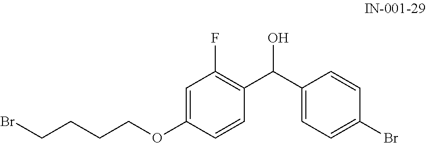 [SMILES: OC(c1ccc(Br)cc1)c1ccc(OCCCCBr)cc1F]

### IN-001-29: (4-(4-bromobutoxy)-2-fluorophenyl)(4-bromophenyl)methanol

Compound IN-001-29 was prepared as it was described for IN-001-29 on using p-bromobenzene (0.85 g, 3.63 mmol), IN-001-28 (1.00 g, 3.63 mmol), 1.6 M n-BuLi solution in hexanes (2.5 mL, 3.99 mmol) and anhydrous THF (30 mL). The product was obtained as a pale yellow oil (1.56 g, 99%) after a double purification by flash chromatography (0-30% hexane-EtOAc). Rf: 0.30 (Hexanex/EtOAc 80/20). ¹H NMR (CDCl₃, δ): 1.93 (m, 2H), 2.04 (m, 2H), 2251 (br, 1H), 3.472 (t, J=6.25 Hz, 2H), 3.959 (t, J=6.25 Hz, 2H), 6.043 (s, 1H), 6.576 (dd, J=12.00 Hz J=2.60 Hz, 1H), 6.664 (dd, J=8.50 Hz, J=2.60 Hz, 1H), 7.268 (d, J=8.51 Hz, 2H), 7.455 (d, J=8.51 Hz, 2H).

### Step 3

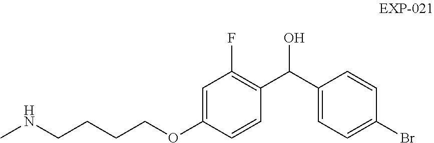 [SMILES: CNCCCCOc1ccc(C(O)c2ccc(Br)cc2)c(F)c1]

### EXP-021. (4-bromophenyl)(2-fluoro-4-(4-(methylamino)butoxy)phenyl)methanol (LZH-00023)

Compound EXP-021 was prepared as it was described for EXP-020 on using IN-001-29 (0.11 g, 0.25 mmol), 40% solution of MeNH₂ (0.07 mL, 0.75 mmol) and dioxane (5 mL). The product was obtained as a pale yellow oil (0.052, 53%) after a double purification by flash chromatography (0-20% CH₂Cl₂-Polar solvent, being polar solvent: MeOH/NH₄OH 5/1). MS (m/z): 382.2 (M+H), LCMS Ret. time: 1.65. Rf: 0.35 (CH₂Cl₂/Polar solvent 80/20, being polar solvent: MeOH/NH₄OH 5/1). ¹H NMR (CDCl₃, δ): 1.608 (quint, J=7.20 Hz, 2H), 1.771 (quint, J=7.95 Hz, 2H), 2.409 (s, 3H), 2.600 (pt, J=7.20 Hz, 2H), 3.903 (pt, J=6.40 Hz, 2H), 6.014 s, 1H), 6.549 (dd, J=12.21 Hz, J=2.65 Hz, 1H), 6.642 (dd, J=8.66 Hz, J=2.55 Hz, 1H), 7.262 (m, 3H), 7.414 (d, J=8.71, 2H).

### Synthesis of Example 22

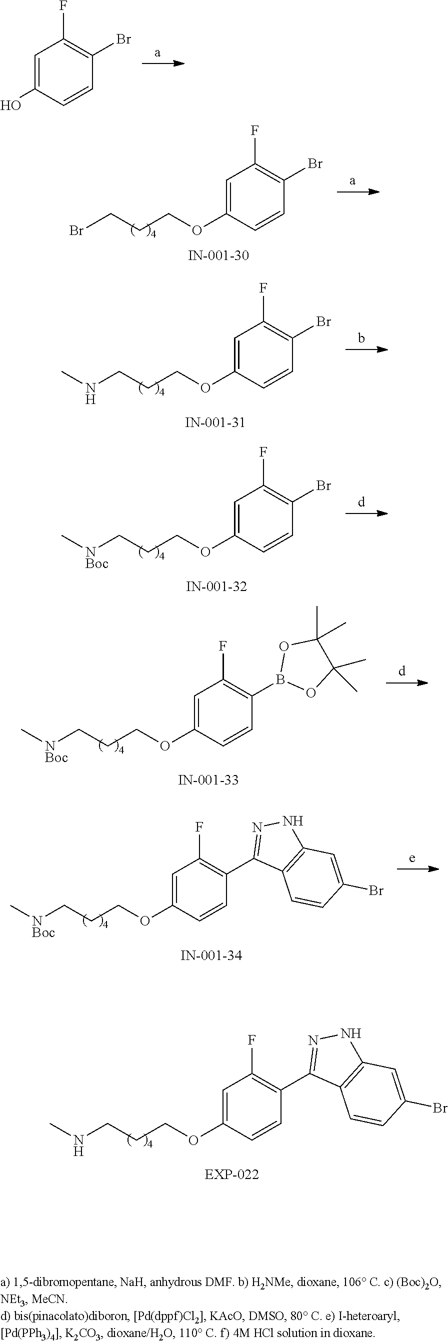

### Step 1

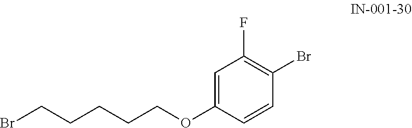 [SMILES: Fc1cc(OCCCCCBr)ccc1Br]

### IN-001-30: 1-bromo-4-(5-bromopentyloxy)-2-fluorobenzene

To an anhydrous DMF (30 mL) solution 4-bromo-3-fluorophenol (2.5 g, 13.09 mmol) and 1,5-dibromopentane (5.3 mL, 39.27 mmol), NaH (0.524 g NaH at 60%, 0.31 g of NaH, 39.27 mmol) was added portionwise. The reaction mixture was stirred for room temperature for 4 h under N₂ atmosphere. Then, the reaction was quenched adding water (60 mL) and the mixture was extracted with EtOAc (3×60 mL). The organic layers were combined and the combined organic phases were washed with water (3×100 mL). The organic phase was dried over Na₂SO₄ and the solvent was removed in vacuo. The crude was purified by flash chromatography (Hexanes-EtOAc 0-5%) and the product was obtained as a colorless oil (3.41 g, 80%). The molecular ion was not detected by LCMS. LCMS ret. time: 2.51. Rf: 0.51 (Hexanes/EtOAc 95/5). ¹H NMR (CDCl₃, δ): 1.61 (m, 2H), 1.81 (m, 2H), 1.93 (m, 2H), 2.43 (pt, J=6.40 Hz, 2H), 3.93 (t, J=6.35 Hz, 2H), 6.59 (ddd, J=8.90 Hz J=2.80 Hz, J=1.2 Hz, 1H), 6.67 (dd, J=10.35 Hz, J=2.80 Hz, 1H), 7.39 (dd, J=8.80 Hz, J=8.05 Hz, 1H).

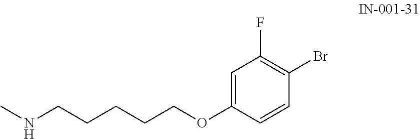 [SMILES: CNCCCCCOc1ccc(Br)c(F)c1]

### IN-001-31: 5-(4-bromo-3-fluorophenoxy)-N-methylpentan-1-amine

A sealed tube was charged with IN-001-01 (2 g, 5.65 mmol), 40% solution of MeNH₂ (1.5 mL, 16.95 mmol) and dioxane (50 mL). The solution was heated at 106° C. for an overnight. After cooling down the reaction mixture at room temperature, the mixture was diluted with EtOAc and water (50 mL each) and then it was transferred to a separation funnel and the mixture was extracted. The organic phase was separated and the aqueous phase was extracted with EtOAc (2×50 mL). The organic phases were combined, dried over Na₂SO₄ and then the solvent was removed in vacuo. The brown oily residue was dried in vacuo for 2 h, affording the target product as a yellow brown solid (2.81 g, 97%). MS (m/z): 290.2 m/z (M+H). LCMS Ret. time: 1.63. The product was used in the next step without further purification.

### Step 3

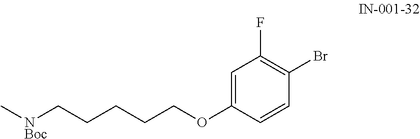 [SMILES: CN(CCCCCOc1ccc(Br)c(F)c1)C(=O)OC(C)(C)C]

### IN-001-32. Tert-butyl 5-(4-bromo-3-fluorophenoxy)pentyl(methyl)carbamate

To solution of the IN-001-31 (2.81 g, 9.68 mmol) in anhydrous MeCN (50 mL), NEt₃ (2.7 mL, 10.65 mmol) was added. Then, under stirring, a solution of Boc₂O (1.32 g, 6.04 mmol) in MeCN (50 mL) was added. The reaction was stirred at room temperature for 2 h. Then, the reaction was quenched adding water (50 mL) and the mixture was transferred to a separation funnel and the aqueous phase was extracted with EtOAc (3×50 mL) and the combined organic layers were washed with brine (1×70 mL). The organic phase was dried over Na₂SO₄ and the solvent was removed in vacuo. The crude was purified by flash chromatography (0-30% Hexanes-EtOAc), affording the target product as a light yellow oil (2.01 g, 53%). MS (m/z): 404.3 (M+H), 348.1 (M-tBu+H), 304.3 (M+Boc+H). LCMS Ret. time: 2.69. Rf: 0.54 (Hexanes/EtOAc 80/20). MS(m/z): 390.5 (M+H), 334.1, (M-tBu+H), 290.1 (M+Boc+H). LCMS Ret. time: 2.46. Rf: 0.36 (Hexanes/EtOAc 90/10). ¹H NMR (CDCl₃, δ): 1.23 (m, 2H), 1.34 (s, 9H), 1.46 (m, 4H), 1.69 (m, 2H), 2.73 (s, 3H), 3.12 (m, 2H), 3.81 (t, J=6.83 Hz, 2H), 6.52 (ddd, J=8.90 Hz, J=3.10 Hz, J=0.5 Hz, 1H), 6.00 (dd, J=10.60 Hz, J=2.90 Hz, 1H), 7.31 (pt, J=8.40 Hz, 1H).

### Step 4

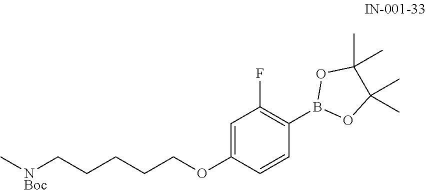

### IN-001-33: tert-butyl-5-(3-fluoro-4-(4,4,5,5-tetramethyl-1,3,2-dioxaborolan-2-yl)phenoxy)-pentyl(methyl)-carbamate

A round-bottom flask was charged with IN-001-32 (1.5 g, 3.99 mmol), KOAc 1.17 g, 11.97 mmol), bis(pinacolato)diboron (1.51 g, 5.98 mmol), [Pd(dppf)Cl₂].DCM (0.32 g, 0.45 mmol). Then, the system was put under vacuum and under vacuum anhydrous DMSO (30 mL) was added via syringe. Then, the system was put under N₂ atmosphere and the reaction was heated at 80° C. for an overnight. After cooling down, the mixture was diluted with EtOAc (80 mL) and the solution was extracted with water (4×60 mL). The organic phase was dried over Na₂SO₄ and the solvent was removed in vacuo and the residue was then purified by flash chromatography (0-40% Hexanes-EtOAc) and after the evaporation of the solvent the product was obtained as a yellow oil (0.736 g, 44%). MS (m/z): 423.2 (M+H), 368.3 (M-tBu+H). LCMS Ret. time: 2.53. Rf: 0.44 (Hexanes/EtOAc 80/20). ¹H NMR (CDCl₃, δ): 1.34 (s, 12H) 1.44 (s, 9H), 1.56 (quint, J=8.05 Hz, 2H), 1.80 (quint, J=6.25 Hz, 2H), 2.83 (s, 3H), 3.21 (br, 2H), 3.95 (t, J=6.15 Hz, 2H), 6.54 (dd, J=11.55 Hz, J=2.05 Hz, 1H), 6.66 (dd, J=8.46 Hz, J=2.35 Hz, 1H), 7.62 (pt, J=7.95 Hz, 1H).

### Step 5

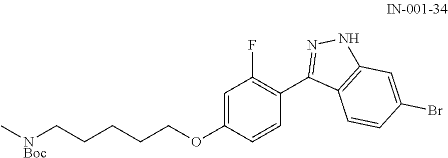 [SMILES: CN(CCCCCOc1ccc(-c2n[nH]c3cc(Br)ccc23)c(F)c1)C(=O)OC(C)(C)C]

### IN-001-34: Tert-butyl-5-(4-(6-bromo-1H-indazol-3-yl)-3-fluorophenoxy)pentyl(methyl)carba-mate

A scintillation vial was charged with the 6-bromo-3iodo-1H-indazole (0.112 g, 0.35 mmol), K₂CO₃ (0.145 g, 1.05 mmol), and [Pd(PPh₃)₄] 1 (0.020 g, 0.02 mmol). Then, the system was put under vacuum and under vacuum 10 mL of a dioxane solution of the boronic ester IN-001-33 (0.199 g, 0.45 mmol) and water (1 mL) were added via syringe. Then, the system was put under N₂ atmosphere and the reaction was heated at 80° C. for an overnight. After this time, the solvent was removed in vacuo and the residue was then purified by flash chromatography (0-90% Hexanes-EtOAc) and after the evaporation of the solvent, the product was obtained as a yellow oil (0.078 g, 44%). MS (m/z): 506.3 (M+H), 450.2 (M-tBu+H), 406.3 (M-Boc+H). LCMS ret. time: 2.55. Rf: 0.33 (Hexanes/EtOAc 70/30 ¹H NMR (CDCl₃, δ): 1.36 (m, 2H), 1.46 (s, 9H), 1.53 (m, 4H), 1.82 (m, 2H), 2.84 (s, 3H), 3.22 (br, 2H), 4.00 (t, J=6.10 Hz, 2H), 4.08 (s, 3H), 6.76 (dd, J=12.31 Hz, J=2.40 Hz, 1H), 6.81 (dd, J=8.60 Hz, J=2.40 Hz, 1H), 7.27 (dd, J=8.60 Hz, J=1.55 Hz, 1H), 7.59 (dd, J=1.75 Hz, J=0.45 Hz, 1H), 7.64 (m, 1H), 7.67 (m, 1H).

### Step 6

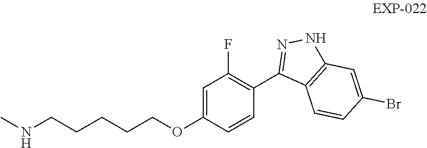 [SMILES: CNCCCCCOc1ccc(-c2n[nH]c3cc(Br)ccc23)c(F)c1]

### EXP-022: 5-(4-(6-bromo-1H-indazol-3-yl)-3-fluorophenoxy)-N-methylpentan-1-amine (LZH-00024)

The N-Boc protected amine IN-001-34 (0.078 g, 0.15 mmol) was dissolved in 4M HCl solution in dioxane (5 mL), and the mixture was stirred for h at room temperature. The reaction was quenched by addition of saturated solution of NaHCO₃ until no more gas was produced and the pH of the solution was about 8 (aprox. 25 mL). Then, the mixture was diluted with EtOAc (25 mL) and the mixture was extracted. The organic phase was separated and the aqueous phase was extracted with EtOAc (2×25 mL). The organic layers were combined and washed with brine (50 mL), dried over Na₂SO₄ and then the solvent was evaporated in vacuo. The solid yellow residue obtained after the evaporation was purified by flash chromatography (0-35% CH₂Cl₂-Polar solvent, being polar solvent MeOH/NH₄OH 5/1) and the target product was isolated as a pale yellow solid (0.036 g, 58%). MS (m/z): 406.0 (M+H). LCMS Ret. time 1.74. Rf: 0.26 (80/20 CH₂Cl₂/Polar solvent, being polar solvent MeOH/NH₄OH 5/1). ¹H NMR (CDCl₃, δ): 1.51 (quint, J=7.15 Hz, 2H), 1.593 (quint, J=7.10 Hz, 2H), 2.45 (s, 3H), 2.64 (t, J=7.10, 2H), 3.99 (t, J=7.10 Hz, 2H), 6.747 (dd, J=12.41 Hz, J=2.40, 1H), 6.791 (dd, J=8.61 Hz, J=2.60 Hz, 1H), 7.256 (dd, J=8.60 Hz, J=1.65 Hz, 1H), 7.65 (m, 3H).

### Synthesis of Example 23

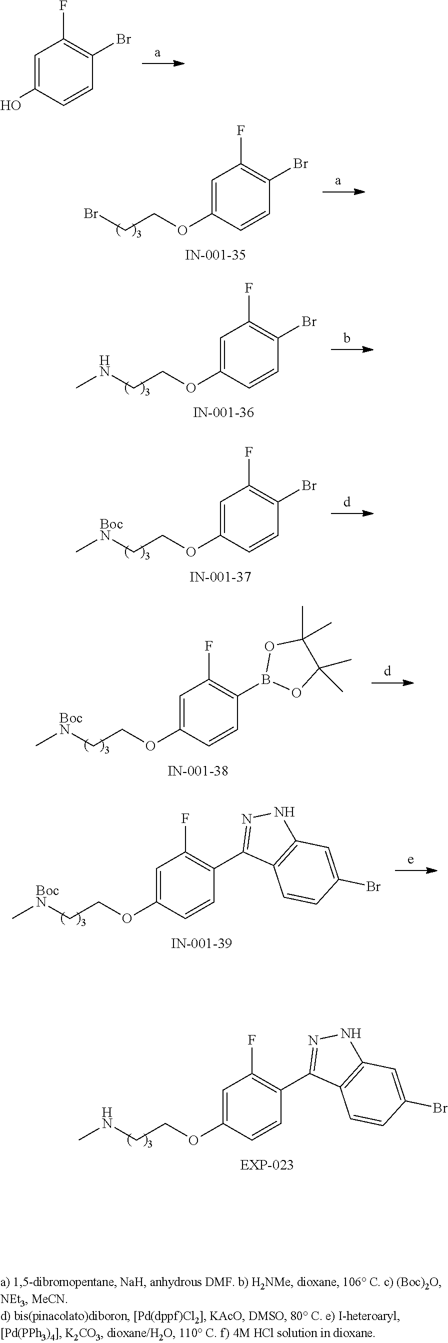

### Step 1

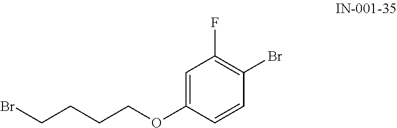 [SMILES: Fc1cc(OCCCCBr)ccc1Br]

### IN-001-35: 1-bromo-4-(4-bromobutoxy)-2-fluorobenzene

Compound IN-001-35 was prepared as it was described for IN-001-30 on using 4-bromo-3-fluorophenol (5 g, 26.18 mmol), 1,4-dibromobutane (9.5 mL, 78.54 mmol), NaH (1.0472 g NaH at 60%, 0.63 g of NaH, 26.18 mmol) and DMF as a solvent (80 mL). The reaction was quenched with water (100 mL) and extracted with EtOAc (3×100 mL) and the organic layer was washed with water (3×150 mL). The crude was purified by flash chromatography (Hexanes-EtOAc 0-5%) and the product was obtained as a colorless oil (6.17, 72%). The molecular ion was not detected by LCMS. LCMS ret. time: 2.40. Rf: 0.51 (Hexanes/EtOAc 95/5). ¹H NMR (CDCl₃, δ): 1.94 (m, 2H), 2.05 (m, 2H), 3.48 (t, J=6.30 Hz, 2H), 3.95 (pt, J=5.55 Hz, 2H), 6.59 (ddd, J=8.90 Hz, J=2.90 Hz, J=1.05 Hz, 1H), 6.68 (dd, J=10.31 Hz, J=2.75 Hz, 1H), 7.39 (dd, J=8.75 Hz, J=7.95 Hz, 1H).

### Step 2

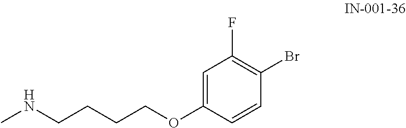

### IN-001-36: 4-(4-bromo-3-fluorophenoxy)-N-methylbutan-1-amine

Compound IN-001-36 was prepared as it was described for IN-001-31 on using IN-001-35 (3.10 g, 9.54 mmol), 40% solution of MeNH₂ (2.5 mL, 28.62 mmol) and dioxane (25 mL), and the product was obtained as a yellow brown solid (2.23 g, 85%). MS (m/z): 276.1 (M+H). LCMS Ret. time: 1.58. The product was used in the next step without further purification.

### Step 3

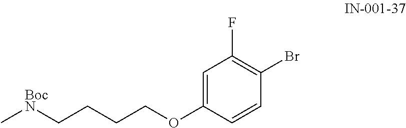 [SMILES: CN(CCCCOc1ccc(Br)c(F)c1)C(=O)OC(C)(C)C]

### IN-001-37: tert-butyl 4-(4-bromo-3-fluorophenoxy)butyl(methyl)carbamate

Compound IN-001-37 was prepared as it was described for IN-001-32 on using the aminoether IN-01-36 (3.80 g, 13.76 mmol), NEt₃ (3.8 mL, 27.52 mmol), Boc₂O (2.78 g, 15.14 mmol) in MeCN (50 mL) as a solvent, and it was obtained as a light yellow oil (4.03 g, 78%) after purification by flash chromatography (0-37% Hexanes-EtOAc). MS (m/z): 376.2 (M+H), 320.1 (M-tBu+H), 276.1 (M+Boc+H). LCMS Ret. time: 2.46. Rf: 0.55 (Hexanes/EtOAc 90/10). ¹H NMR (CDCl₃, δ): 1.23 (m, 2H), 1.34 (s, 9H), 1.46 (m, 4H), 1.69 (m, 2H), 2.73 (s, 3H), 3.12 (m, 2H), 3.81 (t, J=6.83 Hz, 2H), 6.52 (ddd, J=8.90 Hz, J=3.10 Hz, J=0.5 Hz, 1H), 6.00 (dd, J=10.60 Hz, J=2.90 Hz, 1H), 7.31 (pt, J=8.40 Hz, 1H).

### Step 4

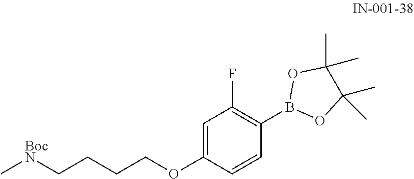

### IN-001-38: Tert-butyl-4-(3-fluoro-4-(4,4,5,5-tetramethyl-1,3,2-dioxaborolan-2-yl)phenoxy)-butyl(methyl)carbamate

The boronic ester IN-001-38 was prepared as it was described for IN-001-33 on using the aryl bromide IN-001-37 (1.5 g, 3.99 mmol), KOAc (1.17 g, 11.97 mmol), bis(pinacolato)diboron (1.51 g, 5.98 mmol), [Pd(dppf)Cl₂].DCM (0.32 g, 0.45 mmol) and anhydrous DMSO (30 mL) as a solvent. The product was obtained as a yellow oil (0.736 g, 44%) after the purification by flash chromatography (0-40% Hexanes-EtOAc). MS (m/z): 423.2 (M+H), 368.3 (M-tBu+H). LCMS Ret. time: 2.53. Rf: 0.44 (Hexanes/EtOAc 80/20). ¹H NMR (CDCl₃, δ): 1.34 (s, 12H) 1.44 (s, 9H), 1.68 (m, 2H), 1.75 (m, 2H), 2.85 (s, 3H), 3.27 (br, 2H), 3.98 (pt, J=6.03 Hz, 2H), 6.54 (dd, J=11.35 Hz, J=2.30 Hz, 1H), 6.66 (dd, J=8.35 Hz, J=2.33 Hz, 1H), 7.63 (dd, J=8.10 Hz, J=7.30 Hz, 1H).

### Step 5

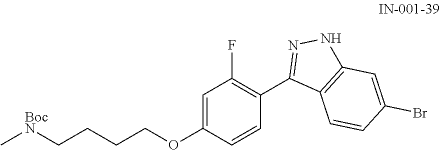 [SMILES: CN(CCCCOc1ccc(-c2n[nH]c3cc(Br)ccc23)c(F)c1)C(=O)OC(C)(C)C]

### IN-001-39. tert-butyl 4-(4-(6-bromo-1H-indazol-3-yl)-3-fluorophenoxy)butyl(methyl)carbamate

Compound IN-001-39 was prepared as it was described for IN-001-34 on using 6-bromo-3-iodo-1-H-indazole (0.112 g, 0.77 mmol), boronic ester IN-001-38 (0.192 g, 0.45 mmol), K₂CO₃ (0.145 g, 1.05 mmol), and [Pd(PPh₃)₄] 1 (0.020 g, 0.02 mmol), and water (1 mL) and dioxane (10 mL) as solvents. The product was obtained as a yellow oil (0.07 g, 41%) after purification by flash chromatography (0-90% Hexanes-EtOAc). MS (m/z): 492.3 (M+H), 436.3 (M-tBu+H), 392.3 (M-Boc+H). LCMS ret. time: 3.11. Rf: 0.33 (Hexanes/EtOAc 70/30). ¹H NMR (CDCl₃, δ): 1.46 (s, 9H), 1.47 (m, 2H), 1.60 (m, 2H), 1.85 (m, 2H), 2.85 (s, 3H), 3.25 (b, 2H), 4.01 (t, J=6.65 Hz, 2H), 6.78 (dd, J=12.61 Hz, J=2.60 Hz, 1H), 6.822 (dd, J=8.50 Hz, J=2.25 Hz, 1H), 7.298 (dd, J=8.71 Hz, J=1.60 Hz, 1H), 7.638 (m, 1H), 7.69 (m, 2H).

### Step 6

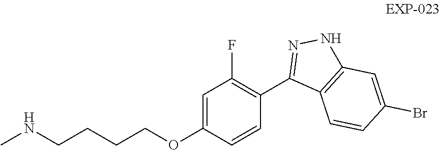 [SMILES: CNCCCCOc1ccc(-c2n[nH]c3cc(Br)ccc23)c(F)c1]

### EXP-023: 4-(4-(6-bromo-1H-indazol-3-yl)-3-fluorophenoxy)-N-methylbutan-1-amine (LZH-00025)

Compound EXP-023 was prepared as it was described for EXP-022 on using IN-001-39 (0.17 g, 0.35 mmol) and 4M HCl solution in dioxane (15 mL). The product was obtained as a white solid (0.100 g, 74%) after purification by flash chromatography (0-35% CH₂Cl₂-Polar solvent, being polar solvent MeOH/NH₄OH 5/1). MS (m/z): 392.1 (M+H). LCMS Ret. time 1.73. Rf: 0.23 (80/20 CH₂Cl₂/Polar solvent, being polar solvent MeOH/NH₄OH 5/1). ¹H NMR (CDCl₃, δ): 1.725 (quint, J=7.30 Hz, 2H), 1.86 (quint, J=6.10 Hz, 2H), 2.488 (s, 3H), 2.713 (t, J=7.30, 2H), 4.02 (t, J=5.45 Hz, 2H), 6.75 (m, 1H), 6.795 (m, 1H), 7.273 (dd, J=8.65 Hz, J=1.80 Hz, 1H), 7.665 (m, 3H).

### Synthesis of Example 24

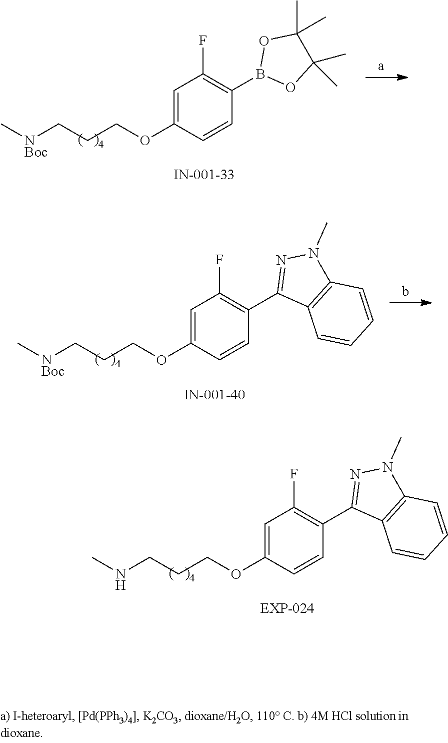

### Step 1

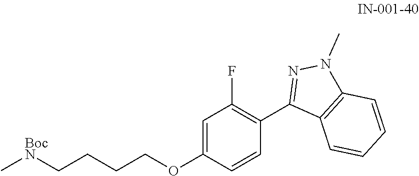 [SMILES: CN(CCCCOc1ccc(-c2nn(C)c3ccccc23)c(F)c1)C(=O)OC(C)(C)C]

### IN-001-40: Ter-butyl-6-(3-fluoro-4-(1-methyl-1H-indazol-3-yl)phenoxy)hexyl(methyl)carba-mate

A scintillation vial was charged with the 3-iodo-1-methyl-1H-indazole (0.232 g, 0.51 mmol), K₂CO₃ (0.166 g, 1.20 mmol), and [Pd(PPh₃)₄] (0.023 g, 0.02 mmol). Then, the system was put under vacuum and under vacuum a dioxane solution of the boronic ester IN-001-33 (0.1032 g, 0.40 mmol in 10 mL of solvent) and water (1 mL) were added via syringe. Then, the system was put under N₂ atmosphere and the reaction was heated at 80° C. for an overnight. After this time, the mixture was diluted with EtOAc (25 mL) and water (25 mL). The mixture was extracted, the phases separated and the aqueous phase was washed with more EtOAc (2×25 mL). The combined organic layers were dried over Na₂SO₄. The solvent was removed in vacuo and the residue was then purified by flash chromatography (0-100% Hexanes-EtOAc) and the product was obtained as a yellow solid (0.126 g, 69%). MS (m/z): 456.1 (M+H). LCMS Ret. time: 2.34. Rf: 0.44 (Hexanes/EtOAc 50/50). ¹H NMR (CDCl₃, δ): 1.38 (m, 2H), 1.46 (s, 9H), 1.55 (m, 4H), 1.84 (3, 2H), 2.85 (s, 3H), 3.23 (br, 2H), 4.03 (st, 2H, J=6.05 Hz), 4.09 (s, 3H) 6.81 (dd, J=11.56 Hz, J=2.30 Hz, 1H), 6.86 (dd, J=8.76 Hz, J=2.30 Hz, 1H), 7.07 (ddd, J=8.30 Hz, J=6.70 Hz, J=1.05 Hz, 1H), 7.30 (ddd, J=8.51 Hz, J=6.50 Hz, J=1.0 Hz, 1H), 7.36 (t, J=8.80 Hz, 1H), 7.47 (d, J=8.80 Hz, 1H), 7.71 (d, J=8.51 Hz, 1H).

### Step 2

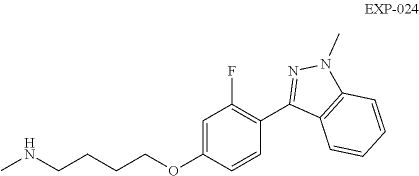 [SMILES: CNCCCCOc1ccc(-c2nn(C)c3ccccc23)c(F)c1]

### EXP-024. 6-(3-fluoro-4-(1-methyl-1H-indazol-3-yl)phenoxy)-N-methylhexan-1-amine (LZH-00026)

The N-Boc protected amine IN-00140 (0.126 g, 0.28 mmol) was dissolved in 4M HCl solution in dioxane (5 mL), and the mixture was stirred for 1 h at room temperature. The reaction was quenched by addition of saturated solution of NaHCO₃ until no more gas was produced and the pH of the solution was about 8 (aprox. 20 mL). Then, the mixture was diluted with EtOAc (20 mL) and the mixture was extracted. The organic phase was separated and the aqueous phase was extracted with EtOAc (2×20 mL). The organic layers were combined and washed with brine (50 mL), dried over Na₂SO₄ and then the solvent was evaporated in vacuo. The residue obtained was purified by flash chromatography (0-100% Hexanes-Polar solvent, being polar solvent EtOAc/MeOH 70/30) and the target product was isolated as a pale yellow solid (0.058 g, 59%). MS (m/z): 356.1 (M+H). LCMS Ret. time 1.47. Rf: 0.16 (EtOAc/MeOH 70/30). ¹H NMR (DMSO-d₆, δ): 1.36 (m, 2H), 1.46 (m, 2H), 1.62 ((m, 2H), 1.76 (m, 2H) 2.66 (s, 3H), 2.94 (pt, 2H), 4.06 (t, J=6.50 Hz, 2H), 4.09 (s, 3H), 6.93 (dd, J=8.51 Hz, J=2.35, 1H), 7.00 (dd, J=12.91 Hz, J=2.45 Hz, 1H), 7.31 (dd, J=8.71 Hz, J=1.60 Hz, 1H), 7.64 (m, 2H), 8.03 (d, J=1.50 Hz, 1H).

### Synthesis of Example 25

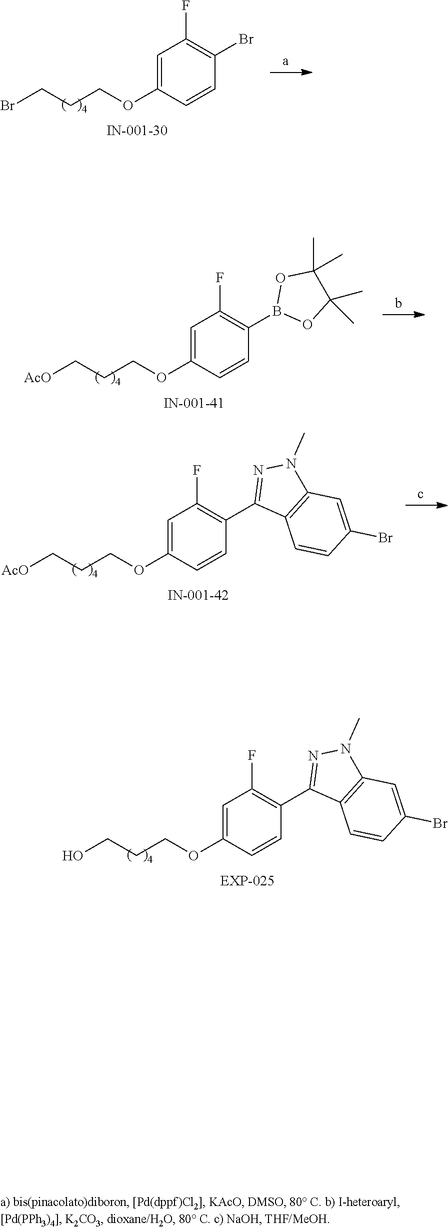

### Step 1

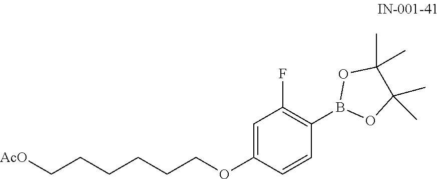 [SMILES: CC(=O)OCCCCCCOc1ccc(B2OC(C)(C)C(C)(C)O2)c(F)c1]

### IN-001-41. 6-(3-fluoro-4-(4,4,5,5-tetramethyl-1,3,2-dioxaborolan-2-yl)phenoxy)hexyl acetate

A round-bottom flask was charged with the aryl bromide IN-001-30 (2.74 g, 7.73 mmol), KOAc (2.27 g, 23.19 mmol), bis(pinacolato)diboron (2.94 g, 11.59 mmol) and [Pd(dppf)Cl₂].DCM (0.63 g, 0.77 mmol). Then, the system was put under vacuum and under vacuum anhydrous DMSO (75 mL) was added via syringe. Then, the system was put under N₂ atmosphere and the reaction was heated at 80° C. for an overnight. After cooling down, the mixture was diluted with EtOAc (160 mL) and the solution was extracted with water (4×100 mL). The organic phase was dried over Na₂SO₄ and the solvent was removed in vacuo and the residue was then purified by flash chromatography (0-40% Hexanes-EtOAc) and after the evaporation of the solvent the product was obtained as a yellow oil (2.81 g, 95%). MS(m/z): 381.2 (M+H), 403.1 (M+Na). LCMS Ret. time: 2.30. Rf: 0.51 (Hexanes/EtOAc 80/20). ¹H NMR (CDCl₃, δ): 1.44 (m, 4H), 1.65 (quint, J=6.75 Hz, 2H), 1.78 (quint, J=6.75 Hz, 2H), 2.04 (s, 3H), 3.95 (t, J=6.15 Hz, 2H), 4.06 (t, J=6.15 Hz, 2H), 6.54 (dd, J=11.50 Hz, J=1.95 Hz, 1H), 6.66 (dd, J=8.50 Hz, J=1.95 Hz, 1H), 7.62 (st, J=7.45 Hz, 1H).

### Step 2

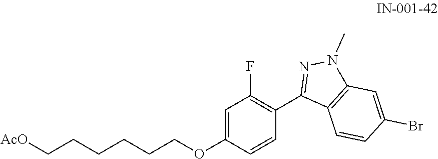 [SMILES: CC(=O)OCCCCCCOc1ccc(-c2nn(C)c3cc(Br)ccc23)c(F)c1]

### IN-001-42. 6-(4-(6-bromo-1-methyl-1H-indazol-3-yl)-3-fluorophenoxy)hexyl acetate

A round-bottom flask was charged with the 6-bromo-3-iodo-1-methyl-1-H-indazole (0.91 g, 2.70 mmol), the boronic ester IN-001-41 (1.40 g, 3.69 mmol), K₂CO₃ (1.12 g, 8.10 mmol), and [Pd(PPh₃)₄] (0.16 g, 0.13 mmol). Then, the system was put under vacuum and under vacuum dioxane (35 mL) and water (3.5 mL) were added via syringe. Then, the system was put under N₂ atmosphere and the reaction was heated at 80° C. for an overnight. After this time, the mixture was diluted with EtOAc (50 mL) and water (50 mL). The mixture was extracted, the phases separated and the aqueous phase was washed with more EtOAc (2×50 mL). The combined organic layers were dried over Na₂SO₄. The solvent was removed in vacuo and the residue was then purified by flash chromatography (0-60% Hexanes-EtOAc) and the product was obtained as a yellow oil (0.516 g, 41%). MS (m/z): 462.9 (M+H). LCMS ret time: 2.41. Rf: 0.32 (Hexanes/EtAcO 80/20). ¹H NMR (CDCl₃, δ): 1.44 (m, 2H), 1.52 (m, 2H), 1.68 (quint, J=6.10 Hz, 2H), 1.83 (quint, J=6.70 Hz, 2H), 2.05 (s, 3H), 4.01 (t, J=6.10 Hz, 2H), 4.08 (t, J=7.15 Hz, 2H), 4.08 (s, 3H), 6.76 (dd, J=12.45 Hz, J=2.55 Hz, 1H), 6.81 (dd, J=8.60 Hz, J=2.55 Hz, 1H), 7.27 (dd, J=7.90 Hz, J=1.65 Hz, 1H), 7.59 (s, 1H), 7.65 (m, 2H).

### Step 3

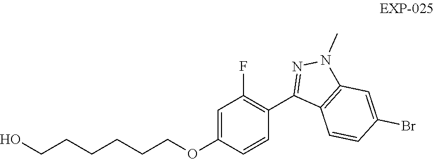 [SMILES: Cn1nc(-c2ccc(OCCCCCCO)cc2F)c2ccc(Br)cc21]

### EXP-025. 6-(4-(6-bromo-1-methyl-1H-indazol-3-yl)-3-fluorophenoxy)hexan-1-ol (LZH-00027)

To a solution of IN-001-42 (0.258 g, 0.56 mmol) in a mixture of THF/MeOH (3 mL/1.5 mL), 50% w/w NaOH solution was added (1 mL) and heated at 50° C. for 5 min. Then, the mixture was diluted with water (20 mL) and the mixture was extracted with EtOAc (3×20 mL). The combined organic layers were dried over Na₂SO₄ and the solvent was removed in vacuo. The residue was purified by flash chromatography (0-100% Hexnes-EtOAc) and the product was obtained as a yellow solid (0.212 g, 90%). MS(m/z): 420.9 (M). Rf: 0.30 (Hexanes/EtOAc 50/50). ¹H NMR (CDCl₃, δ): 1.47 (m, 4H), 1.53 (m, 2H), 1.63 (quint, J=6.00 Hz, 2H), 1.84 (quint, J=6.00 Hz, 2H), 3.68 (t, J=6.00 Hz, 2H), 4.01 (t, J=6.55 Hz, 2H), 4.08 (s, 3H), 6.76 (dd, J=12.31 Hz, J=2.45 Hz, 1H), 6.81 (dd, J=8.75 Hz, J=2.45 Hz, 1H), 7.266 (dd, J=8.51 Hz, J=1.80 Hz, 1H), 7.59 (d, J=1.60 Hz, 1H), 7.65 (m, 2H).

### Synthesis of Example 26

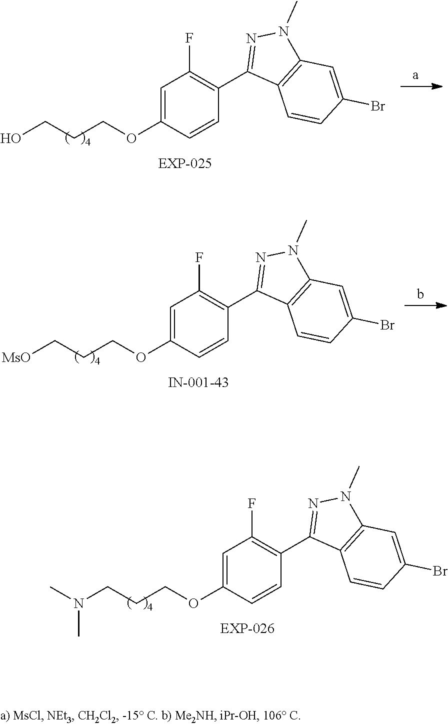

### Step 1

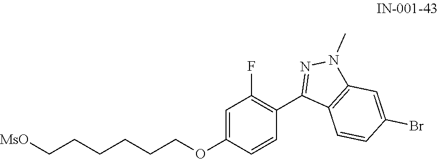 [SMILES: Cn1nc(-c2ccc(OCCCCCCOS(C)(=O)=O)cc2F)c2ccc(Br)cc21]

### IN-001-43: 6-(4-(6-bromo-1-methyl-1H-indazol-3-yl)-3-fluorophenoxy)hexyl methanesulfonate

To a solution of EXP-025 (0.192 g, 0.46 mmol) and NEt₃ (0.19 mL, 1.38 mmol) in CH₂Cl₂ (8 mL), cooled down at −15° C., a 0.1 M solution of MsCl (6.9 mL, 0.69 mmol) was added dropwise. The reaction was stirred at −15° C. for 30 min and then the reaction was warmed up using an ice/water bath, stirring the reaction of other 30 min. The solvent was removed in vacuo, and the residue was used without further purification into the next step, assuming 100% yield (0.228 g). MS (m/z): 498.9 (M+H). LCMS Ret. time: 2.22. Rf: 0.29; Hexanes/EtAcO 80/20.

### Step 2

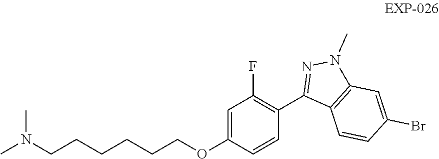 [SMILES: CN(C)CCCCCCOc1ccc(-c2nn(C)c3cc(Br)ccc23)c(F)c1]

### EXP-026: 6-(4-(6-bromo-1-methyl-1H-indazol-3-yl)-3-fluorophenoxy)-N,N-dimethylhexan-1-amine (LZH-00028)

A sealed tube with a solution of 7 (8 mL, 0.071 g/mL, 0.0568 g, 0.11 mmol) in i-PrOH and 40% aqueous solution of dimethylamine (42 μL, 0.33 mmol) was heated at 106° C. for an overnight. After cooling down the reaction mixture at room temperature, the mixture was diluted with EtOAc and water (20 mL each) and then it was transferred to a separation funnel and the mixture was extracted. The organic phase was separated and the aqueous phase was extracted with EtOAc (2×20 mL). The organic phases were combined, dried over Na₂SO₄ and then the solvent was removed in vacuo. The product was obtained as a white solid (0.042 g, 82%) after purification by flash chromatography (0-100% hexanes-polar solvent, being polar solvent EtOAc/MeOH 70/30). The product was obtained as a white solid (0.042, 82%). MS (m/z): 448.0 (M+H). LCMS Ret time: 1.79. ¹H NMR (DMSO-d₆, δ): 1.37 (quint, J=7.90, 2H), 1.46 (quint, J=7.90, 2H), 1.52 (quint, J=7.25, 2H), 1.67 (quint, J=6.65 Hz, 2H), 1.76 (quint, J=6.65 Hz, 2H), 2.66 (s, 6H), 2.95 (m, 2H), 4.06 (t, J=6.65 Hz, 2H), 4.09, (s, 3H), 6.93 (dd, J=8.66 Hz, J=2.55 Hz, 1H), 7.00 (dd, J=12.65 Hz, J=2.55 Hz, 1H), 7.31 (dd, J=8.66 Hz, J=0.95 Hz, 1H), 7.64 (m, 2H), 8.03 (s, 1H).

### Synthesis of Example 27

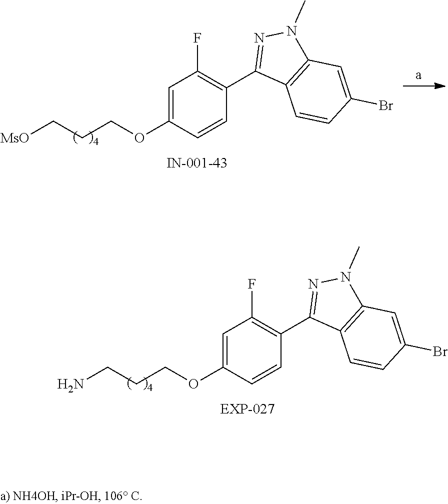

### EXP-027: 6-(4-(6-bromo-1-methyl-1H-indazol-3-yl)-3-fluorophenoxy)hexan-1-amine (LZH-00029)

EXP-027 was prepared as it was described for EXP-026 on using a solution of IN-001-43 (8 mL, 0.071 g/mL, 0.0568 g, 0.11 mmol) in i-PrOH and 28% solution of NH₄OH (0.5 mL). The product was obtained as a white solid (0.038 g, 79%) after purification by flash chromatography (0-100% hexanes-polar solvent, being polar solvent EtOAc/MeOH 70/30). MS (m/z): 420.0 (M+H). LCMS Ret time: 1.72. ¹H NMR (DMSO-d₆, δ): 1.41 (m, 4H), 1.59 (quint, J=7.15, 2H), 1.75 (quint, J=7.70 Hz, 2H), 2.76 (t, J=7.75 Hz, 2H), 4.06 (t, J=6.60 Hz, 2H), 4.08, (s, 3H), 6.927 (dd, J=8.56 Hz, J=2.30 Hz, 1H), 7.00 (dd, J=12.85 Hz, J=2.30 Hz, 1H), 7.31 (dd, J=8.55 Hz, J=1.25 Hz, 1H), 7.64 (m, 2H), 8.03 (s, 1H).

### Synthesis of Example 28

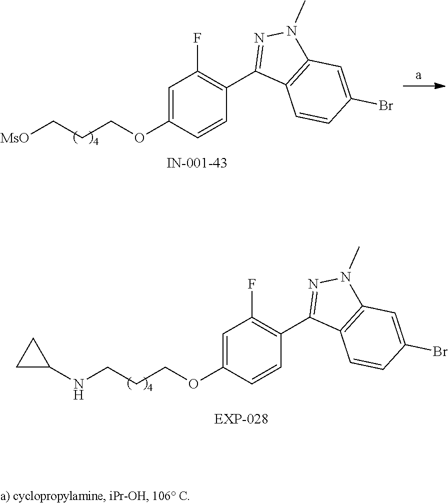

### EXP-028: N-(6-(4-(6-bromo-1-methyl-1H-indazol-3-yl)-3-fluorophenoxy)hexyl)cyclopro-panamine (LZH-00030)

EXP-028 was prepared as it was described for EXP-026 on using a solution of IN-001-43 (8 mL, 0.071 g/mL, 0.0568 g, 0.11 mmol) in i-PrOH and cyclopropylamine (23 μL, 0.33 mmol). The product was obtained as a white solid (0.047, 90%) after purification by flash chromatography (0-100% hexanes-polar solvent, being polar solvent EtOAc/MeOH 70/30). MS (m/z): 460.1 (M+H). LCMS Ret time: 1.82. ¹H NMR (CDCl₃, δ): 0.54 (m, 4H), 1.42 (quint, J=7.75, 2H), 1.51 (quint, J=7.75, 2H), 1.62 (quint, J=7.75 Hz, 2H), 1.82 (t, J=7.75 Hz, 2H), 2.22 (m, 1H), 2.78 (pt, J=7.60, 2H), 3.99 (t, J=6.65 Hz, 2H), 4.08, (s, 3H), 6.75 (dd, J=12.70 Hz, J=2.50 Hz, 1H), 6.80 (dd, J=8.56 Hz, J=2.50 Hz, 1H), 7.258 (dd, J=8.55 Hz, 3=1.55 Hz, 1H), 7.58 (d, J=1.55 Hz, 1H), 7.65 (m, 2H).

### Synthesis of Example 29

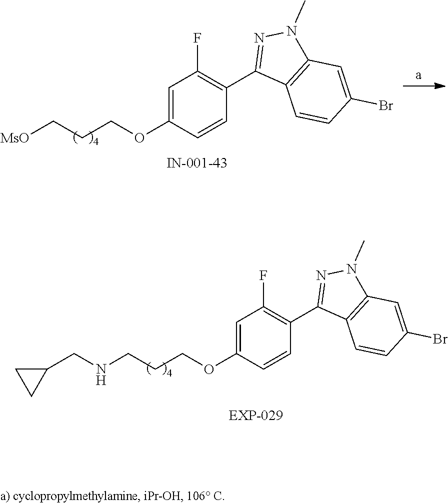

### EXP-029: 6-(4-(6-bromo-1-methyl-1H-indazol-3-yl)-3-fluorophenoxy)-N(cyclopropylmethyl)-hexan-1-amine (LZH-00031)

EXP-029 was prepared as it was described for EXP-026 on using a solution of IN-001-43 (8 mL, 0.071 g/mL, 0.0568 g, 0.11 mmol) in i-PrOH and cyclopropylmethylamine (29 μL, 0.33 mmol). The product was obtained as a white solid (0.047, 87%) after purification by flash chromatography (0-100% hexanes-polar solvent, being polar solvent EtOAc/MeOH 70/30). MS (m/z): 474.1 (M+H). LCMS Ret time: 1.86. ¹H NMR (CDCl₃, δ): 0.44 (m, 2H), 0.69 (m, 2H), 1.99 (m, 1H), 1.51 (m, 4H), 1.82 (quint, J=7.00 Hz, 2H), 1.96 (quint, J=7.00 Hz, 2H), 2.22 (m, 1H), 3.02 (br, 2H), 3.98 (t, J=6.20 Hz, 2H), 4.07 (s, 3H), 6.74 (dd, J=12.01 Hz, J=2.30 Hz, 1H), 6.79 (dd, J=8.15 Hz, J=2.30 Hz, 1H), 7.24 (dd, J=8.56 Hz, J=1.70 Hz, 1H), 7.572 (d, J=1.25 Hz, 1H), 7.63 (m, 2H).

### Synthesis of Example 30

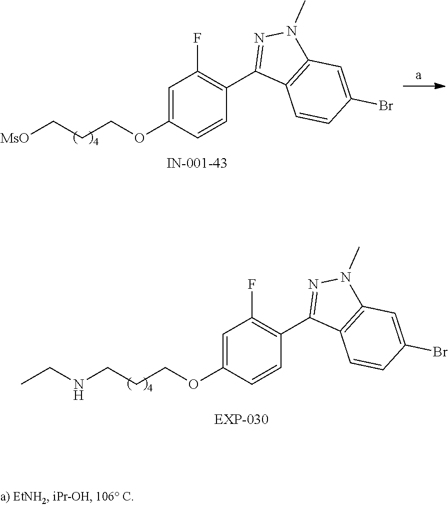

### EXP-030: 6-(4-(6-bromo-1-methyl-1H-indazol-3-yl)-3-fluorophenoxy)-N-ethylhexan-1-amine (LZH-00032)

EXP-030 was prepared as it was described for EXP-026 on using a solution of IN-001-43 (8 mL, 0.071 g/mL, 0.0568 g, 0.11 mmol) in i-PrOH and EtNH₂ (29 μL, 0.33 mmol). The product was obtained as a white solid (0.032 g, 62%) after purification by flash chromatography 0-100% 4CV hexanes-polar solvent, being polar solvent EtOAc/MeOH 70/30). MS: 448.1 m/z (M+H). LCMS Ret. time: 1.79. ¹H NMR (CDCl₃, δ): 1.485 (t, J=6.95 Hz, 3H), 1.526 (m, 2H), 1.808 (quint, J=6.45 Hz, 2H), 1.958 (quint, J=7.85 Hz, 2H), 2.939 (m, 2H), 3.042 (br, 2H), 3.972 (t, J=6.50 Hz, 2H), 4.062 (s, 3H), 4.116 (q, J=6.95 Hz, 2H), 6.731 (dd, J=12.25 Hz, J=2.30 Hz, 1H), 6.777 (dd, J=8.66 Hz, J=2.30 Hz, 1H), 7.236 (dd, J=8.66 Hz, J=1.60 Hz, 1H), 7.565 (d, J=1.40 Hz, 1H), 7.627 (m, 2H).

### Synthesis of Example 31

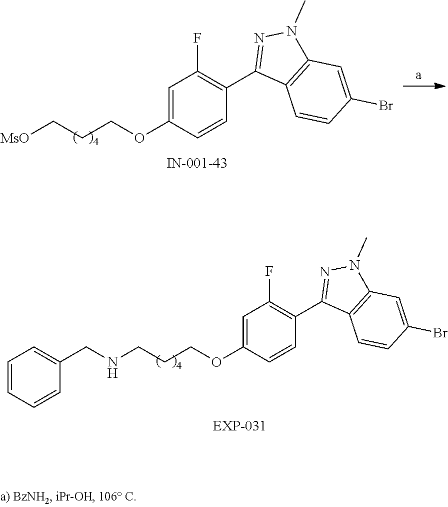

### EXP-031: N-benzyl-6-(4-(6-bromo-1-methyl-1H-indazol-3-yl)-3-fluorophenoxy)hexan-1-amine (LZH-00033)

EXP-031 was prepared as it was described for EXP-026 on using a solution of IN-001-43 (8 mL, 0.071 g/mL, 0.0568 g, 0.11 mmol) in i-PrOH and BzNH₂ (29 μL, 0.33 mmol). The product was obtained as a white solid (0.046 g, 80%) after purification by flash chromatography (0-100% 4CV hexanes-polar solvent, being polar solvent EtOAc/MeOH 70/30). MS: 510.0 m/z (M+H). LCMS Ret. time: 1.92. Rf: 0.37 (EtOAc/MeOH 90/10). ¹H NMR (CDCl₃, δ): 1.441 (m, 4H), 1.769 (m, 2H), 2.731 (pt, J=7.90 Hz, 2H), 3.931 (s, 2H), 3.958 (pt, J=6.35 Hz, 2H), 4.072 (s, 3H), 6.726 (dd, J=12.25 Hz, J=2.65 Hz, 1H), 6.775 (dd, J=8.76 Hz, J=2.65 Hz, 1H), 7.247 (dd, J=8.51 Hz, J=1.45 Hz, 1H), 7.311 (m, 1H), 7.370 (m, 2H), 7.483 (d, J=7.20 Hz, 2H), 7.574 (d, J=1.65 Hz, 1H), 7.63 (m, 2H).

### Synthesis of Example 32

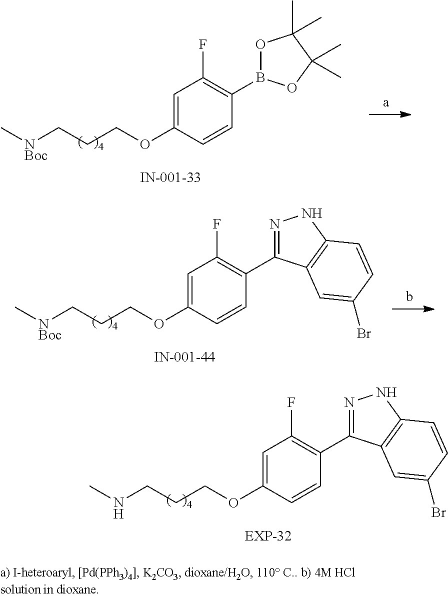

### Step 1

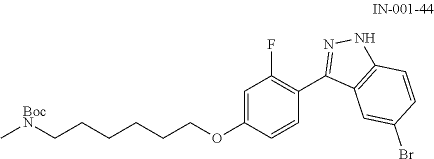 [SMILES: CN(CCCCCCOc1ccc(-c2n[nH]c3ccc(Br)cc23)c(F)c1)C(=O)OC(C)(C)C]

### IN-001-44: tert-butyl6-(4-(5-bromo-1H-indazol-3-yl)-3-fluorophenoxy)hexyl(methyl)carbamate

Compound IN-001-44 was prepared as it was described for IN-001-40 on using 5-bromo-3-iodo-1-H-indazole (0.100 g, 0.31 mmol), boronic ester IN-001-33 (0.154 g, 0.34 mmol), K₂CO₃ (0.123 g, 0.93 mmol), and [Pd(PPh₃)₄] (0.018 g, 0.02 mmol), and water (1 mL) and dioxane (10 mL) as solvents. The product was obtained as a yellow oil (0.056 g, 35%) after purification by flash chromatography (0-30% Hexanes-EtOAc). MS (m/z): 520.1 (M+H), 564.0 (M-tBu+H), 420.1 (M-Boc+H). LCMS ret. time: 2.46. Rf: 0.48 (Hexanes/EtAcO 80/20). ¹H NMR (CDCl₃, δ): 1.37 (m, 2H), 1.46 (s, 9H), 1.53 (m, 4H), 1.82 (m, 2H), 1.94 (br, 2H), 2.85 (s, 3H), 3.22 (br, 2H), 4.01 (t, J=6.40 Hz, 2H), 6.78 (dd, J=12.30 Hz, J=2.75 Hz, 1H), 6.82 (dd, J=8.71 Hz, J=2.25 Hz, 1H), 7.38 (dd, J=8.85 Hz, J=0.55 Hz, 1H), 7.49 (dd, J=8.65 Hz, J=1.85 Hz, 1H), 7.66 (t, J=8.65 Hz, 1H), 7.98 (m, 1H).

### Step 2

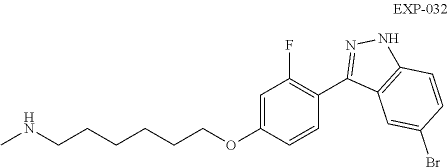 [SMILES: CNCCCCCCOc1ccc(-c2n[nH]c3ccc(Br)cc23)c(F)c1]

### EXP-032: 6-(4-(5-bromo-1H-indazol-3-yl)-3-fluorophenoxy)-N-methylhexan-1-amine (LZH-00034)

EXP-032 was prepared as it was described for EXP-024 on using IN-001-44 (0.056 g, 0.11 mmol) and 4M HCl solution in dioxane (5 mL). The product was obtained as a white solid (0.030 g, 66%) after purification by flash chromatography (0-40% CH₂Cl₂-Polar solvent, being polar solvent MeOH/NH₄OH 5/1). MS (m/z): 420.1 (M+H). LCMS Ret. time 1.63. Rf: 0.33 (80/20 CH₂Cl₂/Polar solvent, being polar solvent MeOH/NH₄OH 5/1). ¹H NMR (CDCl₃, δ): 1.41 (m, 2H), 1.49 (m, 2H), 1.55 (m, 2H), 1.81 (quint, J=6.80 Hz, 2H), 1.926 (quint, J=7.85 Hz, 2H), 2.45 (s, 3H), 2.61 (t, J=7.75 Hz, 2H), 3.985 (t, J=7.75 Hz, 2H), 6.76 (dd, J=12.2 Hz, J=2.85, 1H), 6.80 (dd, J=8.56 Hz, J=2.35 Hz, 1H), 7.37 (d, J=9.01 Hz, 1H), 7.45 (dd, J=9.01 Hz, J=2.10, 1H), 7.64 (t, J=9.01 Hz, 1H), 7.96 (m, 1H).

### Synthesis of Example 33

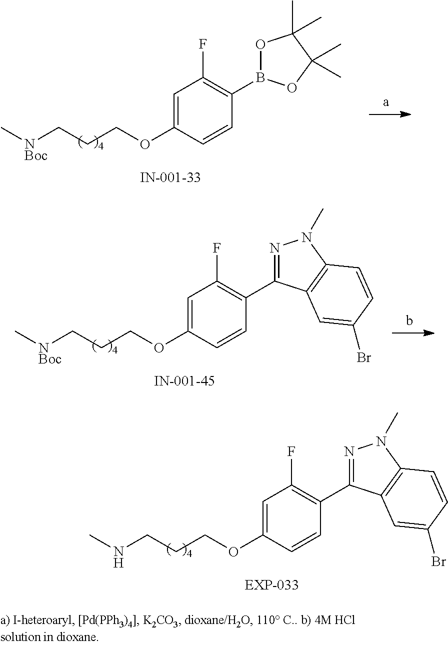

### Step 1

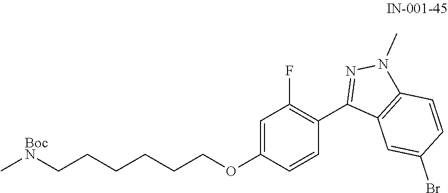 [SMILES: CN(CCCCCCOc1ccc(-c2nn(C)c3ccc(Br)cc23)c(F)c1)C(=O)OC(C)(C)C]

### IN-001-45. tert-butyl6-(4-(5-bromo-1-methyl-1H-indazol-3-yl)-3-fluorophenoxy)hexyl(methyl)-carbamate

Compound IN-001-45 was prepared as it was described for IN-001-40 on using 5-bromo-3-iodo-1-methylindazole (0.100 g, 0.31 mmol), boronic ester IN-001-33 (0.154 g, 0.34 mmol), K₂CO₃ (0.123 g, 0.93 mmol), and [Pd(PPh₃)₄](0.018 g, 0.02 mmol), and water (1 mL) and dioxane (10 mL) as solvents. The product was obtained as a yellow oil (0.056 g, 35%) after purification by flash chromatography (0-30% Hexanes-EtOAc). MS (m/z): 520.1 (M+H), 564.0 (M-tBu+H), 420.1 (M-Boc+H). LCMS ret. time: 2.46. Rf: 0.48 (Hexanes/EtAcO 80/20). ¹H NMR (CDCl₃, δ): 1.36 (m, 2H), 1.46 (s, 9H), 1.53 (m, 4H), 1.82 (m, 2H), 2.84 (s, 3H), 3.22 (br, 2H), 4.00 (t, J=5.00 Hz, 2H), 4.10 (s, 3H), 6.76 (dd, J=12.31 Hz, J=2.40 Hz, 1H), 6.81 (dd, J=8.45 Hz, J=2.30 Hz, 1H), 7.28 (dd, J=8.60 Hz, J=0.50 Hz, 1H), 7.48 (dd, J=8.52 Hz, J=1.75 Hz, 1H), 7.63 (t, J=8.52 Hz, 1H), 7.94 (m, 1H).

### Step 2

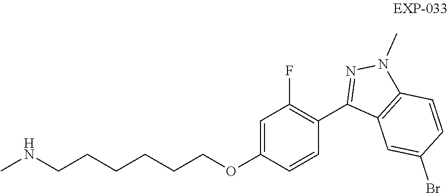 [SMILES: CNCCCCCCOc1ccc(-c2nn(C)c3ccc(Br)cc23)c(F)c1]

### EXP-033: 6-(4-(5-bromo-1-methyl-1H-indazol-3-yl)-3-fluorophenoxy)-N-methylhexan-1-amine (LZH-00035)

EXP-033 was prepared as it was described for EXP-024 on using IN-001-45 (0.056 g, 0.10 mmol) and 4M HCl solution in dioxane (5 mL). The product was obtained as a white solid (0.045 g, 99%) after purification by flash chromatography (0-40% CH₂Cl₂-Polar solvent, being polar solvent MeOH/NH₄OH 5/1). MS (m/z): 434.2 (M+H). LCMS Ret. time 1.75. Rf: 0.4 (80/20 CH₂Cl₂/Polar solvent, being polar solvent MeOH/NH₄OH 5/1). ¹H NMR (CDCl₃, δ): 1.41 (m, 2H), 1.50 (m, 2H), 1.55 (quint, J=7.25 Hz, 2H), 1.81 (quint, J=7.25 Hz, 2H), 2.44 (s, 3H), 2.61 (t, J=7.25 Hz, 2H), 3.99 (t, J=6.45 Hz, 2H), 4.09 (s, 3H), 6.75 (dd, J=12.4 Hz, J=2.60, 1H), 6.80 (dd, J=8.71 Hz, J=2.55 Hz, 1H), 7.77 (d, J=8.96 Hz, 1H), 7.46 (dd, J=8.96 Hz, J=1.70, 1H), 7.62 (t, J=8.96 Hz, 1H), 7.93 (m, 1H).

### Synthesis of Example 34

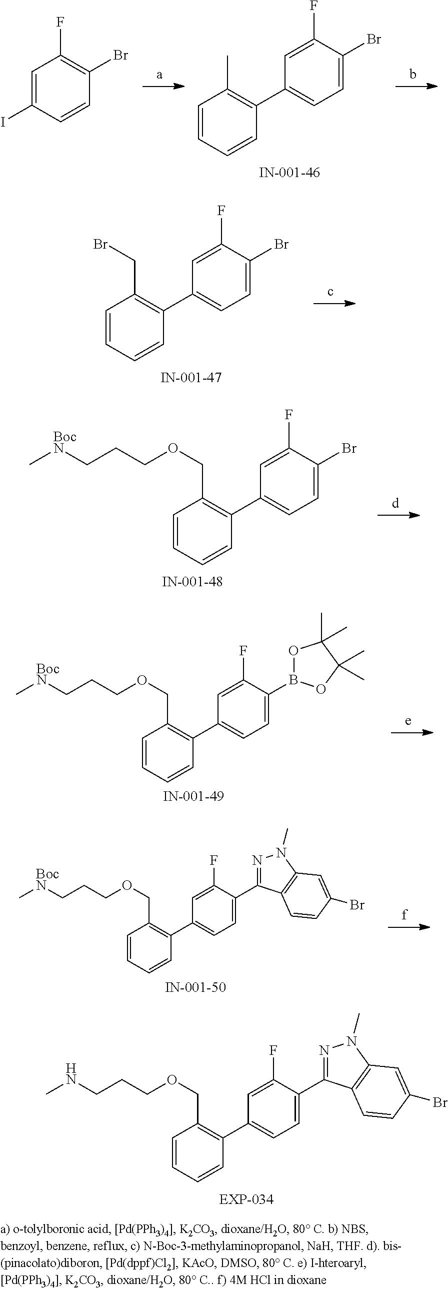

### Step 1

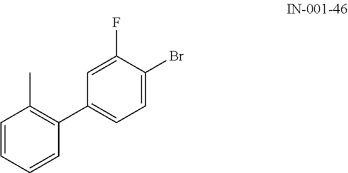

### IN-001-46: 4′-bromo-3′-fluoro-2-methylbiphenyl

A scintillation vial was charged with 1-bromo-2-fluoro-4-iodobenzene (5.00 g, 16.62 mmol), o-tolylboronic acid (2.25 g, 16.62 mmol), K₂CO₃ (6.89 g, 49.86 mmol), and [Pd(PPh₃)₄] (0.96 g, 0.83 mmol). Then, the system was put under vacuum and under vacuum a dioxane/water mixture (18 mL/1.8 mL) was added via syringe. Then, the system was put under N₂ atmosphere and the reaction was heated at 80° C. for an overnight. After this time, the solvent was removed in vacuo and the residue was then purified by flash chromatography (0-5% Hexanes-EtOAc) and after the evaporation of the solvent, the product was obtained as a colorless oil (4.22 g, 96%). The molecular ion was not detected. LCMS ret. time: 2.34. Rf: 0.59 (Hexanes). ¹H NMR (CDCl₃, δ): 2.25 (s, 3H), 6.97 (dd, J=8.25 Hz, J=1.75 Hz, 1H), 7.08 (dd, J=9.55 Hz, J=1.75 Hz, 1H), 7.16 (d, J=6.95H, 1H), 7.24 (m, 3H), 7.65 (pt, J=7.80 Hz, 1H).

### Step 2

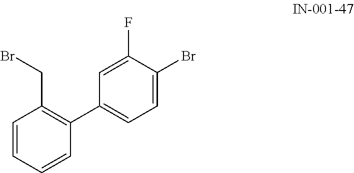 [SMILES: Fc1cc(-c2ccccc2CBr)ccc1Br]

### IN-001-47: 4′-bromo-2-(bromomethyl)-3′-fluorobiphenyl

To a solution of IN-001-46 (4.22 g, 15.92 mmol) and N-bromosuccinimide (2.97 g, 16.72 mmol) in benzene (100 mL), under N₂ atmosphere, benzoyl peroxide (0.77 g, 3.18 mmol) was added. The mixture was stirred at 80° C. for 1 h. Then, the solvent was removed in vacuo, and the residue was redisolved in EtOAc (100 mL) and washed with saturated aqueous solution of NaHCO₃ (100 mL) and brine (100 mL). The organic layer was dried over Na₂SO₄ and the solvent was removed in vacuo. The residue was then purified by flash chromatography using hexanes as eluent and the product was obtained as a white solid (4.00 g, 73%). The molecular ion was not observed. LCMS Ret. time: 2.27. Rf: 0.5 (Hexanes). ¹H NMR (CDCl₃, δ): 4.41 (s, 2H), 7.14 (d, J=8.25 Hz, 1H), 7.23 (m, 2H), 7.36 (t, J=7.15H, 1H), 7.40 (t, J=7.50H, 1H), 7.53 (d, J=7.50 Hz, 1H) 7.63 (t, J=7.50 Hz, 1H).

### Step 3

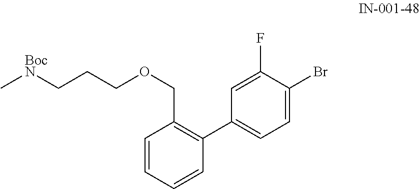 [SMILES: CN(CCCOCc1ccccc1-c1ccc(Br)c(F)c1)C(=O)OC(C)(C)C]

### IN-001-48: tert-butyl 3-((4′-bromo-3′-fluorobiphenyl-2-yl)methoxy)propyl(methyl)carbamate

To an anhydrous THF (60 mL) solution of 9 (2.19 g, 6.37 mmol) and N-Boc-3-methylaminopropanol (1.20 g, 6.37 mmol), NaH (0.38 g NaH at 60%, 0.23 g of NaH, 9.55 mmol) was added portionwise. The reaction mixture was stirred for room temperature for 5 h under N₂ atmosphere. Then, the reaction was quenched adding water (40 mL) and the mixture was extracted with EtOAc (3×60 mL). The organic layers were combined and the combined organic phases were washed with water (3×100 mL). The organic phase was dried over Na₂SO₄ and the solvent was removed in vacuo. The crude was purified by flash chromatography (Hexanes-EtOAc 0-20%) and the product was obtained as a yellow oil (2.12 g, 74%). MS (m/z): 452.0 (M+H), 396.0 (M-tBu+H), 352.0 (M+Boc+H). LCMS Ret. time: 2.46. Rf: 0.55 (Hexanes/EtOAc 80/20). ¹H NMR (CDCl₃, δ): 1.43 (s, 9H), 1.80 (quint, J=7.30 Hz, 2H), 2.83 (s, 1H), 3.27 (br, 2H), 3.43 (7t, J=6.50 Hz, 2H), 4.32 (s, 2H), 7.055 (dd, J=8.30 Hz J=1.85 Hz, 1H), 7.205 (dd, J=9.51 Hz, J=1.85 Hz, 1H), 7.25 (d, J=7.80 Hz, 1H), 7.37 (m, 2H), 7.514 (d, J=7.80 Hz, 1H), 7.58 (dd, J=8.15 Hz, J=7.20 Hz, 1H).

### Step 4

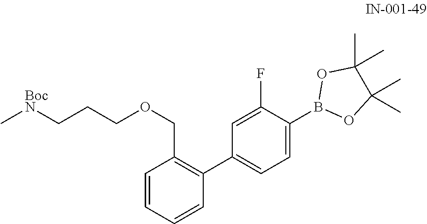

### IN-001-49: tert-butyl 3-((3′-fluoro-4′-(4,5,5-tetramethyl-1,3,2-dioxaborolan-2-yl)biphenyl-2-yl)methoxy)propyl(methyl)carbamate

A round-bottom flask was charged with the aryl bromide IN-001-48 (0.20 g, 0.44 mmol), KOAc (0.13 g, 1.32 mmol), bis(pinacolato)diboron (0.17 g, 0.66 mmol), [Pd(dppf)Cl₂] (0.032 g, 0.04 mmol). Then, the system was put under vacuum and under vacuum anhydrous DMSO (20 mL) was added via syringe. Then, the system was put under N₂ atmosphere and the reaction was heated at 80° C. for an overnight. After cooling down, the mixture was diluted with EtOAc (50 mL) and the solution was extracted with water (50 mL). The aqueous phase was extracted with more EtOAc (2×50 mL). The solvent for the combined organic layers was evaporated to half of the volume and then the EtOAc solution was washed with water (3×50 mL). The organic phase was dried over Na₂SO₄ and the solvent was removed in vacuo and the residue was then purified by flash chromatography (0-30% Hexanes-EtOAc) and after the evaporation of the solvent the product was obtained as a colorless oil (0.10 g, 45%). MS(m/z): 500.2 (M+H), 444.1 (M+H-tBu), 400.2 (M+H-Boc). LCMS Ret. time: 2.55. ¹H NMR (CDCl₃, δ): 1.38 (s, 12H) 1.43 (s, 9H), 1.79 (quint, J=7.15 Hz, 2H), 2.82 (s, 3H), 3.26 (br, 2H), 3.42 (t, J=6.00 Hz, 2H), 4.34 (s, 2H), 7.10 (dd, J=10.30 Hz, J=1.20 Hz, 1H), 7.16 (dd, J=7.45 Hz, J=1.20 Hz, 1H), 7.265 (d, J=7.00 Hz, 1H), 7.365 (m, 2H), 7.821 (d, J=7.20 Hz, 1H) 7.77 (dd, J=7.70 Hz, J=6.50 Hz, 1H).

### Step 5

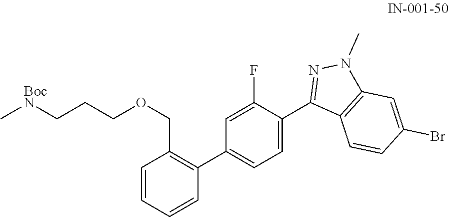 [SMILES: CN(CCCOCc1ccccc1-c1ccc(-c2nn(C)c3cc(Br)ccc23)c(F)c1)C(=O)OC(C)(C)C]

### IN-001-50: tert-butyl 3-((4′-(6-bromo-1-methyl-1H-indazol-3-yl)-3′-fluorobiphenyl-2-yl)methoxy)propyl(methyl)carbamate

A round-bottom flask was charged with the 6-bromo-3-iodo-1-methyl-indazole (0.273 g, 0.81 mmol), boronic ester IN-001-49 (0.27 g, 0.54 mmol), K₂CO₃ (0.224 g, 1.62 mmol), and [Pd(PPh₃)₄] Pd(PPh₃)₄] (0.031 g, 0.03 mmol). Then, the system was put under vacuum and under vacuum a dioxane (18 mL) and water (1.8 mL) were added via syringe. Then, the system was put under N₂ atmosphere and the reaction was heated at 80° C. for an overnight. After this time, the mixture was diluted with EtOAc (25 mL) and water (25 mL). The mixture was extracted, the phases separated and the aqueous phase was washed with more EtOAc (2×25 mL). The combined organic layers were dried over Na₂SO₄. The solvent was removed in vacuo and the residue was then purified by flash chromatography (0-100% Hexanes-EtOAc) and The product was obtained as a yellow oil (0.036 g, 11%) after purification by flash chromatography (0-20% Hexanes-EtOAc). MS (m/z): 582.2 (M+H), 526.1 (M-tBu+H), 482.1 (M-Boc+H). LCMS Ret. time: 2.63. Rf: 0.16 (hexanes/EtOAc 80/20).

### Step 6

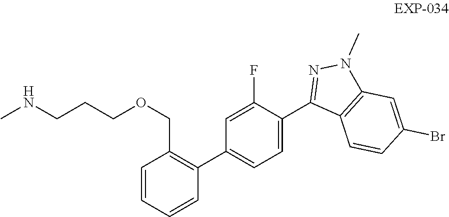 [SMILES: CNCCCOCc1ccccc1-c1ccc(-c2nn(C)c3cc(Br)ccc23)c(F)c1]

### EXP-034: 3-((4′-(6-bromo-1-methyl-1H-indazol-3-yl)-3′-fluorobiphenyl-2-yl)methoxy)-N-methylpropan-1-amine (LZH-00036)

The N-Boc protected amine IN-001-50 (0.036 g, 0.11 mmol) was dissolved in 4M HCl solution in dioxane (5 mL), and the mixture was stirred for 1 h at room temperature. The reaction was quenched by addition of saturated solution of NaHCO₃ until no more gas was produced and the pH of the solution was about 8 (aprox. 20 mL). Then, the mixture was diluted with EtOAc (20 mL) and the mixture was extracted. The organic phase was separated and the aqueous phase was extracted with EtOAc (2×20 mL). The organic layers were combined and washed with brine (50 mL), dried over Na₂SO₄ and then the solvent was evaporated in vacuo. The solid yellow residue obtained after the evaporation was purified by flash chromatography (0-100% Hexanes-Polar solvent, being polar solvent EtOAc/MeOH 70/30) and the product was obtained as a white solid (0.021 g, 70%). MS (m/z): 482.1 (M+H). LCMS Ret. time: 1.88. Rf: 0.20 (EtOAc/MeOH 70/30) ¹H NMR (CDCl₃, δ): 2.12 (quint, J=5.75 Hz, 2H), 2.65 (s, 3H), 3.05 (t, J=8.55 Hz, 2H), 3.55 (t, J=5.75 Hz, 2H), 4.12 (s, 3H), 4.46 (s, 2H), 4.82 (br, 1H), 7.25 (m, 2H), 7.32 (m, 2H), 7.39 (m, 2H), 7.51 (m, 1H), 7.62 (d, J=1.15 Hz, 1H), 7.755 (dd, J=9.00 Hz, J=3.15 Hz, 1H), 7.82 (pt, J=7.45 Hz, 1H).

### Synthesis of Example 35

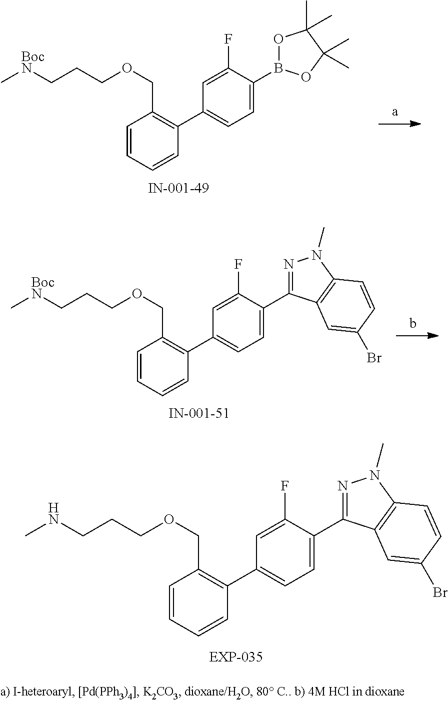

### Step 1

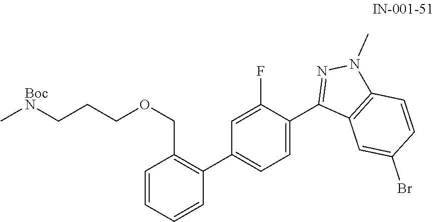 [SMILES: CN(CCCOCc1ccccc1-c1ccc(-c2nn(C)c3ccc(Br)cc23)c(F)c1)C(=O)OC(C)(C)C]

### IN-001-51. tert-butyl 3-((4′-(5-bromo-1-methyl-1H-indazol-3-yl)-3′-fluorobiphenyl-2-yl)methoxy)propyl(methyl)carbamate

Compound IN-001-51 was prepared as it was described for IN-001-50 on using 5-bromo-3-iodo-1-methyl-indazole (0.273 g, 0.81 mmol), boronic ester IN-001-49 (0.27 g, 0.54 mmol), K₂CO₃ (0.224 g, 1.62 mmol), and [Pd(PPh₃)₄](0.031 g, 0.03 mmol), and water (1.8 mL) and dioxane (18 mL) as solvents. The product was obtained as a yellow oil (0.036 g, 11%) after purification by flash chromatography (0-20% Hexanes-EtOAc). MS (m/z): 582.2 (M+H), 526.1 (M-tBu+H), 482.1 (M-Boc+H). LCMS Ret. time: 2.64. Rf: 0.16 (hexanes/EtOAc 80/20).

### Step 2

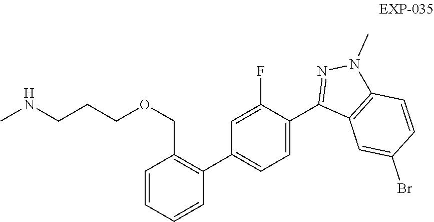 [SMILES: CNCCCOCc1ccccc1-c1ccc(-c2nn(C)c3ccc(Br)cc23)c(F)c1]

### EXP-035: 3-((4′-(5-bromo-1-methyl-1H-indazol-3-yl)-3′-fluorobiphenyl-2-yl)methoxy)-N-methylpropan-1-amine (LZH-00037)

EXP-035 was prepared as it was described for EXP-034 on using IN-001-51 (0.035 g, 0.06 mmol) and 4M HCl solution in dioxane (5 mL). The product was obtained as a white solid (0.023 g, 79%) after purification by flash chromatography (0-100% Hexanes-Polar solvent, being polar solvent EtOAc/MeOH 70/30). MS (m/z): 482.1 (M+H). LCMS Ret. time: 1.87. Rf: 0.20 (EtOAc/MeOH 70/30). ¹H NMR (CDCl₃, δ): 2.12 (quint, J=6.60 Hz, 2H), 2.65 (s, 3H), 3.05 (t, J=7.30 Hz, 2H), 3.54 (t, J=5.55 Hz, 2H), 4.12 (s, 3H), 4.44 (s, 2H), 4.82 (br, 1H), 7.25 (m, 2H), 7.31 (m, 2H), 7.38 (m, 2H), 7.49 (m, 2H), 7.79 (pt, J=7.85 Hz, 1H), 8.02 (pt, J=1.85 Hz, 1H).

### Synthesis of 36

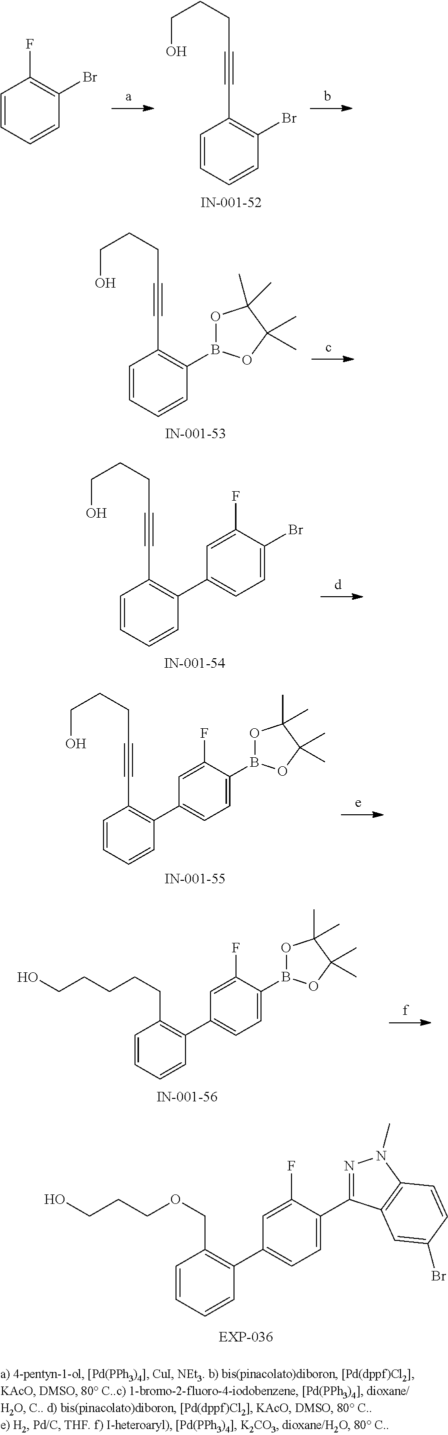

### Step 1

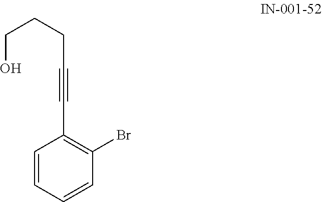 [SMILES: OCCCC#Cc1ccccc1Br]

### IN-001-52: 5-(2-bromophenyl)pent-4-yn-1-ol

A round-bottom flask was charged with CuI (0.14 g, 0.71 mmol) and [Pd(PPh₃)₄] (0.41 g, 0.35 mmol) and the system was put under vacuum and under vacuum, a NEt₃ (25 mL) solution of the 1-bromo-2-iodobenzene (5 g, 17.67 mmol) and a NEt₃ solution (25 mL) of 4-pentyn-1-ol (1.93 g, 22.97 mmol) were added via syringe. Then, the mixture was put under N₂ atmosphere and the mixture was stirred at room temperature for 24 h. After this time, the solvent was removed in vacuo, and the residue was redisolved in EtOAc (50 mL) and the solution was washed with water (3×50 mL). The organic phase was dried over Na₂SO₄ and the solvent was removed in vacuo. The residue was purified by flash chromatography (0-30% Hexanes-EtOAc) and the product was obtained as an orange oil (1.77 g, 20%). The molecular ion was not observed. LCMS Ret. time: 1. 60. Rf: 0.23 (Hexanes/EtOAc 50/50). ¹H NMR (CDCl₃, δ): 1.90 (quint, J=6.65 Hz, 2H), 2.61 (t, J=6.65 Hz, 2H), 3.87 (pt, J=6.65 Hz, 2H), 7.122 (td, J=7.70 Hz, J=1.70 Hz, 1H), 7.227 (td, J=7.50 Hz, J=1.10 Hz, 1H), 7.42 (dd, J=7.50 Hz, J=1.10 Hz, 1H), 7.5 (dd, J=8.00 Hz, J=1.10 Hz, 1H).

### Step 2

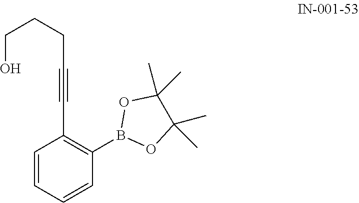 [SMILES: CC1(C)OB(c2ccccc2C#CCCCO)OC1(C)C]

### IN-001-53: 5-(2-(4,4,5,5-tetramethyl-1,3,2-dioxaborolan-2-yl)phenyl)pent-4-yn-1-ol

A round-bottom flask was charged with the aryl bromide IN-001-52 (1.77 g, 7.40 mmol), KOAc (2.18 g, 22.20 mmol), bis(pinacolato)diboron (2.81 g, 11.10 mmol) and [Pd(dppf)Cl₂] (0.54 g, 0.74 mmol). Then, the system was put under vacuum and under vacuum anhydrous DMSO (80 mL) was added via syringe. Then, the system was put under N₂ atmosphere and the reaction was heated at 80° C. for an overnight. After cooling down, the mixture was diluted with EtOAc (160 mL) and the solution was extracted with water (4×100 mL). The organic phase was dried over Na₂SO₄ and the solvent was removed in vacuo and the residue was then purified by flash chromatography (0-30% Hexanes-EtOAc) and after the evaporation of the solvent the product was obtained as a red-orange oil (0.94 g, 44%). MS: 287.2 (M+H). LCMS Ret. time: 1.77. Rf: 0.23 (Hexanes/EtOAc 50/50). ¹H NMR (CDCl₃, δ): 1.36 (s, 12H), 1.87 (quint, J=6.73 Hz, 2H), 2.58 (t, J=6.73 Hz, 2H), 3.87 (pt, J=6.10 Hz, 2H), 7.252 (td, J=7.50 Hz, J=1.30 Hz, 1H), 7.336 (td, J=7.65 Hz, J=1.40 Hz, 1H), 7.41 (d, J=7.75 Hz, 1H), 7.72 (dd, J=7.60 Hz, J=1.30 Hz, 1H).

### Step 3

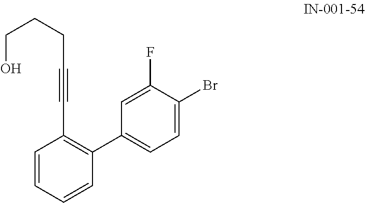 [SMILES: OCCCC#Cc1ccccc1-c1ccc(Br)c(F)c1]

### IN-001-54: 5-(4′-bromo-3′-fluorobiphenyl-2-yl)pent-4-yn-1-ol

A round-bottom flask attached to a condenser was charged with the boronic ester IN-001-53 (0.94 g, 3.28 mmol), 1-bromo-2-fluoro-4-iodobenzene (01.28 g, 4.26 mmol), K₂CO₃ (1.36 g, 9.84 mmol), and [Pd(PPh₃)₄] (0.19 g, 0.16 mmol). Then, the system was put under vacuum and under vacuum dioxane (50 mL) and water (5 mL) were added via syringe. Then, the system was put under N₂ atmosphere and the reaction was heated at 80° C. for 6 h. After this time, the mixture was diluted with EtOAc (50 mL) and water (50 mL). The mixture was extracted, the phases separated and the aqueous phase was washed with more EtOAc (2×50 mL). The combined organic layers were dried over Na₂SO₄. The solvent was removed in vacuo and the residue was then purified by flash chromatography (0-30% Hexanes-EtOAc) and the product was obtained as brown orange oil (0.73 g, 67%). MS (m/z): 355.1 (M+Na), 333.1 (M+H). LCMS ret. time: 2.21. Rf: 0.42 (Hexanes/EtOAc 50/50). ¹H NMR (CDCl₃, δ): 1.85 (quint, J=7.00 Hz, 2H), 2.45 (t, J=7.00 Hz, 2H), 3.66 (m, 2H), 7.22 (dd, J=8.25 Hz, J=0.70 Hz, 1H), 7.31 (m, 3H), 7.406 (dd, J=9.86 Hz, J=2.00 Hz, 1H), 7.50 (m, 2H), 7.58 (dd, J=8.15 Hz, J=7.15 Hz, 1H).

### Step 4

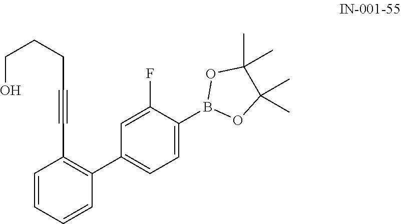 [SMILES: CC1(C)OB(c2ccc(-c3ccccc3C#CCCCO)cc2F)OC1(C)C]

### IN-001-55: 5-(3′-fluoro-4′-(4,4,5,5-tetramethyl-1,3,2-dioxaborolan-2-yl)biphenyl-2-yl)pent-4-yn-1-ol

Compound IN-001-55 was prepared as it was described for IN-001-53 on using IN-001-54 (0.79 g, 2.37 mmol), KOAc (0.70 g, 7.11 mmol), bis(pinacolato)diboron (1.20 g, 4.74 mmol) and [Pd(dppf)Cl₂] (0.26 g, 0.35 mmol) and anhydrous DMSO (30 mL). The product was obtained as a yellow oil (0.64 g, 71%) after purification by flash chromatography (0-45% Hexanes-EtOAc). MS: 381.3 (M+H). LCMS Ret. time: 2.34. Rf: 0.33 (Hexanes/EtOAc 50/50). ¹H NMR (CDCl₃, δ): 1.38 (s, 12H), 1.74 (quint, J=6.40 Hz, 2H), 2.43 (t, J=6.40 Hz, 2H), 3.61 (t, J=6.40 Hz, 2H), 7.29 (m, 2H), 7.33 (m, 3H), 50 (d, J=7.35 Hz, 1H), 7.77 (dd, J=8.00 Hz, J=6.35 Hz, 1H).

### Step 5

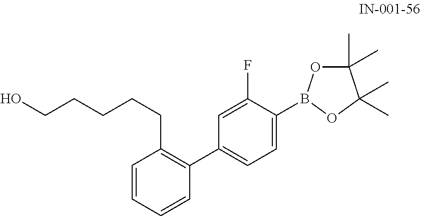 [SMILES: CC1(C)OB(c2ccc(-c3ccccc3CCCCCO)cc2F)OC1(C)C]

### IN-001-56: 5-(3′-fluoro-4′-(4,4,5,5-tetramethyl-1,3,2-dioxaborolan-2-yl)biphenyl-2-yl)pentan-1-ol

A round-bottom flask was charged with the IN-001-55 (0.31 g, 0.82 mmol) and Pd/C (0.0308 g), and the system was put under vacuum. Then, under vacuum, THF (30 mL) was added via syringe. The system was them put under H₂ atmosphere and the reaction was stirred at room temperature for 4 h. After this time, the mixture was then filtered over celite and the solvent was removed in vacuo, affording the target product as a colorless oil (0.31 g, 99%). MS (m/z): 385.3 (M+H). 407.3 (M+Na). LCMS Ret. time: 2.43. Rf: 0.6 (Hexanes/EtOAc 50/50). The ¹H NMR spectrum is fairly clean and it shows the expected signals. ¹H NMR (CDCl₃, δ): 1.39 (s, 12H), 1.46 (m, 4H), 2.58 (m, 2H), 3.55 (t, J=6.40 Hz, 2H), 6.992 (dd, J=10.00 Hz, J=1.30 Hz, 1H), 7.09 (dd, J=7.65 Hz, J=1.30 Hz, 1H), 7.17 (m, 1H), 7.22 (td, J=6.75 Hz, J=1.95 Hz, 1H), 7.29 (m, 2H), 7.762 (dd, J=7.45 Hz, J=6.35 Hz, 1H).

### Step 6

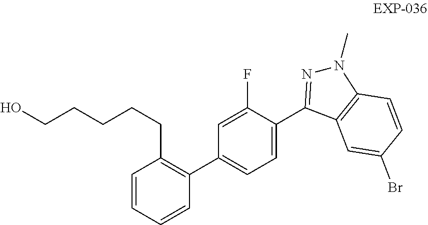 [SMILES: Cn1nc(-c2ccc(-c3ccccc3CCCCCO)cc2F)c2cc(Br)ccc21]

### EXP-036: 5-(4′-(5-bromo-1-methyl-1H-indazol-3-yl)-3′-fluorobiphenyl-2-yl)pentan-1-ol (LZH-00038)

A scintillation vial was charged with the 5-bromo-3-iodo-1-methyl-indazole (0.121 g, 0.36 mmol), K₂CO₃ (0.099 g, 0.72 mmol), and [Pd(PPh₃)₄] (0.01 g, 0.03 mmol). Then, the system was put under vacuum and under vacuum 10 mL of a dioxane solution of the boronic ester IN-001-56 (0.0093 g/mL, 0.093 g, 0.24 mmol) and water (1 mL) were added via syringe. Then, the system was put under N₂ atmosphere and the reaction was heated at 80° C. for 4 h. After this time, the solvent was removed in vacuo and the residue was then purified by flash chromatography (0-45% Hexanes-EtOAc) and after the evaporation of the solvent the product was obtained as colorless oil (0.036 g, 32%). ). MS (m/z): 467. 2 (M+H). LCMS Ret. time 2.60 Rf: 0.33 (Hexanes/EtAcO 50/50. ¹H NMR (CDCl₃, δ): 1.29 (m, 2H), 1.50 (m, 4H), 2.65 (m, 2H), 3.53 (m, 2H), 4.13 (s, 3H), 7.19 (dd, J=11.41 Hz, J=1.50 Hz, 1H), 7.226 (dd, J=7.85 Hz, J=1.70 Hz, 1H), 7.256 (m, 2H), 7.32 (m, 3H), 7.63 (d, J=1.55 Hz, 1H), 7.76 (dd, J=8.76 Hz, J=3.05 Hz, 1H), 7.806 (pt, J=7.65 Hz, 1H).

### Synthesis of Example 37

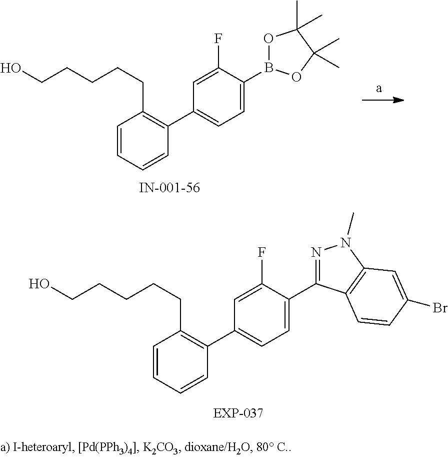 [SMILES: CC1(C)OB(c2ccc(-c3ccccc3CCCCCO)cc2F)OC1(C)C ; Cn1nc(-c2ccc(-c3ccccc3CCCCCO)cc2F)c2ccc(Br)cc21]

### EXP-037: 5-(4′-(6-bromo-1-methyl-1H-indazol-3-yl)-3′-fluorobiphenyl-2-yl)pentan-1-ol (LZH-00039)

Compound EXP-037 was prepared as it was described for EXP-036 on using 6-bromo-3-iodo-1-methyl-indazole (0.121 g, 0.36 mmol), 10 mL of dioxane solution of boronic ester IN-001-56 (0.0093 g/mL, 0.093 g, 0.24 mmol), K₂CO₃ (0.099 g, 0.72 mmol), and [Pd(PPh₃)₄] (0.01 g, 0.03 mmol) and water (1 mL). The product was obtained as a colorless oil (0.036 g, 32%) after purification by flash chromatography (0-45% Hexanes-EtOAc). MS(m/z): 467. 2 (M+H). LCMS Ret. time 2.56. Rf: 0.33 (Hexanes/EtAcO 50/50). ¹H NMR (CDCl₃, δ): 1.30 (m, 2H), 1.50 (m, 4H), 2.65 (m, 2H), 3.55 (t, J=6.55 Hz, 2H), 4.15 (s, 3H), 7.24 (m, 4H), 7.32 (m, 3H), 7.52 (dd, J=8.40 Hz, J=1.45 Hz, 1H), 7.79 (t, J=7.30 Hz, 1H), 8.05 (m, 1H).

### Synthesis of Example 38

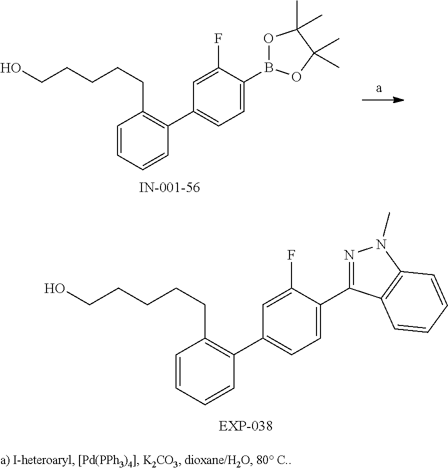 [SMILES: CC1(C)OB(c2ccc(-c3ccccc3CCCCCO)cc2F)OC1(C)C ; Cn1nc(-c2ccc(-c3ccccc3CCCCCO)cc2F)c2ccccc21]

### EXP-038: 5-(3′-fluoro-4′-(1-methyl-1H-indazol-3-yl)biphenyl-2-yl)pentan-1-ol5-(3′-fluoro-4′-(1-methyl-1H-indazol-3-yl)biphenyl-2-yl)pentan-1-ol (LZH-00040)

Compound EXP-038 was prepared as it was described for EXP-036 on using (0.093 g, 0.36 mmol), 10 mL of dioxane solution of boronic ester IN-001-56 (0.0093 g/mL, 0.093 g, 0.24 mmol), K₂CO₃ (0.099 g, 0.72 mmol), and [Pd(PPh₃)₄] (0.01 g, 0.03 mmol) and water (1 mL). The product was obtained as a colorless oil (0.036 g, 38%) after purification by flash chromatography (0-70% Hexanes-EtOAc). MS(m/z): 389.3 (M+H). LCMS Ret. time 2.18. ¹H NMR (CDCl₃, δ): 1.27 (m, 2H), 1.44 (m, 2H), 1.50 (m, 2H), 2.61 (m, 2H), 3.52 (m, 2H), 4.12 (s, 3H), 7.05 (ddd, J=8.35 Hz, J=6.55 Hz, J=0.80 Hz, 1H), 7.23 (m, 7H), 7.46 (pt, J=7.50 Hz, 1H), 7.50 (d, J=8.00 Hz, 1H), 7.76 (dt, J=8.76 Hz, J=0.85 Hz, 1H.

### Synthesis of Example 39

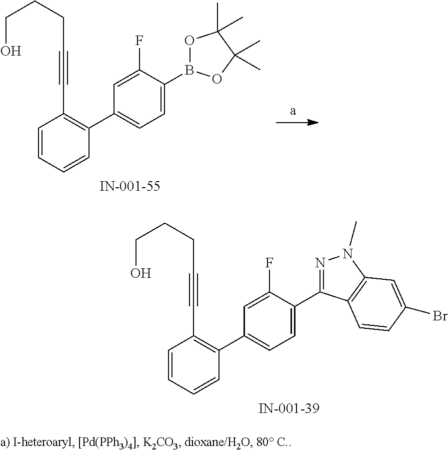 [SMILES: CC1(C)OB(c2ccc(-c3ccccc3C#CCCCO)cc2F)OC1(C)C ; Cn1nc(-c2ccc(-c3ccccc3C#CCCCO)cc2F)c2ccc(Br)cc21]

### EXP-039: 5-(4′-(6-bromo-1-methyl-1H-indazol-3-yl)-3′-fluoro-[1,1′-biphenyl]-2-yl)pent-4-yn-1-ol (LZH-00041)

A scintillation vial was charged with the 6-bromo-3-iodo-1-methyl-indazole (0.121 g, 0.36 mmol), K₂CO₃ (0.112 g, 0.81 mmol), and [Pd(PPh₃)₄] (0.0156 g, 0.01 mmol). Then, the system was put under vacuum and under vacuum 10 mL of a dioxane solution of the boronic ester IN-001-55 (0.0101 g/mL, 0.101 g, 027 mmol) and water (1 mL) were added via syringe. Then, the system was put under N₂ atmosphere and the reaction was heated at 80° C. for 4 h. After this time, the solvent was removed in vacuo and the residue was then purified by flash chromatography (0-45% Hexanes-EtOAc) and after the evaporation of the solvent the product was obtained as yellow oil (0.064 g, 52%). MS(m/Z): 463.2 (M+H). LCMS Ret. time 2.53. Rf: 0.57 (Hexanes/EtAcO 50/50). ¹H NMR (CDCl₃, δ): 1.738 (quint, J=6.75 Hz, 2H), 2.445 (t, J=6.75 Hz, 2H), 3.533 (t, J=6.75 Hz, 2H), 4.113 (s, 3H), 7.32 (m, 2H), 7.382 (m, 2H), 7.472 (m, 2H), 7.525 (dd, J=7.50 Hz, J=1.20 Hz, 1H), 7.626 (dd, J=1.60 Hz, J=0.55 Hz, 1H), 7.75 (dd, J=8.70 Hz, J=3.05 Hz, 1H), 7.81 (pt, J=7.90 Hz,).

### Synthesis of Example 40

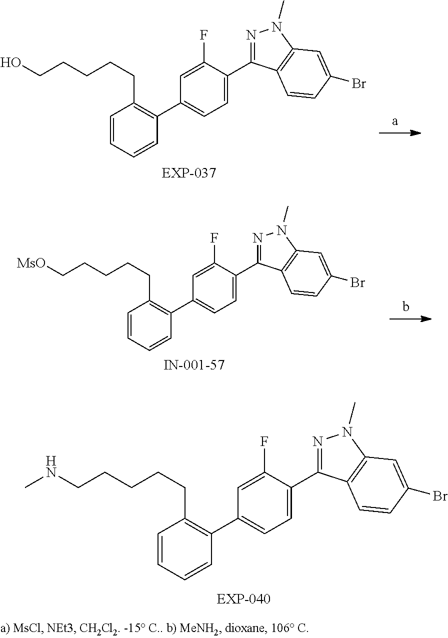 [SMILES: Cn1nc(-c2ccc(-c3ccccc3CCCCCO)cc2F)c2ccc(Br)cc21 ; Cn1nc(-c2ccc(-c3ccccc3CCCCCOS(C)(=O)=O)cc2F)c2ccc(Br)cc21 ; CNCCCCCc1ccccc1-c1ccc(-c2nn(C)c3cc(Br)ccc23)c(F)c1]

### Step 1

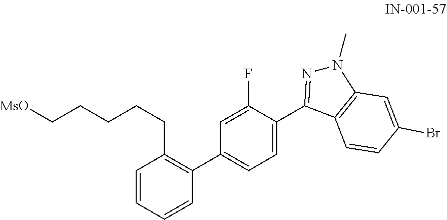 [SMILES: Cn1nc(-c2ccc(-c3ccccc3CCCCCOS(C)(=O)=O)cc2F)c2ccc(Br)cc21]

### IN-001-57. 5-(4′-(6-bromo-1-methyl-1H-indazol-3-yl)-3′-fluorobiphenyl-2-yl)pentylmethane-sulfonate

To a scintillation vial containing a solution of EXP-037 (0.036 g, 0.08 mmol) and NEt₃ (33.5 μL, 0.24 mmol) in CH₂Cl₂ (5 mL), cooled down at −15° C., a 0.1 M solution of MsCl (2.4 mL, 0.24 mmol) was added dropwise. The reaction was stirred at −15° C. for 30 min and then the reaction was warmed up using an ice/water bath, stirring the reaction of other 30 min. The solvent was removed in vacuo, and the residue was used without further purification into the next step, assuming 100% yield (0.042 g). MS: 545.2 m/z (M+H). LCMS Ret. time: 2.67.

### Step 2

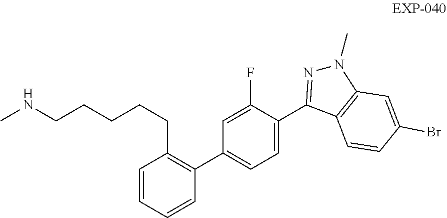 [SMILES: CNCCCCCc1ccccc1-c1ccc(-c2nn(C)c3cc(Br)ccc23)c(F)c1]

### EXP-040: 5-(4′-(6-bromo-1-methyl-1H-indazol-3-yl)-3′-fluorobiphenyl-2-yl)-N-methylpen-tan-1-amine (LZH-00042)

In a sealed tube, a solution of crude IN-001-57 (0.042 g, 0.08 mmol) and 40% aqueous solution of methylamine (1 mL) in dioxane (5 mL) was heated for 6 h. After cooling down the reaction mixture at room temperature, the mixture was diluted with EtOAc and water (20 mL each) and then it was transferred to a separation funnel and the mixture was extracted. The organic phase was separated and the aqueous phase was extracted with EtOAc (2×20 mL). The organic phases were combined, dried over Na₂SO₄ and then the solvent was removed in vacuo. The product was obtained as a white solid (0.018 g, 49%) after purification by flash chromatography (0-100%/o Hexanes-Polar solvent, being polar solvent EtOAc/MeOH 70/30), followed by a purification by reverse phase flash chromatography. MS (m/Z): 480.3 (m/Z). LCMS Ret. time: 2.24. ¹H NMR (CDCl₃, δ): 1.27 (m, 4H), 1.50 (m, 2H), 1.743 (m, 2H), 2.59 (s, 3H), 2.63 (pt, J=7.20 Hz, 2H), 2.80 (pt, J=7.90 Hz, 2H), 4.11, (s, 3H), 7.158 (dd, J=11.16 Hz, J=1.37 Hz, 1H), 7.193 (dd, J=7.75 Hz, J=1.37 Hz, 1H), 7.222 (m, 1H), 7.265 (m, 2H), 7.31 (m, 2H), 7.618 (d, 11.10, 1H), 7.763 (dd, J=13.61 Hz, J=3.05 Hz, 1H), 7.8 (pt, J=7.75 Hz, 1H).

### Synthesis of Example 41

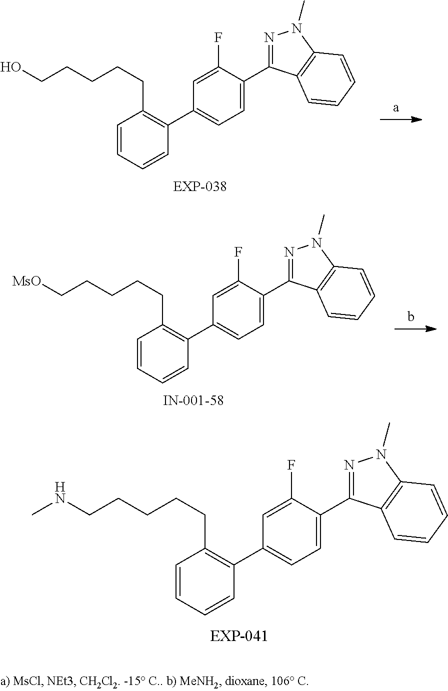 [SMILES: Cn1nc(-c2ccc(-c3ccccc3CCCCCO)cc2F)c2ccccc21 ; Cn1nc(-c2ccc(-c3ccccc3CCCCCOS(C)(=O)=O)cc2F)c2ccccc21 ; CNCCCCCc1ccccc1-c1ccc(-c2nn(C)c3ccccc23)c(F)c1]

### Step 1

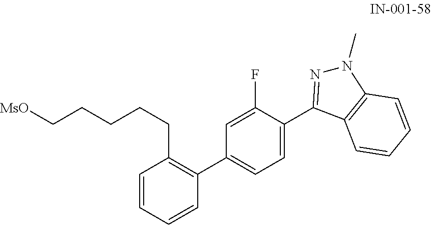 [SMILES: Cn1nc(-c2ccc(-c3ccccc3CCCCCOS(C)(=O)=O)cc2F)c2ccccc21]

### IN-001-58. 5-(3′-fluoro-4′-(1-methyl-1H-indazol-3-yl)biphenyl-2-yl)pentyl methanesulfonate

Compound IN-001-58 was prepared as it was described for IN-001-57 on using EXP-038 (0.036 g, 0.09 mmol), NEt₃ (37.6 μL, 0.27 mmol), CH₂Cl₂ (5 mL) a 0.1 M solution of MsCl (2.6 mL, 0.26 mmol). The crude product was used without further purification into the next step, assuming 100% yield (0.043 g). MS: 467.3 m/z (M+H). LCMS Ret. time: 2.31.

### Step 2

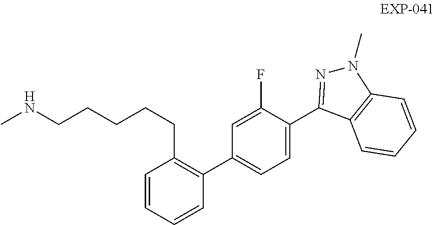 [SMILES: CNCCCCCc1ccccc1-c1ccc(-c2nn(C)c3ccccc23)c(F)c1]

### EXP-041: 5-(3′-fluoro-4′-(1-methyl-1H-indazol-3-yl)biphenyl-2-yl)-N-methylpentan-1-amine (LZH-00043)

Compound EXP-041 was prepared as it was described for EXP-040 on using crude IN-001-58 (0.042 g, 0.09 mmol) and 40% aqueous solution of methylamine (1 mL) in dioxane (5 mL). The product was obtained as a white solid (0.018 g, 50%) after purification by flash chromatography (0-100% Hexanes-Polar solvent, being polar solvent EtOAc/MeOH 70/30), followed by a purification by reverse phase flash chromatography. MS (m/Z): 402.3 (m/z). LCMS Ret. time: 2.03. ¹H NMR (CDCl₃, δ): 1.318 (quint, J=7.61 Hz, 2H), 1.55 (quint, J=7.61 Hz, 2H), 1.761 (quint, J=61 Hz, 2H), 2.562 (s, 3H), 2.652 (pt, J=61 Hz, 2H), 2.818 (pt, J=61 Hz, 2H), 4.18, (s, 3H), 7.114 (ddd, J=8.40 Hz, J=6.45 Hz, J=0.75 Hz, 1H), 7.214 (dd, J=10.75 Hz, J=1.65 Hz, 1H), 7.244 (m, 2H), 7.3 (m, 4H), 7.527 (pt, J=7.65 Hz, 1H), 7.57 (d, J=8.45 Hz, 1H), 7.73 (dt, J=8.75 Hz, J=0.80 Hz, 1H).

### Synthesis of Example 42

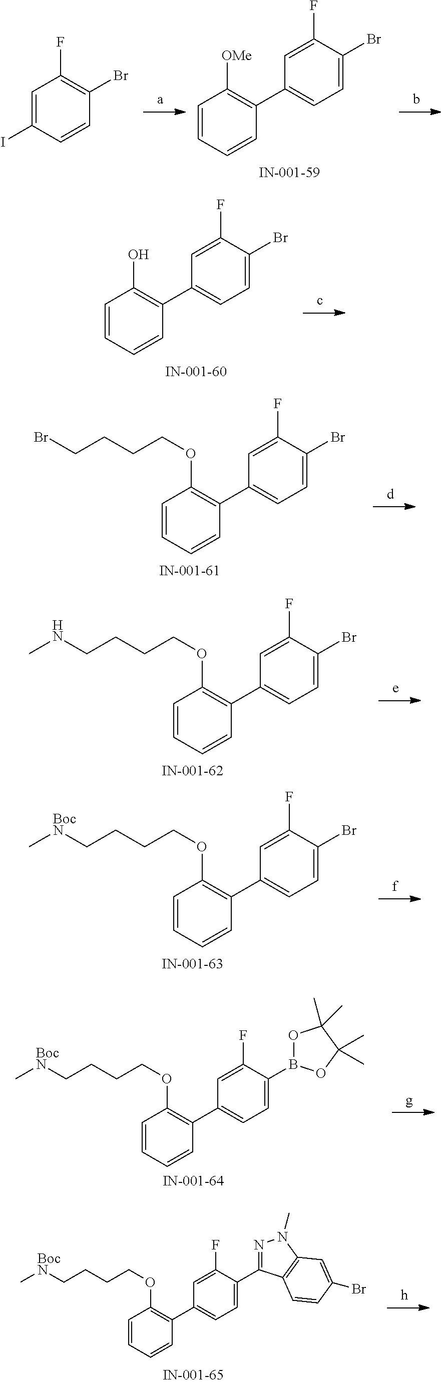

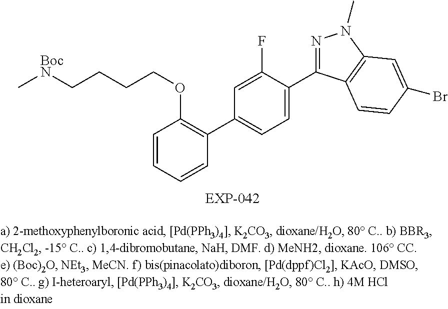 [SMILES: CN(CCCCOc1ccccc1-c1ccc(-c2nn(C)c3cc(Br)ccc23)c(F)c1)C(=O)OC(C)(C)C]

### Step 1

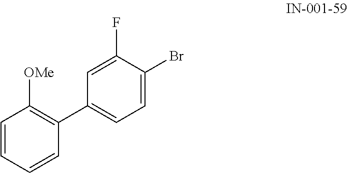 [SMILES: COc1ccccc1-c1ccc(Br)c(F)c1]

### IN-001-59. 4′-bromo-3′-fluoro-2-methoxybiphenyl

Compound IN-001-59 was prepared as it was described for IN-001-46 on using 1-bromo-2-fluoro-4-iodobenzene (3.5 g, 11.63 mmol), 2-methoxyphenilboronic acid (1.76 g, 11.63 mmol), K₂CO₃ (4.82 g, 34.89 mmol), and [Pd(PPh₃)₄](0.67 g, 0.58 mmol), and water (1.8 mL) and dioxane (18 mL) as solvents. The product was obtained as a colorless oil (3.17 g, 97%) after purification by flash chromatography (0-5% Hexanes-EtOAc). The molecular ion was not detected. LCMS ret. time: 2.44. Rf: 0.57 (hexanes). ¹H NMR (CDCl₃, δ): 3.83 (s, 3H), 6.99 (d, J=8.00 Hz, 1H), 7.03 (td, J=8.00 Hz, J=1.25 Hz, 1H), 7.19 (dd, J=7.70H, J=1.55 Hz, 1H), 7.34 (m, 3H), 7.55 (pt, J=7.40 Hz, 1H).

### Step 2

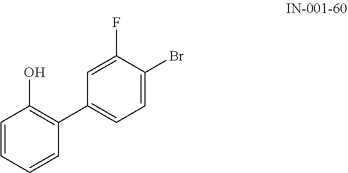 [SMILES: Oc1ccccc1-c1ccc(Br)c(F)c1]

### IN-001-60. 4′-bromo-3′-fluorobiphenyl-2-ol

To a solution of IN-001-59 (3.17 g, 11.28 mmol) in CH₂Cl₂ (50 mL), under N₂ atmosphere, BBr₃ (3.2 mL, 33.84 mmoL) was added dropwise at −15° C. using an ice/acetone bath. The reaction was stirred at low temperature for 20 minutes, an then, the mixture was warmed up at room temperature, stirring for 4 h. The mixture was then cooled down in an ice/water bath, and the reaction was quenched by the careful addition of water (50 mL). The organic phase was separated and the aqueous phase was extracted with more CH₂Cl₂ (2×50 mL). The combined organic layers were dried over Na₂SO₄ and the solvent was removed in vacuo. The residue was used without further purification into the next step assuming 100% yield. The molecular ion was not detected. LCMS ret. time: 2.14. Rf: 0.4 (Hexanes/EtOAc 80/20).

### Step 3

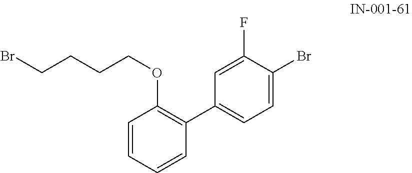 [SMILES: Fc1cc(-c2ccccc2OCCCCBr)ccc1Br]

### IN-001-61. 4′-bromo-2-(4-bromobutoxy)-3′-fluorobiphenyl

To an anhydrous DMF (40 mL) solution of IN-001-60 (3.01 g, 11.27 mmol) 1,4-dibromobutane (4 mL, 33.81 mmol), NaH (0.452 NaH at 60%, 0.271 g of NaH, 11.27 mmol) was added portionwise. The reaction mixture was stirred for room temperature for 4 h under N₂ atmosphere. Then, the reaction was quenched adding water (60 mL) and the mixture was extracted with EtOAc (3×60 mL). The organic layers were combined and the combined organic phases were washed with water (3×100 mL). The organic phase was dried over Na₂SO₄ and the solvent was removed in vacuo. The crude was purified by flash chromatography (100% Hexanes) and the product was obtained as a colorless oil (2.5 g, 55%). The molecular ion was not detected by LCMS. LCMS ret. time: 2.73. Rf: 0.51 (Hexanes). ¹H NMR (CDCl₃, δ): 1.89 (m, 2H), 1.95 (m, 2H), 3.40 (t, J=6.50 Hz, 2H), 4.01 (t, J=6.10 Hz, 2H), 6.97 (d, J=7.85 Hz, 1H), 7.03 (td, J=7.85 Hz, J=1.10 Hz, 1H), 7.18 (dd, J=8.15H, J=2.10 Hz, 1H), 7.294 (d, J=7.60 Hz, 1H) 7.331 (m, 2H), 7.556 (dd, J=8.15 Hz, J=7.25 Hz, 1H).

### Step 4

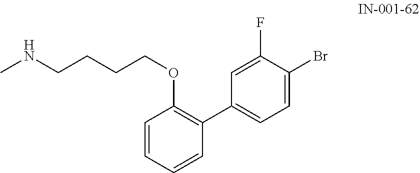 [SMILES: CNCCCCOc1ccccc1-c1ccc(Br)c(F)c1]

### IN-001-62. 4-(4′-bromo-3′-fluorobiphenyl-2-yloxy)-N-methylbutan-1-amine

A sealed tube was charged with the ether IN-001-61 (2.5 g, 6.22 mmol), 40% solution of MeNH₂ (1.6 mL, 18.66 mmol) and iPrOH (40 mL). The solution was heated at 106° C. for 5 h. After cooling down the reaction mixture at room temperature, the mixture was diluted with EtOAc and water (50 mL each) and then it was transferred to a separation funnel and the mixture was extracted. The organic phase was separated and the aqueous phase was extracted with EtOAc (2×50 mL). The organic phases were combined, dried over Na₂SO₄ and then the solvent was removed in vacuo. The brown oily residue was dried in vacuo for 2 h, affording the target product as a light brown oil. The product was used in the next step without further purification. MS(m/z): 352.1 (M+H). LCMS Ret. time: 1.76

### Step 5

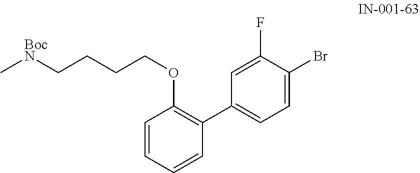 [SMILES: CN(CCCCOc1ccccc1-c1ccc(Br)c(F)c1)C(=O)OC(C)(C)C]

### IN-001-63. tert-butyl 4-(4′-bromo-3′-fluorobiphenyl-2-yloxy)butyl(methyl)carbamate

To solution of the amine IN-001-62 (1.94 g, 5.51 mmol) in anhydrous MeCN (35 mL), NEt₃ (1.5 mL, 10.98 mmol) was added. Then, under stirring, a solution of Boc₂O (1.18 g, 5.40 mmol) in MeCN (15 mL) was added. The reaction was stirred at room temperature for 2 h. Then, the reaction was quenched adding water (50 mL) and the mixture was transferred to a separation funnel and the aqueous phase was extracted with EtOAc (3×50 mL) and the combined organic layers were washed with brine (1×70 mL). The organic phase was dried over Na₂SO₄ and the solvent was removed in vacuo. The crude was purified by flash chromatography (0-15% Hexanes-EtOAc), affording the target product as a light yellow oil (1.04 g, 42%). MS (m/z): 452.2 (M+H), 396.1 (M-tBu+Na), 352.1 (M-tBu+H). LCMS Ret. time: 2.83. Rf: 0.58 (Hexanes/EtOAc 80/20). ¹H NMR (CDCl₃, δ): 1.43 (s, 9H), 1.9 (m, 2H), 1.71 (m, 2H), 2.79 (s, 3H), 3.22 (br, 2H), 3.99 (t, J=6.00 Hz, 2H), 7.02 (t, J=7.65 Hz, 1H), 7.19 (d, J=6.00 Hz, 1H), 7.32 (m, 3H), 7.54 (pt, J=8.05 Hz, 1H).

### Step 6

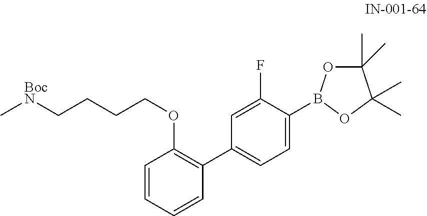

### IN-001-64. tert-butyl 4-(3′-fluoro-4′-(4,4,5,5-tetramethyl-1,3,2-dioxaborolan-2-yl)biphenyl-2-yloxy)butyl(methyl)carbamate

A round-bottom flask was charged with the aryl bromide IN-001-63 (1.04 g, 1.99 mmol), KOAc (0.59 g, 5.97 mmol), bis(pinacolato)diboron (1.52 g, 5.97 mmol) and [Pd(dppf)Cl₂] (0.22 g, 0.30 mmol). Then, the system was put under vacuum and under vacuum anhydrous DMSO (30 mL) was added via syringe. Then, the system was put under N₂ atmosphere and the reaction was heated at 80° C. for an overnight. After cooling down, the mixture was diluted with EtOAc (80 mL) and the solution was extracted with water (4×60 mL). The organic phase was dried over Na₂SO₄ and the solvent was removed in vacuo and the residue was then purified by flash chromatography (0-15% Hexanes-EtOAc) and after the evaporation of the solvent the product was obtained as a colorless oil (0.64 g, 71%). MS (m/z): 500.4 (M+H), 444.3 (M-tBu+H), 400.3 (M-Boc+H). LCMS Ret. time: 2.93. Rf: 0.48 (Hexanes/EtOAc 80/20). ¹H NMR (CDCl₃, δ): 1.37 (s, 12H) 1.43 (br, 9H), 1.59 (m, 2H), 1.70 (m, 2H), 2.77 (s, 3H), 3.20 (b, 2H), 3.98 (t, J=5.40 Hz, 2H), 6.96 (d, J=8.46 Hz, 1H), 7.01 (t, J=7.85 Hz, 1H), 7.257 (dd, J=10.75 Hz, J=1.30 Hz, 1H), 7.31 (m, 3H), 7.74 (dd, J=7.65 Hz, J=6.55 Hz, 1H).

### Step 7

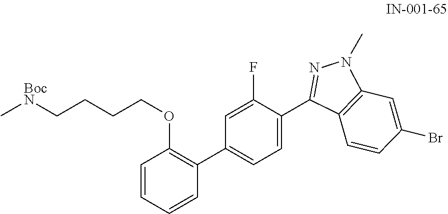 [SMILES: CN(CCCCOc1ccccc1-c1ccc(-c2nn(C)c3cc(Br)ccc23)c(F)c1)C(=O)OC(C)(C)C]

### IN-001-65. tert-butyl4-(4′-(6-bromo-1-methyl-1H-indazol-3-yl)-3′-fluorobiphenyl-2-yloxy)butyl(methyl)carbamate

A scintillation vial was charged with the 6-bromo-3-iodo-1-methyl-indazole (0.096 g, 0.19 mmol), K₂CO₃ (0.079 g, 0.57 mmol), and [Pd(PPh₃)₄] (0.011 g, 0.01 mmol). Then, the system was put under vacuum and under vacuum 5 mL of a dioxane solution of the boronic ester IN-001-64 (0.0192 g/mL, 0.096 g, 0.19 mmol) and water (1 mL) were added via syringe. Then, the system was put under N₂ atmosphere and the reaction was heated at 80° C. for 4 h. After this time, the solvent was removed in vacuo and the residue was then purified by flash chromatography (0-20% Hexanes-EtOAc) and after the evaporation of the solvent the product was obtained as a yellow oil (0.066 g, 59%). MS (m/z): 582.3 (M+H), 526.3 (M-tBu+H), 482.3 (M-Boc+H). LCMS Ret. time: 3.01. Rf. 0.25 (Hexanes/EtOAc 80/20). ¹H NMR (CDCl₃, δ): 1.41 (br, 9H), 1.64 (m, 2H), 1.75 (m, 2H), 2.79 (s, 3H), 3.23 (b, 2H), 4.03 (t, J=6.25 Hz, 2H), 4.12 (s, 3H), 7.00 (d, J=7.65 Hz, 1H), 7.05 (t, J=7.30 Hz, 1H), 7.30 (dd, J=8.65 Hz, J=1.60 Hz, 1H), 7.46 (m, 2H), 7.62 (m, 1H), 7.746 (dd, J=8.60 Hz, J=3.05 Hz, 1H), 7.796 (pt, J=7.80 Hz, 1H).

### Step 8

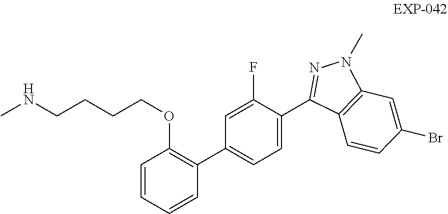 [SMILES: CNCCCCOc1ccccc1-c1ccc(-c2nn(C)c3cc(Br)ccc23)c(F)c1]

### EXP-042: 4-(4′-(6-bromo-1-methyl-1H-indazol-3-yl)-3′-fluorobiphenyl-2-yloxy)-N-methyl-butan-1-amine (LZH-00044)

The N-Boc protected amine IN-001-65 (0.066 g, 0.11 mmol) was dissolved in 4M HCl solution in dioxane (5 mL), and the mixture was stirred for 1 h at room temperature. The reaction was quenched by addition of saturated solution of NaHCO₃ until no more gas was produced and the pH of the solution was about 8 (aprox. 20 mL). Then, the mixture was diluted with EtOAc (20 mL) and the mixture was extracted. The organic phase was separated and the aqueous phase was extracted with EtOAc (2×20 mL). The organic layers were combined and washed with brine (50 mL), dried over Na₂SO₄ and then the solvent was evaporated in vacuo. The solid yellow residue obtained after the evaporation was purified by flash chromatography (0-100% Hexanes-Polar solvent, being polar solvent EtOAc/MeOH 70/30) and by reverse phase flash chromatography and the product was obtained as a white solid (0.024 g, 44%). MS (m/z): 482.2. (M+H) Ret. time LCMS: 2.01. Rf: 0.21 (EtOAc/MeOH 70/30). ¹H NMR (CDCl₃, δ): 1.84 (m, 2H), 1.94 (m, 2H), 2.46 (s, 3H), 2.83 (pt, J=7.75 Hz, 2H), 3.99 (t, J=5.95 Hz, 2H), 4.08 (s, 3H), 6.93 (d, J=8.40 Hz, 1H), 7.052 (t, J=7.40 Hz, 1H), 7.36 (m, 5H), 7.60 (d, J=0.90 Hz, 1H), 7.62 (dd, J=8.55 Hz, J=3.00 Hz, 1H), 7.80 (pt, J=7.60 Hz, 1H).

### Synthesis of Example 43

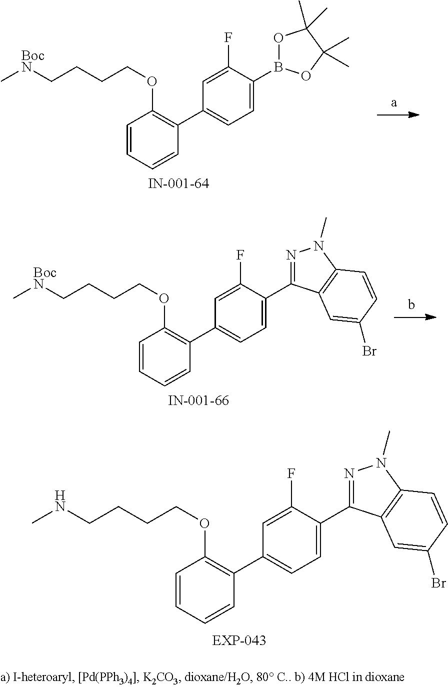

### Step 1

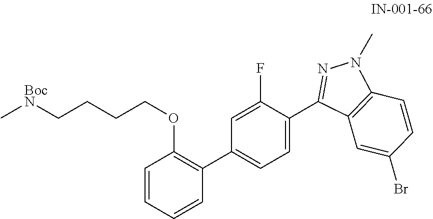 [SMILES: CN(CCCCOc1ccccc1-c1ccc(-c2nn(C)c3ccc(Br)cc23)c(F)c1)C(=O)OC(C)(C)C]

### IN-001-66. tert-butyl 4-(4′-(5-bromo-1-methyl-1H-indazol-3-yl)-3′-fluorobiphenyl-2-yloxy)butyl(methyl)carbamate

Compound IN-001-66 was prepared as it was described for IN-001-65 on using 5-bromo-3-iodo-1-methyl-indazole (0.096 g, 0.28 mmol), 5 mL of dioxane solution of boronic ester IN-001-64 (0.096 g, 0.28 mmol), K₂CO₃ (0.079 g, 0.57 mmol), [Pd(PPh₃)₄] (0.011 g, 0.01 mmol), and water (1 mL). The product was obtained as a pale yellow oil (0.066 g, 59%) after purification by flash chromatography (0-20% Hexanes-EtOAc). MS (m/z): 582.3 (M+H), 526.3 (M-tBu+H), 482.3 (M-Boc+H). LCMS Ret. time: 2.98. Rf: 0.48 (Hexanes/EtOAc 80/20). ¹H NMR (CDCl₃, δ): 1.42 (s, 9H), 1.65 (m, 2H), 1.75 (m, 2H), 2.80 (s, 3H), 3.24 (b, 2H), 4.03 (t, J=4.90 Hz, 2H), 4.145 (s, 3H), 7.00 (d, J=8.66 Hz, 2H), 7.054 (t, J=8.00 Hz, 1H), 7.33 (m, 2H), 7.39 (dd, J=7.65 Hz, J=1.65 Hz 1H), 7.6 (m, 2H), 7.50 (dd, J=8.80 Hz, J=1.40 Hz, 1H), 7.78 (pt, J=7.75 Hz, 1H), 8.03 (m, 1H).

### Step 2

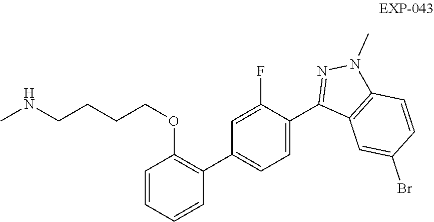 [SMILES: CNCCCCOc1ccccc1-c1ccc(-c2nn(C)c3ccc(Br)cc23)c(F)c1]

### EXP-043: 4-(4′-(5-bromo-1-methyl-1H-indazol-3-yl)-3′-fluorobiphenyl-2-yloxy)-N-methyl-butan-1-amine (LZH-00045)

EXP-043 was prepared as it was described for EXP-042 on using IN-001-66 (0.066 g, 0.11 mmol) and 4M HCl solution in dioxane (5 mL). The product was obtained as a white solid (0.028 g, 51%) after purification by flash chromatography (0-100% Hexanes-Polar solvent, being polar solvent EtOAc/MeOH 70/30) and by reverse phase flash chromatography. MS (m/z): 482.2 (M+H). Ret. time LCMS: 1.98. Rf: 0.21 (EtOAc/MeOH 70/30). ¹H NMR (CDCl₃, δ): 1.84 (m, 2H), 1.94 (m, 2H), 2.49 (s, 3H), 2.84 (m, 2H), 3.995 (t, J=6.30 Hz, 2H), 4.11 (s, 3H), 6.93 (d, J=7.55 Hz, 1H), 7.055 (t, J=7.55 Hz, 1H), 7.36 (m, 5H), 7.49 (dd, J=8.66 Hz, J=1.65 Hz, 1H), 7.78 (pt, J=17.80 Hz, 1H), 8.00 (m, 1H).

### Synthesis of Example 44

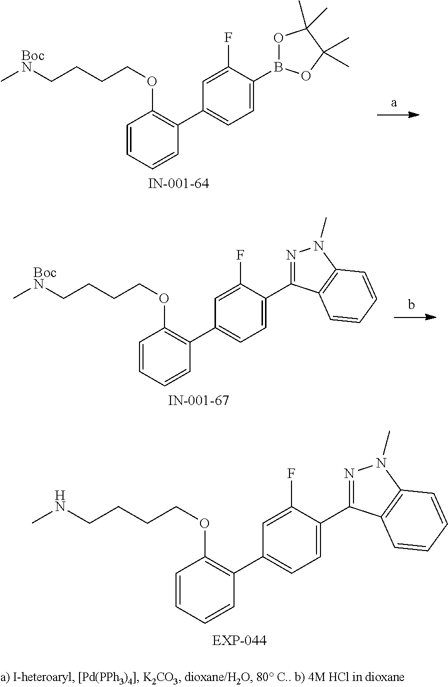

### Step 1

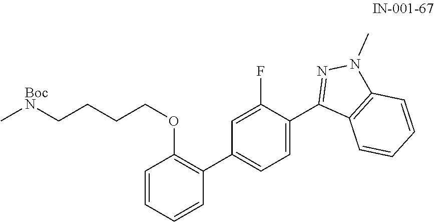 [SMILES: CN(CCCCOc1ccccc1-c1ccc(-c2nn(C)c3ccccc23)c(F)c1)C(=O)OC(C)(C)C]

### IN-001-67. Tert-butyl-4-(3′-fluoro-4′-(1-methyl-1H-indazol-3-yl)biphenyl-2-yloxy)butyl(methyl) carbamate

Compound IN-001-67 was prepared as it was described for IN-001-65 on using 3-iodo-1-methyl-1H-indazole (0.074 g, 0.28 mmol), 5 mL of dioxane solution of boronic ester IN-001-64 (0.096 g, 0.28 mmol), K₂CO₃ (0.079 g, 0.57 mmol), [Pd(PPh₃)₄] (0.011 g, 0.01 mmol), and water (1 mL). The product was obtained as a yellow oil (0.066 g, 68%) after purification by flash chromatography (0-35% Hexanes-EtOAc). MS (m/z): 526.3 (M+Na), 504.4 (M+H), 448.3 (M-tBu+H). LCMS Ret. time: 2.61. ¹H NMR (CDCl₃, δ): 1.41 (s, 9H), 1.66 (m, 2H), 1.79 (m, 2H), 2.79 (s, 3H), 3.26 (pt, J=5.35 Hz, 2H), 4.06 (t, J=6.00 Hz, 2H), 4.17 (s, 3H), 7.02 (d, J=8.05 Hz, 1H), 7.09 (m, 2H), 7.37 (m, 3H), 7.53 (m, 4H), 7.74 (dt, J=8.70 Hz, J=0.85 Hz, 1H).

### Step 2

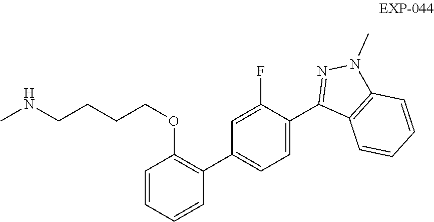 [SMILES: CNCCCCOc1ccccc1-c1ccc(-c2nn(C)c3ccccc23)c(F)c1]

### EXP-044: 4-(3′-fluoro-4′-(1-methyl-1H-indazol-3-yl)biphenyl-2-yloxy)-N-methylbutan-1-amine (LZH-00046)

EXP044 was prepared as it was described for EXP-042 on using IN-001-67 (0.066 g, 0.13 mmol) and 4M HCl solution in dioxane (5 mL). The product was obtained as a white solid (0.036 g, 68%) after purification by flash chromatography (0-100% Hexanes-Polar solvent, being polar solvent EtOAc/MeOH 70/30) and by reverse phase flash chromatography. MS (m/z): 404.3 (M+H). LCMS Ret. time: 1.73. ¹H NMR (CDCl₃, δ): 1.84 (m, 2H), 1.94 (m, 2H), 2.50 (s, 3H), 2.87 (pt, J=6.80 Hz, 2H), 3.99 (t, J=6.05 Hz, 2H), 4.15 (s, 3H), 6.94 (d, J=8.76 Hz, 1H), 7.07 (m, 2H), 7.34 (m, 3H), 7.50 (m, 4H), 7.771 (d, J=8.71 Hz, 1H).

### Synthesis of Example 45

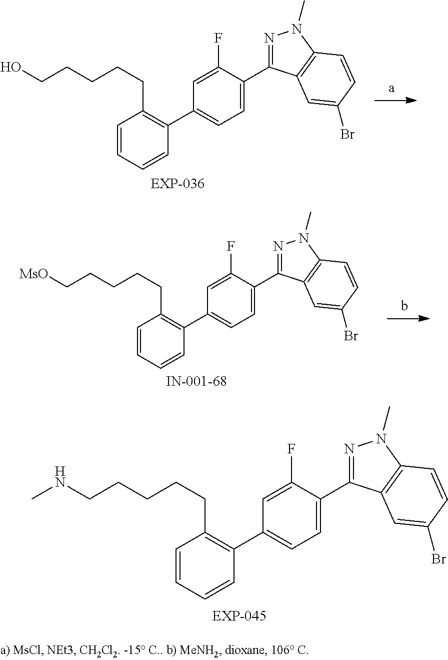 [SMILES: Cn1nc(-c2ccc(-c3ccccc3CCCCCO)cc2F)c2cc(Br)ccc21 ; Cn1nc(-c2ccc(-c3ccccc3CCCCCOS(C)(=O)=O)cc2F)c2cc(Br)ccc21 ; CNCCCCCc1ccccc1-c1ccc(-c2nn(C)c3ccc(Br)cc23)c(F)c1]

### Step 1

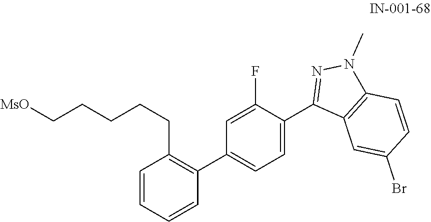 [SMILES: Cn1nc(-c2ccc(-c3ccccc3CCCCCOS(C)(=O)=O)cc2F)c2cc(Br)ccc21]

### IN-001-68: 5-(4′-(5-bromo-1-methyol-1H-indazol-3-yl)-3′-fluorobiphenyl-2-yl)pentylmethane-sulfonate

Compound IN-001-68 was prepared as it was described for IN-001-57 on using EXP-036 (0.042 g, 0.09 mmol), NEt₃ (37.5 μL, 0.27 mmol), CH₂Cl₂ (5 mL) a 0.1 M solution of MsCl (1.4 mL, 0.14 mmol). The crude product was used without further purification into the next step, assuming 100% yield (0.049 g). MS: 545.2 m/z (M+H). LCMS Ret. time: 2.68.

### Step 2

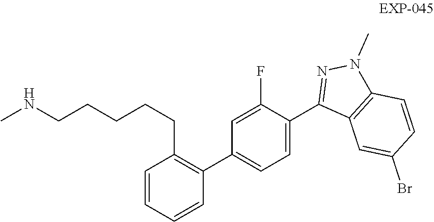 [SMILES: CNCCCCCc1ccccc1-c1ccc(-c2nn(C)c3ccc(Br)cc23)c(F)c1]

### EXP-045: 5-(4′-(5-bromo-1-methyl-1H-indazol-3-yl)-3′-fluorobiphenyl-2-yl)-N-methylpen-tan-1-amine (LZH-00047)

Compound EXP-045 was prepared as it was described for EXP-040 on using crude IN-001-68 (0.049 g, 0.09 mmol) and 40% aqueous solution of methylamine (1 mL) in dioxane (5 mL). The product was obtained as a white solid (0.016 g, 37%) after purification by flash chromatography (0-100% Hexanes-Polar solvent, being polar solvent EtOAc/MeOH 70/30), followed by a purification by reverse phase flash chromatography. MS(m/Z): 480.2 (M+H). LCMS Ret. time: 2.16. ¹H NMR (CDCl₃, δ): 1.29 (quint, J=7.90 Hz, 2H), 1.505 (quint, J=7.90 Hz, 2H), 1.733 (quint, J=7.90 Hz, 2H), 2.578 (s, 3H), 2.63 (pt, 0.7.90 Hz, 2H), 2.80 (pt, J=7.90 Hz, 2H), 4.13, (s, 3H), 7.165 (dd, J=11.00 Hz, J=1.45 Hz, 1H), 7.193 (dd, J=7.55 Hz, J=1.45 Hz, 1H), 7.23 (m, 1H), 7.264 (m, 2H), 7.31 (m, 2H), 7.504 (dd, J=8.76 Hz, J=1.85 Hz, 1H), 7.785 (pt, J=7.60 Hz, 1H), 8.03 (pt, J=2.15 Hz, 1H).

### Synthesis of Example 46

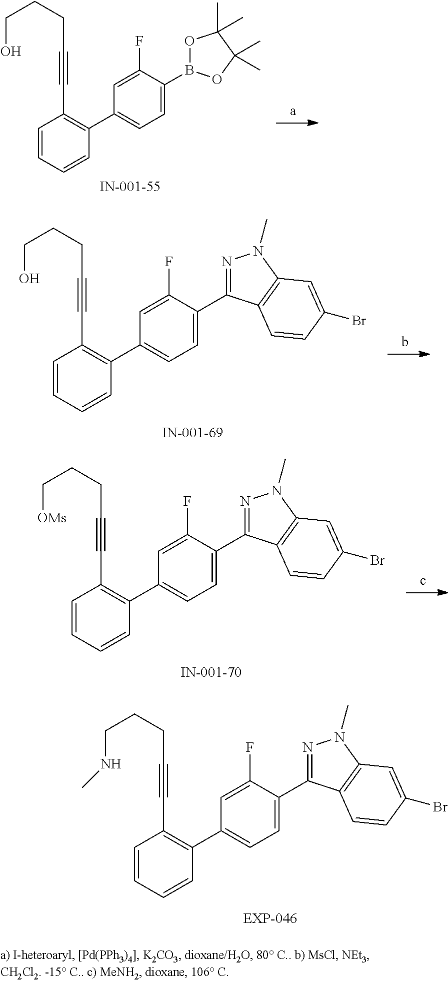 [SMILES: CC1(C)OB(c2ccc(-c3ccccc3C#CCCCO)cc2F)OC1(C)C ; Cn1nc(-c2ccc(-c3ccccc3C#CCCCO)cc2F)c2ccc(Br)cc21 ; Cn1nc(-c2ccc(-c3ccccc3C#CCCCOS(C)(=O)=O)cc2F)c2ccc(Br)cc21 ; CNCCCC#Cc1ccccc1-c1ccc(-c2nn(C)c3cc(Br)ccc23)c(F)c1]

### Step 1

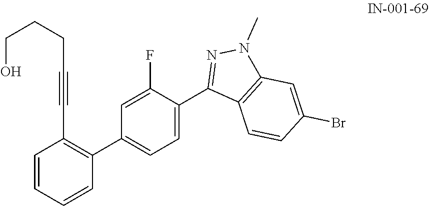 [SMILES: Cn1nc(-c2ccc(-c3ccccc3C#CCCCO)cc2F)c2ccc(Br)cc21]

### IN-001-69: 5-(4′-(6-bromo-1-methyl-1H-indazol-3-yl)-3′-fluorobiphenyl-2-yl)pent-4-yn-1-ol

A scintillation vial was charged with the 6-bromo-3-iodo-1-methyl-indazole (0.121 g, 0.36 mmol), K₂CO₃ (0.112 g, 0.81 mmol), and [Pd(PPh₃)₄] (0.0156 g, 0.01 mmol). Then, the system was put under vacuum and under vacuum 10 mL of a dioxane solution of the boronic ester IN-001-55 (0.0101 g/mL, 0.101 g, 0.27 mmol) and water (1 mL) were added via syringe. Then, the system was put under N₂ atmosphere and the reaction was heated at 80° C. for 4 h. After this time, the solvent was removed in vacuo and the residue was then purified by flash chromatography (0-45% Hexanes-EtOAc) and after the evaporation of the solvent the product was obtained as yellow oil (0.064 g, 52%). MS(m/Z): 463.2 (M+H). LCMS Ret. time 2.53. Rf: 0.57 (Hexanes/EtAcO 50/50). ¹H NMR (CDCl₃, δ): 1.738 (quint, J=6.75 Hz, 2H), 2.445 (t, J=6.75 Hz, 2H), 3.533 (t, J=6.75 Hz, 2H), 4.113 (s, 3H), 7.32 (m, 2H), 7.382 (m, 2H), 7.472 (m, 2H), 7.525 (dd, J=7.50 Hz, J=1.20 Hz, 1H), 7.626 (dd, J=1.60 Hz, J=0.55 Hz, 1H), 7.75 (dd, J=8.70 Hz, J=3.05 Hz, 1H), 7.81 (pt, J=7.90 Hz,).

### Step 2

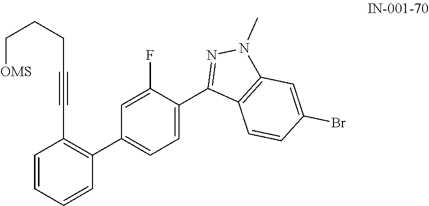

### IN-001-70: 5-(4′-(6-bromo-1-methyl-1H-indazol-3-yl)-3′-fluorobiphenyl-2-yl)pent-4-ynyl methanesulfonate

To a scintillation vial containing a solution of IN-001-69 (0.054 g, 0.12 mmol) and NEt₃ (50 μL, 036 mmol) in CH₂Cl₂ (5 mL), cooled down at −15° C., a 0.1 M solution of MsCl (1.8 mL, 0.18 mmol) was added dropwise. The reaction was stirred at −15° C. for 30 min and then the reaction was warmed up using an ice/water bath, stirring the reaction of other 30 min. The solvent was removed in vacuo, and the residue was used without further purification into the next step, assuming 100% yield (0.063 g). MS: 541.1 m/z (M+H). LCMS Ret. time: 2.59. Rf: 0.48 (Hexanes/EtAcO 50/50).

### Step 3

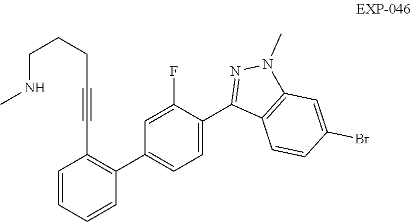 [SMILES: CNCCCC#Cc1ccccc1-c1ccc(-c2nn(C)c3cc(Br)ccc23)c(F)c1]

### EXP-046: 5-(4′-(6-bromo-1-methyl-1H-indazol-3-yl)-3′-fluorobiphenyl-2-yl)-N-methylpent-4-yn-1-amine (LZH-00048)

In a sealed tube, a solution of crude IN-001-70 (0.0631 g, 0.09 mmol) and 40% aqueous solution of methylamine (1 mL) in dioxane (5 mL) was heated for 6 h.

After cooling down the reaction mixture at room temperature, the mixture was diluted with EtOAc and water (20 mL each) and then it was transferred to a separation funnel and the mixture was extracted. The organic phase was separated and the aqueous phase was extracted with EtOAc (2×20 mL). The organic phases were combined, dried over Na₂SO₄ and then the solvent was removed in vacuo. The product was obtained as a white solid (0.016 g, 29%) after purification by flash chromatography (0-100% Hexanes-Polar solvent, being polar solvent EtOAc/MeOH 70/30), followed by a purification by reverse phase flash chromatography. MS (m/z): 476.3 (M+H). LCMS Ret. time: 2.03. ¹H NMR (CDCl₃, δ): 1.28 (m, 2H), 2.05 (m, 2H), 2.55 (s, 3H), 2.826 (m, 2H), 4.147, (s, 3H), 7.35 (m, 2H), 7.418 (m, 2H), 7.454 (dd, J=11.46 Hz, J=10.50 Hz, 1H), 7.497 (dd, J=12.96 Hz, J=1.60 Hz, 1H), 7.539 (d, J=7.40, 1H), 7.65 (d, J=1.40, 1H), 7.748 (dd, J=8.71 Hz, J=2.65 Hz, 1H), 7.816 (pt, J=7.70 Hz, 1H).

### Example 47: Additional Compounds

### (4-(6-bromo-1-ethyl-1H-indazol-3-yl)-3-fluorophenoxy)-N-methylhexan-1-amine (LZH-00049)

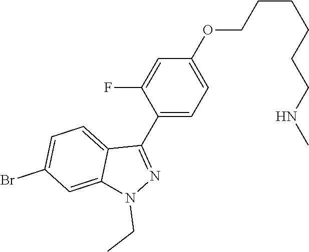 [SMILES: CCn1nc(-c2ccc(OCCCCCCNC)cc2F)c2ccc(Br)cc21]

### Step 1: 6-bromo-1-ethyl-3iodo-1H-indazole

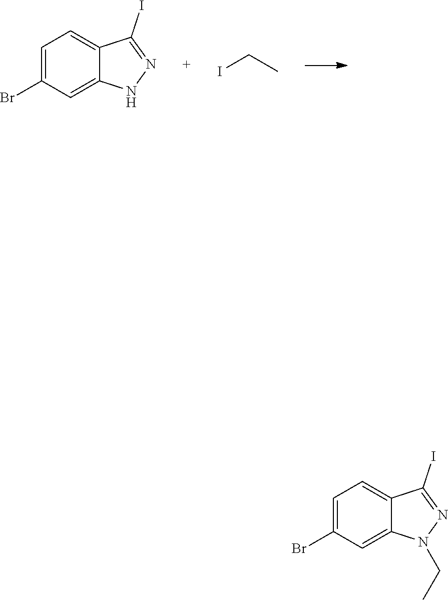

A mixture of 6-bromo-3-iodo-1H-indazole (1.50 g, 4.64 mmol), iodoethane (0.76 g, 4.88 mmol), and K₂CO₃ (0.96 g, 6.97 mmol) in acetonitrile (0.2 M) was stirred at reflux for 5 hr. The reaction was worked up by the filtration through celite pad and washed with more EtOAc untile no more compound washed out. The filtrate was evaporated. The residue was purified by flash chromatography (0-20% EtOAc/hexane) to afford the titled compound as a white solid (0.87 g). LCMS (+ESI): Rt=2.36 min, (M+H)⁺=350.8 and 352.8.

### Step 2: tert-butyl (6-(4-(6-bromo-1-ethyl-1H-indazol-3-yl)-3-fluorophenoxy)hexyl)(methyl)carbamate

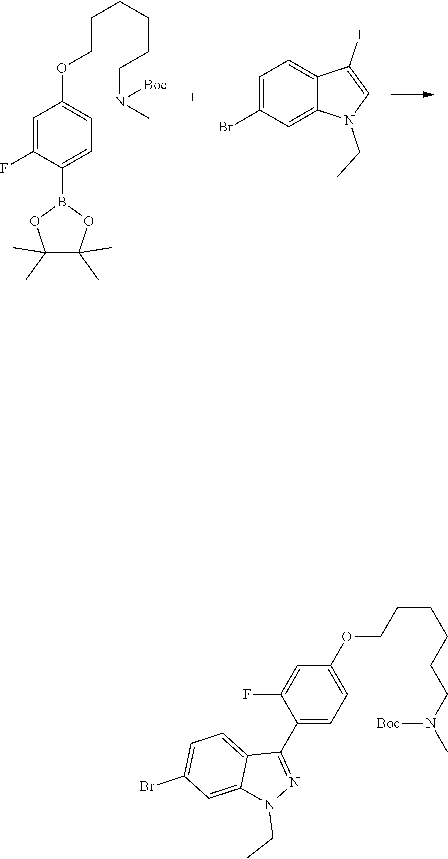 [SMILES: CN(CCCCCCOc1ccc(B2OC(C)(C)C(C)(C)O2)c(F)c1)C(=O)OC(C)(C)C ; CCn1cc(I)c2ccc(Br)cc21 ; CCn1nc(-c2ccc(OCCCCCCN(C)C(=O)OC(C)(C)C)cc2F)c2ccc(Br)cc21]

To a mixture of tert-butyl (6-(3-fluoro-4-(4,4,5,5-tetramethyl-1,3,2-dioxaborolan-2-yl)phenoxy)hexyl)(methyl)carbamate (0.30 g, 0.66 mmol), 6-bromo-1-ethyl-3-iodo-1H-indazole (0.233 g, 0.66 mmol), K₃PO₄ (0.423 g, 1.99 mmol), and Pd(dppf)Cl₂ (0.049 g, 0.07 mmol) in a reaction vial under vacuum was added toluene (3.32 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound as an off-white solid, which is not very pure; thus further purified by reverse phase flash chromatography with C₁₈ column (35-100% MeCN/water) to afford the titled compound as a gum (90.0 mg). LCMS (+ESI): Rt=3.05 min, (M+H)⁺=548.2 and 550.2.

### Step 3: (4-(6-bromo-1-ethyl-1H-indazol-3-yl)-3-fluorophenoxy)-N-methylhexan-1-amine

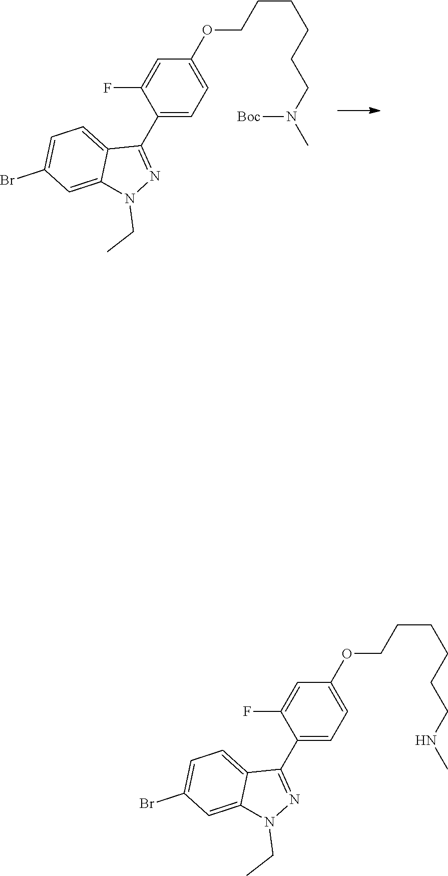 [SMILES: CCn1nc(-c2ccc(OCCCCCCN(C)C(=O)OC(C)(C)C)cc2F)c2ccc(Br)cc21 ; CCn1nc(-c2ccc(OCCCCCCNC)cc2F)c2ccc(Br)cc21]

A solution of tert-butyl (6-(4-(6-bromo-1-ethyl-1H-indazol-3-yl)-3-fluorophenoxy)hexyl)(methyl)carbamate (0.09 g) in 1.5 mL TFA/DCM (1/1) was stirred at rt for 2 hr. The solvent was evaporated and the residue was diluted with EtOAc and washed with Na₂CO₃ solution, dried over Na₂SO₄, evaporated to afford a crude which was further purified by reverse phase flash chromatography with C₁₈ column (10-100% Solvent B/Solvent A, while solvent A is 0.1% of HCOOH in distilled water and Solvent B is 0.1% of HCOOH in MeCN) to afford the titled compound (70.0 mg). LCMS (+ESI): Rt=2.31 min, (M+H)⁺=448.2 and 450.2.

### 6-(4-(6-bromo-1-propyl-1H-indazol-3-yl)-3-fluorophenoxy)-N-methylhexan-1-amine (LZH-00050)

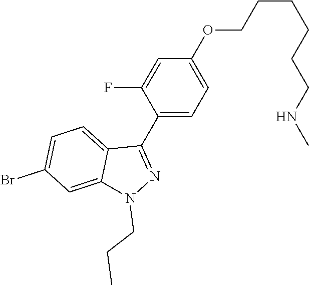 [SMILES: CCCn1nc(-c2ccc(OCCCCCCNC)cc2F)c2ccc(Br)cc21]

### Step 1: 6-bromo-3-iodo-1-propyl-1H-indazole

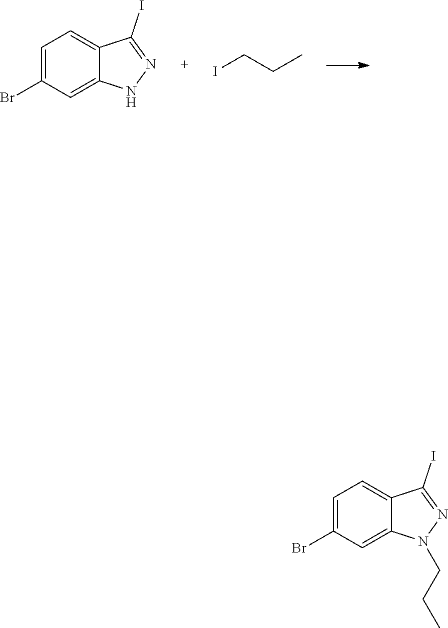

A mixture of 6-bromo-3-iodo-1H-indazole (1.50 g, 4.64 mmol), 1-iodopropane (0.83 g, 4.88 mmol), and K₂CO₃ (0.96 g, 6.97 mmol) in acetonitrile (0.2 M) was stirred at reflux for 5 hr. The reaction was worked up by the filtraation through celite pad and washed with more EtOAc untile no more compound washed out. The filtrate was evaporated. The residue was purified by flash chromatography (0-20% EtOAc/hexane) to afford the titled compound as a yellowish oil (0.78 g). LCMS (+ESI): Rt=2.56 min, (M+H)⁺=354.8 and 356.8.

### Step 2: tert-butyl (6-(4-(6-bromo-1-propyl-1H-indazol-3-yl)-3-fluorophenoxy)hexyl)(methyl)carbamate

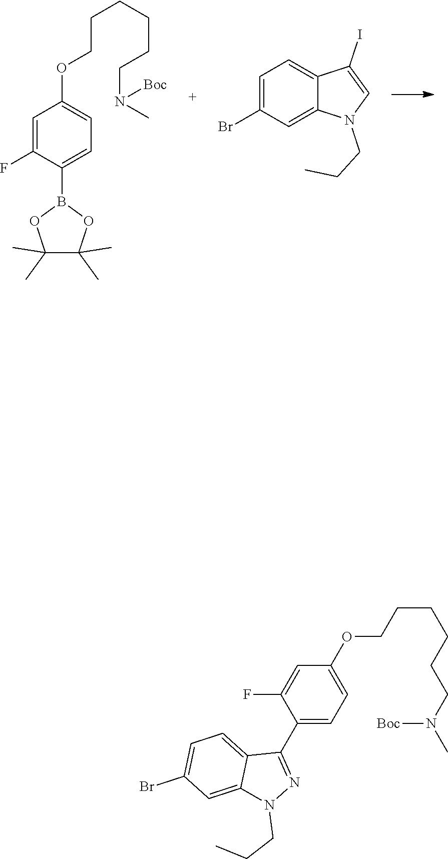 [SMILES: CN(CCCCCCOc1ccc(B2OC(C)(C)C(C)(C)O2)c(F)c1)C(=O)OC(C)(C)C ; CCCn1cc(I)c2ccc(Br)cc21 ; CCCn1nc(-c2ccc(OCCCCCCN(C)C(=O)OC(C)(C)C)cc2F)c2ccc(Br)cc21]

To a mixture of tert-butyl (6-(3-fluoro-4-(4,4,5,5-tetramethyl-1,3,2-dioxaborolan-2-yl)phenoxy)hexyl)(methyl)carbamate (0.30 g, 0.66 mmol), 6-bromo-1-ethyl-3-iodo-1H-indazole (0.233 g, 0.66 mmol), K₃PO₄ (0.423 g, 1.99 mmol), and Pd(dppf)Cl₂ (0.049 g, 0.07 mmol) in a reaction vial under vacuum was added toluene (3.32 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound as an off-white solid, which is not very pure. The residue was purified by reverse phase flash chromatography with C₁₈ column (35-100% MeCN/water) to afford the titled compound as a gum (92 mg). LCMS (+ESI): Rt=3.14 min, (M+H)⁺=562.3 and 564.2.

### Step 3: 6-(4-(6-bromo-1-propyl-1H-indazol-3-yl)-3-fluorophenoxy)-N-methylhexan-1-amine

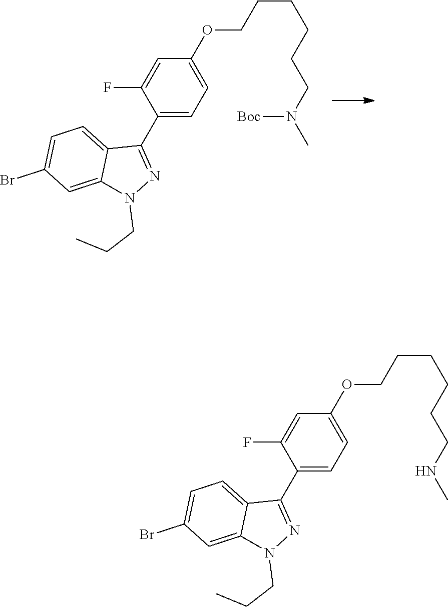 [SMILES: CCCn1nc(-c2ccc(OCCCCCCN(C)C(=O)OC(C)(C)C)cc2F)c2ccc(Br)cc21 ; CCCn1nc(-c2ccc(OCCCCCCNC)cc2F)c2ccc(Br)cc21]

A solution of tert-butyl (6-(4-(6-bromo-1-propyl-1H-indazol-3-yl)-3-fluorophenoxy)hexyl)(methyl)carbamate (0.092 g) in 1.5 mL TFA/DCM (1/1) was stirred at rt for 2 hr. The solvent was evaporated and the residue was diluted with EtOAc and washed with Na₂CO₃ solution, dried over Na₂SO₄, evaporated to afford a crude which was further purified by reverse phase flash chromatography with C is column (10-100% Solvent A/Solvent B, while solvent A is 0.1% of HCOOH in distilled water and Solvent B is 0.1% of HCOOH in MeCN) to afford the titled compound (71.0 mg). LCMS (+ESI): Rt=2.15 min, (M+H)⁺=462.2 and 464.1.

### 6-(4-(6-bromo-2-methyl-2H-indazol-3-yl)-3-fluorophenoxy)-N-methylhexan-1-amine (LZH-00051)

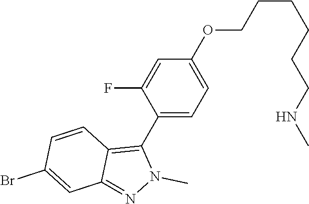 [SMILES: CNCCCCCCOc1ccc(-c2c3ccc(Br)cc3nn2C)c(F)c1]

### Step 1: tert-butyl (6-(4-(6-bromo-2-methyl-2H-indazol-3-yl)-3-fluorophenoxy)hexyl)(methyl)carbamate

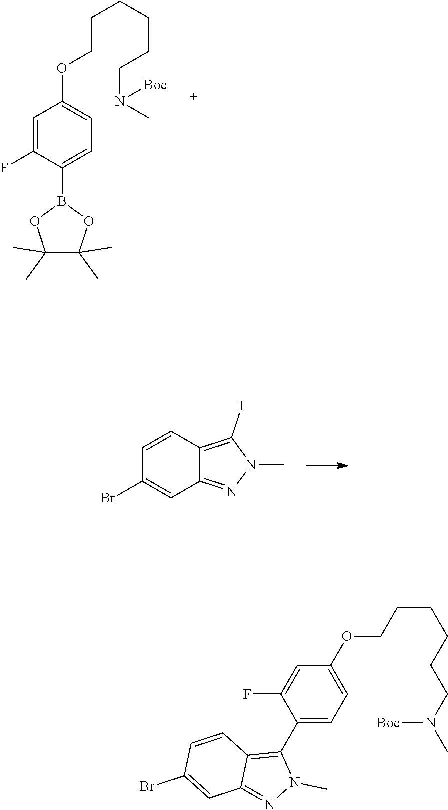 [SMILES: CN(CCCCCCOc1ccc(B2OC(C)(C)C(C)(C)O2)c(F)c1)C(=O)OC(C)(C)C ; Cn1nc2cc(Br)ccc2c1I ; CN(CCCCCCOc1ccc(-c2c3ccc(Br)cc3nn2C)c(F)c1)C(=O)OC(C)(C)C]

To a mixture of tert-butyl (6-(3-fluoro-4-(4,4,5,5-tetramethyl-1,3,2-dioxaborolan-2-yl)phenoxy)hexyl)(methyl)carbamate (0.30 g, 0.66 mmol), 6-bromo-3-iodo-2-methyl-2H-indazole (0.224 g, 0.66 mmol), K₃PO₄ (0.423 g, 1.99 mmol), and Pd(dppf)Cl₂ (0.049 g, 0.07 mmol) in a reaction vial under vacuum was added toluene (3.32 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound as an off-white solid, which is not very pure. The residue was purified by reverse phase flash chromatography with C₁₈ column (35-100% MeCN/water) to afford the titled compound as a gum (37.0 mg). LCMS (+ESI): Rt=3.05 min, (M+H)⁺=534.2 and 536.3.

### Step 2

### 6-(4-(6-bromo-2-methyl-2H-indazol-3-yl)-3-fluorophenoxy)-N-methylhexan-1-amine

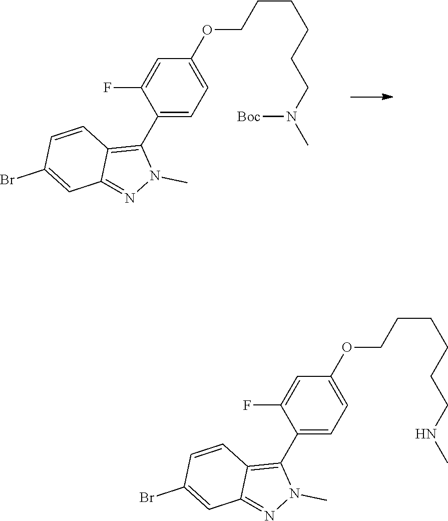 [SMILES: CN(CCCCCCOc1ccc(-c2c3ccc(Br)cc3nn2C)c(F)c1)C(=O)OC(C)(C)C ; CNCCCCCCOc1ccc(-c2c3ccc(Br)cc3nn2C)c(F)c1]

A solution of tert-butyl (6-(4-(6-bromo-2-methyl-2H-indazol-3-yl)-3-fluorophenoxy)hexyl)(methyl)carbamate (0.036 g) in 1.5 mL TFA/DCM (1/1) was stirred at rt for 2 hr. The solvent was evaporated and the residue was diluted with EtOAc and washed with Na₂CO₃ solution, dried over Na₂SO₄, evaporated to afford a crude which was further purified by reverse phase flash chromatography with C₁₈ column (10-100% Solvent A/Solvent B, while solvent A is 0.1% of HCOOH in distilled water and Solvent B is 0.1% of HCOOH in MeCN) to afford the titled compound (27.0 mg). LCMS (+ESI): Rt=1.92 min, (M+H)⁺=434.2 and 436.2.

### (4-(6-bromo-1H-indazol-3-yl)-3-fluorophenyl)methanol (LZH-00052)

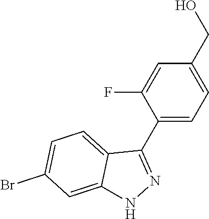 [SMILES: OCc1ccc(-c2n[nH]c3cc(Br)ccc23)c(F)c1]

### Step 1: 6-bromo-3-iodo-1-(methylsulfonyl)-1H-indazole

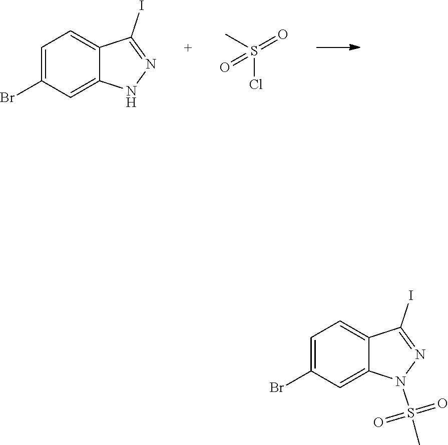

To a mixture of 6-bromo-3-iodo-1H-indazole (11 g, 34.06 mmol) and NEt₃ (0.48 g, 4.75 mmol) in THF (21 mL) stired in an ice/water bath, MsCl (0.65 g, 4.71 mmol) was added dropwise. After the addition, the mixture was stirred at rt for an overnight. The reaction was worked up by the addition of water and extracted with EtOAc, dried over Na₂SO₄, filtered and evaporated. The residue was purified by flash chromatography (0-20% EtOAc/hexanes) to afford the titled compound (1.41 g) as an off-white solid. ¹HNMR (500.3 MHz, CDCl₃): δ (ppm) 8.63 (s, 1H), 7.54 (d, 1H), 7.35 (d, 1H), 2.77 (s, 3H).

### Step 2: 4-(6-bromo-1H-indazol-3-yl)-3-fluorobenzaldehyde

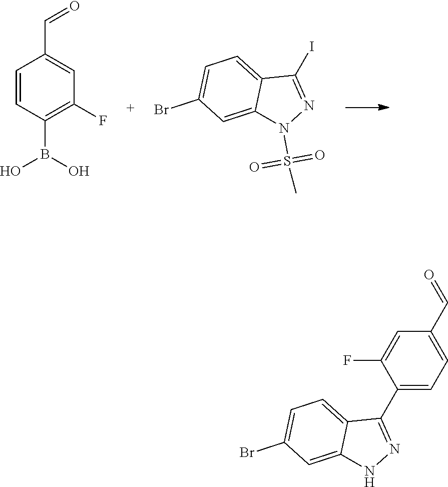 [SMILES: O=Cc1ccc(B(O)O)c(F)c1 ; CS(=O)(=O)n1nc(I)c2ccc(Br)cc21 ; O=Cc1ccc(-c2n[nH]c3cc(Br)ccc23)c(F)c1]

To a mixture of (2-fluoro-4-formylphenyl)boronic acid (0.25 g, 1.49 mmol), 6-bromo-3-iodo-1-(methylsulfonyl)-1H-indazole (0.597 g, 1.49 mmol), K₂CO₃ (0.616 g, 4.47 mmol), and Pd(dppf)Cl₂ (0.109 g, 0.15 mmol) in a reaction vial under vacuum was added 1,4-dioxane/water (4:1, 8.0 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-40% EtOAc/hexanes) to afford the titled compound as an off-white solid (127 mg), which is not very pure but use directly in next step. LCMS (+ESI): Rt=2.04 min, (M+H)⁺=319.0 and 321.0.

### Step 3: (4-(6-bromo-1H-indazol-3-yl)-3-fluorophenyl)methanol

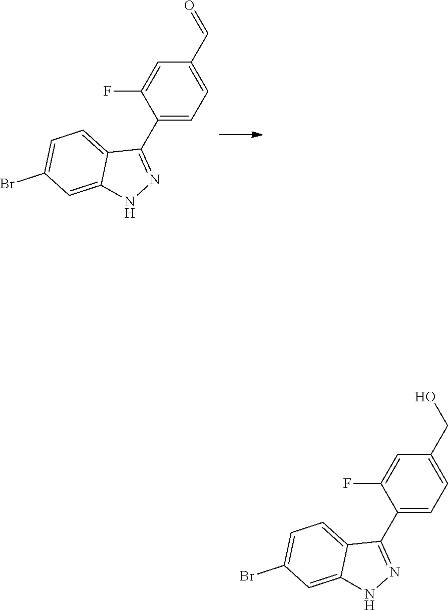 [SMILES: O=Cc1ccc(-c2n[nH]c3cc(Br)ccc23)c(F)c1 ; OCc1ccc(-c2n[nH]c3cc(Br)ccc23)c(F)c1]

To a mixture of 4-(6-bromo-1H-indazol-3-yl)-3-fluorobenzaldehyde (0.127 g, 0.40 mmol) in a reaction vial with a solvent mixture (MeOH/THF 4/1, 8 mL) was added NaBH₄ (41 mg, 1.19 mmol). After the addition, the mixture was stirred at room temperature for 30 min. The reaction was worked up by the addition of water and extracted with EtOAc, dried over Na₂SO₄, filtered and evaporated. The residue was purified by flash chromatography (0-40% EtOAc/hexanes) to afford the titled compound as an off-white solid, which is not very pure but can be further purified by reverse phase flash chromatography (15.5 g of C₁₈ column, 35-100% MeCN/H₂O) to afford the titled compound (48 mg) as a white solid. LCMS (+ESI): Rt=1.81 min, (M+H)⁺=321.0 and 323.1.

### N1-(4-(6-bromo-1-(methylsulfonyl)-1H-indazol-3-yl)-3-fluorobenzyl)-N1,N2-dimethylethane-1,2-diamine (LZH-00053)

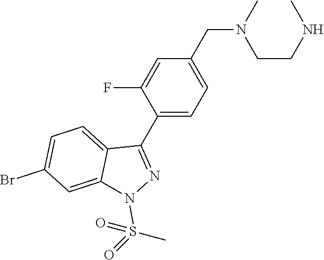

### Step 1: N1-(4-(6-bromo-1-(methylsulfonyl)-1H-indazol-3-yl)-3-fluorobenzyl)-N1,N2-dimethylethane-1,2-diamine

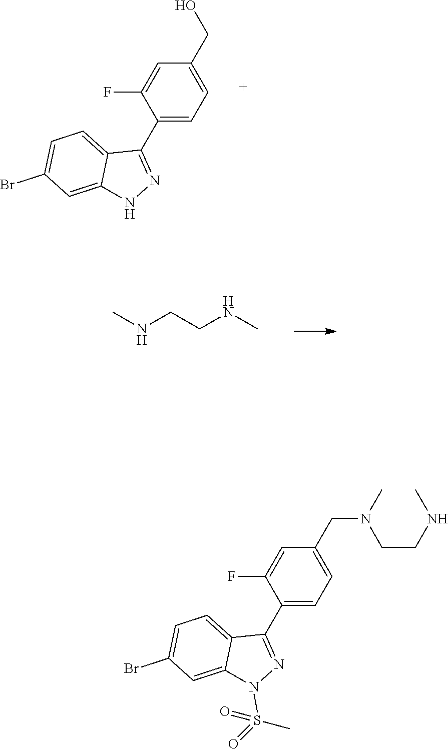

To a solution of (4-(6-bromo-1H-indazol-3-yl)-3-fluorophenyl)methanol (0.044 g, 0.14 mmol) and NEt₃ (0.055 g, 0.55 mmol) in DCM (3 mL) stirred in an ice/water bath, was added MsCl (0.035 g, 0.30 mmol). The mixture was stirred at rt for 2 hr. The reaction was worked up by the addition of sat NaHCO₃/H₂O. The organic layer was separated, dried over Na₂SO₄, filtered, and evaporated. The residue was redissolved in 4 mL of 1,4-dioxane and the the solution was added N1,N2-dimethylethane-1,2-diamine (0.362 g, 4.11 mmol). The mixture was reacted at 60° C. for 2 hr. The solvent was evaporated and the residue was treated with EtOAc, washed with sat NaHCO₃/H₂O, dried over Na₂SO₄, filtered and evaporated. The residue was purified by reverse phase flash chromatography with C₁₈ column (10-100% Solvent A/Solvent B, while solvent A is 0.1% of HCOOH in distilled water and Solvent B is 0.1% of HCOOH in MeCN) to afford the titled compound (6.0 mg) as a white solid. LCMS (+ESI): Rt=1.39 min, (M+H)⁺=469.0 and 471.0.

### 6-(4-(6-bromo-1-methyl-1H-indazol-3-yl)phenoxy)-N-methylhexan-1-amine (LZH-00054)

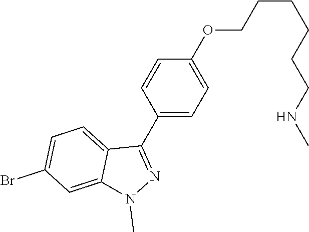 [SMILES: CNCCCCCCOc1ccc(-c2nn(C)c3cc(Br)ccc23)cc1]

### Step 1: 1-((6-bromohexyl)oxy)-4-iodobenzene

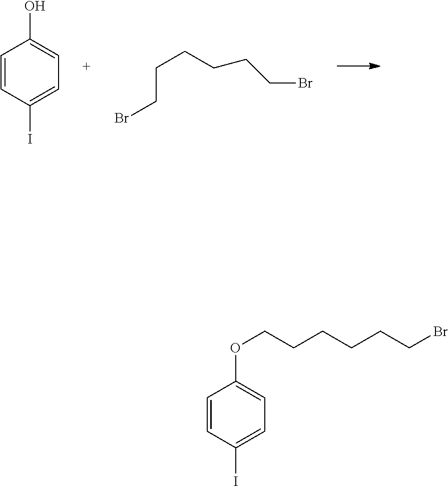 [SMILES: Oc1ccc(I)cc1 ; BrCCCCCCBr ; BrCCCCCCOc1ccc(I)cc1]

To a mixture of 4-iodophenol (11 g, 50 mmol) in DMF (250 mL) stired in an ice/water bath, NaH (2.2 g, 55 mmol) was added portion wise. After the addition, the mixture was stirred at rt for 30 min. To the mixture thus obtained, 1,6-dibromohexane (36.59 g, 149.98 mmol) was added dropwise. After the addition, the mixture was stirred at rt for an overnight. The solvent was evaporated and the residue was diluted with EtOAc and filtered through a silica gel plug, and washed with more EtOAc until no more compound out. The filtrate was evaporated to afford a crude which was further purified by flash chromatography (0-5% EtOAc/hexane) to afford the titled compound (14.53 g) as a white solid. ¹HNMR (500.3 MHz, CDCl₃): δ (ppm) 7.53 (d, 2H), 6.67 (d, 2H), 3.92 (t, 2H), 3.41 (t, 2H), 1.86-1.92 (m, 2H), 1.75-1.82 (m, 2H), 1.46-1.53 (m, 4H).

### Step 2: tert-butyl (6-(4-iodophenoxy)hexyl)(methyl)carbamate

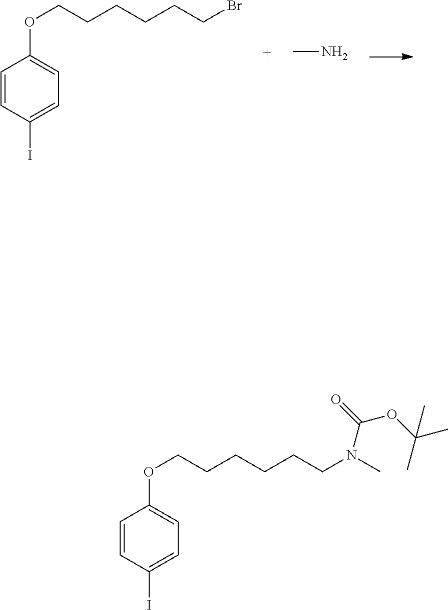

A mixture of 1-((6-bromohexyl)oxy)-4-iodobenzene (14.5 g, 37.85 mmol), 40% Methylamine in water (39.68 g, 511 mmol) in 1,4-dioxane (95 mL) was stirred at 70° C. overnight. The solvent was evaporated and the residue was diluted with EtOAc and filtered through a silica gel plug and washed with more EtOAc until no more compound out. Evaporated to afford a crude which was further purified by flash chromatography. LCMS (+ESI): Rt=2.42 min, (M+H)⁺=434.1.

### Step 3: tert-butyl methyl(6-(4-(4,4,5,5-tetramethyl-1,3,2-dioxaborolan-2-yl)phenoxy)hexyl)carbamate

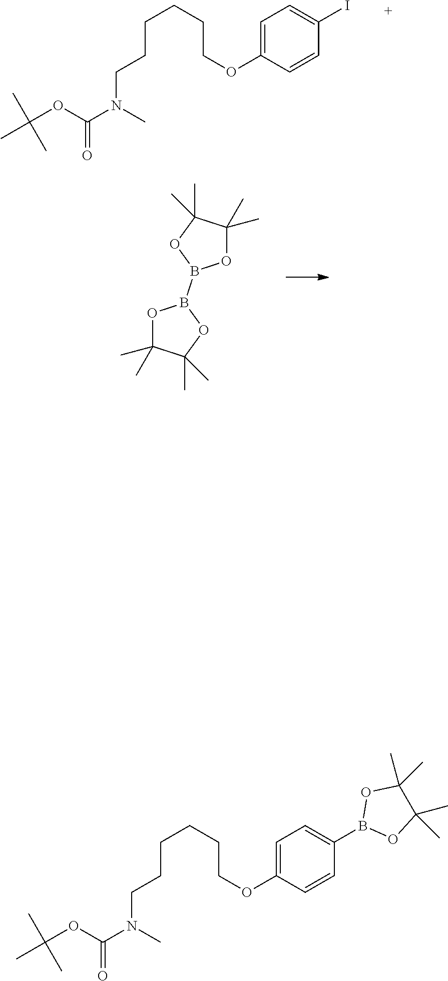

To a mixture of tert-butyl (6-(4-iodophenoxy)hexyl)(methyl)carbamate (3.70 g, 8.54 mmol), 4,4,4′,4′,5,5,5′,5′-octamethyl-2,2′-bi(1,3,2-dioxaborolane) (0.233 g, 0.66 mmol), KOAc (3.77 g, 38.42 mmol), and Pd(dppf)Cl₂ (0.625 g, 0.85 mmol) in a reaction vial under vacuum was added DMSO (42 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound as an off-white solid, which is not very pure but use directly in next step. LCMS (+ESI): Rt=2.42 min, (M+H)⁺=434.3.

### Step 4: tert-butyl (6-(4-(6-bromo-1-methyl-H-indazol-3-yl)phenoxy)hexyl)(methyl)carbamate

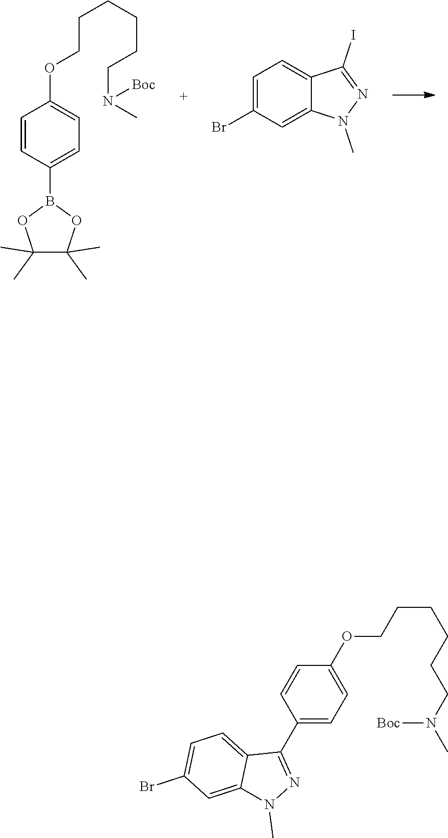 [SMILES: CN(CCCCCCOc1ccc(B2OC(C)(C)C(C)(C)O2)cc1)C(=O)OC(C)(C)C ; Cn1nc(I)c2ccc(Br)cc21 ; CN(CCCCCCOc1ccc(-c2nn(C)c3cc(Br)ccc23)cc1)C(=O)OC(C)(C)C]

To a reaction vial containing tert-butyl methyl(6-(4-(4,4,5,5-tetramethyl-1,3,2-dioxaborolan-2-yl)phenoxy)hexyl)carbamate (0.3 g, 0.69 mmol), 6-bromo-3-iodo-1-methyl-1H-indazole (0.23 g, 0.69 mmol), K₂CO₃ (0.29 g, 2.08 mmol), and Pd(dppf)Cl₂ (0.051 g, 0.07 mmol) under vacuum, 3.5 mL of 1,4-dioxane/water (4:1) was added via a syringe. After the addition, a nitrogen balloon was applied to the system and the mixture was stirred at 100° C. for an overnight.he reaction was worked up by the addition of water and extracted with EtOAc, dried over Na₂SO₄, filtered and evaporated. The residue was purified by flash chromatography (0-20% EtOAc/hexanes) to afford the titled compound as an off-white solid, which is not very pure. The residue was purified by reverse phase flash chromatography with C₁₈ column (35-100% MeCN/water) to afford the titled compound as a gum (140 mg). LCMS(+ESI): Rt=2.99 min, (M+H)⁺=516.2 and 518.2.

### Step 5: 6-(4-(6-bromo-1-methyl-1H-indazol-3-yl)phenoxy)-N-methylhexan-1-amine

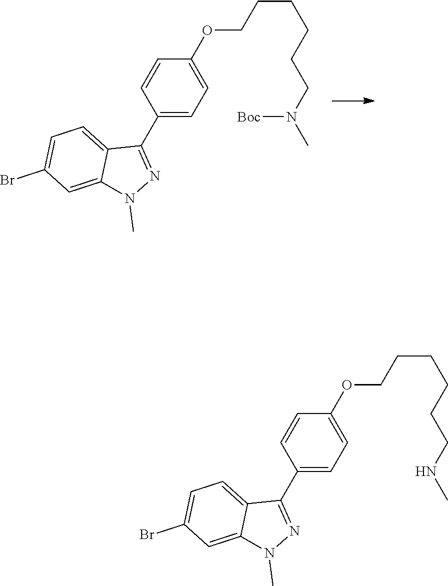 [SMILES: CN(CCCCCCOc1ccc(-c2nn(C)c3cc(Br)ccc23)cc1)C(=O)OC(C)(C)C ; CNCCCCCCOc1ccc(-c2nn(C)c3cc(Br)ccc23)cc1]

A solution of tert-butyl (6-(4-(6-bromo-1-methyl-1H-indazol-3-yl)phenoxy)hexyl)(methyl)carbamate (0.140 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (120.0 mg). LCMS (+ESI): Rt=2.05 min, (M+H)⁺=416.1 and 418.1.

### 6-(4-(6-bromo-1H-indazol-3-yl)phenoxy)-N-methylhexan-1-amine (LZH-00055)

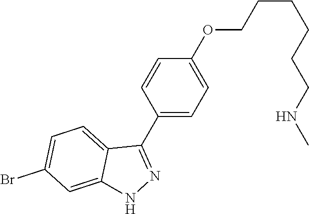 [SMILES: CNCCCCCCOc1ccc(-c2n[nH]c3cc(Br)ccc23)cc1]

### Step 1: tert-butyl (6-(4-(6-bromo-1H-indazol-3-yl)phenoxy)hexyl)(methyl)carbamate

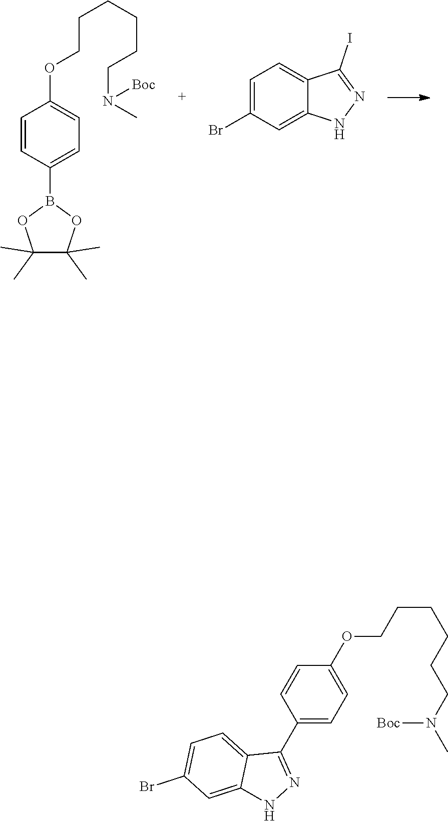 [SMILES: CN(CCCCCCOc1ccc(B2OC(C)(C)C(C)(C)O2)cc1)C(=O)OC(C)(C)C ; Brc1ccc2c(I)n[nH]c2c1 ; CN(CCCCCCOc1ccc(-c2n[nH]c3cc(Br)ccc23)cc1)C(=O)OC(C)(C)C]

To a reaction vial containing tert-butyl methyl(6-(4-(4,4,5,5-tetramethyl-1,3,2-dioxaborolan-2-yl)phenoxy)hexyl)carbamate (0.3 g, 0.69 mmol), 6-bromo-3-iodo-1H-indazole (0.22 g, 0.69 mmol), K₂CO₃ (0.29 g, 2.08 mmol), and Pd(dppf)Cl₂ (0.051 g, 0.07 mmol) under vacuum, 3.5 mL of 1,4-dioxane/water (4:1) was added via a syringe. After the addition, a nitrogen balloon was applied to the system and the mixture was stirred at 100° C. for an overnight.he reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure. The residue was purified by reverse phase flash chromatography with C₁₈ column (35-100% MeCN/water) to afford the titled compound as a gum (47 mg). LCMS (+ESI): Rt=2.73 min, (M+H)⁺=502.1 and 504.1.

### Step 2: 6-(4-(6-bromo-1H-indazol-3-yl)phenoxy)-N-methylhexan-1-amine

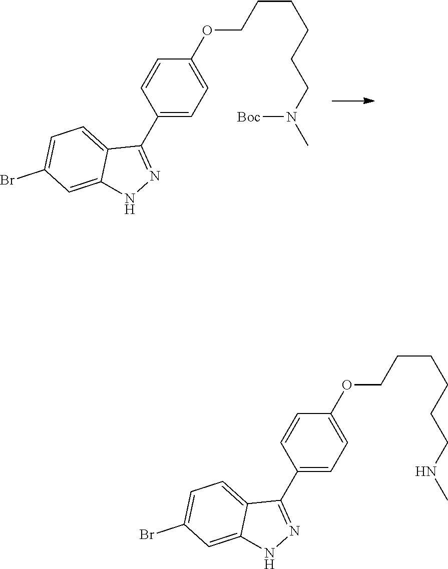 [SMILES: CN(CCCCCCOc1ccc(-c2n[nH]c3cc(Br)ccc23)cc1)C(=O)OC(C)(C)C ; CNCCCCCCOc1ccc(-c2n[nH]c3cc(Br)ccc23)cc1]

A solution of tert-butyl (6-(4-(6-bromo-1H-indazol-3-yl)phenoxy)hexyl)(methyl)carbamate (0.047 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (37.0 mg). LCMS (+ESI): Rt=1.92 min, (M+H)⁺=402.1 and 404.1.

### 3-(2-(4-(6-bromo-1-methyl-1H-indazol-3-yl)-3-fluorophenoxy)ethoxy)-N-methylpropan-1-amine (LZH-00056)

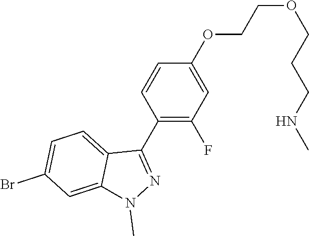 [SMILES: CNCCCOCCOc1ccc(-c2nn(C)c3cc(Br)ccc23)c(F)c1]

### Step 1: 2-(4-bromo-3-fluorophenoxy)ethanol

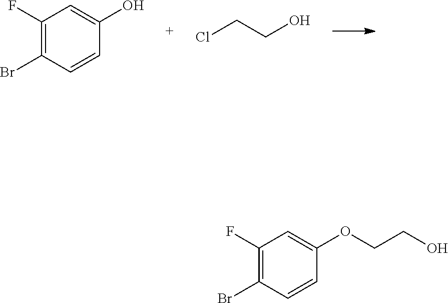

A mixture of 4-bromo-3-fluorophenol (9.55 g, 50 mmol), 2-chloroethanol (6.04 g, 75.0 mmol), and NaOH (3.0 g, 75 mmol) in H₂O (20 mL) was stirred at 60° C. overnight. The solvent was evaporated and the residue was diluted with EtOAc and filtered through a silica gel plug and washed with more EtOAc until no more compound out. Evaporated to afford a crude which was further purified by flash chromatography (0-60% EtOAc/hexanes) to afford the titled compound as an oil (8.23 g). LCMS (+ESI): Rt=1.40 min, (M+H)⁺=235.0 and 237.1.

### Step 2: 1-bromo-4-(2-(3-chloropropoxy)ethoxy)-2-fluorobenzene

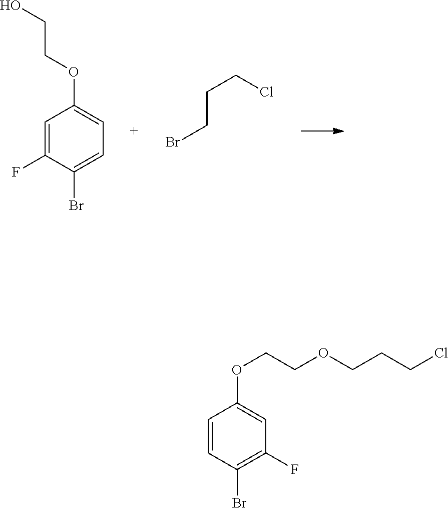 [SMILES: OCCOc1ccc(Br)c(F)c1 ; ClCCCBr ; Fc1cc(OCCOCCCCl)ccc1Br]

A mixture of 2-(4-bromo-3-fluorophenoxy)ethanol (3 g, 12.76 mmol), 1-bromo-3-chloropropane (12.0567 g, 76.58 mmol), Bu₄NHSO₄ (0.13 g, 0.38 mmol), and NaOH (50% in water) (3.999 g, 99.98 mmol) in a sealed tube was stirred at 65° C. for an overnight. The mixture was diluted with EtOAc and washed with water. The organic layer was separated, dried over Na₂SO₄, filtered, and evaporated to afford a crude which was further purified by flash chromatography (0-15% EtOAc/hexanes) to afford the titled compound (3.64 g) as an oil. LCMS (+ESI): Rt=2.31 min, (M+Na)⁺=332.9 and 335.2.

### Step 3: tert-butyl (3-(2-(4-bromo-3-fluorophenoxy)ethoxy)propyl)(methyl)carbamate

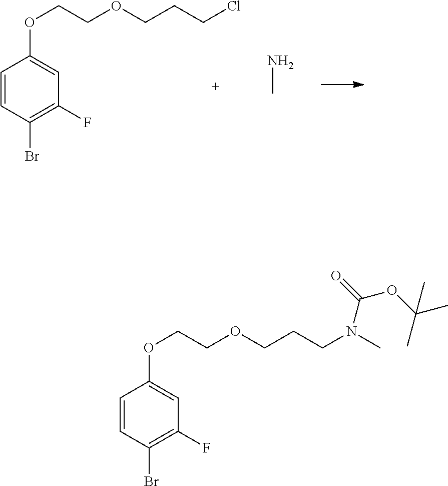

A mixture of 1-bromo-4-(2-(3-chloropropoxy)ethoxy)-2-fluorobenzene (3.64 g, 11.89 mmol), MeNH₂ (40% in water, 27.7 g, 357 mmol), in 1,4-dioxane (60 mL) was stirred at 60° C. overnight. The solvent was evaporated and the residue was diluted with water and extracted with DCM, dried over Na₂SO₄, filtered and evaporated to afford a crude which was used directly. The crude thus obtained was redissolved in EtOAc (30 mL), and then Boc anyhydride (2.85 g, 13.08 mmol) was added in small portion. The mixture was stirred at rt overnight. The solvent was evaporated and the residue was purified by flash chromatography (0-30% EtOAc/hexanes) to afford the titled compound (3.82 g) as a oil. ¹HNMR (500.3 MHz, CDCl₃): δ (ppm) 7.39 (t, 1H), 6.72 (dd, 1H), 6.60-6.64 (m, 1H), 4.08 (t, 2H), 3.76 (t, 2H), 3.53 (t, 2H), 2.86 (brs, 2H), 2.85 (brs, 3H), 1.84-1.80 (m, 2H), 1.45 (s, 9H).

### Step 4: tert-butyl (3-(2-(3-fluoro-4-(4,4,5,5-tetramethyl-1,3,2-dioxaborolan-2-yl)phenoxy)ethoxy)propyl)(methyl)carbamate

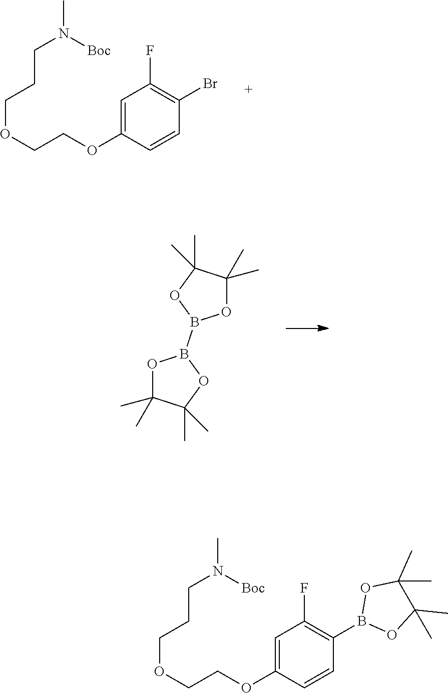

To a mixture of tert-butyl (3-(2-(4-bromo-3-fluorophenoxy)ethoxy)propyl)(methyl)carbamate (3.25 g, 8.0 mmol), 4,4,4′,4′,5,5,5′,5′-octamethyl-2,2′-bi(1,3,2-dioxaborolane) (3.047 g, 12.0 mmol), KOAc (1.963 g, 20.0 mmol), and Pd(dppf)Cl₂ (0.293 g, 0.40 mmol) in a RBF under vacuum was added 1,4-dioxane (40 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-30% EtOAc/hexanes) to afford the titled compound (3.45 g) as a wax. LCMS (+ESI): Rt=2.51 min, (M+H)⁺=454.3.

### Step 5: tert-butyl (3-(2-(4-(6-bromo-1-methyl-1H-indazol-3-yl)-3-fluorophenoxy)ethoxy)propyl)(methyl)carbamate

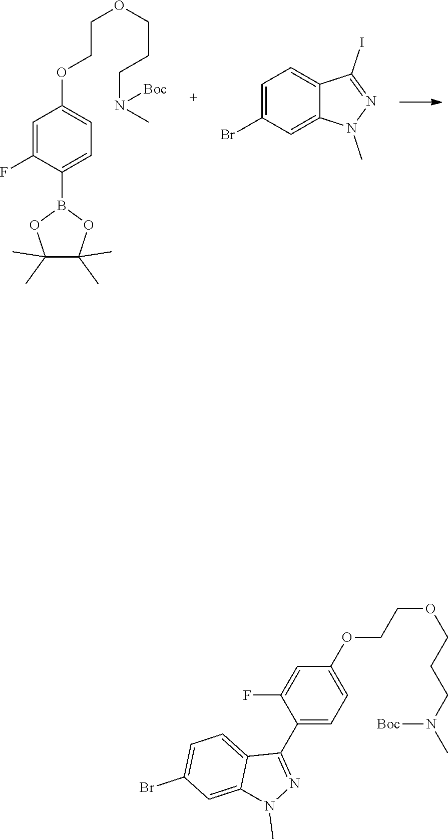 [SMILES: CN(CCCOCCOc1ccc(B2OC(C)(C)C(C)(C)O2)c(F)c1)C(=O)OC(C)(C)C ; Cn1nc(I)c2ccc(Br)cc21 ; CN(CCCOCCOc1ccc(-c2nn(C)c3cc(Br)ccc23)c(F)c1)C(=O)OC(C)(C)C]

To a mixture of tert-butyl (3-(2-(3-fluoro-4-(4,4,5,5-tetramethyl-1,3,2-dioxaborolan-2-yl)phenoxy)ethoxy)propyl)(methyl)carbamate (0.25 g, 0.55 mmol), 6-bromo-3-iodo-1-methyl-1H-indazole (0.186 g, 0.55 mmol), K₂CO₃ (0.229 g, 1.65 mmol), and Pd(dppf)Cl₂ (0.041 g, 0.06 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-35% EtOAc/hexanes) to afford the titled compound as an off-white solid, which is not very pure; thus further purified by reverse phase flash chromatography with C₁₈ column (35-100% MeCN/water) to afford the titled compound as a wax (38.0 mg). LCMS (+ESI): Rt=2.62 min, (M+H)⁺=536.2 and 538.4.

### Step 6: 3-(2-(4-(6-bromo-1-methyl-1H-indazol-3-yl)-3-fluorophenoxy)ethoxy)-N-methylpropan-1-amine

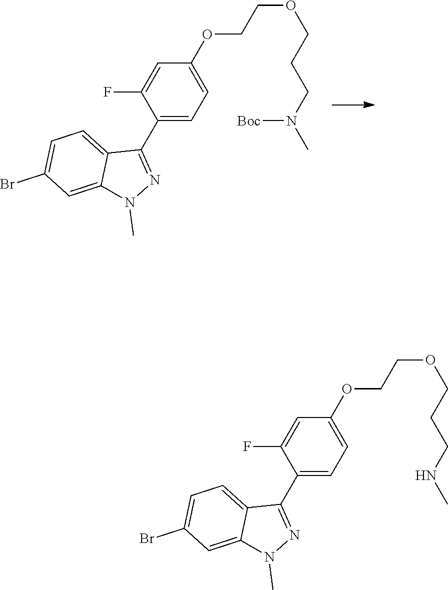 [SMILES: CN(CCCOCCOc1ccc(-c2nn(C)c3cc(Br)ccc23)c(F)c1)C(=O)OC(C)(C)C ; CNCCCOCCOc1ccc(-c2nn(C)c3cc(Br)ccc23)c(F)c1]

A solution of tert-butyl (3-(2-(4-(6-bromo-1-methyl-1H-indazol-3-yl)-3-fluorophenoxy)ethoxy)propyl)(methyl)carbamate (0.058 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (38.0 mg). LCMS (+ESI): Rt=1.91 min, (M+H)⁺=436.1 and 438.1.

### 3-(2-(4-(6-bromo-1H-indazol-3-yl)-3-fluorophenoxy)ethoxy)-N-methylpropan-1-amine (LZH-00057)

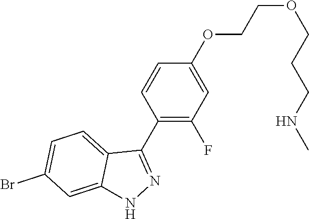 [SMILES: CNCCCOCCOc1ccc(-c2n[nH]c3cc(Br)ccc23)c(F)c1]

### Step 1: tert-butyl (3-(2-(4-(6-bromo-1-methyl-1H-indazol-3-yl)-3-fluorophenoxy)ethoxy)propyl)(methyl)carbamate

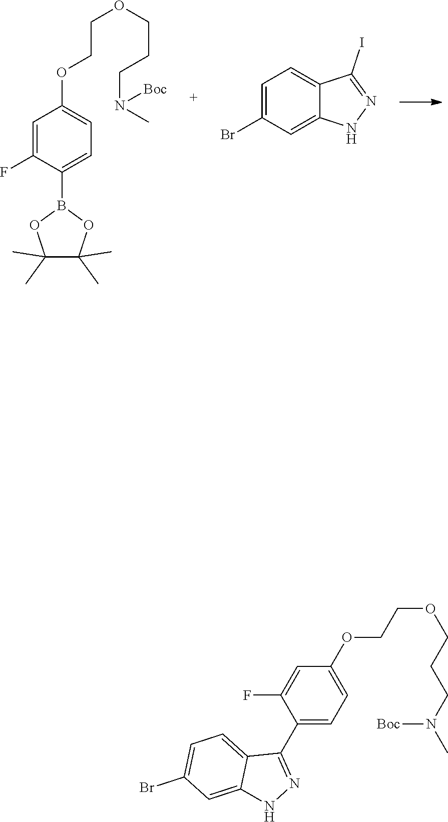 [SMILES: CN(CCCOCCOc1ccc(B2OC(C)(C)C(C)(C)O2)c(F)c1)C(=O)OC(C)(C)C ; Brc1ccc2c(I)n[nH]c2c1 ; CN(CCCOCCOc1ccc(-c2n[nH]c3cc(Br)ccc23)c(F)c1)C(=O)OC(C)(C)C]

To a mixture of tert-butyl (3-(2-(3-fluoro-4-(4,4,5,5-tetramethyl-1,3,2-dioxaborolan-2-yl)phenoxy)ethoxy)propyl)(methyl)carbamate (0.25 g, 0.55 mmol), 6-bromo-3-iodo-1H-indazole (0.178 g, 0.66 mmol), K₃PO₄ (0.225 g, 1.66 mmol), and Pd(dppf)Cl₂ (0.041 g, 0.06 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-35% EtOAc/hexanes) to afford the titled compound as an off-white solid, which is not very pure; thus further purified by reverse phase flash chromatography with C₁₈ column (35-100% MeCN/water) to afford the titled compound as a wax (45.0 mg). LCMS (+ESI): Rt=2.40 min, (M+H)⁺=522.3 and 524.4.

### Step 2: 3-(2-(4-(6-bromo-H-indazol-3-yl)-3-fluorophenoxy)ethoxy)-N-methylpropan-1-amine

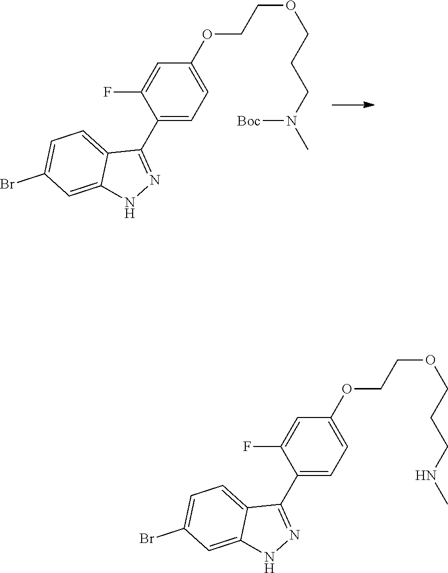 [SMILES: CN(CCCOCCOc1ccc(-c2n[nH]c3cc(Br)ccc23)c(F)c1)C(=O)OC(C)(C)C ; CNCCCOCCOc1ccc(-c2n[nH]c3cc(Br)ccc23)c(F)c1]

A solution of tert-butyl (3-(2-(4-(6-bromo-1H-indazol-3-yl)-3-fluorophenoxy)ethoxy)propyl)(methyl)carbamate (0.063 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (50.0 mg). LCMS (+ESI): Rt=1.77 min, (M+H)⁺=422.0 and 424.0.

### 2-(3-(4-(6-bromo-1-methyl-1H-indazol-3-yl)-3-fluorophenoxy)propoxy)-N-methylethanamine (LZH-00058)

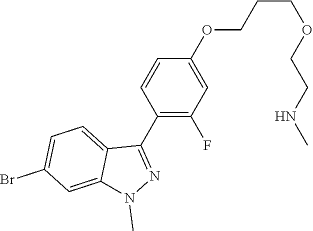 [SMILES: CNCCOCCCOc1ccc(-c2nn(C)c3cc(Br)ccc23)c(F)c1]

### Step 1: 3-(4-bromo-3-fluorophenoxy)propan-1-ol

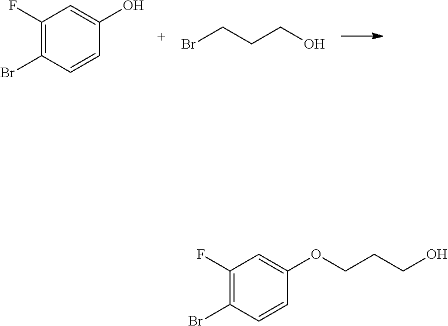 [SMILES: Oc1ccc(Br)c(F)c1 ; OCCCBr ; OCCCOc1ccc(Br)c(F)c1]

To a mixture of 4-bromo-3-fluorophenol (9.55 g, 50 mmol) and 3-bromopropan-1-ol (8.3394 g, 60 mmol) in MeCN (100 mL) stired in an ice/water bath, K₂CO₃ (10.35 g, 75 mmol) was added in one portion. After the addition, the mixture was stirred at reflux for an overnight. The solvent was evaporated and the residue was diluted with EtOAc and filtered through a silica gel plug and washed with more EtOAc until no more compound out. The filtrate was evaporated to afford a crude which was further purified by flash chromatography (0-60% EtOAc/hexanes) to afford the titled compound as an oil (11.2 g). LCMS (+ESI): Rt=1.53 min, (M+H)⁺=249.1 and 251.1.

### Step 2: tert-butyl 2-(3-(4-bromo-3-fluorophenoxy)propoxy)acetate

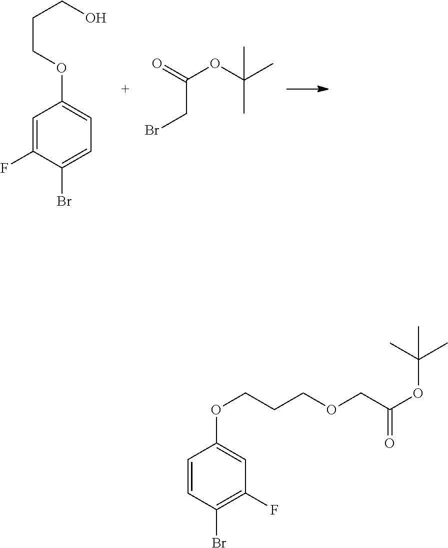

To a mixture of 3-(4-bromo-3-fluorophenoxy)propan-1-ol (2 g, 8.03 mmol) tert-butyl 2-bromoacetate (2.35 g, 12.04 mmol) and Bu₄NHSO₄ (0.27 g, 0.8 mmol) in toluene (8 mL) stired in an ice/water bath, 50% of NaOH (7.71 g, 192.71 mmol) was added dropwise. After the addition, the mixture was stirred at rt overnight. The reactin was worked up by the addition of water and extracted with EtOAc, dried over Na₂SO₄, filtered, and evaporated to afford a crude which was further purified by flash chromatography (0-10% EtOAc/hexanes) to afford the titled compound (1.51 g) as an oil. LCMS (+ESI): Rt=2.43 min, (M+Na)⁺=385.1 and 387.0.

### Step 3: 2-(3-(4-bromo-3-fluorophenoxy)propoxy)ethanol

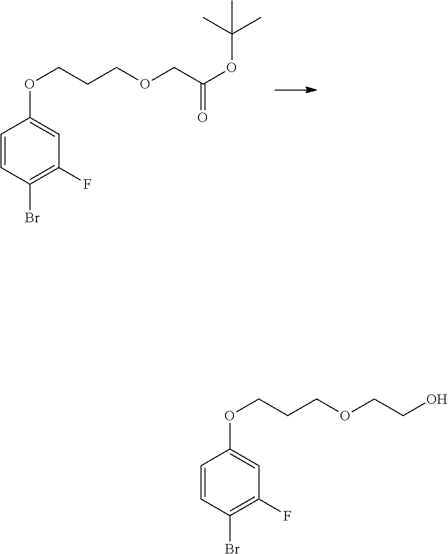 [SMILES: CC(C)(C)OC(=O)COCCCOc1ccc(Br)c(F)c1 ; OCCOCCCOc1ccc(Br)c(F)c1]

A mixture of tert-butyl 2-(3-(4-bromo-3-fluorophenoxy)propoxy)acetate (1.51 g, 7.90 mmol) in TFA/DCM (1:1.8 mL) was stirred at rt for 3 hr. The solvent was evaporated and the residue was diluted with EtOAc/toluene and evaporated again. The residue thus obtained was redissolved in THF (8 mL) and to this solution under N₂ atmosphere, was added BH₃.SMe₂ dropwise. The mixture was then stirred at 55° C. overnight. The reaction was worked up by the addition of water dropwise. The mixture was extracted with EtOAc, dried over Na₂SO₄, filtered and evaporated. The residue was purified by flash chromatography (0-50% EtOAc/hexanes) to afford the titled compound (0.91 g) as a wax. LCMS (+ESI): Rt=1.87 min, (M+Na)⁺=315.0 and 317.0.

### Step 4: tert-butyl (2-(3-(4-bromo-3-fluorophenoxy)propoxy)ethyl)(methyl)carbamate

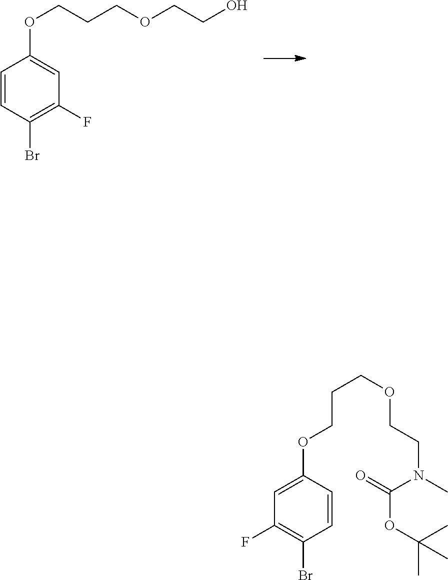 [SMILES: OCCOCCCOc1ccc(Br)c(F)c1 ; CN(CCOCCCOc1ccc(Br)c(F)c1)C(=O)OC(C)(C)C]

To a mixture of 2-(3-(4-bromo-3-fluorophenoxy)propoxy)ethanol (0.91 g, 3.1 mmol) and NEt₃ (0.38 g, 3.73 mmol) in DCM (21 mL) stired in an ice/water bath, MsCl (0.43 g, 3.73 mmol) was added dropwise. After the addition, the mixture was stirred at rt for 2 hr. The solvent was evaporated and the residue was redissolved in 34 mL of 1,4-dioxane and transferred into a sealed tube. MeNH₂ (40% in water, 31.7 g, 408 mmol) was added. The mixture was heated at 60° C. overnight and the solvent was evaporated. The residue was diluted with water and extracted with DCM, dried over Na₂SO₄, filtered and evaporated to afford a crude which was used directly. The crude thus obtained was redissolved in EtOAc (34 mL), and then Boc anyhydride (3.27 g, 14.98 mmol) was added in small portion. The mixture was stirred at rt overnight. The solvent was evaporated and the residue was purified by flash chromatography (0-30% EtOAc/hexanes) to afford the titled compound (1.04 g) as a oil. LCMS (+ESI): Rt=2.37 min, (M+Na)⁺=428.1 and 430.1.

### Step 5: tert-butyl (2-(3-(3-fluoro-4-(4,4,5,5-tetramethyl-1,3,2-dioxaborolan-2-yl)phenoxy)propoxy)ethyl)(methyl)carbamate

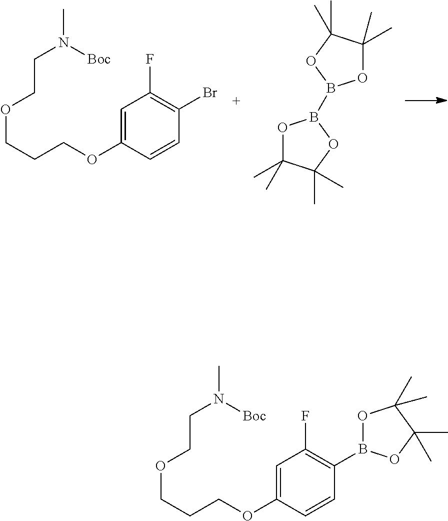

To a mixture of tert-butyl (2-(3-(4-bromo-3-fluorophenoxy)propoxy)ethyl)(methyl)carbamate (1.04 g, 2.56 mmol), 4,4,4′,4′,5,5,5′,5′-octamethyl-2,2′-bi(1,3,2-dioxaborolane) (1.30 g, 5.12 mmol), KOAc (0.63 g, 6.40 mmol), and Pd(dppf)Cl₂ (0.18 g, 0.26 mmol) in a RBF under vacuum was added DMSO (40 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound (1.03 g) as a wax. LCMS (+ESI): Rt=2.56 min, (M+H)⁺=454.5.

### Step 6: tert-butyl (2-(3-(4-(6-bromo-1-methyl-1H-indazol-3-yl)-3-fluorophenoxy)propoxy)ethyl)(methyl)carbamate

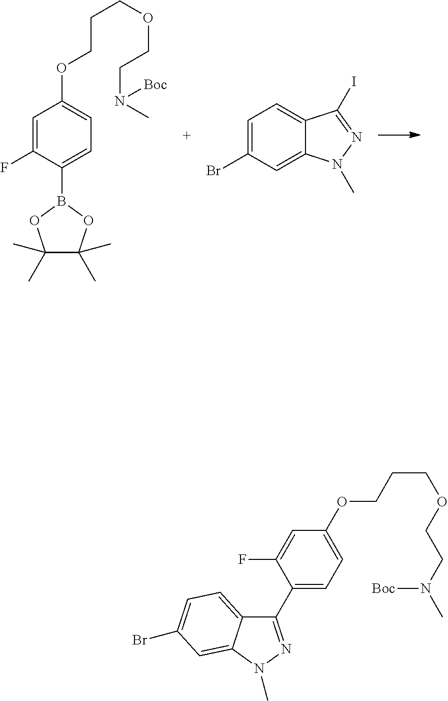 [SMILES: CN(CCOCCCOc1ccc(B2OC(C)(C)C(C)(C)O2)c(F)c1)C(=O)OC(C)(C)C ; Cn1nc(I)c2ccc(Br)cc21 ; CN(CCOCCCOc1ccc(-c2nn(C)c3cc(Br)ccc23)c(F)c1)C(=O)OC(C)(C)C]

To a mixture of tert-butyl (2-(3-(3-fluoro-4-(4,4,5,5-tetramethyl-1,3,2-dioxaborolan-2-yl)phenoxy)propoxy)ethyl)(methyl)carbamate (0.25 g, 0.55 mmol), 6-bromo-3-iodo-1-methyl-1H-indazole (0.186 g, 0.55 mmol), K₂CO₃ (0.229 g, 1.65 mmol), and Pd(dppf)Cl₂ (0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-35% EtOAc/hexanes) to afford the titled compound as an off-white solid, which is not very pure; thus further purified by reverse phase flash chromatography with C₁₈ column (35-100% MeCN/water) to afford the titled compound as a wax (80.0 mg). LCMS (+ESI): Rt=2.74 min, (M+H)⁺=536.2 and 538.2.

### Step 7: 2-(3-(4-(6-bromo-1-methyl-1H-indazol-3-yl)-3-fluorophenoxy)propoxy)-N-methylethanamine

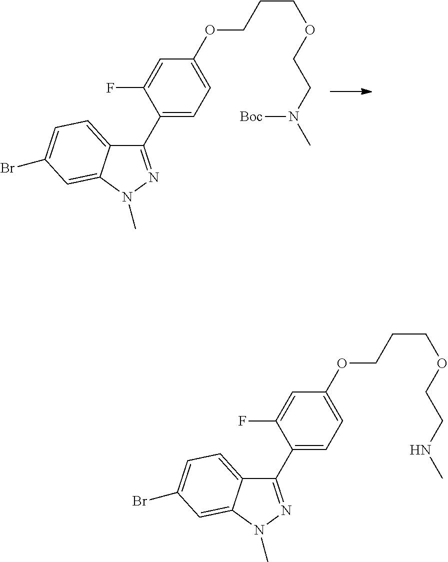 [SMILES: CN(CCOCCCOc1ccc(-c2nn(C)c3cc(Br)ccc23)c(F)c1)C(=O)OC(C)(C)C ; CNCCOCCCOc1ccc(-c2nn(C)c3cc(Br)ccc23)c(F)c1]

A solution of tert-butyl (2-(3-(4-(6-bromo-1-methyl-1H-indazol-3-yl)-3-fluorophenoxy)propoxy)ethyl)(methyl)carbamate (0.080 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (61.0 mg). LCMS (+ESI): Rt=1.99 min, (M+H)⁺=436.1 and 438.1.

### 2-(3-(4-(6-bromo-1-methyl-1H-indazol-3-yl)-3-fluorophenoxy)propoxy)-N-methylethanamine (LZH-00059)

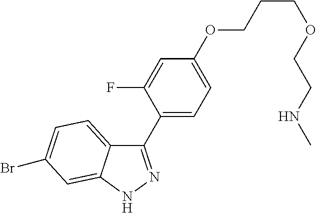 [SMILES: CNCCOCCCOc1ccc(-c2n[nH]c3cc(Br)ccc23)c(F)c1]

### Step 1: tert-butyl (2-(3-(4-(6-bromo-1H-indazol-3-yl)-3-fluorophenoxy)propoxy)ethyl)(methyl)carbamate

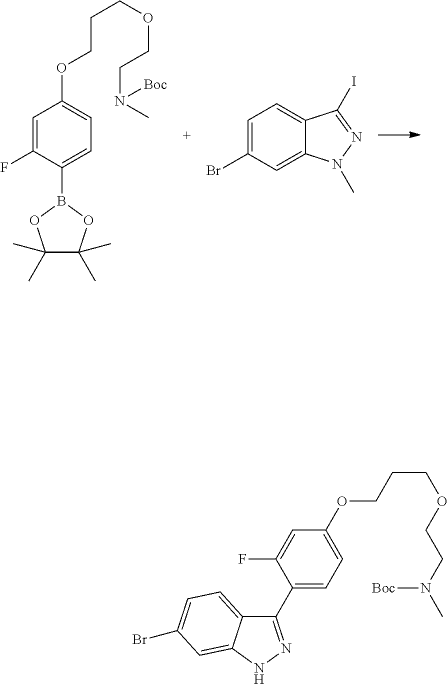 [SMILES: CN(CCOCCCOc1ccc(B2OC(C)(C)C(C)(C)O2)c(F)c1)C(=O)OC(C)(C)C ; Cn1nc(I)c2ccc(Br)cc21 ; CN(CCOCCCOc1ccc(-c2n[nH]c3cc(Br)ccc23)c(F)c1)C(=O)OC(C)(C)C]

To a mixture of tert-butyl (2-(3-(3-fluoro-4-(4,4,5,5-tetramethyl-1,3,2-dioxaborolan-2-yl)phenoxy)propoxy)ethyl)(methyl)carbamate (0.25 g, 0.55 mmol), 6-bromo-3-iodo-1H-indazole (0.178 g, 0.55 mmol), K₂CO₃ (0.229 g, 1.65 mmol), and Pd(dppf)Cl₂ (0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (79.0 mg). LCMS (+ESI): Rt=2.48 min, (M+H)⁺=522.2 and 524.2.

### Step 2: 2-(3-(4-(6-bromo-1H-indazol-3-yl)-3-fluorophenoxy)propoxy)-N-methylethanamine

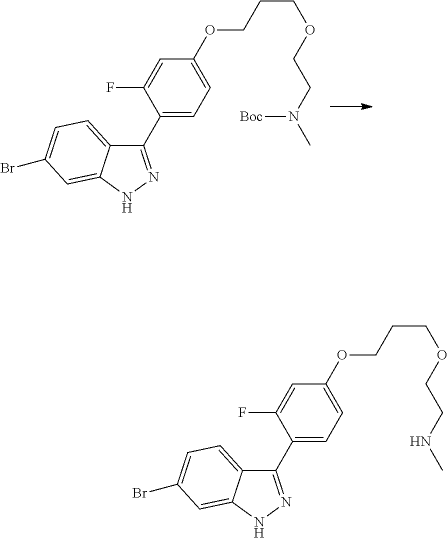 [SMILES: CN(CCOCCCOc1ccc(-c2n[nH]c3cc(Br)ccc23)c(F)c1)C(=O)OC(C)(C)C ; CNCCOCCCOc1ccc(-c2n[nH]c3cc(Br)ccc23)c(F)c1]

A solution of tert-butyl (2-(3-(4-(6-bromo-1H-indazol-3-yl)-3-fluorophenoxy)propoxy)ethyl)(methyl)carbamate (0.079 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (61.0 mg). LCMS (+ESI): Rt=1.88 min, (M+H)⁺=422.1 and 424.0.

### (4-(6-bromo-1-(4-methoxybenzyl)-1H-indazol-3-yl)-3-fluorophenyl)methanol (LZH-00060)

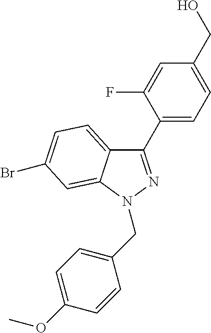 [SMILES: COc1ccc(Cn2nc(-c3ccc(CO)cc3F)c3ccc(Br)cc32)cc1]

### Step 1: 6-bromo-3-iodo-1-(4-methoxybenzyl)-1H-indazole

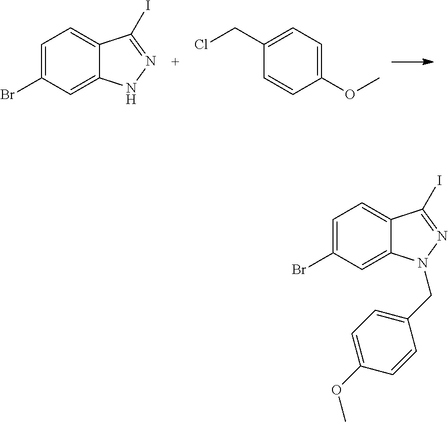

To a mixture of 6-bromo-3-iodo-1H-indazole 322.93, 9.7 g, 30.04 mmol), 1-(chloromethyl)-4-methoxybenzene (5.18 g, 33.04 mmol) in MeCN (150 mL) stirred at rt was added K₂CO₃ (11.82 g, 45.06 mmol) and the resulting mixture was stirred at rt 75° C. overnight. The mixture was filtered, washed with EtOAc, and evaporated to afford a crude which was further purified by flash chromatography (0-10% EtOAc/hexanes) to afford the titled compound (5.16 g) as a white solid. LCMS (+ESI): Rt=2.30 min, (M+H)⁺=443.0 and 445.0.

### Step 2: 4-(6-bromo-1-(4-methoxybenzyl)-1H-indazol-3-yl)-3-fluorobenzaldehyde

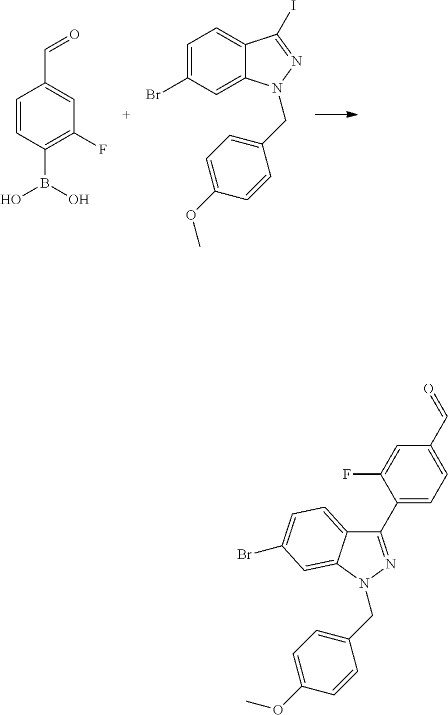 [SMILES: O=Cc1ccc(B(O)O)c(F)c1 ; COc1ccc(Cn2nc(I)c3ccc(Br)cc32)cc1 ; COc1ccc(Cn2nc(-c3ccc(C=O)cc3F)c3ccc(Br)cc32)cc1]

To a mixture of (2-fluoro-4-formylphenyl)boronic acid (0.85 g, 5.06 mmol), 6-bromo-3-iodo-1-(4-methoxybenzyl)-1H-indazole (1.68 g, 3.80 mmol), K₂CO₃ (2.10 g, 15.18 mmol), and Pd(dppf)Cl₂ (0.556 g, 0.75 mmol) in a reaction vial under vacuum was added 1,4-dioxane/water (4:1, 25.0 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-40% EtOAc/hexanes) to afford the a non-titled compound as an off-white solid (490 mg), which is not very pure but use directly in next step. LCMS (+ESI): Rt=2.52 min, (M+H)⁺=439.1 and 441.1.

### Step 3: (4-(6-bromo-1-(4-methoxybenzyl)-1H-indazol-3-yl)-3-fluorophenyl methanol

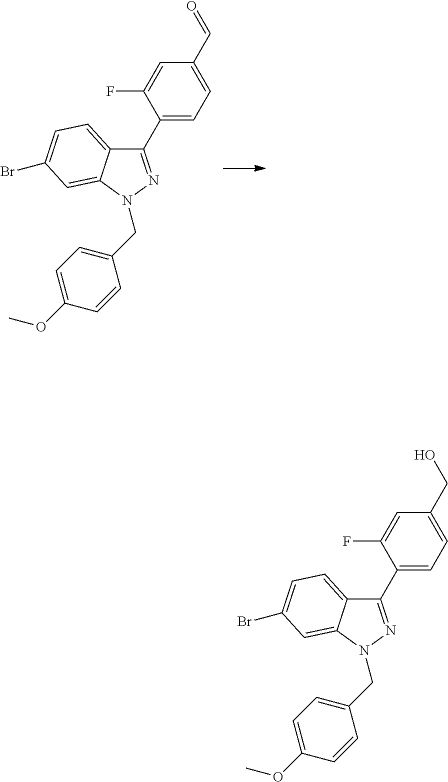 [SMILES: COc1ccc(Cn2nc(-c3ccc(C=O)cc3F)c3ccc(Br)cc32)cc1 ; COc1ccc(Cn2nc(-c3ccc(CO)cc3F)c3ccc(Br)cc32)cc1]

To a mixture of 4-(6-bromo-1-(4-methoxybenzyl)-1H-indazol-3-yl)-3-fluorobenzaldehyde (0.49 g, 1.12 mmol) in THF/MeOH (1:1, 11.0 mL), was added NaBH₄ (114 mg, 3.35 mmol) portionwise. After the addition, the mixture was stirred at room temperature for two hour. The reaction was worked up by the addition of water and extracted with EtOAc, dried over Na₂SO₄, filtered and evaporated. The residue was purified by flash chromatography (0-40% EtOAc/hexanes) to afford the titled compound as a syrup, then purified by reverse phase flash chromatography with C₁₈ column (10-100% MeCN/water) to afford the titled compound (363.0 mg). LCMS (+ESI): Rt=2.25 min, (M+H)⁺=441.0 and 443.0.

### 2-(4-(6-bromo-1-methyl-1H-indazol-3-yl)-3-fluorophenoxy)-N-methylethanamine (LZH-00061)

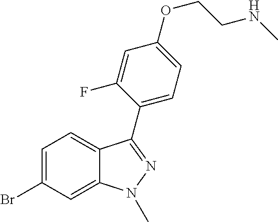 [SMILES: CNCCOc1ccc(-c2nn(C)c3cc(Br)ccc23)c(F)c1]

### Step 1: tert-butyl (2-(4-bromo-3-fluorophenoxy)ethyl)(methyl)carbamate

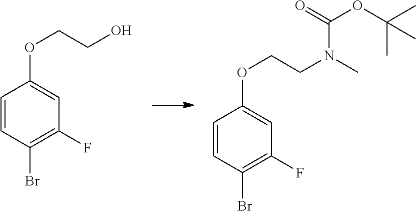 [SMILES: OCCOc1ccc(Br)c(F)c1 ; CN(CCOc1ccc(Br)c(F)c1)C(=O)OC(C)(C)C]

To a mixture of 2-(4-bromo-3-fluorophenoxy)ethanol (3.2 g, 13.61 mmol) and NEt₃ (1.65 g, 16.34 mmol) in DCM (34 mL) stired in an ice/water bath, MsCl (1.8716 g, 16.34 mmol) was added portion wise. After the addition, the mixture was stirred at rt for 2 hr. The solvent was evaporated and the residue was redissolved in 34 mL of 1,4-dioxane and transferred into a sealed tube. MeNH₂ (40% in water, 31.7 g, 408 mmol) was added. The mixture was heated at 60° C. overnight and the solvent was evaporated. The residue was diluted with water and extracted with DCM, dried over Na₂SO₄, filtered and evaporated to afford a crude which was used directly. The crude thus obtained was redissolved in EtOAc (34 mL), and then Boc anyhydride (3.27 g, 14.98 mmol) was added in small portion. The mixture was stirred at rt overnight. The solvent was evaporated and the residue was purified by flash chromatography (0-30% EtOAc/hexanes) to afford the titled compound (3.82 g) as a oil. LCMS (+ESI): Rt=2.37 min, (M+Na)⁺=370.1 and 371.9.

### Step 2: tert-butyl (2-(3-fluoro-4-(4,4,5,5-tetramethyl-1,3,2-dioxaborolan-2-yl)phenoxy)ethyl)methyl)carbamate

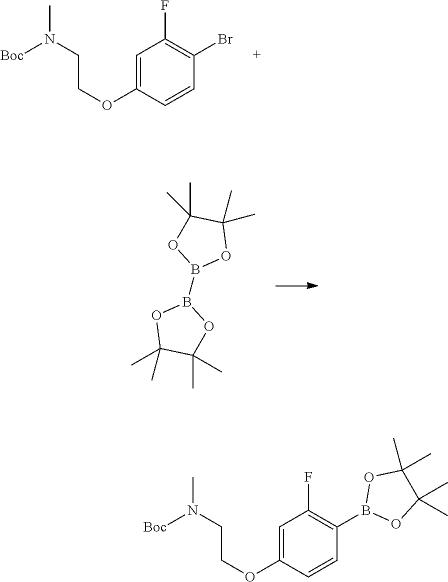

To a mixture of tert-butyl (2-(4-bromo-3-fluorophenoxy)ethyl)(methyl)carbamate (1.338 g, 3.84 mmol), 4,4,4′,4′,5,5,5′,5′-octamethyl-2,2′-bi(1,3,2-dioxaborolane) (1.952 g, 7.69 mmol), KOAc (0.943 g, 9.61 mmol), and Pd(dppf)Cl₂ 0.281 g, 0.38 mmol) in a RBF under vacuum was added DMSO (19 mL). After the addition, a nitrogen balloon was applied to the system and then the mixture was heated to 90° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound (1.27 g) as an oil like material. LCMS (+ESI): Rt=2.47 min, (M+H))⁺=396.3.

### Step 3: tert-butyl (2-(4-(6-bromo-1-methyl-1H-indazol-3-yl)-3-fluorophenoxy)ethyl)(methyl)carbamate

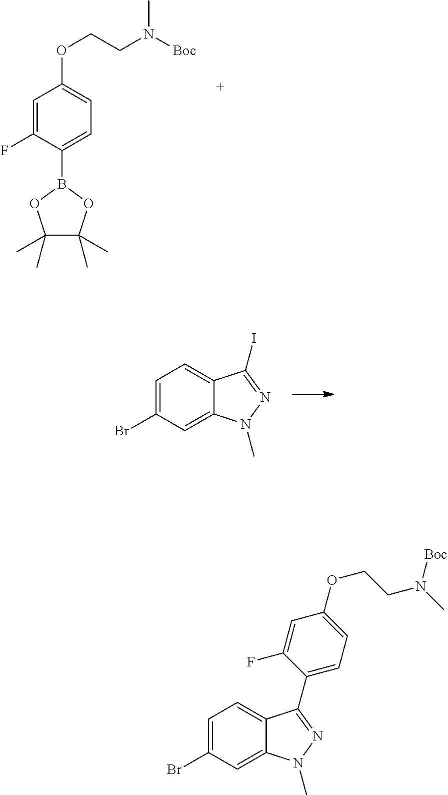 [SMILES: CN(CCOc1ccc(B2OC(C)(C)C(C)(C)O2)c(F)c1)C(=O)OC(C)(C)C ; Cn1nc(I)c2ccc(Br)cc21 ; CN(CCOc1ccc(-c2nn(C)c3cc(Br)ccc23)c(F)c1)C(=O)OC(C)(C)C]

To a mixture of tert-butyl (2-(3-fluoro-4-(4,4,5,5-tetramethyl-1,3,2-dioxaborolan-2-yl)phenoxy)ethyl)(methyl)carbamate (0.22 g, 0.55 mmol), 6-bromo-3-iodo-1-methyl-1H-indazole (0.178 g, 0.55 mmol), K₂CO₃ (0.229 g, 1.65 mmol), and Pd(dppf)Cl₂ 0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (44.0 mg). LCMS (+ESI): Rt=2.67 min, (M+H)⁺=478.2 and 480.2.

### Step 4: 2-(4-(6-bromo-1-methyl-1H-indazol-3-yl)-3-fluorophenoxy)-N-methylethanamine

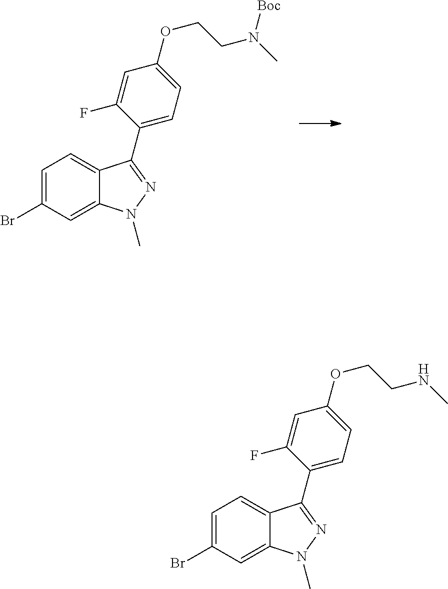 [SMILES: CN(CCOc1ccc(-c2nn(C)c3cc(Br)ccc23)c(F)c1)C(=O)OC(C)(C)C ; CNCCOc1ccc(-c2nn(C)c3cc(Br)ccc23)c(F)c1]

A solution of tert-butyl (2-(4-(6-bromo-1-methyl-1H-indazol-3-yl)-3-fluorophenoxy)ethyl)(methyl)carbamate (0.044 g) in 1.5 mL TFA/DCM (1/I) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (30.0 mg). LCMS (+ESI): Rt=1.80 min, (M+H)⁺=378.0 and 380.0.

### 3-(4-(6-bromo-1-methyl-1H-indazol-3-yl)-3-fluorophenoxy)N-methylpropan-1-amine (LZH-00062)

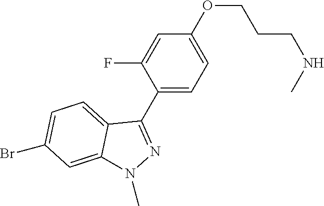 [SMILES: CNCCCOc1ccc(-c2nn(C)c3cc(Br)ccc23)c(F)c1]

### Step 1: tert-butyl (3-(4-bromo-3-fluorophenoxy)propyl)(methyl)carbamate

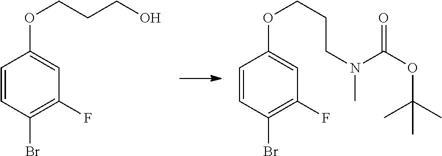 [SMILES: OCCCOc1ccc(Br)c(F)c1 ; CN(CCCOc1ccc(Br)c(F)c1)C(=O)OC(C)(C)C]

To a mixture of 3-(4-bromo-3-fluorophenoxy)propan-1-ol (2.90 g, 11.64 mmol) and NEt₃ (1.41 g, 13.97 mmol) in DCM (29 mL) stired in an ice/water bath, MsCl (1.60 g, 13.97 mmol) was added dropwise. After the addition, the mixture was stirred at rt for 2 hr. The solvent was evaporated and the residue was redissolved in 34 mL of 1,4-dioxane and transferred into a sealed tube. MeNH₂ (40% in water, 27.1 g, 349 mmol) was added. The mixture was heated at 60° C. overnight and the solvent was evaporated. The residue was diluted with water and extracted with DCM, dried over Na₂SO₄, filtered and evaporated to afford a crude which was used directly. The crude thus obtained was redissolved in EtOAc (29 mL), and then Boc anyhydride (3.27 g, 14.98 mmol) was added in small portion. The mixture was stirred at rt overnight. The solvent was evaporated and the residue was purified by flash chromatography (0-30% EtOAc/hexanes) to afford the titled compound (3.65 g) as a oil. LCMS (+ESI): Rt=2.46 min, (M+Na)⁺=384.1 and 386.1.

### Step 2: tert-butyl (3-(3-fluoro-4-(4,4,5,5-tetramethyl-1,3,2-dioxaborolan-2-yl)phenoxy)propyl)(methyl)carbamate

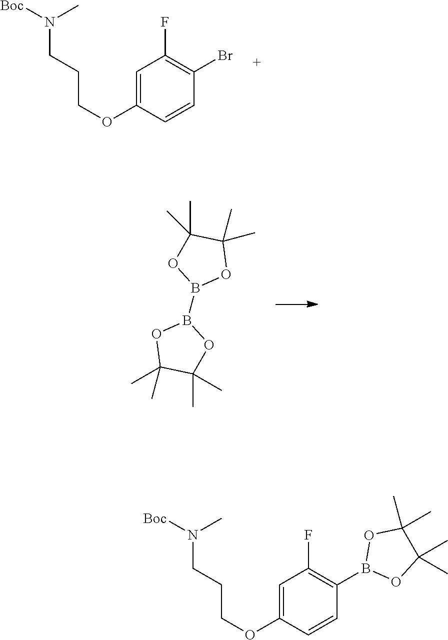

To a mixture of tert-butyl (3-(4-bromo-3-fluorophenoxy)propyl)(methyl)carbamate (1.392 g, 3.84 mmol), 4,4,4′,4′,5,5,5′,5′-octamethyl-2,2′-bi(1,3,2-dioxaborolane) (1.952 g, 7.69 mmol), KOAc (0.943 g, 9.61 mmol), and Pd(dppf)Cl₂ (0.281 g, 0.38 mmol) in a RBF under vacuum was added DMSO (19 mL). After the addition, a nitrogen balloon was applied to the system and then the mixture was heated to 90° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound (1.32 g) as an oil like material. LCMS (+ESI): Rt=2.47 min, (M+H)⁺=410.4.

### Step 3: tert-butyl (3-(4-(6-bromo-1-methyl-1H-indazol-3-yl)-3-fluorophenoxy)propyl)(methyl)carbamate

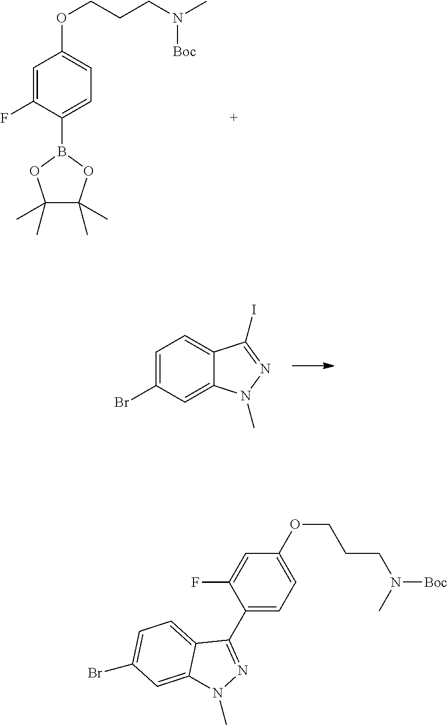 [SMILES: CN(CCCOc1ccc(B2OC(C)(C)C(C)(C)O2)c(F)c1)C(=O)OC(C)(C)C ; Cn1nc(I)c2ccc(Br)cc21 ; CN(CCCOc1ccc(-c2nn(C)c3cc(Br)ccc23)c(F)c1)C(=O)OC(C)(C)C]

To a mixture of tert-butyl (3-(3-fluoro-4-(4,4,5,5-tetramethyl-1,3,2-dioxaborolan-2-yl)phenoxy)propyl)(methyl)carbamate (0.225 g, 0.55 mmol), 6-bromo-3-iodo-1-methyl-1H-indazole (0.178 g, 0.55 mmol), K₂CO₃ (0.229 g, 1.65 mmol), and Pd(dppf)Cl₂ (0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (40.0 mg). LCMS (+ESI): Rt=2.67 min, (M+H)⁺=492.2 and 494.1.

### Step 4: 3-(4-(6-bromo-1-methyl-1H-indazol-3-yl)-3-fluorophenoxy)-N-methylpropan-1-amine

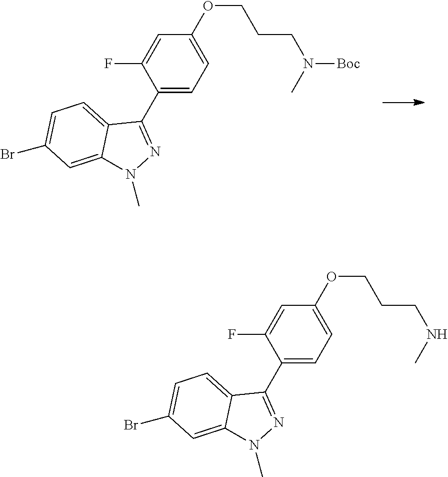 [SMILES: CN(CCCOc1ccc(-c2nn(C)c3cc(Br)ccc23)c(F)c1)C(=O)OC(C)(C)C ; CNCCCOc1ccc(-c2nn(C)c3cc(Br)ccc23)c(F)c1]

A solution of tert-butyl (3-(4-(6-bromo-1-methyl-1H-indazol-3-yl)-3-fluorophenoxy)propyl)(methyl)carbamate (0.044 g) in 1.5 mL TFA/DCM (1/1) was stirred at rt for 2 hr. The solvent was evaporated and the residue was co-evaporated with toluene. The residue was further purified by flash chromatography (0-20% Solvent B/DCM, while solvent B is 18% of ammonia in MeOH) to afford the titled compound (33.0 mg). LCMS (+ESI): Rt=1.80 min, (M+H)⁺=392.1 and 394.0.

### 2-(4-(6-bromo-1H-indazol-3-yl)-3-fluorophenoxy)-N-methylethanamine (LZH-00063)

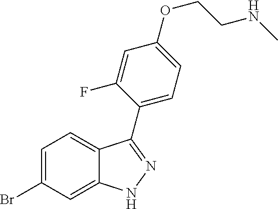 [SMILES: CNCCOc1ccc(-c2n[nH]c3cc(Br)ccc23)c(F)c1]

### Step 1: tert-butyl (2-(4-(6-bromo-1H-indazol-3-yl)-3-fluorophenoxy)ethyl)(methyl)carbamate

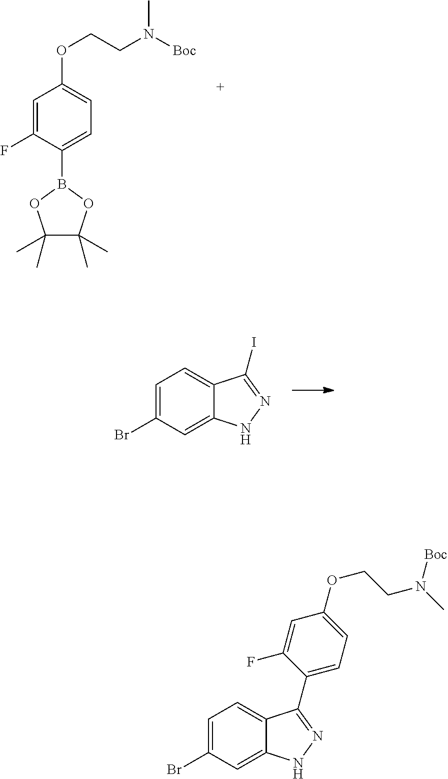 [SMILES: CN(CCOc1ccc(B2OC(C)(C)C(C)(C)O2)c(F)c1)C(=O)OC(C)(C)C ; Brc1ccc2c(I)n[nH]c2c1 ; CN(CCOc1ccc(-c2n[nH]c3cc(Br)ccc23)c(F)c1)C(=O)OC(C)(C)C]

To a mixture of tert-butyl (2-(3-fluoro-4-(4,4,5,5-tetramethyl-1,3,2-dioxaborolan-2-yl)phenoxy)ethyl)(methyl)carbamate (0.22 g, 0.55 mmol), 6-bromo-3-iodo-1H-indazole (0.178 g, 0.55 mmol), K₂CO₃ (0.229 g, 1.65 mmol), and Pd(dppf)Cl₂ (0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (40.0 mg). LCMS (+ESI): Rt=2.42 min, (M+H)⁺=464.2 and 466.2.

### Step 2: 2-(4-(6-bromo-1H-indazol-3-yl)-3-fluorophenoxy)-N-methylethanamine

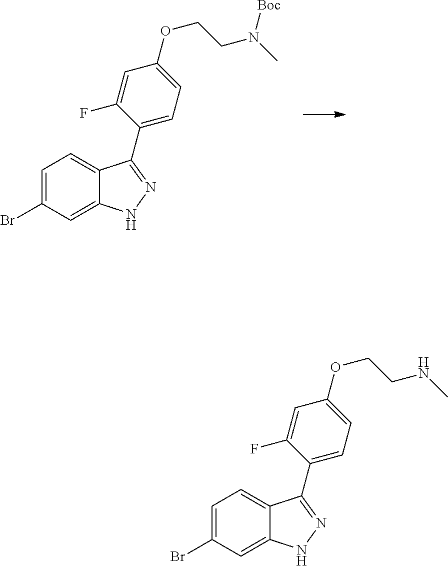 [SMILES: CN(CCOc1ccc(-c2n[nH]c3cc(Br)ccc23)c(F)c1)C(=O)OC(C)(C)C ; CNCCOc1ccc(-c2n[nH]c3cc(Br)ccc23)c(F)c1]

A solution of tert-butyl (2-(4-(6-bromo-1H-indazol-3-yl)-3-fluorophenoxy)ethyl)(methyl)carbamate (0.033 g) in 1.5 mL TFA/DCM (1/1) was stirred at rt for 2 hr. The reaction was worked up by the evaporation of volatile and the residue was treated sat NaHCO₃/H₂O. The reaction mixture was extracted with EtOAc, dried over Na₂SO₄, filtered, and evaporated. The residue was purified by flash chromatography (0-100% solvent B/DCM, while solvent B is a solvent mixture of MeOH/amonia (4:1)) to afford the titled compound as a gummy solid (20.0 mg). LCMS (+ESI): Rt=1.68 min, (M+H)⁺=364.0 and 366.0.

### 3-(4-(6-bromo-1H-indazol-3-yl)-3-fluorophenoxy)-N-methylpropan-1-amine (LZH-00064)

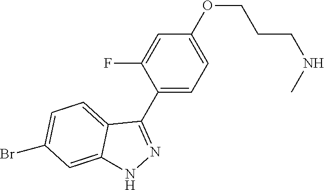 [SMILES: CNCCCOc1ccc(-c2n[nH]c3cc(Br)ccc23)c(F)c1]

### Step 1: tert-butyl (3-(4-(6-bromo-1H-indazol-3-yl)-3-fluorophenoxy)propyl)(methyl)carbamate

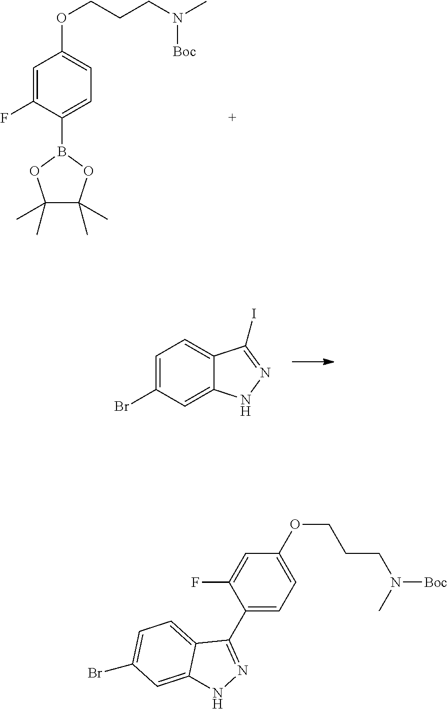 [SMILES: CN(CCCOc1ccc(B2OC(C)(C)C(C)(C)O2)c(F)c1)C(=O)OC(C)(C)C ; Brc1ccc2c(I)n[nH]c2c1 ; CN(CCCOc1ccc(-c2n[nH]c3cc(Br)ccc23)c(F)c1)C(=O)OC(C)(C)C]

To a mixture of tert-butyl (3-(3-fluoro-4-(4,4,5,5-tetramethyl-1,3,2-dioxaborolan-2-yl)phenoxy)propyl)(methyl)carbamate (0.225 g, 0.55 mmol), 6-bromo-3-iodo-1H-indazole (0.178 g, 0.55 mmol), K₂CO₃ (0.229 g, 1.65 mmol), and Pd(dppf)Cl₂ (0.121 g, 0.17 mmol) in a reaction vial under vacuum was added 1,4-dioxane/water (4:1, 2.77 mL). After the addition, a nitrogen balloon was applied to the system and then the mixture was heated to 100° C. and stirred at this temperature overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (43.0 mg). LCMS (+ESI): Rt=2.42 min, (M+H)⁺=478.2 and 480.2.

### Step 2: 3-(4-(6-bromo-1H-indazol-3-yl)-3-fluorophenoxy)-N-methylpropan-1-amine

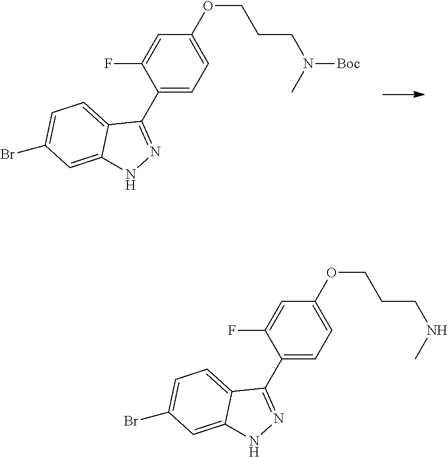 [SMILES: CN(CCCOc1ccc(-c2n[nH]c3cc(Br)ccc23)c(F)c1)C(=O)OC(C)(C)C ; CNCCCOc1ccc(-c2n[nH]c3cc(Br)ccc23)c(F)c1]

A solution of tert-butyl (3-(4-(6-bromo-1H-indazol-3-yl)-3-fluorophenoxy)propyl)(methyl)carbamate (0.043 g) in 1.5 mL TFA/DCM (1/1) was stirred at rt for 2 hr. The reaction was worked up by the evaporation of volatile and the residue was treated sat NaHCO₃/H₂O. The reaction mixture was extracted with EtOAc, dried over Na₂SO₄, filtered, and evaporated. The residue was purified by flash chromatography (0-100% solvent B/DCM, while solvent B is a solvent mixture of MeOH/amonia (4:1)) to afford the titled compound as a gummy solid (34.0 mg). LCMS (+ESI): Rt=1.75 min, (M+H)⁺=378.1 and 380.0.

### N1-(4-(6-bromo-1-(4-methoxybenzyl)-1H-indazol-3-yl)-3-fluorobenzyl)-N1,N2-dimethylethane-1,2-diamine (LZH-00065)

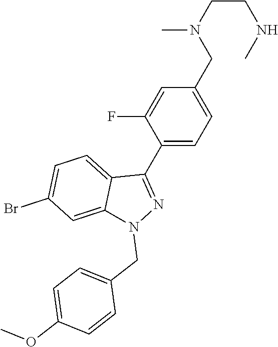 [SMILES: CNCCN(C)Cc1ccc(-c2nn(Cc3ccc(OC)cc3)c3cc(Br)ccc23)c(F)c1]

### Step 1: N1-(4-(6-bromo-1-(4-methoxybenzyl)-1H-indazol-3-yl)-3-fluorobenzyl)-N1,N2-dimethylethane-1,2-diamine

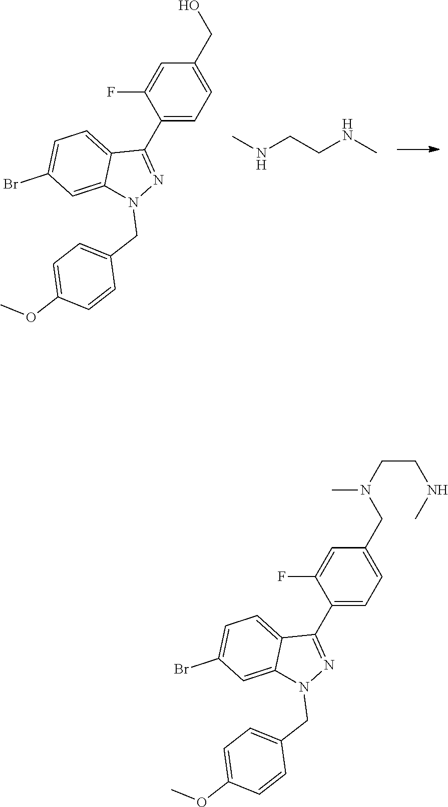

To a mixture of (4-(6-bromo-1-(4-methoxybenzyl)-1H-indazol-3-yl)-3-fluorophenyl)methanol (0.37 g, 0.84 mmol) and NEt₃ (0.1694 g, 1.68 mmol) in DCM (9 mL) stired in an ice/water bath, MsCl (0.1153 g, 1.01 mmol) was added dropwise. After the addition, the mixture was stirred rt for 2 hr. The solvent was evaporated and the residue was redissolved in 10 mL of 1,4-dioxane. To the mixture thus obtained N1,N2-dimethylethane-1,2-diamine (0.74 g. 8.38 mmol) was added and the mixture was heated at 100° C. for 2 hr. The reaction was worked up by the addition of sat NaHCO₃/H₂O. The reaction mixture was extracted with EtOAc, dried over Na₂SO₄, filtered, and evaporated. The residue was purified by flash chromatography (0-100% solvent B/DCM, while solvent B is a solvent mixture of MeOH/amonia (4:1)) to afford the titled compound as a gummy solid (220 mg). LCMS (+ESI): Rt=1.82 min, (M+H)⁺=511.2 and 513.2.

### N1-(4-(6-bromo-1H-indazol-3-yl)-3-fluorobenzyl)-N1,N2-dimethylethane-1,2-diamine (LZH-00066)

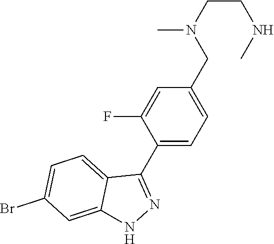 [SMILES: CNCCN(C)Cc1ccc(-c2n[nH]c3cc(Br)ccc23)c(F)c1]

### Step 1: N1-(4-(6-bromo-1H-indazol-3-yl)-3-fluorobenzyl)-N1,N2-dimethylethane-1,2-diamine

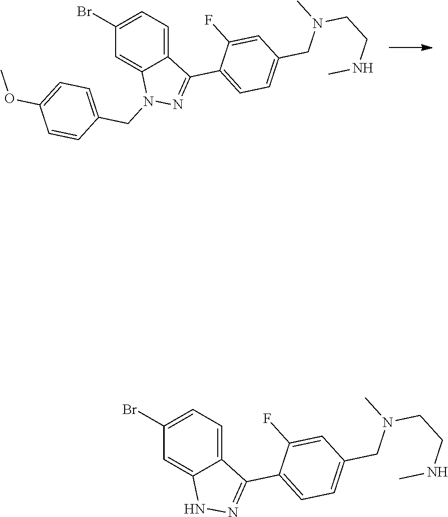 [SMILES: CNCCN(C)Cc1ccc(-c2nn(Cc3ccc(OC)cc3)c3cc(Br)ccc23)c(F)c1 ; CNCCN(C)Cc1ccc(-c2n[nH]c3cc(Br)ccc23)c(F)c1]

To a mixture of N1-(4-(6-bromo-1-(4-methoxybenzyl)-1H-indazol-3-yl)-3-fluorobenzyl)-N1,N2-dimethylethane-1,2-diamine (0.22 g, 0.43 mmol), PhOMe (0.138 g, 1.29 mmol) in a reaction vial, was added a 1:1 mixture of TFA/DCM (4.3 mL). The mixture was stirred at room temperature overnight. LCMS indicated that no reaction. Thus the solvent was evaporated and the residue was redissolved in 2 mL of TFA. The mixture was stirred at 100° C. overnight. The reaction mixture was evaporated. The residue was treated with sat NaHCO₃/H₂O and extracted with EtOAc, dried over Na₂SO₄, filtered, and evaporated. The residue was purified by flash chromatography (0-100% solvent B/DCM, while solvent B is a solvent mixture of MeOH/amonia (4:1)) to afford the titled compound as a gummy solid (127 mg). LCMS (+ESI): Rt=1.64 min, (M+H)⁺=391.0 and 393.1.

### (S)-6-bromo-3-(2-fluoro-4-(3-(pyrrolidin-2-ylmethoxy)propoxy)phenyl)-1H-indazole (LZH-00067)

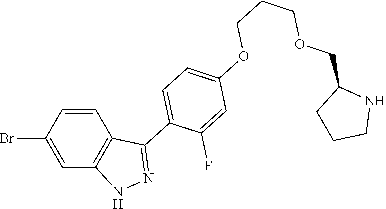 [SMILES: Fc1cc(OCCCOC[C@@H]2CCCN2)ccc1-c1n[nH]c2cc(Br)ccc12]

### Step 1: (S)-tert-butyl 2-((3-chloropropoxy)methyl)pyrrolidine-1-carboxylate

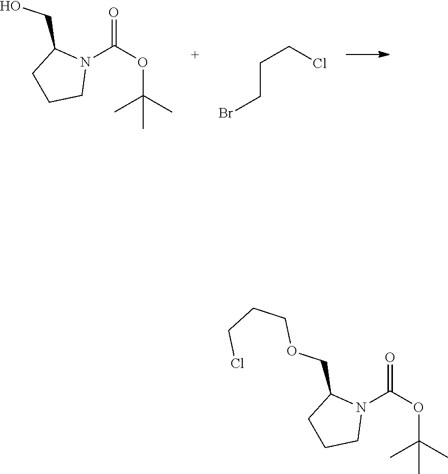 [SMILES: CC(C)(C)OC(=O)N1CCC[C@H]1CO ; ClCCCBr ; CC(C)(C)OC(=O)N1CCC[C@H]1COCCCCl]

A mixture of (S)-tert-butyl 2-(hydroxymethyl)pyrrolidine-1-carboxylate (5.0 g, 24.84 mmol), 1-bromo-3-chloropropane (23.47 g, 149.06 mmol), Bu₄NHSO₄ (0.253 g, 0.75 mmol), and NaOH (7.78 g, 50%, 97.30 mmol) in a reaction vial was stirred at 80° C. for 6 hr. The reaction was worked up by the addition of water and extracted with EtOAc. The extraction was evaporated and the residue was mixed with 75 mL of water and evaporated again to remove most of the water. The residue was then dissolved in EtOAc, dried over Na₂SO₄, filtered and evaporated to afford a crude (syrup) which contained the titled compound (6.0 g) based on HNMR and LCMS and a side product (O-allyl). The ratio looks like 3:1 in favor of the titled compound. No further purification, used directly in next step.

### Step 2: (S)-tert-butyl 2-((3-(4-bromo-3-fluorophenoxy)propoxy)methyl)pyrrolidine-1-carboxylat

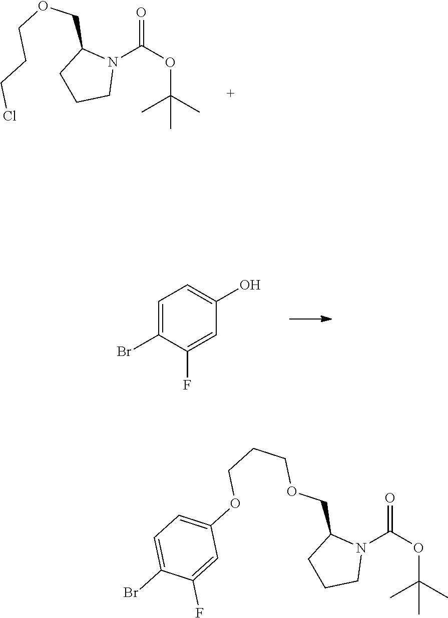 [SMILES: CC(C)(C)OC(=O)N1CCC[C@H]1COCCCCl ; Oc1ccc(Br)c(F)c1 ; CC(C)(C)OC(=O)N1CCC[C@H]1COCCCOc1ccc(Br)c(F)c1]

A mixture of (S)-tert-butyl 2-((3-chloropropoxy)methyl)pyrrolidine-1-carboxylate (6.0 g, 21.60 mmol), 4-bromo-3-fluorophenol (4.13 g, 21.60 mmol), NaI (3.24 g, 21.60 mmol), and K₂CO₃ (8.94 g, 64.80 mmol) in a sealed tube with MeCN (108 mL) was stirred at 80° C. for 3 days. The reaction was worked up by the addition of water. The reaction mixture was extracted with EtOAc/hexanes (1:1), dried over Na₂SO₄, filtered, and evaporated. The residue was purified by flash chromatography (0-15% EtOAc/hexanes) to afford the titled compound as a syrup (2.27 g). LCMS (+ESI): Rt=2.62 min, (M+Na)⁺=454.1 and 456.1.

### Step 3: (S)-tert-butyl 2-((3-(3-fluoro-4-(4,4,5,5-tetramethyl-1,3,2-dioxaborolan-2-yl)phenoxy)propoxy)methyl)pyrrolidine-1-carboxylate

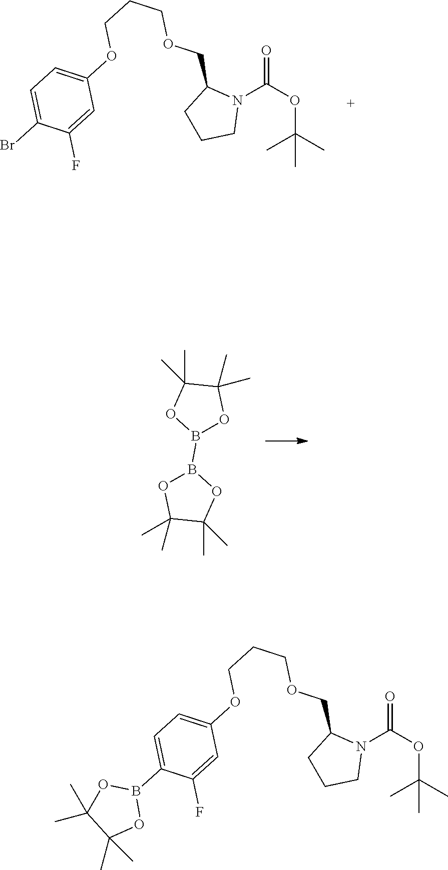

To a mixture of (S)-tert-butyl 2-((3-(4-bromo-3-fluorophenoxy)propoxy)methyl)pyrrolidine-1-carboxylate (2.27 g, 6.27 mmol), 4,4,4′,4′,5,5,5′,5′-octamethyl-2,2′-bi(1,3,2-dioxaborolane) (3.18 g, 12.53 mmol), KOAc (1.54 g, 15.67 mmol), and Pd(dppf)Cl₂ (0.45 g, 0.63 mmol) in a round-bottom-flask under vacuum was added DMSO (31 mL). After the addition, a nitrogen balloon was applied to the system and then the mixture was heated to 90° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound as an oil like material. LCMS (+ESI): Rt=2.76 min, (M+H)⁺=480.3.

### Step 4: (S)-tert-butyl 2-((3-(4-(6-bromo-1H-indazol-3-yl)-3-fluorophenoxy)propoxy)methyl)pyrrolidine-1-carboxylate

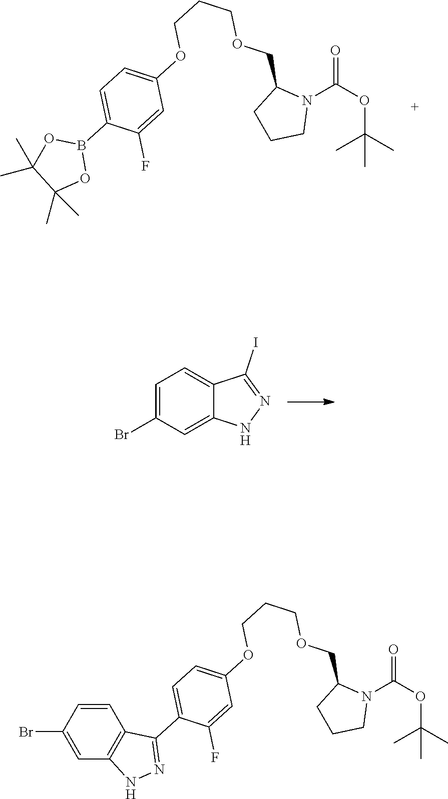 [SMILES: CC(C)(C)OC(=O)N1CCC[C@H]1COCCCOc1ccc(B2OC(C)(C)C(C)(C)O2)c(F)c1 ; Brc1ccc2c(I)n[nH]c2c1 ; CC(C)(C)OC(=O)N1CCC[C@H]1COCCCOc1ccc(-c2n[nH]c3cc(Br)ccc23)c(F)c1]

To a reaction vial containing (S)-tert-butyl 2-((3-(3-fluoro-4-(4,4,5,5-tetramethyl-1,3,2-dioxaborolan-2-yl)phenoxy)propoxy)methyl)pyrrolidine-1-carboxylate (0.25 g, 0.52 mmol), 6-bromo-3-iodo-1H-indazole (0.1684 g, 0.52 mmol), Pd(dppf)Cl₂ (0.0382 g, 0.05 mmol), and K₂CO₃ (0.2161 g, 1.56 mmol) under vacuum, 2.6 mL of 1,4-dioxane/water (4:1) was added via a syringe. After the addition, a nitrogen balloon was applied to the system and the mixture was stirred at 105° C. for 2 days. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (26.0 mg). LCMS (+ESI): Rt=2.68 min, (M+H)⁺=548.1 and 550.3.

### Step 5: (S)-6-bromo-3-(2-fluoro-4-(3-(pyrrolidin-2-ylmethoxy)propoxy)phenyl)-1H-indazole

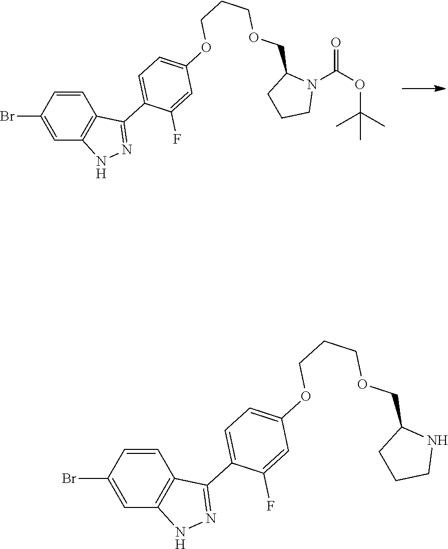 [SMILES: CC(C)(C)OC(=O)N1CCC[C@H]1COCCCOc1ccc(-c2n[nH]c3cc(Br)ccc23)c(F)c1 ; Fc1cc(OCCCOC[C@@H]2CCCN2)ccc1-c1n[nH]c2cc(Br)ccc12]

To a reaction vial containing (S)-tert-butyl 2-((3-(4-(6-bromo-1H-indazol-3-yl)-3-fluorophenoxy)propoxy)methyl)pyrrolidine-1-carboxylate (0.026 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a solid (22 mg). LCMS (+ESI): Rt=1.74 min, (M+H)⁺=448.1 and 450.1.

### (S)-6-bromo-3-(2-fluoro-4-(3-(pyrrolidin-2-ylmethoxy)propoxy)phenyl)-1-methyl-1H-indazole (LZH-00068)

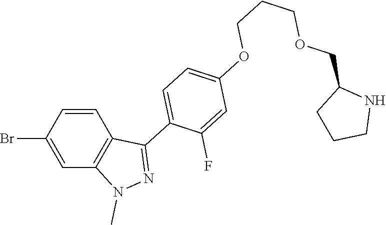 [SMILES: Cn1nc(-c2ccc(OCCCOC[C@@H]3CCCN3)cc2F)c2ccc(Br)cc21]

### Step 1: (S)-tert-butyl 2-((3-(4-(6-bromo-1-methyl-1H-indazol-3-yl)-3-fluorophenoxy)propoxy)methyl)pyrrolidine-1-carboxylate

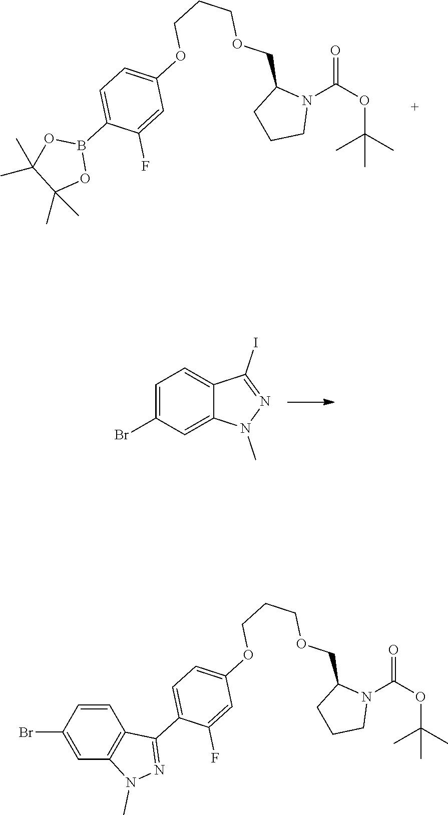 [SMILES: CC(C)(C)OC(=O)N1CCC[C@H]1COCCCOc1ccc(B2OC(C)(C)C(C)(C)O2)c(F)c1 ; Cn1nc(I)c2ccc(Br)cc21 ; Cn1nc(-c2ccc(OCCCOC[C@@H]3CCCN3C(=O)OC(C)(C)C)cc2F)c2ccc(Br)cc21]

To a reaction vial containing (S)-tert-butyl 2-((3-(3-fluoro-4-(4,4,5,5-tetramethyl-1,3,2-dioxaborolan-2-yl)phenoxy)propoxy)methyl)pyrrolidine-1-carboxylate (0.25 g, 0.52 mmol), 6-bromo-3-iodo-1-methyl-1H-indazole (0.176 g, 0.52 mmol), Pd(dppf)Cl₂ (0.038 g, 0.05 mmol), and K₂CO₃ (0.216 g, 1.56 mmol) under vacuum, 2.6 mL of 1,4-dioxane/water (4:1) was added via a syringe. After the addition, a nitrogen balloon was applied to the system and the mixture was stirred at 95° C. overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (63.0 mg). LCMS (+ESI): Rt=2.92 min, (M+H)⁺=562.1 and 564.1.

### Step 2: (S)-6-bromo-3-(2-fluoro-4-(3-(pyrrolidin-2-ylmethoxy)propoxy)phenyl)-1-methyl-1H-indazole

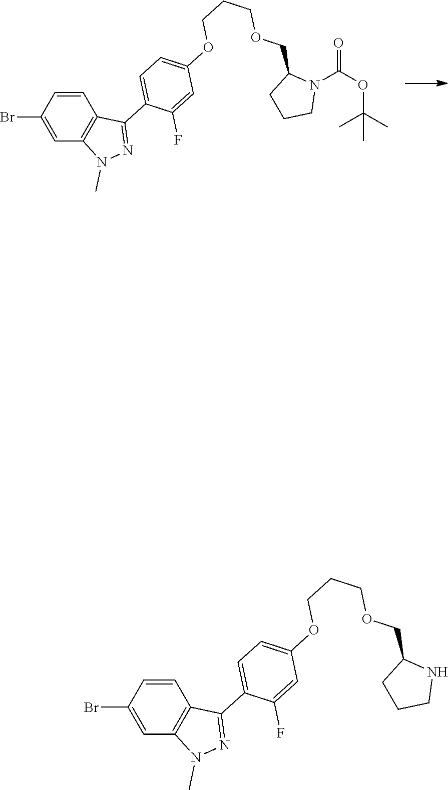 [SMILES: Cn1nc(-c2ccc(OCCCOC[C@@H]3CCCN3C(=O)OC(C)(C)C)cc2F)c2ccc(Br)cc21 ; Cn1nc(-c2ccc(OCCCOC[C@@H]3CCCN3)cc2F)c2ccc(Br)cc21]

To a reaction vial containing (S)-tert-butyl 2-((3-(4-(6-bromo-1-methyl-1H-indazol-3-yl)-3-fluorophenoxy)propoxy)methyl)pyrrolidine-1-carboxylate (0.062 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a gummy solid (51 mg). LCMS (+ESI): Rt=1.82 min, (M+H)⁺=462.1 and 464.1.

### (R)-6-bromo-3-(2-fluoro-4-(3-(pyrrolidin-2-ylmethoxy)propoxy)phenyl)-1H-indazole (LZH-00069)

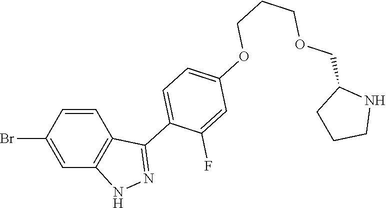 [SMILES: Fc1cc(OCCCOC[C@H]2CCCN2)ccc1-c1n[nH]c2cc(Br)ccc12]

### Step 1: (R)-tert-butyl 2-((3-chloropropoxy)methyl)pyrrolidine-1-carboxylate

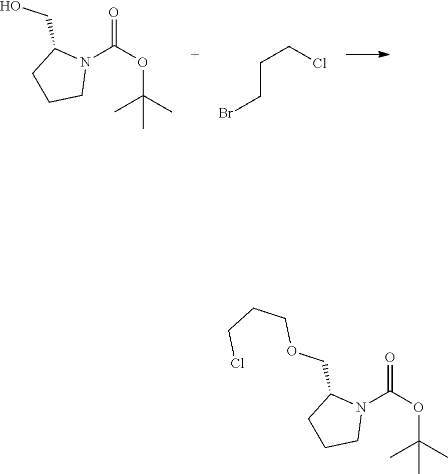 [SMILES: CC(C)(C)OC(=O)N1CCC[C@@H]1CO ; ClCCCBr ; CC(C)(C)OC(=O)N1CCC[C@@H]1COCCCCl]

A mixture of (R)-tert-butyl 2-(hydroxymethyl)pyrrolidine-1-carboxylate (5.0 g, 24.84 mmol), 1-bromo-3-chloropropane (23.47 g, 149.06 mmol), Bu₄NHSO₄ (0.253 g, 0.75 mmol), and NaOH (7.78 g, 50%, 97.30 mmol) in a reaction vial was stirred at 80° C. for 6 hr. The reaction was worked up by the addition of water and extracted with EtOAc. The extraction was evaporated and the residue was mixed with 75 mL of water and evaporated again to remove most of the water. The residue was then dissolved in EtOAc, dried over Na₂SO₄, filtered and evaporated to afford a crude (syrup) which contained the titled compound (3.64 g) based on HNMR and LCMS and a side product (O-allyl). The ratio looks like 3:1 in favor of the titled compound. No further purification, used directly in next step.

### Step 2: (R)-tert-butyl 2-((3-(4-bromo-3-fluorophenoxy)propoxy)methyl)pyrrolidine-1-carboxylate

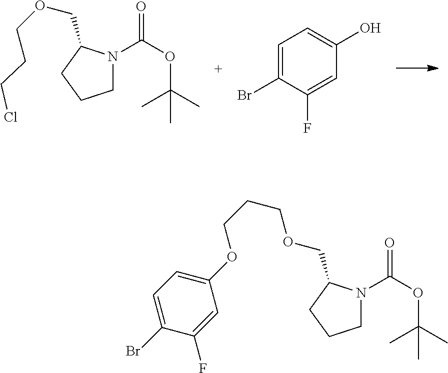 [SMILES: CC(C)(C)OC(=O)N1CCC[C@@H]1COCCCCl ; Oc1ccc(Br)c(F)c1 ; CC(C)(C)OC(=O)N1CCC[C@@H]1COCCCOc1ccc(Br)c(F)c1]

A mixture of (R)-tert-butyl 2-((3-chloropropoxy)methyl)pyrrolidine-1-carboxylate (3.64 g, 13.10 mmol), 4-bromo-3-fluorophenol (2.50 g, 13.10 mmol), NaI (1.96 g, 13.10 mmol), and K₂CO₃ (5.42 g, 39.31 mmol) in a sealed tube with MeCN (65 mL) was stirred at 80° C. for 3 days. The reaction was worked up by the addition of water. The reaction mixture was extracted with EtOAc/hexanes (1:1), dried over Na₂SO₄, filtered, and evaporated. The residue was purified by flash chromatography (0-15% EtOAc/hexanes) to afford the titled compound as a syrup (1.45 g). LCMS (+ESI): Rt=2.64 min, (M+Na)⁺=454.1 and 456.1.

### Step 3: (R)-tert-butyl 2-((3-(3-fluoro-4-(4,4,5,5-tetramethyl-1,3,2-dioxaborolan-2-yl)phenoxy)propoxy)methyl)pyrrolidine-1-carboxylate

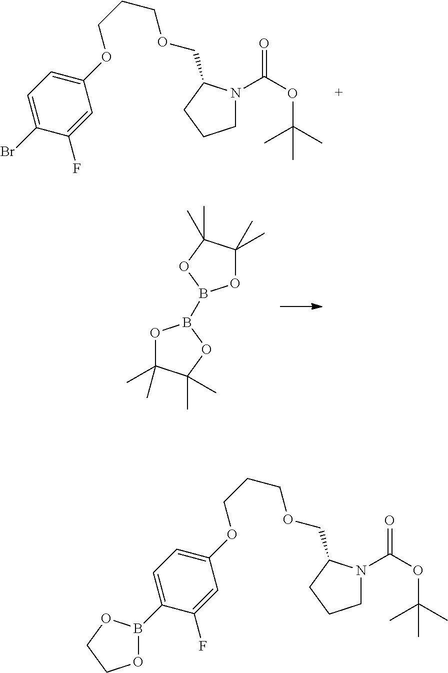

To a reaction vial containing (R)-tert-butyl 2-((3-(4-bromo-3-fluorophenoxy)propoxy)methyl)pyrrolidine-1-carboxylate (1.45 g, 4.01 mmol), 4,4,4′,4′,5,5,5′,5′-octamethyl-2,2′-bi(1,3,2-dioxaborolane) (2.04 g, 8.03 mmol), [Pd(dppf)Cl₂] (0.2936 g, 0.4 mmol), and KOAc (0.9845 g, 10.03 mmol) under vacuum, DMSO (20 mL) was added via a syringe. After the addition, a nitrogen balloon was applied to the system and the mixture was stirred at 95° C. overnight. After the addition, a nitrogen balloon was applied to the system and then the mixture was heated to 90° C. and stirred at this temperature overnight. The reaction was worked up by the addition of water and extracted with EtOAc, dried over Na₂SO₄, filtered and evaporated. The residue was purified by flash chromatography (0-20% EtOAc/hexanes) to afford the titled compound (1.45 g) as an oil like material. LCMS (+ESI): Rt=2.77 min, (M+H)⁺=480.2.

### Step 4: (R)-tert-butyl 2-((3-(4-(6-bromo-1H-indazol-3-yl)-3-fluorophenoxy)propoxy)methyl)pyrrolidine-1-carboxylate

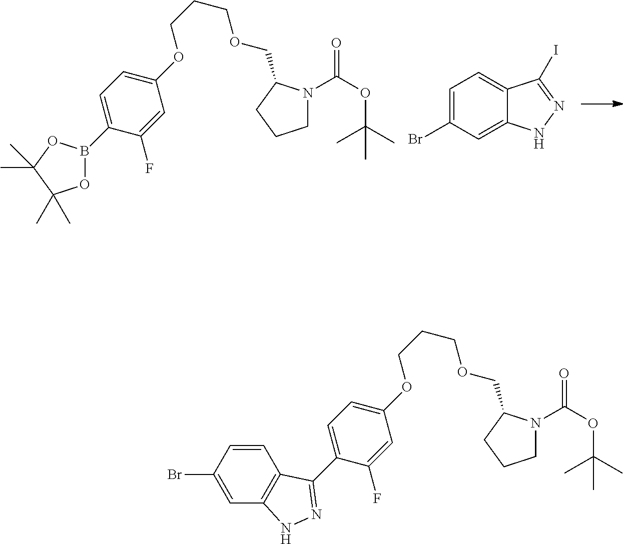

To a reaction vial containing (R)-tert-butyl 2-((3-(3-fluoro-4-(4,4,5,5-tetramethyl-1,3,2-dioxaborolan-2-yl)phenoxy)propoxy)methyl)pyrrolidine-1-carboxylate (0.25 g, 0.52 mmol), 6-bromo-3-iodo-1H-indazole (0.1684 g, 0.52 mmol), [Pd(dppf)Cl₂] (0.0382 g, 0.05 mmol), and K₂CO₃ (0.2161 g, 1.56 mmol) under vacuum, 1,4-dioxane/water (4:1) was added via a syringe. After the addition, a nitrogen balloon was applied to the system and the mixture was stirred at 105° C. for 2 days. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax (14.0 mg). LCMS (+ESI): Rt=2.63 min, (M+H)⁺=548.1 and 550.1.

### Step 5: (R)-6-bromo-3-(2-fluoro-4-(3-(pyrrolidin-2-ylmethoxy)propoxy)phenyl)-1H-indazole

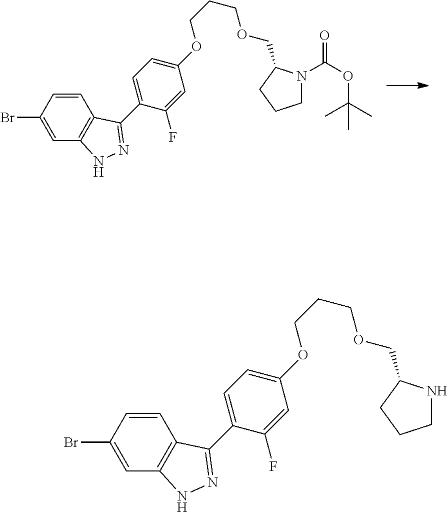 [SMILES: CC(C)(C)OC(=O)N1CCC[C@@H]1COCCCOc1ccc(-c2n[nH]c3cc(Br)ccc23)c(F)c1 ; Fc1cc(OCCCOC[C@H]2CCCN2)ccc1-c1n[nH]c2cc(Br)ccc12]

To a reaction vial containing (R)-tert-butyl 2-((3-(4-(6-bromo-1H-indazol-3-yl)-3-fluorophenoxy)propoxy)methyl)pyrrolidine-1-carboxylate (0.014 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a solid (10 mg). LCMS (+ESI): Rt=1.74 min, (M+H)⁺=448.1 and 450.4.

### (R)-6-bromo-3-(2-fluoro-4-(3-(pyrrolidin-2-ylmethoxy)propoxy)phenyl)-1-methyl-1H-indazole (LZH-00070)

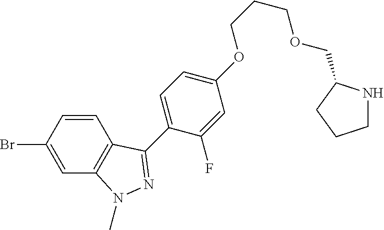 [SMILES: Cn1nc(-c2ccc(OCCCOC[C@H]3CCCN3)cc2F)c2ccc(Br)cc21]

### Step 1: (R)-tert-butyl 2-((3-(4-(6-bromo-1-methyl-1H-indazol-3-yl)-3-fluorophenoxy)propoxy)methyl)pyrrolidine-1-carboxylate

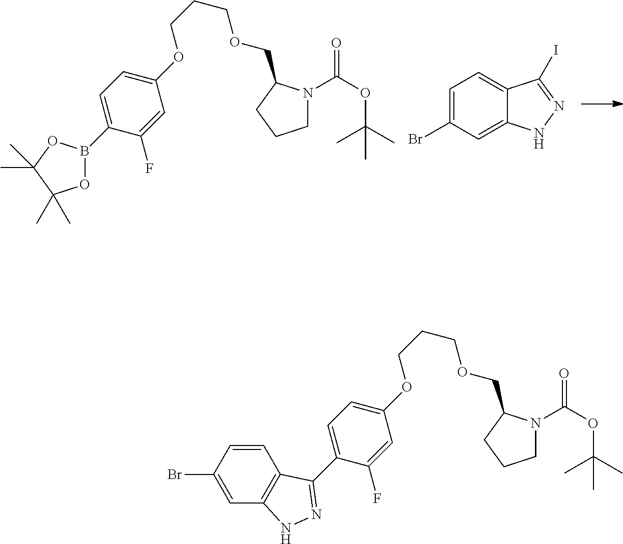

To a reaction vial containing (R)-tert-butyl 2-((3-(3-fluoro-4-(4,4,5,5-tetramethyl-1,3,2-dioxaborolan-2-yl)phenoxy)propoxy)methyl)pyrrolidine-1-carboxylate (0.25 g, 0.52 mmol), 6-bromo-3-iodo-1-methyl-1H-indazole (0.176 g, 0.52 mmol), Pd(dppf)Cl₂ (0.038 g, 0.05 mmol), and K₂CO₃ (0.216 g, 1.56 mmol) under vacuum, 2. 6 mL of 1,4-dioxane/water (4:1) was added via a syringe. After the addition, a nitrogen balloon was applied to the system and the mixture was stirred at 95° C. overnight. The reaction was worked up by the addition of water and extracted with EtOAc, dried over Na₂SO₄, filtered and evaporated. The residue was purified by flash chromatography (0-55% EtOAc/hexanes) to afford the titled compound as an off-white solid, which is not very pure; thus further purified by reverse phase flash chromatography with C₁₈ column (35-100% MeCN/water) to afford the titled compound as a wax as a wax (32.0 mg). LCMS (+ESI): Rt=2.92 min, (M+H)⁺=562.3 and 564.2.

### Step 2: (R)-6-bromo-3-(2-fluoro-4-(3-(pyrrolidin-2-ylmethoxy)propoxy)phenyl)-1-methyl-1H-indazole

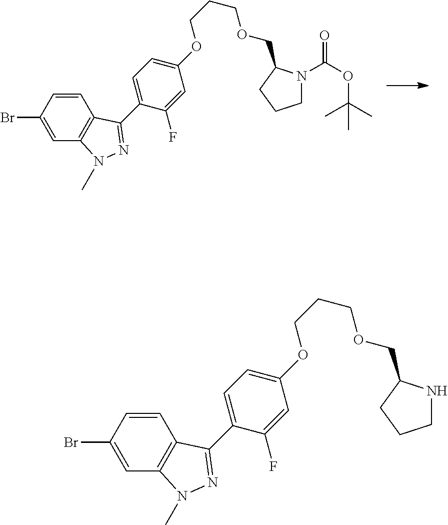 [SMILES: Cn1nc(-c2ccc(OCCCOC[C@@H]3CCCN3C(=O)OC(C)(C)C)cc2F)c2ccc(Br)cc21 ; Cn1nc(-c2ccc(OCCCOC[C@@H]3CCCN3)cc2F)c2ccc(Br)cc21]

To a reaction vial containing (R)-tert-butyl 2-((3-(4-(6-bromo-1-methyl-1H-indazol-3-yl)-3-fluorophenoxy)propoxy)methyl)pyrrolidine-1-carboxylate (0.032 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a solid (26 mg). LCMS (+ESI): Rt=1.82 min, (M+H)⁺=462.1 and 464.1.

### (S)—N-(3-(4-(6-bromo-1H-indazol-3-yl)-3-fluorophenoxy)propyl)-N-methylpyrrolidine-2-carboxamide (LZH-00071)

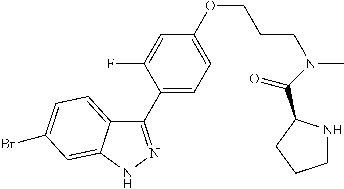 [SMILES: CN(CCCOc1ccc(-c2n[nH]c3cc(Br)ccc23)c(F)c1)C(=O)[C@@H]1CCCN1]

### Step 1: (S)-tert-butyl 2-(I H-imidazole-1-carbonyl)pyrrolidine-1-carboxylate

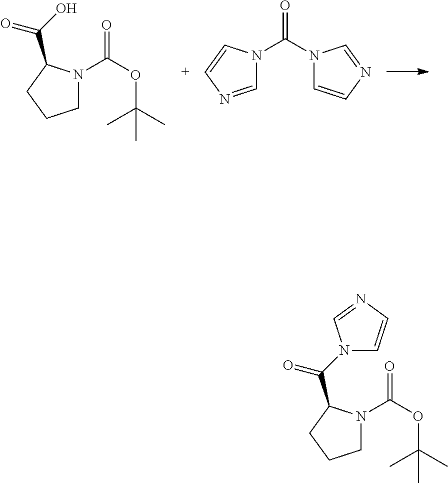 [SMILES: CC(C)(C)OC(=O)N1CCC[C@H]1C(=O)O ; O=C(n1ccnc1)n1ccnc1 ; CC(C)(C)OC(=O)N1CCC[C@H]1C(=O)n1ccnc1]

To a mixture of (S)-1-(tert-butoxycarbonyl)pyrrolidine-2-carboxylic acid (2.37 g, 11 mmol) in THF (15 mL) stirred at rt, 1,1′-Carbonyldiimidazole (1.62 g, 10.01 mmol) was added portion wise. After the addition, the mixture was stirred at rt for an overnight. The volume was adjusted to 20 mL by addition of more THF and the titled compound was afford as a 0.5 M stock solution for further use.

### Step 2: (S)-tert-butyl 2-((3-(4-(6-bromo-1H-indazol-3-yl)-3-fluorophenoxy)propyl)(methyl)carbamoyl)pyrrolidine-1-carboxylate

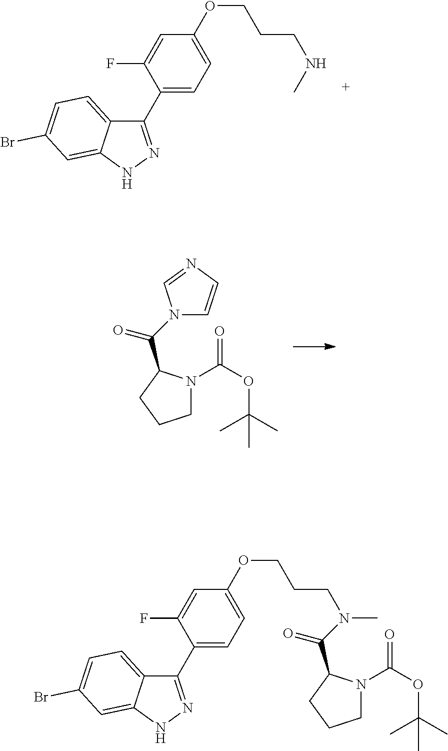 [SMILES: CNCCCOc1ccc(-c2n[nH]c3cc(Br)ccc23)c(F)c1 ; CC(C)(C)OC(=O)N1CCC[C@H]1C(=O)n1ccnc1 ; CN(CCCOc1ccc(-c2n[nH]c3cc(Br)ccc23)c(F)c1)C(=O)[C@@H]1CCCN1C(=O)OC(C)(C)C]

To a mixture of 3-(4-(6-bromo-H-indazol-3-yl)-3-fluorophenoxy)-N-methylpropan-1-amine (0.083 g, 0.22 mmol) in THF (2 mL) stired rt, (S)-tert-butyl 2-(1H-imidazole-1-carbonyl)pyrrolidine-1-carboxylate (0.57 mL, 0.5 M in THF, 0.29 mmol) was added dropwise. After the addition, the mixture was stirred at rt overnight. The LCMS show that the reaction was not completed. Thus the mixture was heated at 65° C. overnight. The reaction was worked up by the addition of 1 M NaOH and the mixture was extracted with EtOAc, dried over Na₂SO₄, filtered and evaporated. The residue was purified by flash chromatography (0-75% EtOAc/hexanes) to afford the titled compound, which is not very pure; thus it was further purified by reverse phase flash chromatography with C₁₈ column (35-100% MeCN/water) to afford the titled compound as a wax (43.0 mg). LCMS (+ESI): Rt=2.31 min, (M+H)⁺=575.2 and 577.1.

### Step 3: (S)—N-(3-(4-(6-bromo-1H-indazol-3-yl)-3-fluorophenoxy)propyl)-N-methylpyrrolidine-2-carboxamide

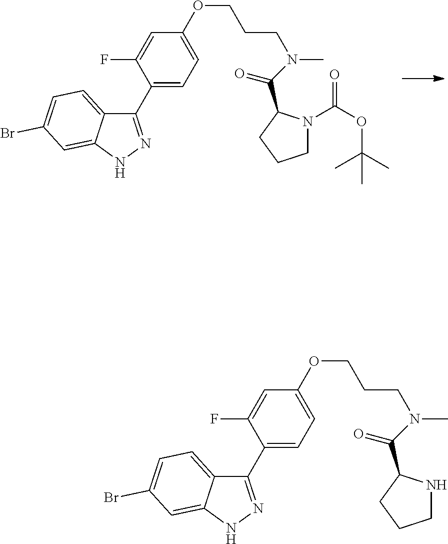 [SMILES: CN(CCCOc1ccc(-c2n[nH]c3cc(Br)ccc23)c(F)c1)C(=O)[C@@H]1CCCN1C(=O)OC(C)(C)C ; CN(CCCOc1ccc(-c2n[nH]c3cc(Br)ccc23)c(F)c1)C(=O)[C@@H]1CCCN1]

To a reaction vial containing (S)-tert-butyl 2-((3-(4-(6-bromo-1H-indazol-3-yl)-3-fluorophenoxy)propyl)(methyl)carbamoyl)pyrrolidine-1-carboxylate (0.043 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a glassy like gum (35 mg). LCMS (+ESI): Rt=1.66 min, (M+H)⁺=475.2 and 477.1.

### (R)—N-(3-(4-(6-bromo-1H-indazol-3-yl)-3-fluorophenoxy)propyl)-N-methylpyrrolidine-2-carboxamide (LZH-00072)

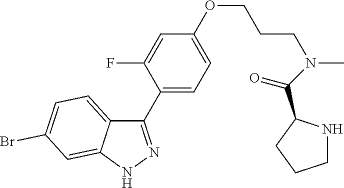 [SMILES: CN(CCCOc1ccc(-c2n[nH]c3cc(Br)ccc23)c(F)c1)C(=O)[C@@H]1CCCN1]

### Step 1: (R)-tert-butyl 2-(1H-imidazole-1-carbonyl)pyrrolidine-1-carboxylate

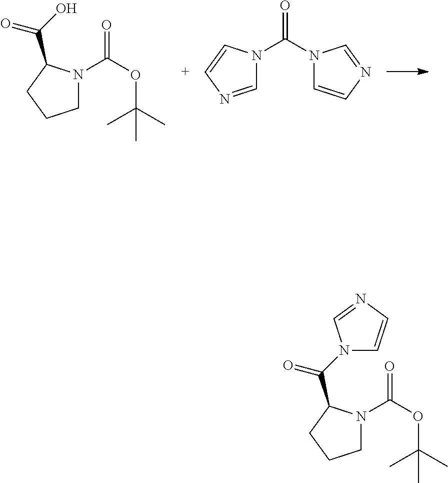 [SMILES: CC(C)(C)OC(=O)N1CCC[C@H]1C(=O)O ; O=C(n1ccnc1)n1ccnc1 ; CC(C)(C)OC(=O)N1CCC[C@H]1C(=O)n1ccnc1]

To a mixture of (R)-1-(tert-butoxycarbonyl)pyrrolidine-2-carboxylic acid (2.37 g, 11 mmol) in THF (15 mL) stirred at rt, 1,1′-Carbonyldiimidazole (1.62 g, 10.01 mmol) was added portion wise. After the addition, the mixture was stirred at rt for an overnight. The volume was adjusted to 20 mL by addition of more THF and the titled compound was afford as a 0.5 M stock solution for further use.

### Step 2: (R)-tert-butyl 2-((3-(4-(6-bromo-1H-indazol-3-yl)-3-fluorophenoxy)propyl)(methyl)carbamoyl)pyrrolidine-1-carboxylate

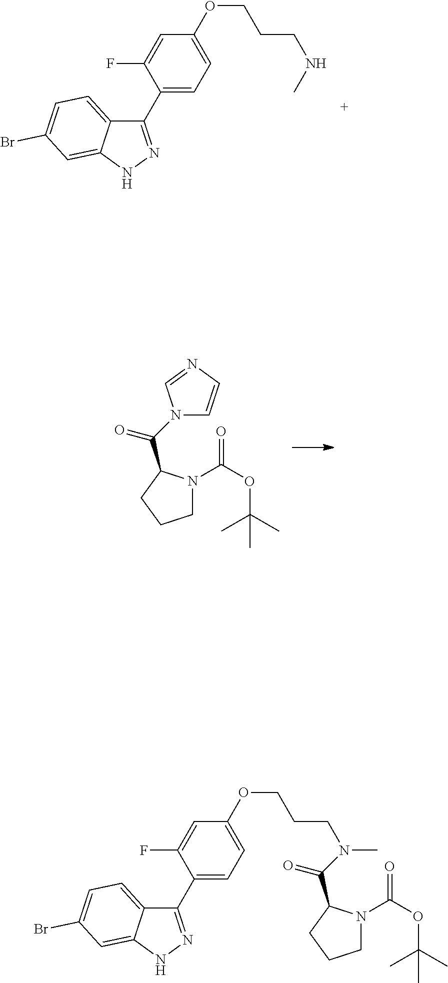 [SMILES: CNCCCOc1ccc(-c2n[nH]c3cc(Br)ccc23)c(F)c1 ; CC(C)(C)OC(=O)N1CCC[C@H]1C(=O)n1ccnc1 ; CN(CCCOc1ccc(-c2n[nH]c3cc(Br)ccc23)c(F)c1)C(=O)[C@@H]1CCCN1C(=O)OC(C)(C)C]

To a mixture of 3-(4-(6-bromo-1H-indazol-3-yl)-3-fluorophenoxy)-N-methylpropan-1-amine (0.083 g, 0.22 mmol) in THF (2 mL) stired it, (R)-tert-butyl 2-(1H-imidazole-1-carbonyl)pyrrolidine-1-carboxylate (0.57 mL, 0.5 M in THF, 0.29 mmol) was added dropwise. After the addition, the mixture was heated at 65° C. overnight. The reaction was worked up by the addition of 1 M NaOH and the mixture was extracted with EtOAc, dried over Na₂SO₄, filtered and evaporated. The residue was purified by flash chromatography (0-75% EtOAc/hexanes) to afford the titled compound, which is not very pure; thus it was further purified by reverse phase flash chromatography with C₁₈ column (35-100% MeCN/water) to afford the titled compound as a wax (73.0 mg). LCMS (+ESI): Rt=2.30 min, (M+H)⁺=575.2 and 577.1.

### Step 3: (R)—N-(3-(4-(6-bromo-1H-indazol-3-yl)-3-fluorophenoxy)propyl)-N-methylpyrrolidine-2-carboxamide

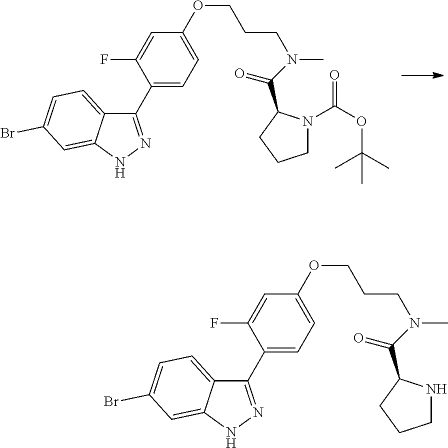 [SMILES: CN(CCCOc1ccc(-c2n[nH]c3cc(Br)ccc23)c(F)c1)C(=O)[C@@H]1CCCN1C(=O)OC(C)(C)C ; CN(CCCOc1ccc(-c2n[nH]c3cc(Br)ccc23)c(F)c1)C(=O)[C@@H]1CCCN1]

To a reaction vial containing (R)-tert-butyl 2-((3-(4-(6-bromo-1H-indazol-3-yl)-3-fluorophenoxy)propyl)(methyl)carbamoyl)pyrrolidine-1-carboxylate (0.073 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a glassy gum (50 mg). LCMS (+ESI): Rt=1.67 min, (M+H)⁺=475.1 and 477.1.

### N-(3-(4-(6-bromo-1H-indazol-3-yl)-3-fluorophenoxy)propyl)-N-methyl-2-(methylamino)acetamide (LZH-00073)

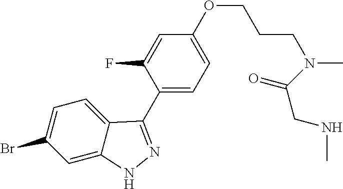

### Step 1: tert-butyl (2-(1H-imidazol-1-yl)-2-oxoethyl)(methyl)carbamate

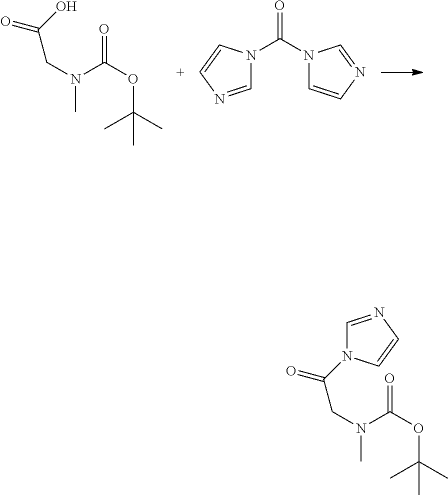 [SMILES: CN(CC(=O)O)C(=O)OC(C)(C)C ; O=C(n1ccnc1)n1ccnc1 ; CN(CC(=O)n1ccnc1)C(=O)OC(C)(C)C]

To a mixture of 2-((tert-butoxycarbonyl)(methyl)amino)acetic acid (2.08 g, 11 mmol) in THF (15 mL) stired at rt, 1,1′-Carbonyldiimidazole (1.62 g, 10.01 mmol) was added portion wise. After the addition, the mixture was stirred at rt for an overnight. The volume was adjusted to 20 mL by addition of more THF and the titled compound was afford as a 0.5 M stock solution for further use.

### Step 2: tert-butyl (2-((3-(4-(6-bromo-1H-indazol-3-yl)-3-fluorophenoxy)propyl)(methyl)amino)-2-oxoethyl)(methyl)carbamate

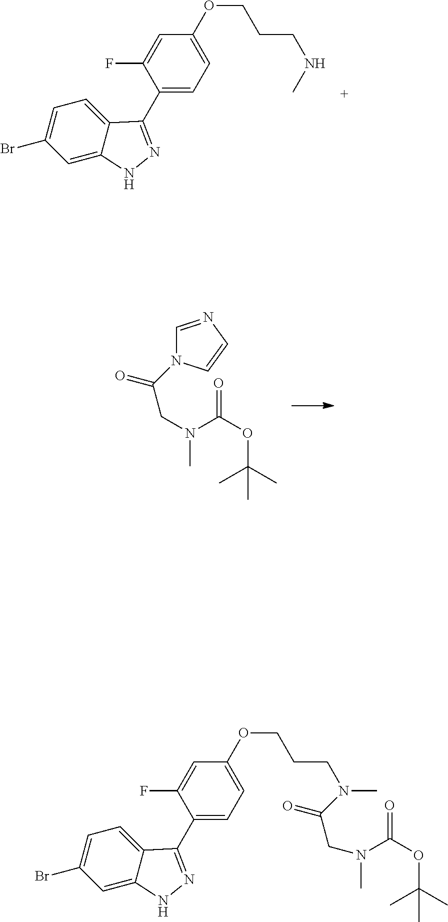 [SMILES: CNCCCOc1ccc(-c2n[nH]c3cc(Br)ccc23)c(F)c1 ; CN(CC(=O)n1ccnc1)C(=O)OC(C)(C)C ; CN(CCCOc1ccc(-c2n[nH]c3cc(Br)ccc23)c(F)c1)C(=O)CN(C)C(=O)OC(C)(C)C]

To a mixture of 3-(4-(6-bromo-1H-indazol-3-yl)-3-fluorophenoxy)-N-methylpropan-1-amine (0.083 g, 0.22 mmol) in THF (2 mL) stired at rt, tert-butyl (2-(I H-imidazol-1-yl)-2-oxoethyl)(methyl)carbamate (0.57 mL, 0.5 M in THF, 0.29 mmol) was added dropwise. After the addition, the mixture was heated at 65° C. overnight. The reaction was worked up by the addition of 1 M NaOH and the mixture was extracted with EtOAc, dried over Na₂SO₄, filtered and evaporated. The residue was purified by flash chromatography (0-75% EtOAc/hexanes) to afford the titled compound, which is not very pure; thus it was further purified by reverse phase flash chromatography with C is column (35-100% MeCN/water) to afford the titled compound as a wax (51.0 mg). LCMS (+ESI): Rt=2.31 min, (M+1H)⁺=549.2 and 551.1.

### Step 3: N-(3-(4-(6-bromo-1H-indazol-3-yl)-3-fluorophenoxy)propyl)-N-methyl-2-(methylamino)acetamide

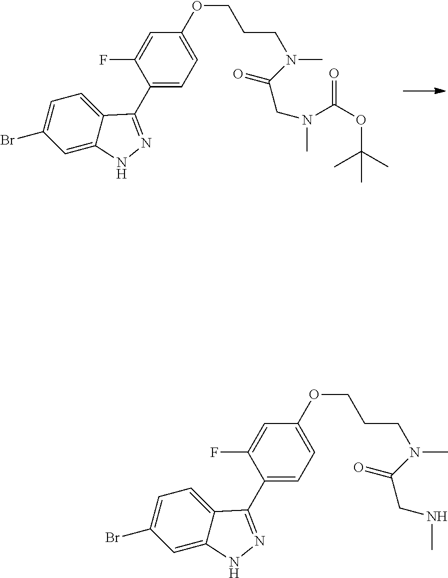 [SMILES: CN(CCCOc1ccc(-c2n[nH]c3cc(Br)ccc23)c(F)c1)C(=O)CN(C)C(=O)OC(C)(C)C ; CNCC(=O)N(C)CCCOc1ccc(-c2n[nH]c3cc(Br)ccc23)c(F)c1]

To a reaction vial containing tert-butyl (2-((3-(4-(6-bromo-1H-indazol-3-yl)-3-fluorophenoxy)propyl)(methyl)amino)-2-oxoethyl)(methyl)carbamate (0.051 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a glassy gum (41 mg). LCMS (+ESI): Rt=1.66 min, (M+H)⁺=449.1, and 451.0.

### (S)—N-(2-((4-(6-bromo-1H-indazol-3-yl)-3-fluorobenzyl)(methyl)amino)ethyl)-N-methylpyrrolidine-2-carboxamide (LZH-00074)

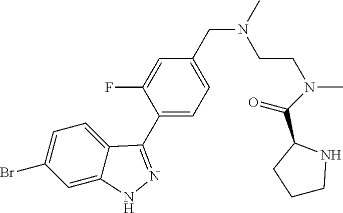 [SMILES: CN(CCN(C)C(=O)[C@@H]1CCCN1)Cc1ccc(-c2n[nH]c3cc(Br)ccc23)c(F)c1]

### Step 1: (S)-tert-butyl 2-((2-((4-(6-bromo-1H-indazol-3-yl)-3-fluorobenzyl)(methyl)amino)ethyl)(methyl)carbamoyl)pyrrolidine-1-carboxylate

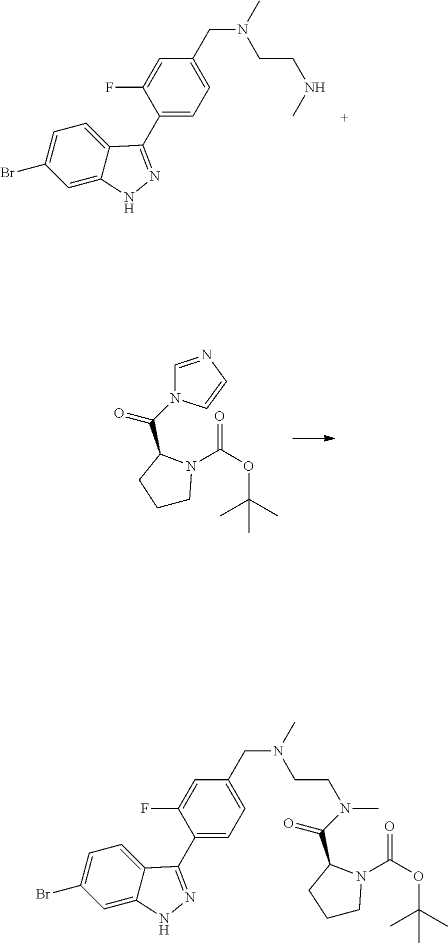 [SMILES: CNCCN(C)Cc1ccc(-c2n[nH]c3cc(Br)ccc23)c(F)c1 ; CC(C)(C)OC(=O)N1CCC[C@H]1C(=O)n1ccnc1 ; CN(CCN(C)C(=O)[C@@H]1CCCN1C(=O)OC(C)(C)C)Cc1ccc(-c2n[nH]c3cc(Br)ccc23)c(F)c1]

To a mixture of N1-(4-(6-bromo-1H-indazol-3-yl)-3-fluorobenzyl)-N1,N2-dimethylethane-1,2-diamine (0.033 g, 0.08 mmol) in THF (2 mL) stired rt, (S)-tert-butyl 2-(1H-imidazole-1-carbonyl)pyrrolidine-1-carboxylate (0.22 mL, 0.5 M in THF, 0.11 mmol) was added dropwise. After the addition, the mixture was stirred at reflux overnight. The reaction was worked up by the addition of 1 M NaOH and the mixture was extracted with EtOAc, dried over Na₂SO₄, filtered and evaporated. The residue was purified by flash chromatography (0-100% solvent B/hexanes, while solvent B is a solvent mixture of NEt₃/MeOH/EtOAc in a ratio of 1/10/39) to afford the titled compound, which is not very pure; thus it was further purified by reverse phase flash chromatography with C₁₈ column (35-100% MeCN/water) to afford the titled compound as a wax (15.0 mg). LCMS (+ESI): Rt=2.03 min, (M+H)⁺=588.2 and 590.1.

### Step 2: (S)—N-(2-((4-(6-bromo-1H-indazol-3-yl)-3-fluorobenzyl)(methyl)amino)ethyl)-N-methylpyrrolidine-2-carboxamide

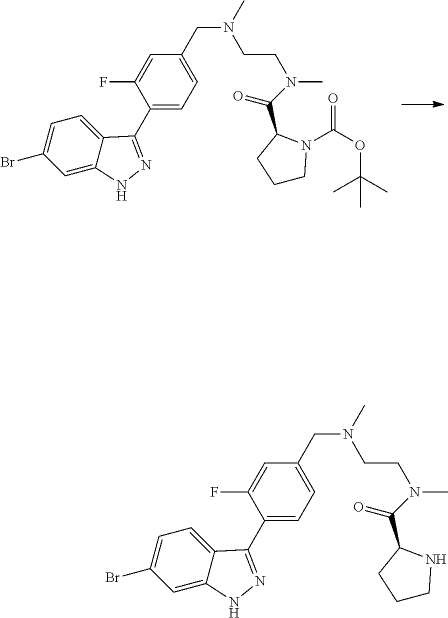 [SMILES: CN(CCN(C)C(=O)[C@@H]1CCCN1C(=O)OC(C)(C)C)Cc1ccc(-c2n[nH]c3cc(Br)ccc23)c(F)c1 ; CN(CCN(C)C(=O)[C@@H]1CCCN1)Cc1ccc(-c2n[nH]c3cc(Br)ccc23)c(F)c1]

To a reaction vial containing (S)-tert-butyl 2-((2-((4-(6-bromo-1H-indazol-3-yl)-3-fluorobenzyl)(methyl)amino)ethyl)(methyl)carbamoyl)pyrrolidine-1-carboxylate (0.015 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a gummy solid (12 mg). LCMS (+ESI): Rt=1.46 min, (M+H)⁺=488.1 and 490.1.

### (R)—N-(2-((4-(6-bromo-1H-indazol-3-yl)-3-fluorobenzyl)(methyl)amino)ethyl)-N-methylpyrrolidine-2-carboxamide (LZH-00075)

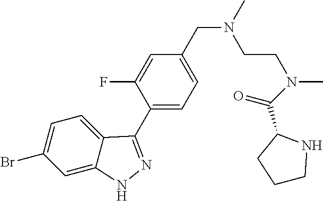 [SMILES: CN(CCN(C)C(=O)[C@H]1CCCN1)Cc1ccc(-c2n[nH]c3cc(Br)ccc23)c(F)c1]

### Step 1: (R)-tert-butyl 2-((2-((4-(6-bromo-1H-indazol-3-yl)-3-fluorobenzyl)(methyl)amino)ethyl)(methyl)carbamoyl)pyrrolidine-1-carboxylate

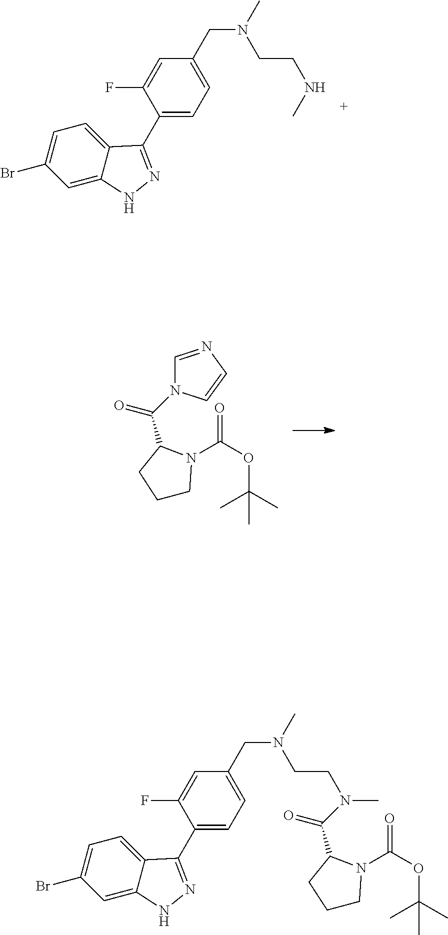

To a mixture of N1-(4-(6-bromo-1H-indazol-3-yl)-3-fluorobenzyl)-N1,N2-dimethylethane-1,2-diamine (0.033 g, 0.08 mmol) in THF (2 mL) stired rt, (R)-tert-butyl 2-(1H-imidazole-1-carbonyl)pyrrolidine-1-carboxylate (0.22 mL, 0.5 M in THF, 0.11 mmol) was added dropwise. After the addition, the mixture was stirred at reflux overnight. The reaction was worked up by the addition of 1 M NaOH and the mixture was extracted with EtOAc, dried over Na₂SO₄, filtered and evaporated. The residue was purified by flash chromatography (0-100% solvent B/hexanes, while solvent B is a solvent mixture of NEt₃/MeOH/EtOAc in a ratio of 1/10/39) to afford the titled compound, which is not very pure; thus it was further purified by reverse phase flash chromatography with C₁₈ column (35-100% MeCN/water) to afford the titled compound as a wax (7.0 mg). LCMS (+ESI): Rt=2.08 min, (M+H)⁺=588.1 and 590.1.

### Step 2: (R)—N-(2-((4-(6-bromo-1H-indazol-3-yl)-3-fluorobenzyl)(methyl)amino)ethyl)-N-methylpyrrolidine-2-carboxamide

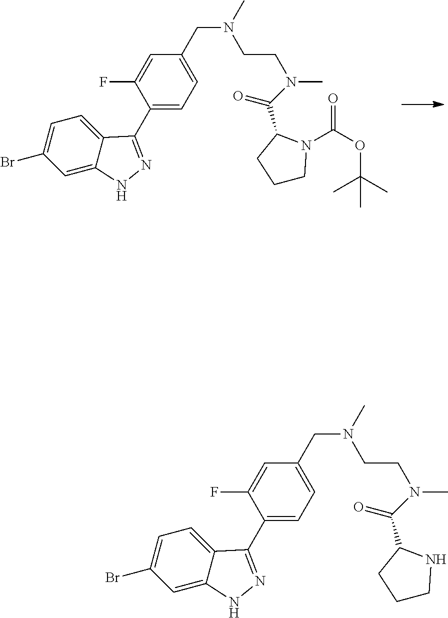

To a reaction vial containing (R)-tert-butyl 2-((2-((4-(6-bromo-1H-indazol-3-yl)-3-fluorobenzyl)(methyl)amino)ethyl)(methyl)carbamoyl)pyrrolidine-1-carboxylate (0.007 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a glassy gum (3.0 mg). LCMS (+ESI): Rt=1.49 min, (M+H)⁺=488.2 and 490.1.

### N-(2-((4-(6-bromo-1H-indazol-3-yl)-3-fluorobenzyl)methyl)amino)ethyl)-N-methyl-2-(methylamino)acetamide (LZH-00076)

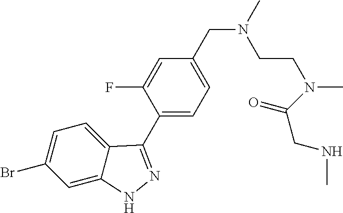 [SMILES: CNCC(=O)N(C)CCN(C)Cc1ccc(-c2n[nH]c3cc(Br)ccc23)c(F)c1]

### Step 1: tert-butyl (2-((2-((4-(6-bromo-1H-indazol-3-yl)-3-fluorobenzyl)(methyl)amino)ethyl)(methyl)amino)-2-oxoethyl)(methyl)carbamate

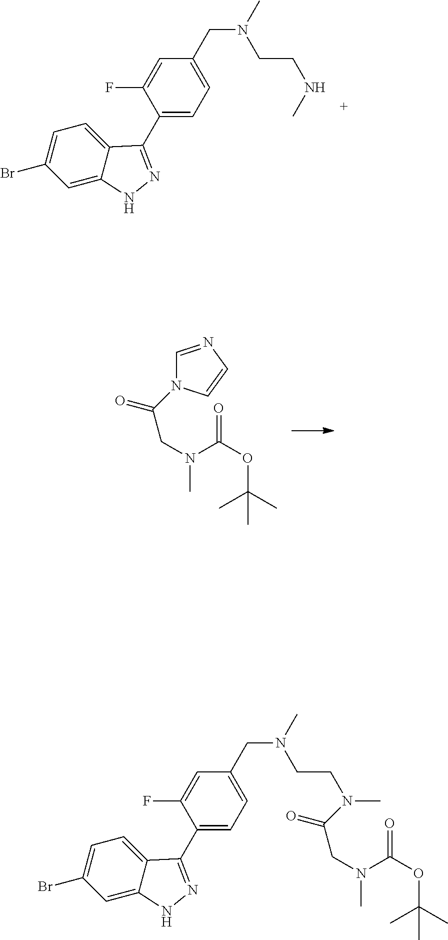 [SMILES: CNCCN(C)Cc1ccc(-c2n[nH]c3cc(Br)ccc23)c(F)c1 ; CN(CC(=O)n1ccnc1)C(=O)OC(C)(C)C ; CN(CCN(C)C(=O)CN(C)C(=O)OC(C)(C)C)Cc1ccc(-c2n[nH]c3cc(Br)ccc23)c(F)c1]

To a mixture of N1-(4-(6-bromo-1H-indazol-3-yl)-3-fluorobenzyl)-N1,N2-dimethylethane-1,2-diamine (0.033 g, 0.08 mmol) in THF (2 mL) stired rt, tert-butyl (2-(1H-imidazol-1-yl)-2-oxoethyl)(methyl)carbamate (0.22 mL, 0.5 M in THF, 0.11 mmol) was added dropwise. After the addition, the mixture was stirred at reflux overnight. The reaction was worked up by the addition of 1 M NaOH and the mixture was extracted with EtOAc, dried over Na₂SO₄, filtered and evaporated. The residue was purified by flash chromatography (0-100% solvent B/hexanes, while solvent B is a solvent mixture of NEt₃/MeOH/EtOAc in a ratio of 1/10/39) to afford the titled compound, which is not very pure; thus it was further purified by reverse phase flash chromatography with C₁₈ column (35-100% MeCN/water) to afford the titled compound as a wax (10.0 mg). LCMS (+ESI): Rt=3.74 min, (M+H)⁺=562.1 and 564.1.

### Step 2: N-(2-((4-(6-bromo-1H-indazol-3-yl)-3-fluorobenzyl)(methyl)amino)ethyl)-N-methyl-2-(methylamino)acetamide

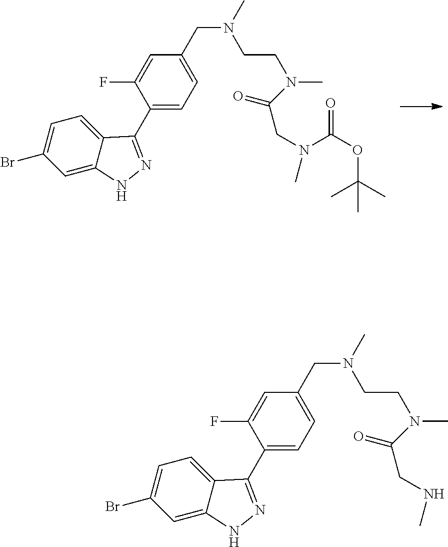 [SMILES: CN(CCN(C)C(=O)CN(C)C(=O)OC(C)(C)C)Cc1ccc(-c2n[nH]c3cc(Br)ccc23)c(F)c1 ; CNCC(=O)N(C)CCN(C)Cc1ccc(-c2n[nH]c3cc(Br)ccc23)c(F)c1]

To a reaction vial containing tert-butyl (2-((2-((4-(6-bromo-1H-indazol-3-yl)-3-fluorobenzyl)(methyl)amino)ethyl)(methyl)amino)-2-oxoethyl)(methyl)carbamate (0.010 g), TFA/DCM (2 mL, 1:1) was added via a syringe. After the addition, the mixture was stirred at rt for 2 hr. The reaction mixture was evaporated. The residue was treated with sat NaHCO₃/H₂O, extracted with EtOAc, dried over Na₂SO₄, filtered and evaporated. The residue was purified by flash chromatography (0-20% Solvent B/DCM, while Solvent B is a mixture of ammonia/MeOH (1:5)) to afford the titled compound as a gummy solid (7.0 mg). LCMS (+ESI): Rt=1.49 min, (M+H)⁺=462.1 and 464.1.

### Example 48: Evaluation of the Synthesized Compounds for their Effects Upon CFTR Modeling to Access the Synthesized Compounds

The CFTR NBD1-NBD2 dimeric model was generated through protein-protein docking. Specifically, NBD1 and NBD2 were taken from the high-resolution cryo-EM structure of the human CFTR. A FFT-based rigid protein-protein docking method (MDockPP) was employed to generate putative binding poses of the NBD1-NBD2 complex, which were ranked by ITScorePP scoring function. AutoDock Vina (designed and implemented by Dr. Oleg Trott in the Molecular Graphics Lab at The Scripps Research Institute) was then used to dock two ATP molecules into the modeled head-to-tail structure of the NBD1-NBD2 complex. The docking results were visually inspected, keeping the binding pose of each ATP molecule that was most consistent with the experimentally confirmed specific interactions with NBD1 and NBD2. This modeled NBD1-NBD2-ATP1-ATP2 complex structure was used for docking/screening the compounds described herein.

The atomic structures of the molecules of the compounds described herein are generated through with the program Avogadro. Virtual screening/docking of small molecules were performed with the program, AutoDock Vina. Specifically, the score of each compound was assessed with the scoring function provided by Vina. The specific action mechanism was analyzed (e.g., identification of the critical residues on NBD1-NBD2 that interact with a compound).

Currently no energy scoring function can provide a threshold for binding. Namely, it is difficult to give a scoring value, above which a molecule is predicted not to bind. It is usually the relative scores that skilled artisan use. The more negative the score, the stronger the binding that is predicted. Moreover, the scores can only be used to predict qualitatively the binding affinities of these molecules, because the accuracy of the prediction is limited by the energy scoring function (currently, there is no excellent scoring function to predict binding affinities), the accuracy of the protein model, and the potential protein conformational changes. In contrast, the action mechanism, which is predicted based on the docking poses, is more reliable.

### Western Blot Analysis

The amount of CFTR proteins in the cell membrane can be determined with Western blot analysis. Like all membrane proteins in the cell, the CFTR protein is synthesized in the endoplasmic reticulum (ER), translocated to the Golgi apparatus for glycosylation before trafficked to the plasma membrane. Because of the addition of complex sugar molecules to CFTR, the molecular weight of “mature” or post-Golgi CFTR in the plasma membrane is higher than that of newly synthesized proteins in the ER. Thus, in Western blot, it is possible to discern two distinct bands: band C representing high molecular weight, mature CFTR, and band B low molecular weight, immature CFTR. While wild-type proteins are present mostly as band C, Class II disease-associated mutations such as delF508 causes a drastic diminution of band C, resulting in an effective decrease of N in the formula N×Pₒ×i. The effects of the reagents (or CFTR correctors) that enhance band C can thus be quantitatively assessed with the Western blot analysis. By the same token, any interactions between different compounds, with positive or negative impact on band C, can be determined with this assay.

Western blot experiments were carried out in both transient expression system (CHO cells) and a stable cell line (C127 cells). CHO cells, grown in 35 mm dishes, were transfected with delF508-CFTR construct in pcDNA plasmids using X-tremeGENE (Roche). Six hours after transfection, tested compounds were added to the medium. In case of delF508-CFTR-expressing stable C127 cells, compounds were added to the medium at about 30% confluence. In both cases, cells were lysed 18 hours post drug treatment using 1×SDS loading buffer. Cell lysates were sheared by pushed through 18G needles. Whole cell lysate were separated in 4˜20% gradient gels (Bio-Rad Laboratories) and transferred onto nitrocellulose membranes. The membranes were blocked with 5% milk in TBST buffer (20 mM Tris, 137 mM NaCl, 0.1% Tween 20) at 4° C. overnight. The membranes were then probed with anti-CFTR antibody (AB596 from the Cystic Fibrosis Foundation) and anti-vimentin antibody as a control (Santa Cruz Biotechnology) at room temperature for two hours. The membranes were washed with TBST five times and then incubated with anti-mouse IgG, HRP-linked antibody (Cell Signaling Technology) at room temperature for one hour. The membranes were washed three times with TBST and developed with chemiluminescence reagent (Thermo Scientific). The luminescence was detected and quantified by a Molecular Image Chemi Doc (Bio-Rad Laboratories).

### Patch-Clamp

Patch-clamp is considered the gold standard for assessing directly ion channel function in real time. By attaching a glass microelectrode on the surface of a cell, it is possible to record the currents generated by channels proteins that are present in this tiny “patch” of cell membranes. Since millions of ions pass through a single ion channel pore per second, the resulting microscopic currents from a single channel protein molecule can be detected with modem patch-clamp amplifiers. As ion channels (a conductor in electricity term) are arranged in parallel in the cell membrane, it is also possible to assay a group of ion channels by monitoring macroscopic currents representing the sum of currents from individual channels. Microscopic recordings allow artisans to examine exquisitely the effects of compounds on the Pₒ and kinetic parameters of CFTR channels; whereas macroscopic recordings provide an assay not only to quantify the overall effects of compounds on N×Pₒ×i, but also monitor the stability of the CFTR currents in real time.

### Cell Culture and Transfection

Chinese hamster ovary (CHO) cells were grown at 37° C. in Dulbecco's modified Eagle's medium supplemented with 10% fetal bovine serum. Cells were subcultured into 35-mm tissue culture dishes 1 day before transfection. PolyFect transfection reagent (QIAGEN) was used to cotransfect CFTR cDNA and pEGFP-C3 (Takara Bio Inc.), which encodes green fluorescence proteins, into CHO cells. After transfection, cells were incubated at 27° C. for at least 2 days before electrophysiological experiments were performed.

### Electrophysiological Recordings

Glass chips carrying the transfected cells were transferred to a chamber located on the stage of an inverted microscope (IX51; Olympus). Borosilicate capillary glasses were used to make patch pipettes using a two-stage micropipette puller (PP-81, Narishige, Japan). The pipettes were polished with a homemade microforge to a resistance of 2-4 MΩ in the bath solution. Once the seal resistance reached >40 GΩ, membrane patches were excised into an inside-out mode. Subsequently, the pipette was moved to the outlet of a three-barrel perfusion system and perfused with 25 IU PKA and 2 mM ATP until the CFTR current reached a steady state. A patch-clamp amplifier (EPC10, HEKA, Germany) was used to record electrophysiological data at room temperature. The data were filtered online at 100 Hz with an eight-pole Bessel filter (LPF-8; Warner Instruments) and digitized to a computer at a sampling rate of 500 Hz. Solution changes were effected with a fast solution change system (SF-77B; Warner Instruments) that has a dead time around 30 ms (Tsai et al., J. Gen. Physiol. 2009). Membrane potential was held at −30 or −50 mV for all recordings.

### Testing of Synthesized Compounds and the Results

Various LZH compounds were tested for their effects upon various CFTRs, alone or in combination with Ivacaftor (i.e., VX-770) or Lumacaftor (VX-809), with the methods described above.

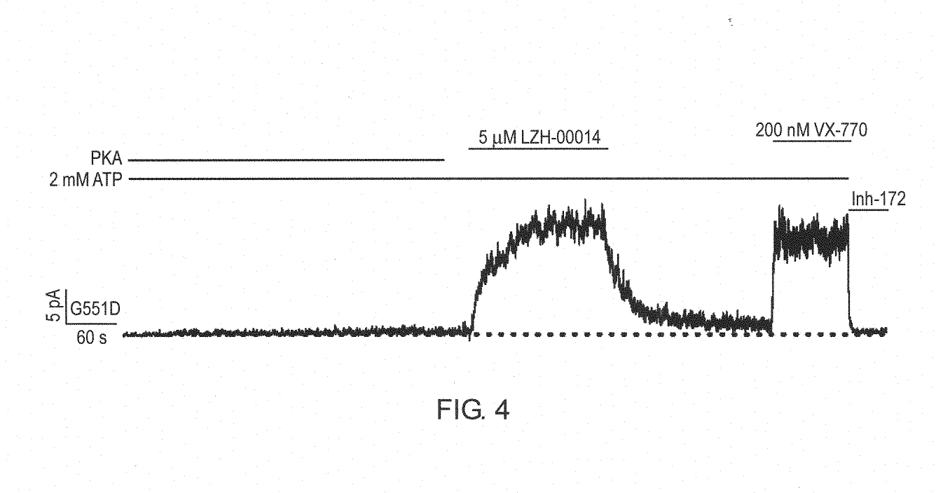

As shown in FIG. 4, a real-time recording of G551 D-CFTR currents shows minimal current activation by PKA and ATP, but the currents can be greatly potentiated by LZH-00014. Removal of LZH-00014 resulted in a slow return of the current to the pre-stimulated level. An addition of Ivacaftor (i.e., VX-770) increased the current to approximately the same level as LZH-00014 and the current can be completely eliminated by a specific CFTR inhibitor (Inh-172, Ma et al., J. Clin. Invest., 2002).

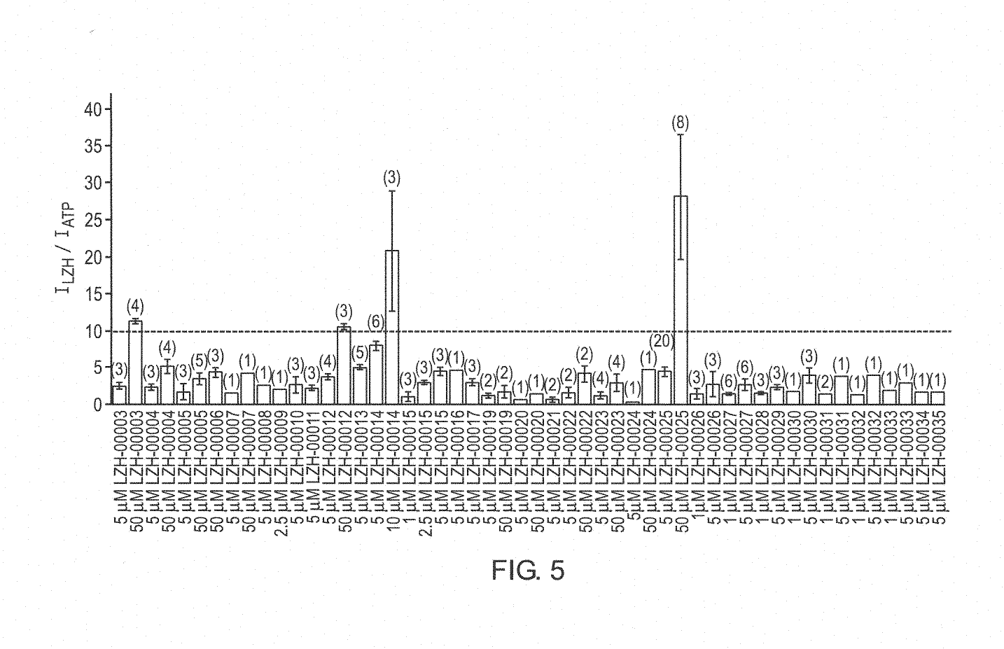

Data presented in FIG. 5 suggest that several compounds are at least as potent as, or even more potent than, Ivacaftor (VX-770) in increasing G551D-CFTR currents.

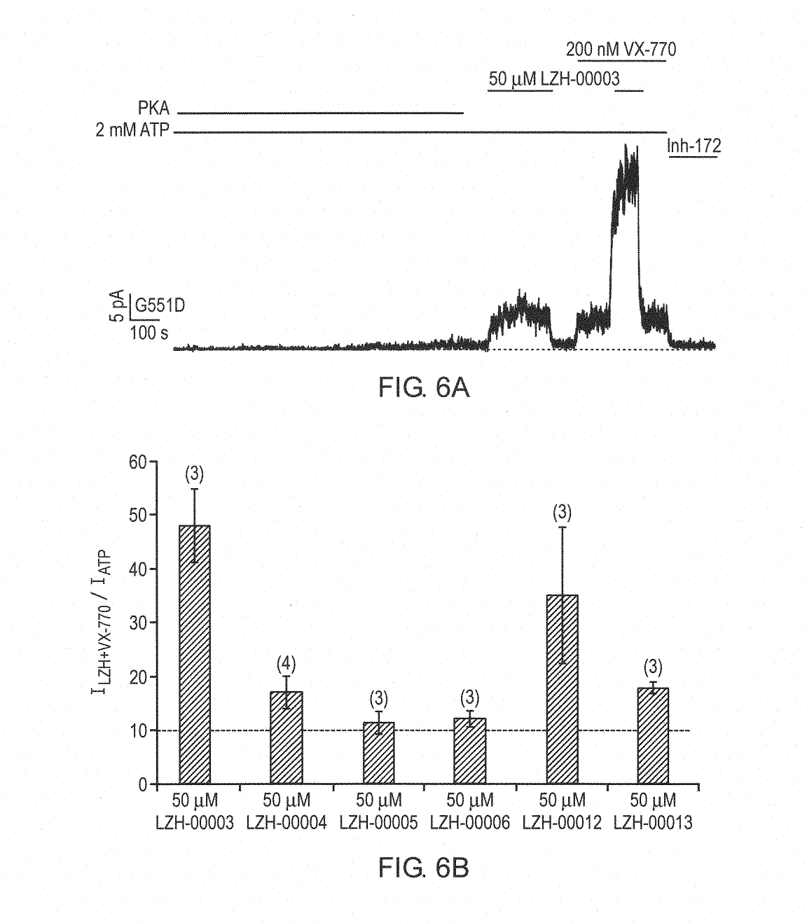

As shown in FIGS. 6A and 6B, LZH-00003 can further increase G551D-CFTR currents in the presence of a maximally effective concentration of Ivacaftor. Such an observation suggests that the LZH compounds and Ivacaftor potentiate CFTR activity through distinct mechanisms (i.e., pharmacological synergism). Additionally, ˜50-fold enhancement of G551D-CFTR can be attained by a combination of LZH-00003 and VX-770. These data suggest that it is possible to completely rectify the dysfunction of G551D-CFTR to a level that is expected to functionally cure patients carrying this mutation.

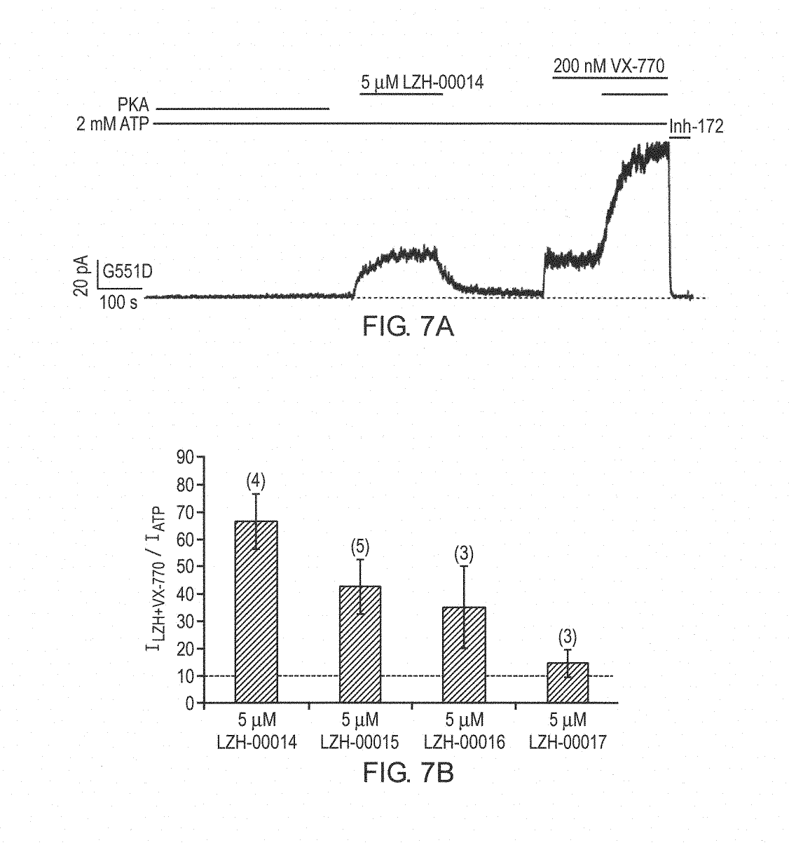

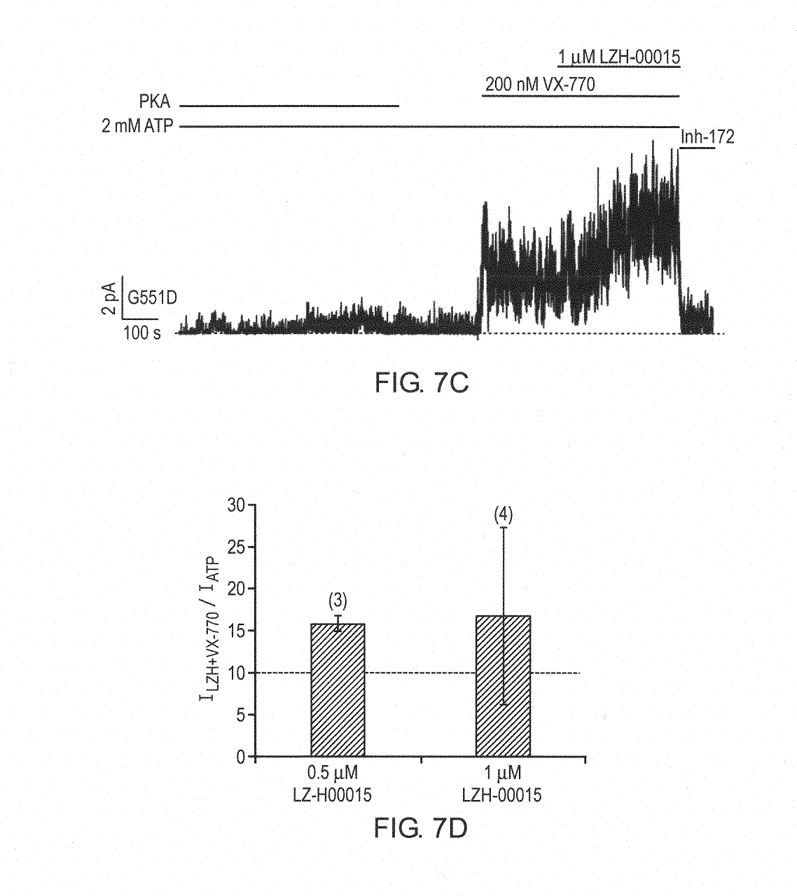

The data presented in FIG. 7 suggest LZH-00014 has a higher potency than LZH-00003, because the concentration of LZH-00014 to achieve a similar effect as LZH-00003 is 10-fold lower than the concentration of LZH-00003 (FIG. 6A and FIG. 7A). It is further noted, given FIG. 7D, that even at 1 μM concentration of LZH-00015, a significant synergism with VX-770 can be observed.

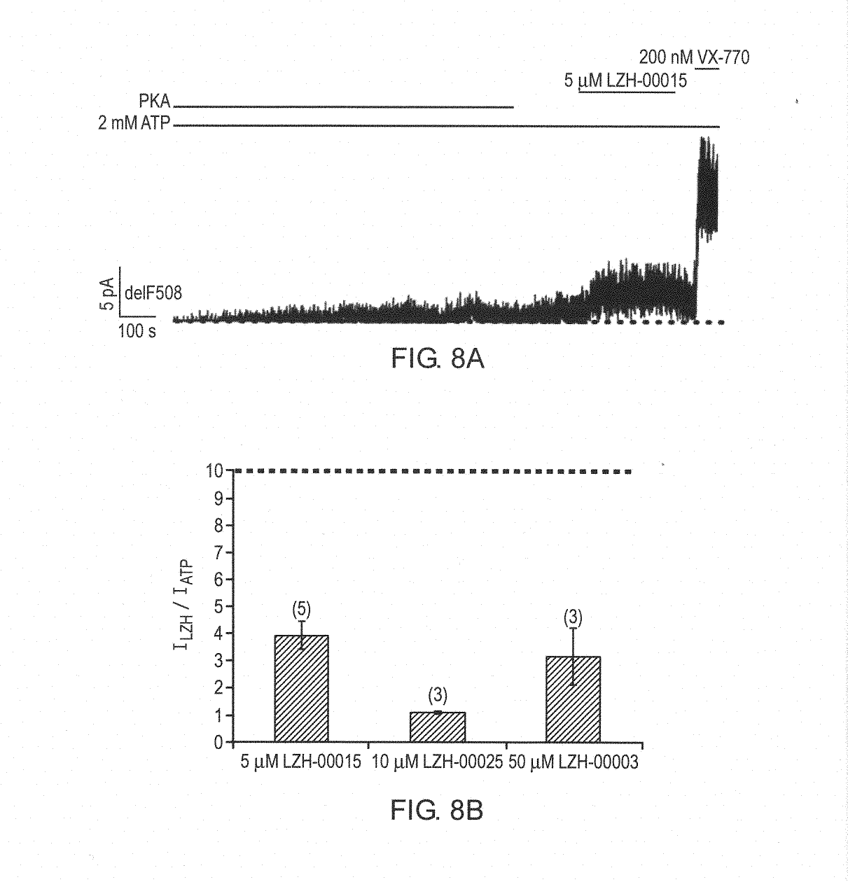

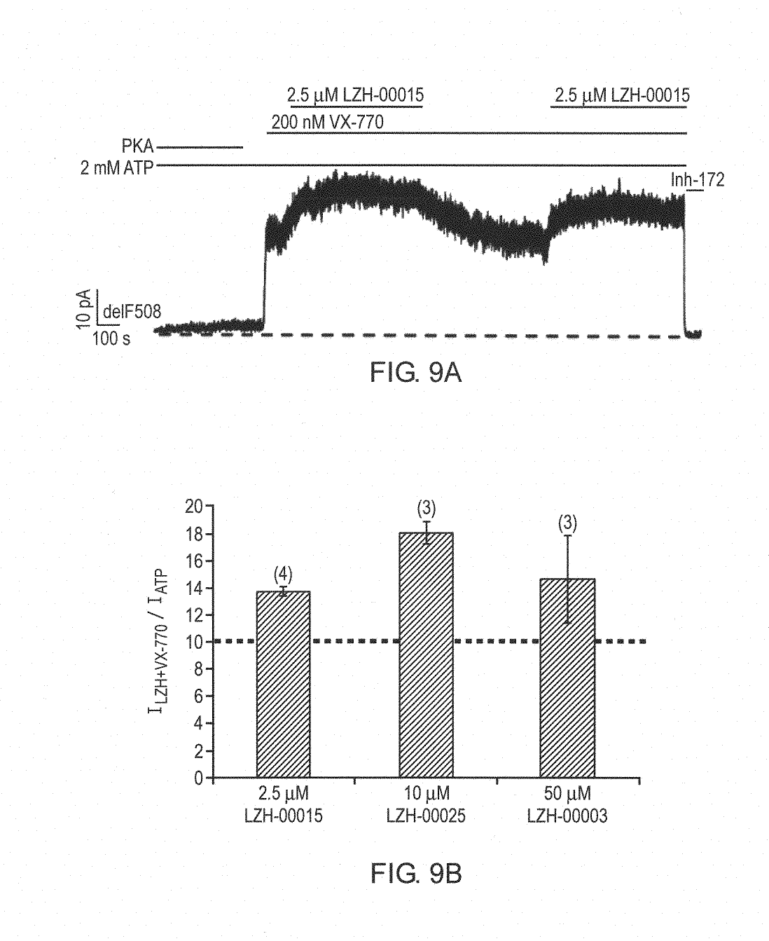

As shown in FIG. 8, several compounds are capable of increasing delF508-CFTR currents. The levels of the increase are at least 50% lower compared to that of Ivacaftor (VX-770). Data presented in FIG. 9 indicate synergistic effects of the LZH compounds and Ivacaftor (VX-770) on delF508-CFTR. Since the Pₒ of delF508-CFTR in the presence of Ivacaftor is already above 0.4 (Kopeikin et al., J. Cys. Fibro. 2014), additional increase of the Pₒ by the LZH compounds is limited. However, this further enhancement of the Pₒ in the presence of the maximally effective concentration of Ivacaftor does indicate a pharmacological synergism as seen with G551D-CFTR in FIG. 6.

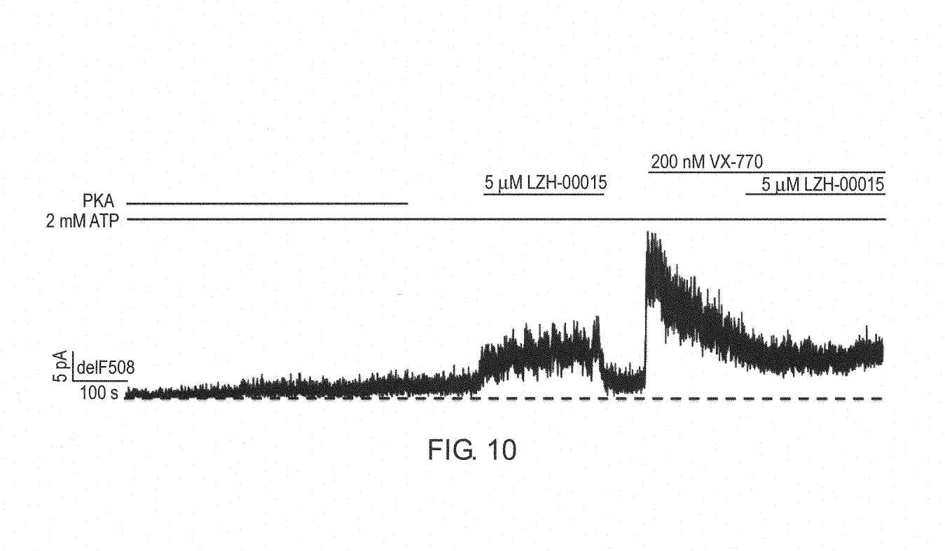

The effects of LZH compound upon delF508-CFTR were further characterized using exemplary compound LZH-00015, as illustrated in FIG. 10. One characteristic feature of plasma membrane delF508-CFTR is its instability over time. The mutant channels “rundown” easily. This spontaneous decay of delF508-CFTR channel function is irreversible and has been documented in the literature (e.g., Meng et al., J. Biol. Chem. 2017). In addition, it is known that this rundown is accelerated by Ivacaftor (VX-770) treatment. In contrast, as seen in this real-time current trace of the delF508-CFTR channels in FIG. 10, there is virtually no rundown of the current in the presence of LZH-00015, but the current immediately starts to decline upon the application of Ivacaftor. Data in FIG. 10 further indicate that the addition of LZH-00015 in the continuous presence of Ivacaftor stops the rundown process and stabilizes the current.

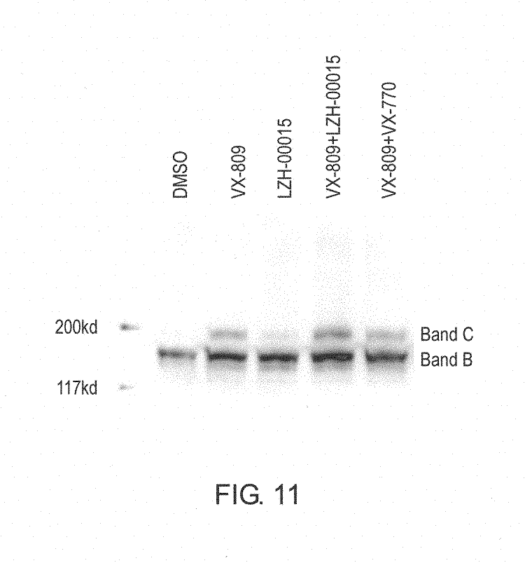

The results shown in FIG. 10 suggest that, contrary to Ivacaftor, the LZH compounds can stabilize the function of delF508-CFTR in the cell membrane. It is known that delF508-CFTR exhibits multiple biochemical defects. For example, in addition to trafficking and gating defects, the half-life of delF508-CFTR proteins in the cell membrane is dramatically shortened (Lukacs et al., J. Biol. Chem. 1993). Both trafficking and stability defects effectively reduce the number of mature delF508-CFTR proteins in the cell membrane. It is also documented that while Lumacaftor (VX-809) improves delF508-CFTR trafficking (Van Goor et al., PNAS 2011), Ivacaftor dampens this effect presumably by destabilizing delF508-CFTR in the plasma membrane (Veit et al., Sci. Transl. Med. 2015). Contrary to Ivacaftor, which diminishes the effect of Lumacaftor, LZH-00014 (data not shown) or LZH-00015 actually enhances the effect of Lumacaftor. In the Western blot shown in FIG. 11, delF508-CFTR shows negligible Band C when treated with DMSO, but the intensity of Band C is increased by 3 μM VX-809 (Lumacaftor). This effect of VX-809 on membrane expression of delF508-CFTR is further enhanced by 500 nM LZH-00015.

### Example 49: Further Characterization of Selected LZH Compounds' Effects Upon CFTR Materials and Methods

### Mutagenesis and Channel Expression

Disease-causing mutations (G551D and delF508) were made with PCR-based site-directed mutagenesis using the Pfu Ultra II (Agilent Technologies). Both constructs were confirmed by DNA sequencing (DNA core; University of Missouri-Columbia), and amplified using Invitrogen Plasmid Miniprep Kit. Chinese hamster ovary (CHO) cell line from American Type Culture Collection (Manassas, Va., USA) was grown at 37° C. and 95% 02-5% CO₂ in Dulbecco's modified Eagle's medium (Life Technologies, Inc., Rockville, Md., USA) containing 10% fetal bovine serum (Harlan Biosciences, Madison, Wis., USA). The cDNA constructs of CFTR (G551D and delF508) were co-transfected with peGFP-C3 (Takara Bio Inc.) encoding the green fluorescent protein into CHO cells using PolyFect transfection reagent (QIAGEN). The transfected cells were transferred into 35 mm tissue culture dishes containing one layer of sterilized glass chips for cells to grow on. The transfected cells were incubated at 27° C. for 2-7 days before electrophysiological experiments were performed.

### Reagents and Electrophysiology

All patch-clamp experiments were carried out in the excised inside-out configuration. Micropipettes made of borosilicate capillary glass were pulled with a two-stage vertical puller (Narishige) and then fire-polished with a homemade microforge to reach a pipet resistance of 2-4 MΩ when the pipettes were filled with a standard inside-out pipet solution that contains: 140 mM N-Methyl-D-glucamine-Cl (NMDG-Cl), 2 mM MgCl₂, 5 mM CaCl₂) and 10 mM HEPES, adjusted to pH 7.4 with NMDG. A glass chip with transfected cells grown on was placed into a chamber on the stage of an inverted microscope (Olympus) and continuously perfused with a bath solution (145 mM NaCl, 5 mM KCl, 2 mM MgCl₂, 1 mM CaCl₂, 5 mM glucose, 5 mM HEPES, and 20 mM sucrose, adjusted to pH 7.4 with NaOH). Immediately after a membrane patch reached a seal resistance of >40 GΩ, it was excised and continuously perfused with a standard perfusate (150 mM NMDG-Cl, 10 mM EGTA, 10 mM HEPES, 8 mM Tris, and 2 mM MgCl₂, adjusted to pH 7.4 with NMDG).

Experiments were conducted at room temperature (22-24° C.). Current signals were acquired with a patch-clamp amplifier (EPC9, HEKA), filtered at 100 Hz, digitized online at 500 Hz with Pulse software (version 8.53, HEKA) and captured onto a hard disk. Fast solution exchange was achieved with a commercial solution exchange system (SF-77B Perfusion Fast-Step, Warner Instruments).

Solutions containing 2 mM ATP (Sigma-Aldrich) were made with the standard perfusate. To keep the reducing environment for PKA (Sigma-Aldrich), 2.67 mM dithiothreitol (DTT) (Sigma-Aldrich) were routinely added to the standard perfusate containing 32 IU/mL PKA and 2 mM ATP. CFTRinh-172 (Inh-172) were kindly provided by Dr. Robert Bridges (Department of Physiology and Biophysics, Rosalind Franklin University, Chicago, Ill.) with support from the Cystic Fibrosis Foundation Therapeutics. VX-770 was purchased from Selleckchem.

For Western blotting experiments, delF508 stably-transfected C127 cells were cultured in DMEM with 10% FBS in 6-well plates. Different tested compounds were added to the culture medium within 24 hours of passage to the desired concentration. Cells were lysed 18 hours post drug treatment using 1×SDS loading buffer. Cell lysates were sheared by pushed through 18G needles. Whole cell lysates were separated in 4-20% gradient gels (Bio-Rad Laboratories) and transferred onto nitrocellulose membranes. The membranes were blocked with 5% milk in TBST buffer (20 mM Tris, 137 mM NaCl, 0.1% Tween 20) at 4° C. overnight. The membranes were then probed with anti-CFTR antibody (AB660 from CFTR foundation) and anti-vimentin antibody as a loading control (Santa Cruz Biotechnology) at room temperature for two hours. The membranes were washed with TBST five times and then incubated with anti-mouse IgG, HRP linked antibody (Cell Signaling Technology) at room temperature for one hour. The membranes were washed three times with TBST and developed with chemiluminescence reagent (Thermo Scientific). The luminescence was detected by a Molecular Image Chemi Doc (Bio-Rad Laboratories).

### Data Analysis

Current traces recorded at negative voltage were presented in all figures as upward deflections for the sake of clear presentation. Once digitized, current traces were analyzed with Igor Pro software (Wavemetrics, USA). Specifically, mean current amplitudes were measured before and after addition of CFTR potentiators. Fold increase was defined as the ratio between the mean current amplitude in the presence of the drug and that in its absence. The surface expression of delF508-CFTR was assessed as Band C in Western blot assays and corrected to the house keeping protein (vimentin) (as shown in FIG. 14). The effects (fold increases) of different compounds were normalized to the surface expression level in the presence of DMSO (dimethyl sulfoxide). All results are presented as means±SEM; N is the number of experiments.

### Results

To test the effects of selected LZH compounds (e.g., LZH-00014, LZH-00015 and LZH-00025) as CFTR potentiators, when used as single agents, CFTR (G551 D or delF508) currents were first activated in excised inside-out patches with protein kinase A (PKA) and ATP. Once the current reached a steady level, the perfusate was changed to one with ATP only to ensure a stable activity of phosphorylated CFTR is attained. LZH compounds were acutely added in the presence of ATP until a new steady-state was observed.

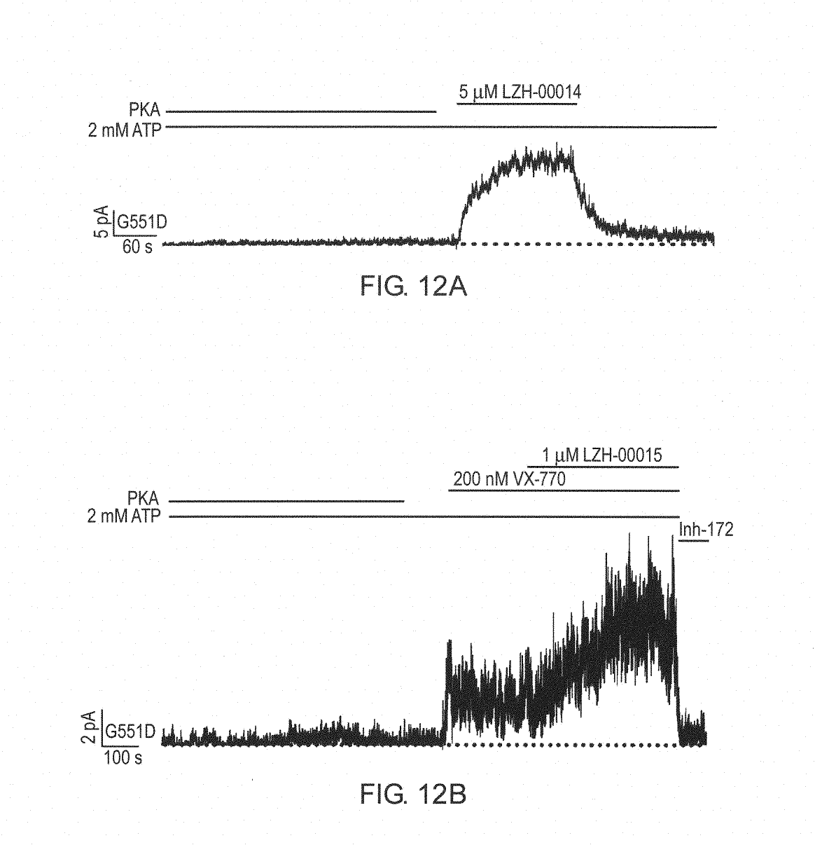

FIG. 12A shows a representative recording of such experiments for LZH-00014 on G551D-CFTR. After the patch was fully phosphorylated by PKA and ATP, the addition of 5 μM LZH-00014 dramatically increased the current of G551D-CFTR with a fold increase of 16.0±5.1, N=5. The removal of LZH-00014 shows a slow current decay back to the pre-stimulated level.

To test whether LZH compounds in combination with Ivacaftor (VX-770), an FDA-approved CFTR potentiator for the treatment of patients with cystic fibrosis, can synergistically increase the current of G551D-CFTR, the protocol described above was slightly modified. As shown in FIG. 12B, VX-770 was first added after G551D-CFTR channels were activated by PKA and ATP. The effect of LZH compounds was then tested by adding LZH compounds in the continuous presence of VX-770. FIG. 12B shows that 200 nM VX-770 increased the mean current G551D-CFTR by around 10 folds, while the addition of 1 μM LZH-00015 in the presence of VX-770 further increased the current. These data suggest that LZH compounds (e.g., LZH-00014, LZH-00015 and LZH-00025) can serve as CFTR potentiators as single agents (FIG. 12A) as well as work synergistically in combination with VX-770 (FIG. 12B). As shown in FIG. 12B, the potentiated G551D-CFTR currents can be abolished by a specific CFTR inhibitor (Inh-172), demonstrating the selectivity of the LZH compound for the CFTR target.

The following table (Table 1) summarizes data obtained from three different LZH compounds (at indicated concentrations) on G551D-CFTR:

TABLE 1
Summary of the fold increases of G551D-CFTR currents by
selected LZH compounds

| A. Single | | | |
|---|---|---|---|
| application | 1 μM | 5 μM | 20 μM |
| LZH-00014 | 4.5 ± 0.7, N = 4 | 16.0 ± 5.1, N = 5 | 12.7 ± 4.3, N = 3 |
| LZH-00015 | 1.0 ± 0.7, N = 3 | 4.5 ± 0.5, N = 3 | 3.3 ± 0.8, N = 3 |
| LZH-00025 | 2.1 ± 0.2, N = 11 | 4.9 ± 0.7, N = 14 | 7.4 ± 0.5, N = 8 |

| B. Combined | | | |
|---|---|---|---|
| application | 0.2 μM | 1 μM | 5 μM |
| LZH-00014 | 6.5 ± 1.0, N = 4 | 7.6 ± 1.6, N = 4 | 66.6 ± 10.3, N = 4 |
| LZH-00015 | 5.8 ± 0.9, N = 5 | 16.7 ± 10.6, | 42.5 ± 10.0, N = 5 |
| | | N = 4 | |
| LZH-00025 | 8.0 ± 1.0, N = 3 | 11.0 ± 2.9, N = 4 | 9.7 ± 0.7, N = 4 |

The top four rows show a summary of the fold increases of G551D-CFTR currents by LZH-00014, LZH-00015, LZH-00025. The magnitude of potentiation for G551 D-CFTR was calculated as fold increase (ratio of currents with and without LZH compounds, I_{LZH+ATP}/I_{ATP}). It is to be noted that, by increasing the Pₒ of G551D-CFTR by 8-10 folds, Ivacaftor improves the function of G551D-CFTR to <10% of wild-type channels (Jih et al., PNAS 2013), which is a value not sufficient to reach the 25% threshold needed to completely restore normal physiological function of CFTR (Davis, Ped. Rev., 2001). The bottom four rows show a summary of the synergistic effects between the LZH compounds and VX-770. The magnitude of potentiation for G551 D-CFTR was calculated as fold increase (ratio of currents with and without LZH compounds, I_{LZH+ATP+VX-770}/I_{ATP}). It is to be noted that a ˜66-fold enhancement of G551D-CFTR can be attained by a combination of LZH-00014 and VX-770. The observed dramatic enhancement suggests that it may be possible to completely rectify the dysfunction of G551D-CFTR, i.e., to a level that is expected to functionally cure patients carrying this mutation.

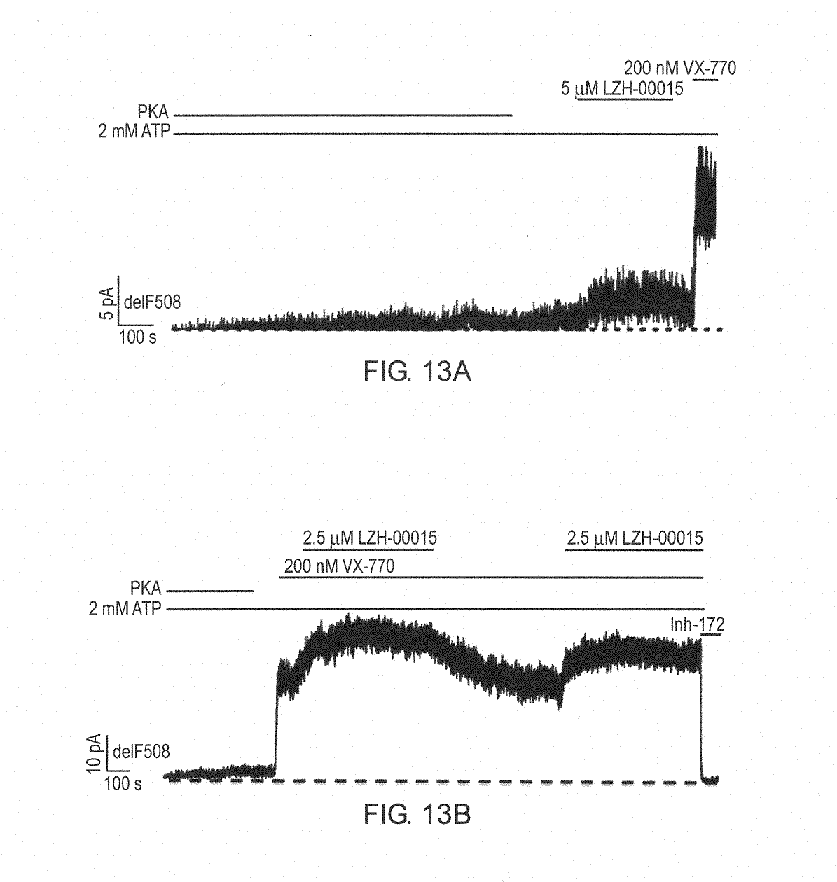

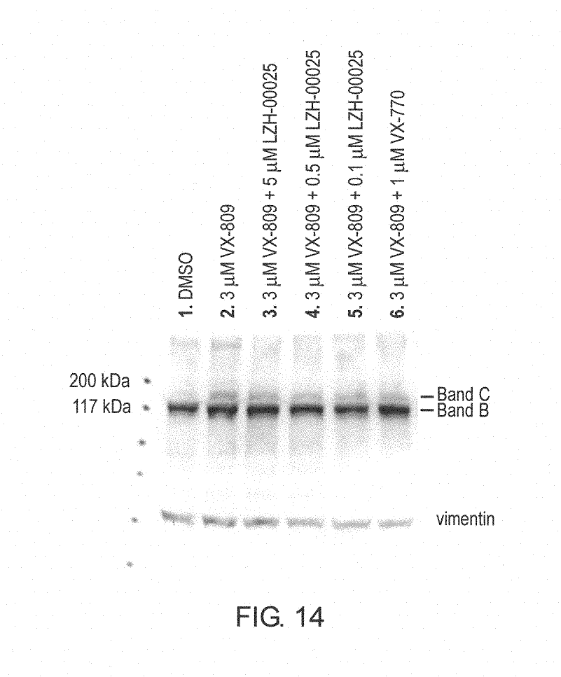

Similar experiments were carried out to examine the effects of selected LZH compounds as CFTR potentiators on delF508-CFTR. A representative recording of delF508-CFTR in FIG. 13A shows that after the patch is fully phosphorylated by PKA and ATP, the addition of 5 μM LZH-00015 resulted in a 3-fold current increase (3.4±0.4, N=7). FIG. 13B shows synergistic effects of LZH-00015 and Ivacaftor (200 nM VX-770) on delF508-CFTR. According to FIG. 13B, (1) 200 nM VX-770 increased the mean current of delF508-CFTR by ˜10 folds; and (2) the addition of 2.5 μM LZH-00015 in the presence of VX770 resulted in an overall 15-fold current increase of that in the presence of 2 mM ATP. These data suggest that LZH compounds (e.g., LZH-00014, LZH-00015 and LZH-00025) can act as CFTR potentiators for delF508-CFTR (FIG. 13A) and exert synergistic potentiating effect on delF508-CFTR together with VX-770. As shown in FIG. 13B, the potentiated delF508-CFTR currents can be abolished by specific CFTR inhibitor (Inh-172), demonstrating the selectivity of the LZH compounds for the CFTR target. It is to be noted that, since the Pₒ of delF508-CFTR in the presence of Ivacaftor is already above 0.4 (Kopeikin et al., J. Cys. Fibro. 2014), additional increase of the Pₒ by the LZH compounds might be limited. Nevertheless, this further enhancement of the Pₒ in the presence of the maximally effective concentration of Ivacaftor (200 nM) does indicate a pharmacological synergism as seen with G551D-CFTR (FIG. 12B).

The following table (Table 2) summarizes data obtained from three different LZH compounds (at indicated concentrations) on delF508-CFTR:

TABLE 2
Summary of the fold increases of delF508-CFTR currents by
selected LZH compounds

| A. Single | |
|---|---|
| application | 5 μM |
| LZH-00014 | 7.4 ± 1.6, N = 5 |
| LZH-00015 | 3.4 ± 0.4, N = 7 |
| LZH-00025 | 3.6 ± 0.9, N = 4 |

| B. Combined | | |
|---|---|---|
| application | 0.5 μM | 2.5 μM |
| LZH-00014 | 6.0 ± 1.6, N = 3 | 7.9 ± 0.7, N = 3 |
| LZH-00015 | 5.9 ± 1.3, N = 3 | 15.9 ± 1.0, N = 3 |
| LZH-00025 | 7.3 ± 0.8, N = 3 | 7.7 ± 2.0, N = 3 |

The top four rows show a summary of the fold increases of delF508-CFTR currents by LZH-00014, LZH-00015, LZH-00025. The magnitude of potentiation for delF508-CFTR was calculated as fold increase (ratio of currents with and without LZH compounds, I_{LZH+ATP}/I_{ATP}). The bottom four rows show a summary of the synergistic effects between the LZH compounds and VX-770. The magnitude of potentiation for delF508-CFTR was calculated as fold increase (ratio of currents with and without LZH compounds, I_{LZH+ATP+VX-770}/I_{ATP}).

As discussed in Example 48 above, it is known that delF508-CFTR exhibits multiple biochemical defects. For example, in addition to trafficking and gating defects, the half-life of delF508-CFTR proteins in the cell membrane is dramatically shortened (Lukacs et al., J. Biol. Chem. 1993). Both trafficking and stability defects effectively reduce the number of mature delF508-CFTR proteins in the cell membrane. The effects of the LZH compounds and Lumacaftor (VX-809) on membrane expression of delF508-CFTR were further characterized. For this, effects of the LZH-00025 and Lumacaftor (VX-809) on membrane expression of delF508-CFTR are shown in FIG. 14.

To evaluate the effect of LZH compounds on delF508-CFTR surface expression, the final surface expression of delF508-CFTR with DMSO added to the cell-culture C127 plate was used as the control, which shows low expression in Band C (FIG. 14, lane 1). Consistent with the reported effect of Lumacaftor (VX-809) that improves delF508-CFTR trafficking (Van Goor et al., PNAS 2011), 3 μM VX-809 indeed increased Band C (FIG. 14, lane 2). Meanwhile, additional application of Ivacaftor (VX-770) dampens this effect presumably by destabilizing delF508-CFTR in the plasma membrane (Veit et al., Sci. Transl. Med. 2015) (FIG. 14, lane 6). Contrary to Ivacaftor (VX-770), which diminishes the effect of Lumacaftor, LZH-00025 actually retains or even enhances the effect of Lumacaftor (FIG. 14, lanes 3-5) when image signal is normalized to the housekeeping signal given by vimentin and the control signal (FIG. 14, lane 1).

A summary of the improved surface expression of delF508-CFTR by selected LZH compounds in Western blot analysis is further provided in Table 3:

TABLE 3
Summary of the improved surface expression of delF508-CFTR by
LZH compounds in Western blot

| | | | | LZH-00025 |
|---|---|---|---|---|
| Lane | Treatment | LZH-00014 | LZH-00015 | (FIG. 14) |
| 1 | DMSO | 100 | 100 | 100 |
| 2 | 3 μM VX-809 | 577 ± 136, N = 5 | 302 ± 71, N = 4 | 300 ± 32, N = 4 |
| 3 | 3 μM VX-809 + | 609 ± 185, N = 5 | 356 ± 98, N = 4 | 300 ± 42, N = 4 |
| | 5 μM LZH compound | | | |
| 4 | 3 μM VX-809 + | 617 ± 63, N = 5 | 347 ± 106, N = 4 | 323 ± 14, N = 4 |
| | 0.5 μM LZH compound | | | |
| 5 | 3 μM VX-809 + | 670 ± 211, N = 5 | 395 ± 99, N = 4 | 263 ± 27, N = 4 |
| | 0.1 μM LZH compound | | | |
| 6 | 3 μM VX-809 + | 334 ± 81, N = 5 | 182 ± 57, N = 4 | 172 ± 50, N = 4 |
| | 1 μM VX-770 | | | |

Similar to what is described above, using the surface expression of delF508-CFTR in the presence of DMSO as control with 100 surface expression, 3 μM VX-809 improves the delF508-CFTR trafficking and surface expression. The additional application of LZH compounds at different concentrations show similar or better surface expression compared with that in the presence of 3 μM VX-809 (Table 3, lane 2). On the contrary, the addition of 114M VX-770 to 3 μM VX-809 indeed dampens the effect of VX-809 consistent with the previous studies (Table 3, lane 6).

The above data show that, contrary to Ivacaftor that diminishes the effect of Lumacaftor (Table 3, lane 6), LZH-00014, LZH-00015, or LZH-00025 actually retains or even enhances the effect of Lumacaftor (Table 3, lanes 3-5), stabilizing the presence of mature membrane form of CFTR. See also FIG. 14, lanes 3-5.

In summary, the group of compounds described herein function as CFTR potentiators by themselves, as the data showed that they effectively potentiate the activity of G551 D or delF508-CFTR. The maximal effect of some these compounds exceeds that of Ivacaftor. They also show clear pharmacological synergism when applied together with Ivacaftor, suggesting that the described compounds and Ivacaftor work through distinct mechanisms. Additionally, the data presented also reveal an unexpected dual function of these compounds. It was found that the delF508-CFTR channels, treated with exemplary compounds, are more stable in the cell membrane than those treated with Ivacaftor. Biochemical experiments showed a clear synergism with Lumacaftor. The data presented here suggest that the compounds described herein, having dual function of CFTR potentiator/stabilizer, may be used as therapeutic agents for a majority of CF patients, alone or in combination with other therapeutic agent(s).

The disclosures of each and every patent, patent application, and publication cited herein are hereby incorporated herein by reference in their entirety.

While specific examples have been discussed, the above Specification is illustrative and not restrictive. Many variations of the described compounds and methods will become apparent to those skilled in the art upon review of this Specification. The full scope of the compounds and methods should be determined by reference to the claims, along with their full scope of equivalents, and the Specification, along with such variations.

We claim:

1. A compound of Formula (1), a pharmaceutically acceptable salt, a pharmaceutically acceptable hydrate, a pharmaceutically acceptable solvate, a pharmaceutically acceptable clathrate, or a pharmaceutically acceptable polymorph thereof, 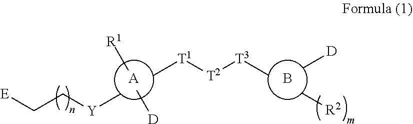
wherein
Ring A is selected from phenyl, six-membered aromatic ring with 1, 2, or 3 nitrogen ring atoms, or a five-membered ring, aromatic or non-aromatic, with 1, 2, or 3 heteroatoms independently selected from O, S, or N;
Ring B is a bicyclic ring system, aromatic or non-aromatic, with 7, 8, 9, or 10 ring atoms with 1 to 4 heteroatoms selected from O, S, or N;
each T¹, T², and T³ are independently absent, or independently selected from C(R^{CT})₂, C(O), S(O)₀₋₂ or NR^{NT}, wherein R^{CT} with R^{CT}, or R^{CT} with R^{NT}, may join together to form a three-, four-, or five-membered aliphatic ring; or R^{CT} and R^{NT} are each independently selected from H, CH₂OH, C₁₋₄alkyl, C₂₋₆alkenyl, CF₃, or (R^{CT})₂ is ═CHR³, where R³ is independently selected from H, CH₂OH, C₁₋₄alkyl, or C₂₋₆alkenyl;
Y is selected from a bond, O, S, NR³, or —C(O)NR³;
D are each independently selected from F, CF₃, CHₐF₍₃₋ₐ₎, Cl, Br, CN, NO₂, OR⁴, OCF₃, or OC(O)R⁵, where a is 1 or 2; R⁴ is H, C₁₋₄alkyl, or R⁵; and R⁵ is (CH₂)ⱼR⁶ where j is an integer from 3 to 8, and R⁶ is H or E, and optionally one or more adjacent CH₂ units is replaced with O, S, or NR³, and/or one or more hydrogens of the CH₂ units is substituted with F, Cl, Br, CN, OR⁴, OH, or NHR³;
E is N(R³)₂;
R¹ and R² are independently selected from C₁₋₆ alkyl, five- to nine-membered heteroaryl, phenyl, napthyl, —OR⁴, —N(R³)₂, —SR⁴, —SO₂R⁴, —SO₂N(R³)₂, —NR³SO₂R⁴, —NR³C(O)OR⁴, —NR³C(O)R⁴, —C(O)OR⁴, —C(O)N(R³)², —OC(═O)R⁴, —C(═O)R⁴, and m is an integer from 1 to 4; and
n is an integer from 1-7, and optionally one or more CH₂ units is replaced with O, S, or NR³, and/or one or more hydrogens of the CH₂ units is substituted with F, Cl, Br, CN, OR⁴, OH, or NHR³.

2. The compound of claim 1, wherein each of T¹, T², and T³ are absent and Ring A is directly connected to Ring B, and wherein Ring A is an aromatic six-membered ring structure and Ring B is a bicyclic ring system having 9 ring atoms with 1 to 2 N ring atoms.

3. The compound of claim 2, wherein said compound is selected from the group consisting of:

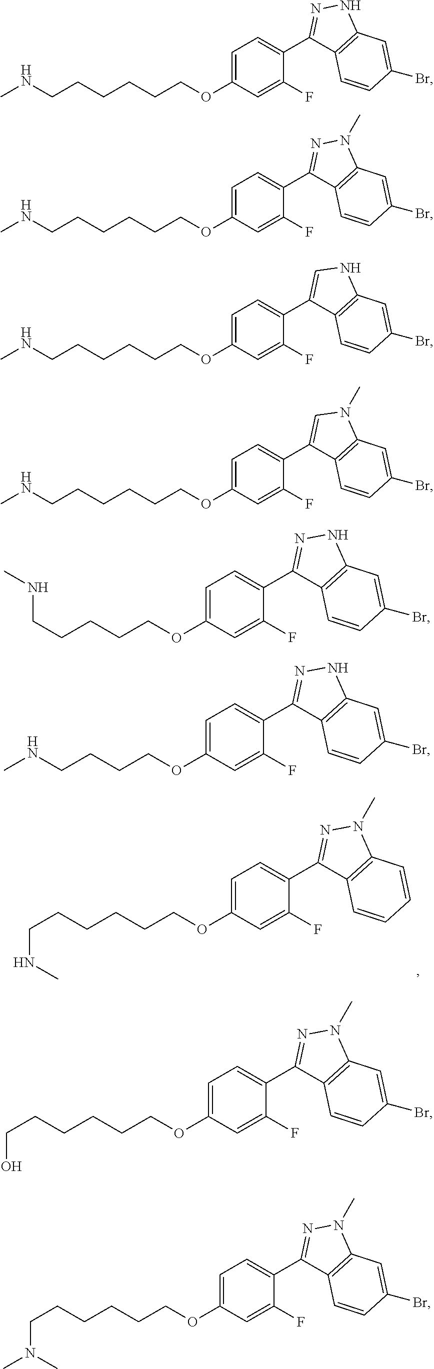

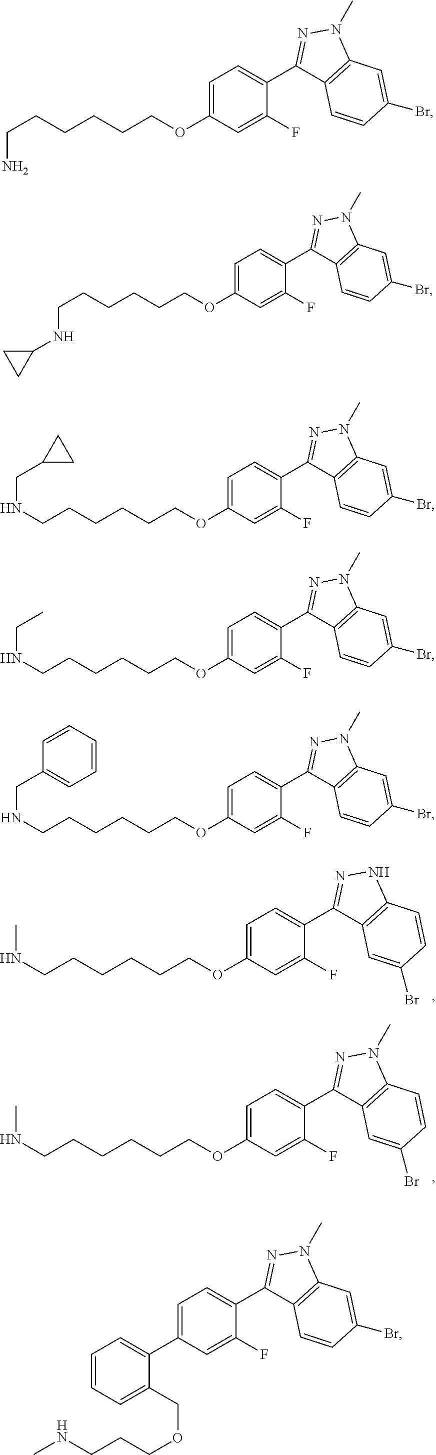

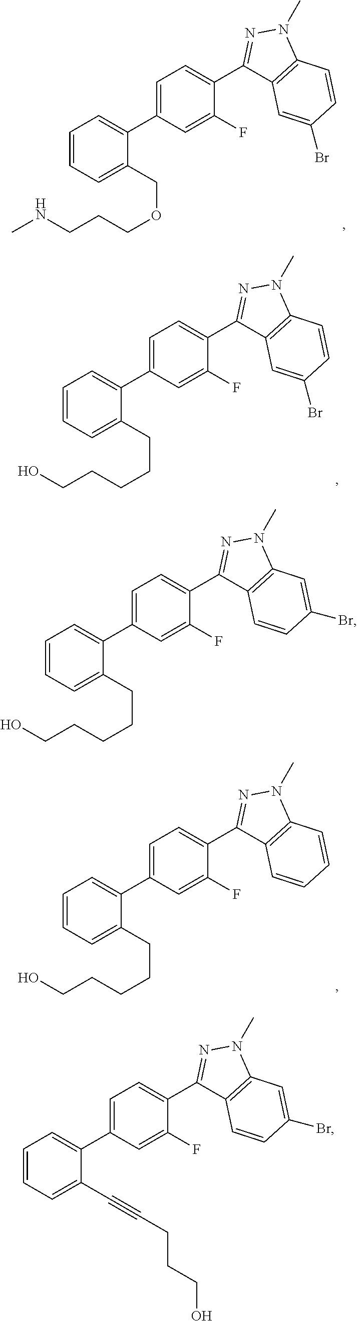 [SMILES: CNCCCOCc1ccccc1-c1ccc(-c2nn(C)c3ccc(Br)cc23)c(F)c1 ; Cn1nc(-c2ccc(-c3ccccc3CCCCCO)cc2F)c2cc(Br)ccc21 ; Cn1nc(-c2ccc(-c3ccccc3CCCCCO)cc2F)c2ccc(Br)cc21 ; Cn1nc(-c2ccc(-c3ccccc3CCCCCO)cc2F)c2ccccc21 ; Cn1nc(-c2ccc(-c3ccccc3C#CCCCO)cc2F)c2ccc(Br)cc21]

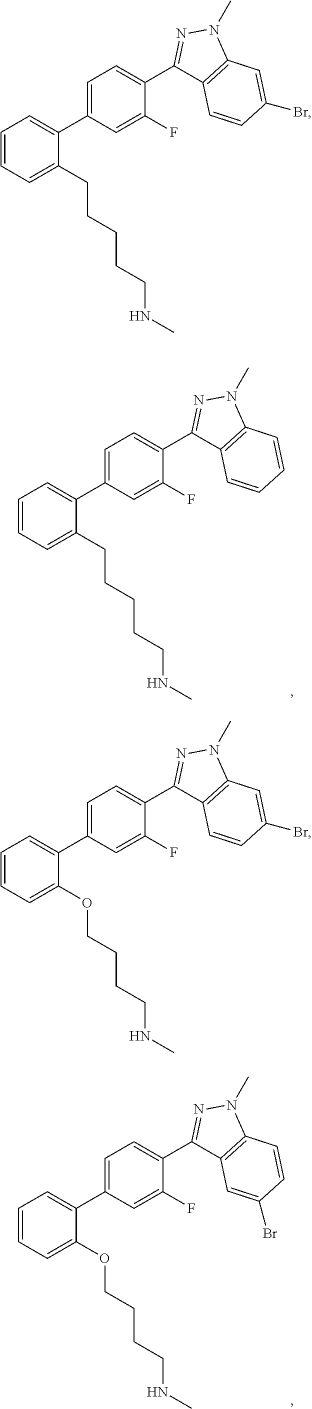

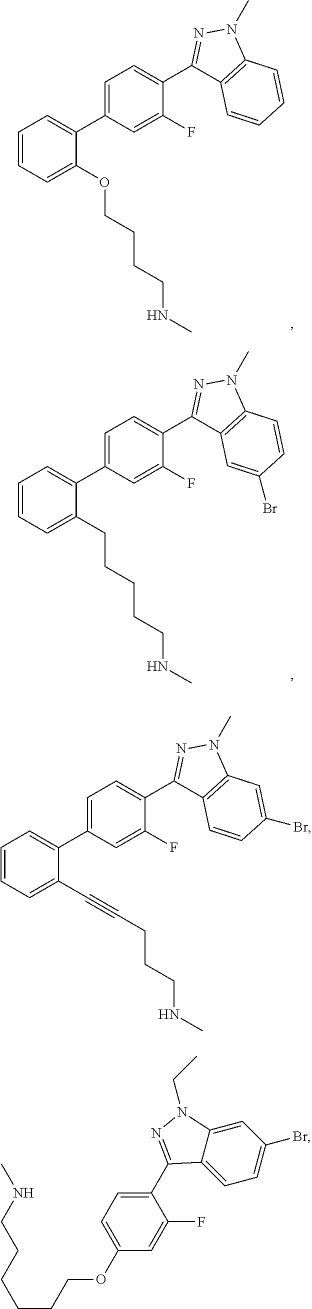

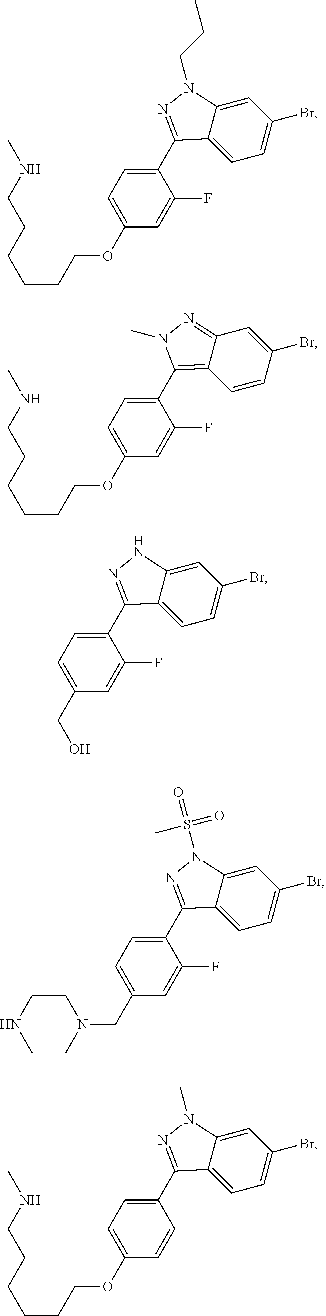 [SMILES: CCCn1nc(-c2ccc(OCCCCCCNC)cc2F)c2ccc(Br)cc21 ; CNCCCCCCOc1ccc(-c2c3ccc(Br)cc3nn2C)c(F)c1 ; OCc1ccc(-c2n[nH]c3cc(Br)ccc23)c(F)c1 ; CNCCN(C)Cc1ccc(-c2nn(S(C)(=O)=O)c3cc(Br)ccc23)c(F)c1 ; CNCCCCCCOc1ccc(-c2nn(C)c3cc(Br)ccc23)cc1]

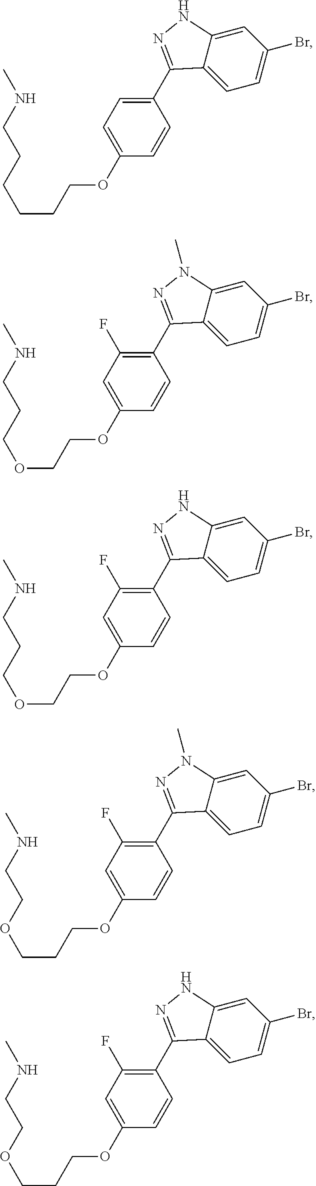 [SMILES: CNCCCCCCOc1ccc(-c2n[nH]c3cc(Br)ccc23)cc1 ; CNCCCOCCOc1ccc(-c2nn(C)c3cc(Br)ccc23)c(F)c1 ; CNCCCOCCOc1ccc(-c2n[nH]c3cc(Br)ccc23)c(F)c1 ; CNCCOCCCOc1ccc(-c2nn(C)c3cc(Br)ccc23)c(F)c1 ; CNCCOCCCOc1ccc(-c2n[nH]c3cc(Br)ccc23)c(F)c1]

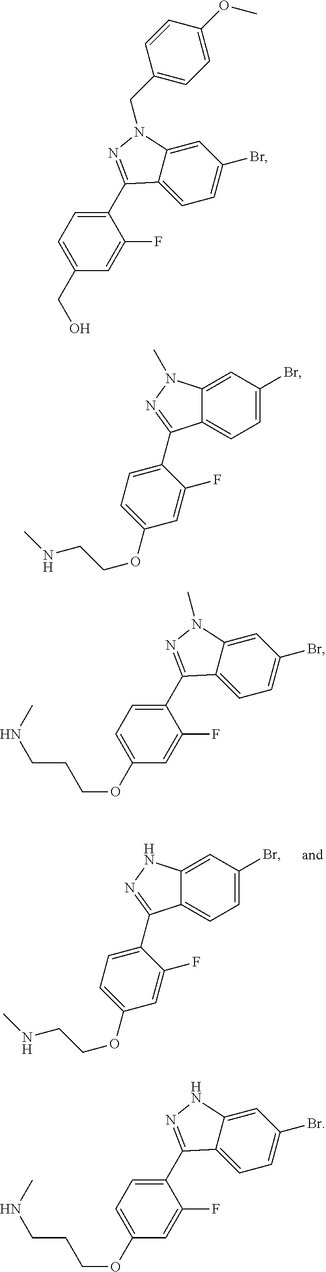

4. A composition for enhancing cystic fibrosis transmembrane conductance regulator (CFTR) activity comprising an effective amount of the compound of claim 1, the pharmaceutically acceptable salt, the pharmaceutically acceptable hydrate, the pharmaceutically acceptable solvate, the pharmaceutically acceptable clathrate, or the pharmaceutically acceptable polymorph thereof.

5. A method of treating a CFTR-mediated disease by enhancing cystic fibrosis transmembrane conductance regulator (CFTR) activity or expression in the cells of a subject in need thereof comprising administering to the subject a composition comprising an effective amount of a compound of Formula (1), a pharmaceutically acceptable salt, a pharmaceutically acceptable hydrate, a pharmaceutically acceptable solvate, a pharmaceutically acceptable clathrate, or a pharmaceutically acceptable polymorph thereof, 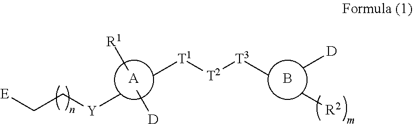
wherein
Ring A is selected from phenyl, six-membered aromatic ring with 1,2, or 3 nitrogen ring atoms, or a five-membered ring, aromatic or non-aromatic, with 1, 2, or 3 heteroatoms independently selected from O, S, or N;
Ring B is a mono or bicyclic ring system, aromatic or non-aromatic, with 5, 6, 7, 8, 9, or 10 ring atoms with 1 to 4 heteroatoms selected from O, S, or N;
each T¹, T², and T³ are independently absent, or independently selected from C(R^{CT})₂, C(O), S(O)₀₋₂ or NR^{NT}, wherein R^{CT} with R^{CT}, or R^{CT} with R^{NT}, may join together to form a three-, four-, or five-membered aliphatic ring; or R^{CT} and R^{NT} are each independently selected from H, CH₂OH, C₁₋₄alkyl, C₂₋₆alkenyl, CF₃, or (R^{CT})₂ is ═CHR³, where R³ is independently selected from H, CH₂OH, C₁₋₄alkyl, or C₂₋₆alkenyl;
Y is selected from a bond, O, S, NR³, or —C(O)NR³;
D are each independently selected from F, CF₃, CHₐF₍₃₋ₐ₎, Cl, Br, CN, NO₂, OR⁴, OCF₃, or OC(O)R⁵, where a is 1 or 2; R⁴ is H, C₁₋₄alkyl, or R⁵; and R⁵ is (CH₂)ⱼR⁶ where j is an integer from 3 to 8, and R⁶ is H or E, and optionally one or more adjacent CH₂ units is replaced with O, S, or NR³, and/or one or more hydrogens of the CH₂ units is substituted with F, Cl, Br, CN, OR⁴, OH, or NHR³;
E is N(R³)₂;
R¹ and R² are independently selected from C₁₋₆ alkyl, five- to nine-membered heteroaryl, phenyl, napthyl, —OR⁴, —N(R³)₂, —SR⁴, —SO₂R⁴, —SO₂N(R³)₂, —NR³SO₂R⁴, —NR³C(O)OR⁴, —NR³C(O)R⁴, —C(O)OR⁴, —C(O)N(R³)₂, —OC(═O)R⁴, —C(═O)R⁴, and m is an integer from 1 to 4; and
n is an integer from 1-7, and optionally one or more CH₂ units is replaced with O, S, or NR³, and/or one or more hydrogens of the CH₂ units is substituted with F, Cl, Br, CN, OR⁴, OH, or NHR³.

6. The method of claim 5, wherein the compound has each of T¹, T², and T³ being absent and Ring A being directly connected to Ring B, and wherein Ring A is an aromatic six-membered ring structure and Ring B is a bicyclic ring system having 9 ring atoms with 1 to 2 N ring atoms.

7. The method of claim 5, wherein the subject comprises a mutant CFTR, and wherein the activity of the mutant CFTR is enhanced as a result of administering the composition.

8. The method of claim 7, wherein the mutant CFTR comprises at least one mutation selected from the group consisting of a Class I mutation, a Class II mutation, a Class III mutation, a Class IV mutation, a Class V mutation, a Class VI mutation, and combinations thereof.

9. The method of claim 8, wherein the mutant CFTR comprises at least one Class II mutation or one Class III mutation.

10. The method of claim 9, wherein the mutant CFTR is a delF508 CFTR, and wherein the delF508 CFTR activity is enhanced as a result of administering the composition.

11. The method of claim 9, wherein the mutant CFTR is a G551D-CFTR, and wherein the G551D-CFTR activity is enhanced as a result of administering the composition.

12. The method of claim 5, wherein administering the composition is through a route selected from oral, intravenous, intraperitoneal, intramuscular, transdermal, subcutaneous, topical, sublingual, intravascular, intramammary, rectal means, and combinations thereof.

13. The method of claim 5, wherein the compound, the pharmaceutically acceptable salt, the pharmaceutically acceptable hydrate, the pharmaceutically acceptable solvate, the pharmaceutically acceptable clathrate, or the pharmaceutically acceptable polymorph thereof, is administered as the sole active agent.

14. The method of claim 5, further comprising administering to the subject one or more therapeutic agents.

15. The method of claim 14, wherein the one or more therapeutic agents are capable of modulating CFTR activity or expression.

16. The method of claim 14, wherein the one or more therapeutic agents are selected from the group consisting of:
a) an effective amount of Ivacaftor;
b) an effective amount of Lumacaftor; and
c) an effective amount of a combination of Ivacaftor and Lumacaftor.

17. The method of claim 14, wherein the composition and the one or more therapeutic agents are administered at substantially the same time.

18. The method of claim 5, wherein the subject is suffering from a disease associated with decreased CFTR activity.

19. The method of claim 18, wherein the disease is cystic fibrosis.

20. The method of claim 5, wherein the subject is a human patient.
